SEQUENCE LISTING (2059 sequences)

SEQ ID NO: 1 - DNA, 10 bp, Artificial Sequence
  primer
agacgtcgtt

SEQ ID NO: 2 - DNA, 10 bp, Artificial Sequence
  primer
gaggcgatat

SEQ ID NO: 3 - DNA, 10 bp, Artificial Sequence
  primer
gtgcgaacgt

SEQ ID NO: 4 - DNA, 10 bp, Artificial Sequence
  primer
ttatactgcc

SEQ ID NO: 5 - DNA, 10 bp, Artificial Sequence Sequence
  primer
caagttcgca

SEQ ID NO: 6 - DNA, 10 bp, Artificial Sequence
  primer
acaaggtagt

SEQ ID NO: 7 - DNA, 10 bp, Artificial Sequence
  primer
acacagcgac

SEQ ID NO: 8 - DNA, 10 bp, Artificial Sequence
  primer
ttaccgatgt

SEQ ID NO: 9 - DNA, 10 bp, Artificial Sequence
  primer
cacagagtcg

SEQ ID NO: 10 - DNA, 10 bp, Artificial Sequence
  primer
ttcagcgcgt

SEQ ID NO: 11 - DNA, 10 bp, Artificial Sequence
  primer
aggaccgtga

SEQ ID NO: 12 - DNA, 10 bp, Artificial Sequence
  primer
gtctgttcgc

SEQ ID NO: 13 - DNA, 10 bp, Artificial Sequence
  primer
acctgtccac

SEQ ID NO: 14 - DNA, 10 bp, Artificial Sequence
  primer
ccgcaatgac

SEQ ID NO: 15 - DNA, 10 bp, Artificial Sequence
  primer
ctgccgatca

SEQ ID NO: 16 - DNA, 10 bp, Artificial Sequence
  primer
tacacggagc

SEQ ID NO: 17 - DNA, 10 bp, Artificial Sequence
  primer
ccgcattcat

SEQ ID NO: 18 - DNA, 10 bp, Artificial Sequence
  primer
gactctagac

SEQ ID NO: 19 - DNA, 10 bp, Artificial Sequence
  primer
ggagaactta

SEQ ID NO: 20 - DNA, 10 bp, Artificial Sequence
  primer
tccggtatgc

SEQ ID NO: 21 - DNA, 10 bp, Artificial Sequence
  primer
ggtcaggagt

SEQ ID NO: 22 - DNA, 10 bp, Artificial Sequence
  primer
acattggcag

SEQ ID NO: 23 - DNA, 10 bp, Artificial Sequence
  primer
cgtagactgc

SEQ ID NO: 24 - DNA, 10 bp, Artificial Sequence
  primer
agactgtact

SEQ ID NO: 25 - DNA, 10 bp, Artificial Sequence
  primer
tagacgcagt

SEQ ID NO: 26 - DNA, 10 bp, Artificial Sequence
  primer
ccgataatct

SEQ ID NO: 27 - DNA, 10 bp, Artificial Sequence
  primer
gagagctagt

SEQ ID NO: 28 - DNA, 10 bp, Artificial Sequence
  primer
gtaccgcgtt

SEQ ID NO: 29 - DNA, 10 bp, Artificial Sequence
  primer
gacttgcgca

SEQ ID NO: 30 - DNA, 10 bp, Artificial Sequence
  primer
cgtgattgcg

SEQ ID NO: 31 - DNA, 10 bp, Artificial Sequence
  primer
atcgtctctg

SEQ ID NO: 32 - DNA, 10 bp, Artificial Sequence
  primer
cgtagctacg

SEQ ID NO: 33 - DNA, 10 bp, Artificial Sequence
  primer
gccgaatagt

SEQ ID NO: 34 - DNA, 10 bp, Artificial Sequence
  primer
gtacctaggc

SEQ ID NO: 35 - DNA, 10 bp, Artificial Sequence
  primer
gcttacatga

SEQ ID NO: 36 - DNA, 10 bp, Artificial Sequence
  primer
tccacgtagt

SEQ ID NO: 37 - DNA, 10 bp, Artificial Sequence
  primer
agaggccatc

SEQ ID NO: 38 - DNA, 10 bp, Artificial Sequence
  primer
cggtgatgct

SEQ ID NO: 39 - DNA, 10 bp, Artificial Sequence
  primer
cactgtgctt

SEQ ID NO: 40 - DNA, 10 bp, Artificial Sequence
  primer
catgatggct

SEQ ID NO: 41 - DNA, 10 bp, Artificial Sequence
  primer
gccacacatg

SEQ ID NO: 42 - DNA, 10 bp, Artificial Sequence
  primer
cacacactgt

SEQ ID NO: 43 - DNA, 10 bp, Artificial Sequence
  primer
cagaatcata

SEQ ID NO: 44 - DNA, 10 bp, Artificial Sequence
  primer
atcgtctacg

SEQ ID NO: 45 - DNA, 10 bp, Artificial Sequence
  primer
cgagcaatac

SEQ ID NO: 46 - DNA, 10 bp, Artificial Sequence
  primer
acaagcgcac

SEQ ID NO: 47 - DNA, 10 bp, Artificial Sequence
  primer
gcttagatgt

SEQ ID NO: 48 - DNA, 10 bp, Artificial Sequence
  primer
tgcattctgg

SEQ ID NO: 49 - DNA, 10 bp, Artificial Sequence
  primer
tgtcggacca

SEQ ID NO: 50 - DNA, 10 bp, Artificial Sequence
  primer
aggcactcgt

SEQ ID NO: 51 - DNA, 10 bp, Artificial Sequence
  primer
ctgcatgtga

SEQ ID NO: 52 - DNA, 10 bp, Artificial Sequence
  primer
accacgccta

SEQ ID NO: 53 - DNA, 10 bp, Artificial Sequence
  primer
gaggtcgtac

SEQ ID NO: 54 - DNA, 10 bp, Artificial Sequence
  primer
aatactctgt

SEQ ID NO: 55 - DNA, 10 bp, Artificial Sequence
  primer
tgccaactga

SEQ ID NO: 56 - DNA, 10 bp, Artificial Sequence
  primer
cctgttcggt

SEQ ID NO: 57 - DNA, 10 bp, Artificial Sequence
  primer
gtagagagtt

SEQ ID NO: 58 - DNA, 10 bp, Artificial Sequence
  primer
tacagcgtaa

SEQ ID NO: 59 - DNA, 10 bp, Artificial Sequence
  primer
tgacgtgatg

SEQ ID NO: 60 - DNA, 10 bp, Artificial Sequence
  primer
agacgtcggt

SEQ ID NO: 61 - DNA, 10 bp, Artificial Sequence
  primer
cgctaggttc

SEQ ID NO: 62 - DNA, 10 bp, Artificial Sequence
  primer
gccttatagc

SEQ ID NO: 63 - DNA, 10 bp, Artificial Sequence
  primer
ccttcgatct

SEQ ID NO: 64 - DNA, 10 bp, Artificial Sequence
  primer
aggcaacgtg

SEQ ID NO: 65 - DNA, 10 bp, Artificial Sequence
  primer
tgagcggtgt

SEQ ID NO: 66 - DNA, 10 bp, Artificial Sequence
  primer
gtgtcgaacg

SEQ ID NO: 67 - DNA, 10 bp, Artificial Sequence
  primer
cgatgttgcg

SEQ ID NO: 68 - DNA, 10 bp, Artificial Sequence
  primer
aacaagacac

SEQ ID NO: 69 - DNA, 10 bp, Artificial Sequence
  primer
gatgctggtt

SEQ ID NO: 70 - DNA, 10 bp, Artificial Sequence
  primer
accggtagtc

SEQ ID NO: 71 - DNA, 10 bp, Artificial Sequence
  primer
gtgactagca

SEQ ID NO: 72 - DNA, 10 bp, Artificial Sequence
  primer
agcctatatt

SEQ ID NO: 73 - DNA, 10 bp, Artificial Sequence
  primer
tcgtgagctt

SEQ ID NO: 74 - DNA, 10 bp, Artificial Sequence
  primer
acactatggc

SEQ ID NO: 75 - DNA, 10 bp, Artificial Sequence
  primer
gactctgtcg

SEQ ID NO: 76 - DNA, 10 bp, Artificial Sequence
  primer
tcgatgatgc

SEQ ID NO: 77 - DNA, 10 bp, Artificial Sequence
  primer
cttggacact

SEQ ID NO: 78 - DNA, 10 bp, Artificial Sequence
  primer
ggctgatcgt

SEQ ID NO: 79 - DNA, 10 bp, Artificial Sequence
  primer
actcacaggc

SEQ ID NO: 80 - DNA, 10 bp, Artificial Sequence
  primer
atgtgcgtac

SEQ ID NO: 81 - DNA, 10 bp, Artificial Sequence
  primer
caccatcgat

SEQ ID NO: 82 - DNA, 10 bp, Artificial Sequence
  primer
agccattaac

SEQ ID NO: 83 - DNA, 10 bp, Artificial Sequence
  primer
aatcgactgt

SEQ ID NO: 84 - DNA, 10 bp, Artificial Sequence
  primer
aatactagcg

SEQ ID NO: 85 - DNA, 10 bp, Artificial Sequence
  primer
tcgtcactga

SEQ ID NO: 86 - DNA, 10 bp, Artificial Sequence
  primer
caggctctta

SEQ ID NO: 87 - DNA, 10 bp, Artificial Sequence
  primer
ggtcggtgat

SEQ ID NO: 88 - DNA, 10 bp, Artificial Sequence
  primer
cattaggcgt

SEQ ID NO: 89 - DNA, 10 bp, Artificial Sequence
  primer
actcgcgagt

SEQ ID NO: 90 - DNA, 10 bp, Artificial Sequence
  primer
ttccgaataa

SEQ ID NO: 91 - DNA, 10 bp, Artificial Sequence
  primer
tgagcatcgt

SEQ ID NO: 92 - DNA, 10 bp, Artificial Sequence
  primer
gccacgtaac

SEQ ID NO: 93 - DNA, 10 bp, Artificial Sequence
  primer
gaactacatg

SEQ ID NO: 94 - DNA, 10 bp, Artificial Sequence
  primer
tcgtgaggac

SEQ ID NO: 95 - DNA, 10 bp, Artificial Sequence
  primer
gcggccttaa

SEQ ID NO: 96 - DNA, 10 bp, Artificial Sequence
  primer
gctaaggacc

SEQ ID NO: 97 - DNA, 10 bp, Artificial Sequence
  primer
atagccatta

SEQ ID NO: 98 - DNA, 10 bp, Artificial Sequence
  primer
cagtaatcat

SEQ ID NO: 99 - DNA, 10 bp, Artificial Sequence
  primer
actccttaat

SEQ ID NO: 100 - DNA, 10 bp, Artificial Sequence
  primer
tcgaacatta

SEQ ID NO: 101 - DNA, 10 bp, Artificial Sequence
  primer
attatgaggt

SEQ ID NO: 102 - DNA, 10 bp, Artificial Sequence
  primer
aatcttagag

SEQ ID NO: 103 - DNA, 10 bp, Artificial Sequence
  primer
ttagatgatg

SEQ ID NO: 104 - DNA, 10 bp, Artificial Sequence
  primer
tacatatctg

SEQ ID NO: 105 - DNA, 10 bp, Artificial Sequence
  primer
tccttaatca

SEQ ID NO: 106 - DNA, 10 bp, Artificial Sequence
  primer
gttgagatta

SEQ ID NO: 107 - DNA, 10 bp, Artificial Sequence
  primer
tgttaacgta

SEQ ID NO: 108 - DNA, 10 bp, Artificial Sequence
  primer
catacagtaa

SEQ ID NO: 109 - DNA, 10 bp, Artificial Sequence
  primer
cttatacgaa

SEQ ID NO: 110 - DNA, 10 bp, Artificial Sequence
  primer
agatctatgt

SEQ ID NO: 111 - DNA, 10 bp, Artificial Sequence
  primer
aagacttagt

SEQ ID NO: 112 - DNA, 10 bp, Artificial Sequence
  primer
tgcgcaataa

SEQ ID NO: 113 - DNA, 10 bp, Artificial Sequence
  primer
ttggccatat

SEQ ID NO: 114 - DNA, 10 bp, Artificial Sequence
  primer
tattacgagg

SEQ ID NO: 115 - DNA, 10 bp, Artificial Sequence
  primer
ttatgatcgc

SEQ ID NO: 116 - DNA, 10 bp, Artificial Sequence
  primer
aacttaggag

SEQ ID NO: 117 - DNA, 10 bp, Artificial Sequence
  primer
tcacaatcgt

SEQ ID NO: 118 - DNA, 10 bp, Artificial Sequence
  primer
gagtatatgg

SEQ ID NO: 119 - DNA, 10 bp, Artificial Sequence
  primer
atcaggacaa

SEQ ID NO: 120 - DNA, 10 bp, Artificial Sequence
  primer
gtactgatag

SEQ ID NO: 121 - DNA, 10 bp, Artificial Sequence
  primer
cttatactcg

SEQ ID NO: 122 - DNA, 10 bp, Artificial Sequence
  primer
taacggacta

SEQ ID NO: 123 - DNA, 10 bp, Artificial Sequence
  primer
gcgttgtata

SEQ ID NO: 124 - DNA, 10 bp, Artificial Sequence
  primer
cttaagtgct

SEQ ID NO: 125 - DNA, 10 bp, Artificial Sequence
  primer
atacgactgt

SEQ ID NO: 126 - DNA, 10 bp, Artificial Sequence
  primer
actgttatcg

SEQ ID NO: 127 - DNA, 10 bp, Artificial Sequence
  primer
aatcttgacg

SEQ ID NO: 128 - DNA, 10 bp, Artificial Sequence
  primer
acatcacctt

SEQ ID NO: 129 - DNA, 10 bp, Artificial Sequence
  primer
ggtatagtac

SEQ ID NO: 130 - DNA, 10 bp, Artificial Sequence
  primer
ctaatccaca

SEQ ID NO: 131 - DNA, 10 bp, Artificial Sequence
  primer
gcaccttatt

SEQ ID NO: 132 - DNA, 10 bp, Artificial Sequence
  primer
attgacggta

SEQ ID NO: 133 - DNA, 10 bp, Artificial Sequence
  primer
gacatatggt

SEQ ID NO: 134 - DNA, 10 bp, Artificial Sequence
  primer
gatagtcgta

SEQ ID NO: 135 - DNA, 10 bp, Artificial Sequence
  primer
caattatcgc

SEQ ID NO: 136 - DNA, 10 bp, Artificial Sequence
  primer
cttaggtgat

SEQ ID NO: 137 - DNA, 10 bp, Artificial Sequence
  primer
catactactg

SEQ ID NO: 138 - DNA, 10 bp, Artificial Sequence
  primer
taacgcgaat

SEQ ID NO: 139 - DNA, 10 bp, Artificial Sequence
  primer
caagttacga

SEQ ID NO: 140 - DNA, 10 bp, Artificial Sequence
  primer
aatctcaagg

SEQ ID NO: 141 - DNA, 10 bp, Artificial Sequence
  primer
gcaatcatca

SEQ ID NO: 142 - DNA, 10 bp, Artificial Sequence
  primer
tgtaacgttc

SEQ ID NO: 143 - DNA, 10 bp, Artificial Sequence
  primer
tatcgttggt

SEQ ID NO: 144 - DNA, 10 bp, Artificial Sequence
  primer
cgcttaagat

SEQ ID NO: 145 - DNA, 10 bp, Artificial Sequence
  primer
ttagaactgg

SEQ ID NO: 146 - DNA, 10 bp, Artificial Sequence
  primer
gtcataacgt

SEQ ID NO: 147 - DNA, 10 bp, Artificial Sequence
  primer
agagcagtat

SEQ ID NO: 148 - DNA, 10 bp, Artificial Sequence
  primer
caacatcact

SEQ ID NO: 149 - DNA, 10 bp, Artificial Sequence
  primer
cagaagctta

SEQ ID NO: 150 - DNA, 10 bp, Artificial Sequence
  primer
aactaacgtg

SEQ ID NO: 151 - DNA, 10 bp, Artificial Sequence
  primer
ttataccgct

SEQ ID NO: 152 - DNA, 10 bp, Artificial Sequence
  primer
gaattcgaga

SEQ ID NO: 153 - DNA, 10 bp, Artificial Sequence
  primer
ttacgtaacc

SEQ ID NO: 154 - DNA, 10 bp, Artificial Sequence
  primer
gcatggttaa

SEQ ID NO: 155 - DNA, 10 bp, Artificial Sequence
  primer
gcacctaatt

SEQ ID NO: 156 - DNA, 10 bp, Artificial Sequence
  primer
tgtaggttgt

SEQ ID NO: 157 - DNA, 10 bp, Artificial Sequence
  primer
ccatctggaa

SEQ ID NO: 158 - DNA, 10 bp, Artificial Sequence
  primer
ttcgcgttga

SEQ ID NO: 159 - DNA, 10 bp, Artificial Sequence
  primer
aaccgaggtt

SEQ ID NO: 160 - DNA, 10 bp, Artificial Sequence
  primer
gtacgctgtt

SEQ ID NO: 161 - DNA, 10 bp, Artificial Sequence
  primer
agtatcctgg

SEQ ID NO: 162 - DNA, 10 bp, Artificial Sequence
  primer
ggttgtacag

SEQ ID NO: 163 - DNA, 10 bp, Artificial Sequence
  primer
acgtacacca

SEQ ID NO: 164 - DNA, 10 bp, Artificial Sequence
  primer
tgtcgagcaa

SEQ ID NO: 165 - DNA, 10 bp, Artificial Sequence
  primer
gtcgtgttac

SEQ ID NO: 166 - DNA, 10 bp, Artificial Sequence
  primer
gtgcaatagg

SEQ ID NO: 167 - DNA, 10 bp, Artificial Sequence
  primer
actcgatgct

SEQ ID NO: 168 - DNA, 10 bp, Artificial Sequence
  primer
gaatcgcgta

SEQ ID NO: 169 - DNA, 10 bp, Artificial Sequence
  primer
cggtcattgt

SEQ ID NO: 170 - DNA, 10 bp, Artificial Sequence
  primer
atcaggcgat

SEQ ID NO: 171 - DNA, 10 bp, Artificial Sequence
  primer
gtaagatgcg

SEQ ID NO: 172 - DNA, 10 bp, Artificial Sequence
  primer
ggtctcttga

SEQ ID NO: 173 - DNA, 10 bp, Artificial Sequence
  primer
tcctcgctaa

SEQ ID NO: 174 - DNA, 10 bp, Artificial Sequence
  primer
ctgcgtgata

SEQ ID NO: 175 - DNA, 10 bp, Artificial Sequence
  primer
catactcgtc

SEQ ID NO: 176 - DNA, 10 bp, Artificial Sequence
  primer
atctgagctc

SEQ ID NO: 177 - DNA, 10 bp, Artificial Sequence
  primer
acggatagtg

SEQ ID NO: 178 - DNA, 10 bp, Artificial Sequence
  primer
actgcaatgc

SEQ ID NO: 179 - DNA, 10 bp, Artificial Sequence
  primer
taacgacgtg

SEQ ID NO: 180 - DNA, 10 bp, Artificial Sequence
  primer
tagactgtcg

SEQ ID NO: 181 - DNA, 10 bp, Artificial Sequence
  primer
cagcacttca

SEQ ID NO: 182 - DNA, 10 bp, Artificial Sequence
  primer
aacattcgcc

SEQ ID NO: 183 - DNA, 10 bp, Artificial Sequence
  primer
actagtgcgt

SEQ ID NO: 184 - DNA, 10 bp, Artificial Sequence
  primer
acgctgttct

SEQ ID NO: 185 - DNA, 10 bp, Artificial Sequence
  primer
cgtcgaatgc

SEQ ID NO: 186 - DNA, 10 bp, Artificial Sequence
  primer
ctctgacggt

SEQ ID NO: 187 - DNA, 10 bp, Artificial Sequence
  primer
gtcgccatgt

SEQ ID NO: 188 - DNA, 10 bp, Artificial Sequence
  primer
ggtccacgtt

SEQ ID NO: 189 - DNA, 10 bp, Artificial Sequence
  primer
cgagcgactt

SEQ ID NO: 190 - DNA, 10 bp, Artificial Sequence
  primer
ttgacgcgtg

SEQ ID NO: 191 - DNA, 10 bp, Artificial Sequence
  primer
ctgagagcct

SEQ ID NO: 192 - DNA, 10 bp, Artificial Sequence
  primer
cgcgctaact

SEQ ID NO: 193 - DNA, 10 bp, Artificial Sequence
  primer
ggtcgtcaag

SEQ ID NO: 194 - DNA, 10 bp, Artificial Sequence
  primer
aggttgacca

SEQ ID NO: 195 - DNA, 10 bp, Artificial Sequence
  primer
taacggcaac

SEQ ID NO: 196 - DNA, 10 bp, Artificial Sequence
  primer
gaggctggat

SEQ ID NO: 197 - DNA, 10 bp, Artificial Sequence
  primer
gtgcacacct

SEQ ID NO: 198 - DNA, 10 bp, Artificial Sequence
  primer
tgaggaccag

SEQ ID NO: 199 - DNA, 10 bp, Artificial Sequence
  primer
tacttgcgag

SEQ ID NO: 200 - DNA, 10 bp, Artificial Sequence
  primer
aactgtgaga

SEQ ID NO: 201 - DNA, 10 bp, Artificial Sequence
  primer
ctccatcaac

SEQ ID NO: 202 - DNA, 10 bp, Artificial Sequence
  primer
cggactgtta

SEQ ID NO: 203 - DNA, 10 bp, Artificial Sequence
  primer
taggacagtc

SEQ ID NO: 204 - DNA, 10 bp, Artificial Sequence
  primer
agaggacaca

SEQ ID NO: 205 - DNA, 10 bp, Artificial Sequence
  primer
acattcgcgg

SEQ ID NO: 206 - DNA, 10 bp, Artificial Sequence
  primer
gcttactgca

SEQ ID NO: 207 - DNA, 10 bp, Artificial Sequence
  primer
caatacgtaa

SEQ ID NO: 208 - DNA, 10 bp, Artificial Sequence
  primer
agacttgcgc

SEQ ID NO: 209 - DNA, 10 bp, Artificial Sequence
  primer
gagcggtgtt

SEQ ID NO: 210 - DNA, 10 bp, Artificial Sequence
  primer
cgtgagaggt

SEQ ID NO: 211 - DNA, 10 bp, Artificial Sequence
  primer
aatccgtcag

SEQ ID NO: 212 - DNA, 10 bp, Artificial Sequence
  primer
atacgtaccg

SEQ ID NO: 213 - DNA, 10 bp, Artificial Sequence
  primer
aactgattcc

SEQ ID NO: 214 - DNA, 10 bp, Artificial Sequence
  primer
ctgagcgtac

SEQ ID NO: 215 - DNA, 10 bp, Artificial Sequence
  primer
gtcggattcg

SEQ ID NO: 216 - DNA, 10 bp, Artificial Sequence
  primer
gccgaccata

SEQ ID NO: 217 - DNA, 10 bp, Artificial Sequence
  primer
gcagaactaa

SEQ ID NO: 218 - DNA, 10 bp, Artificial Sequence
  primer
ctaacgaccg

SEQ ID NO: 219 - DNA, 10 bp, Artificial Sequence
  primer
gctggaccat

SEQ ID NO: 220 - DNA, 10 bp, Artificial Sequence
  primer
gacgcggtta

SEQ ID NO: 221 - DNA, 10 bp, Artificial Sequence
  primer
agtggtgagc

SEQ ID NO: 222 - DNA, 10 bp, Artificial Sequence
  primer
caggcagtca

SEQ ID NO: 223 - DNA, 10 bp, Artificial Sequence
  primer
tctgacgtca

SEQ ID NO: 224 - DNA, 10 bp, Artificial Sequence
  primer
tacatgacgt

SEQ ID NO: 225 - DNA, 10 bp, Artificial Sequence
  primer
tgaggcaacc

SEQ ID NO: 226 - DNA, 10 bp, Artificial Sequence
  primer
caactgcagt

SEQ ID NO: 227 - DNA, 10 bp, Artificial Sequence
  primer
cggagatacg

SEQ ID NO: 228 - DNA, 10 bp, Artificial Sequence
  primer
cttcgcaagt

SEQ ID NO: 229 - DNA, 10 bp, Artificial Sequence
  primer
ctggcatacg

SEQ ID NO: 230 - DNA, 10 bp, Artificial Sequence
  primer
taacgttcgc

SEQ ID NO: 231 - DNA, 10 bp, Artificial Sequence
  primer
ccggcgttaa

SEQ ID NO: 232 - DNA, 10 bp, Artificial Sequence
  primer
acaagacgcc

SEQ ID NO: 233 - DNA, 10 bp, Artificial Sequence
  primer
ccattagact

SEQ ID NO: 234 - DNA, 10 bp, Artificial Sequence
  primer
gtctgtgaca

SEQ ID NO: 235 - DNA, 10 bp, Artificial Sequence
  primer
ggcattggac

SEQ ID NO: 236 - DNA, 10 bp, Artificial Sequence
  primer
tcttcgcacg

SEQ ID NO: 237 - DNA, 10 bp, Artificial Sequence
  primer
tagcctgtgc

SEQ ID NO: 238 - DNA, 10 bp, Artificial Sequence
  primer
cactgaccta

SEQ ID NO: 239 - DNA, 10 bp, Artificial Sequence
  primer
ccgcacgatt

SEQ ID NO: 240 - DNA, 10 bp, Artificial Sequence
  primer
atagcacacg

SEQ ID NO: 241 - DNA, 10 bp, Artificial Sequence
  primer
gcacgtcata

SEQ ID NO: 242 - DNA, 10 bp, Artificial Sequence
  primer
aagccgttgg

SEQ ID NO: 243 - DNA, 10 bp, Artificial Sequence
  primer
cggaccgtta

SEQ ID NO: 244 - DNA, 10 bp, Artificial Sequence
  primer
tacacagcgt

SEQ ID NO: 245 - DNA, 10 bp, Artificial Sequence
  primer
cggacttcag

SEQ ID NO: 246 - DNA, 10 bp, Artificial Sequence
  primer
tagaacgtca

SEQ ID NO: 247 - DNA, 10 bp, Artificial Sequence
  primer
ggcattggag

SEQ ID NO: 248 - DNA, 10 bp, Artificial Sequence
  primer
ggcactcgtt

SEQ ID NO: 249 - DNA, 10 bp, Artificial Sequence
  primer
gtaccgttaa

SEQ ID NO: 250 - DNA, 10 bp, Artificial Sequence
  primer
aatacgtgtc

SEQ ID NO: 251 - DNA, 10 bp, Artificial Sequence
  primer
ccattgacgt

SEQ ID NO: 252 - DNA, 10 bp, Artificial Sequence
  primer
cgtgaatcgc

SEQ ID NO: 253 - DNA, 10 bp, Artificial Sequence
  primer
atcaacgcgg

SEQ ID NO: 254 - DNA, 10 bp, Artificial Sequence
  primer
cgccaaggta

SEQ ID NO: 255 - DNA, 10 bp, Artificial Sequence
  primer
agaagacgcc

SEQ ID NO: 256 - DNA, 10 bp, Artificial Sequence
  primer
ccgcatagtc

SEQ ID NO: 257 - DNA, 10 bp, Artificial Sequence
  primer
cttatatgtg

SEQ ID NO: 258 - DNA, 10 bp, Artificial Sequence
  primer
ggtctcatcg

SEQ ID NO: 259 - DNA, 10 bp, Artificial Sequence
  primer
ccaccatgtc

SEQ ID NO: 260 - DNA, 10 bp, Artificial Sequence
  primer
acgaatgtgt

SEQ ID NO: 261 - DNA, 10 bp, Artificial Sequence
  primer
ggtagtaaca

SEQ ID NO: 262 - DNA, 10 bp, Artificial Sequence
  primer
gccacttaat

SEQ ID NO: 263 - DNA, 10 bp, Artificial Sequence
  primer
atattgcgcc

SEQ ID NO: 264 - DNA, 10 bp, Artificial Sequence
  primer
gaccaatagt

SEQ ID NO: 265 - DNA, 10 bp, Artificial Sequence
  primer
aacaacacgg

SEQ ID NO: 266 - DNA, 10 bp, Artificial Sequence
  primer
atagccgatg

SEQ ID NO: 267 - DNA, 10 bp, Artificial Sequence
  primer
cgagagcata

SEQ ID NO: 268 - DNA, 10 bp, Artificial Sequence
  primer
cgagacatga

SEQ ID NO: 269 - DNA, 10 bp, Artificial Sequence
  primer
cgccaagtta

SEQ ID NO: 270 - DNA, 10 bp, Artificial Sequence
  primer
ttataatcgc

SEQ ID NO: 271 - DNA, 10 bp, Artificial Sequence
  primer
tagaagtgca

SEQ ID NO: 272 - DNA, 10 bp, Artificial Sequence
  primer
ggaggcatgt

SEQ ID NO: 273 - DNA, 10 bp, Artificial Sequence
  primer
gccacttcga

SEQ ID NO: 274 - DNA, 10 bp, Artificial Sequence
  primer
tccacggtac

SEQ ID NO: 275 - DNA, 10 bp, Artificial Sequence
  primer
caactatgca

SEQ ID NO: 276 - DNA, 10 bp, Artificial Sequence
  primer
caaggaggac

SEQ ID NO: 277 - DNA, 10 bp, Artificial Sequence
  primer
gaggtaccta

SEQ ID NO: 278 - DNA, 10 bp, Artificial Sequence
  primer
gagcgcataa

SEQ ID NO: 279 - DNA, 10 bp, Artificial Sequence
  primer
tcgtcacgtg

SEQ ID NO: 280 - DNA, 10 bp, Artificial Sequence
  primer
aactgtgaca

SEQ ID NO: 281 - DNA, 10 bp, Artificial Sequence
  primer
tccacgtgag

SEQ ID NO: 282 - DNA, 10 bp, Artificial Sequence
  primer
acactgctct

SEQ ID NO: 283 - DNA, 10 bp, Artificial Sequence
  primer
tacggtgagc

SEQ ID NO: 284 - DNA, 10 bp, Artificial Sequence
  primer
cggactaagt

SEQ ID NO: 285 - DNA, 10 bp, Artificial Sequence
  primer
aagccacgtt

SEQ ID NO: 286 - DNA, 10 bp, Artificial Sequence
  primer
caattactcg

SEQ ID NO: 287 - DNA, 10 bp, Artificial Sequence
  primer
tctggccata

SEQ ID NO: 288 - DNA, 10 bp, Artificial Sequence
  primer
tcaggctagt

SEQ ID NO: 289 - DNA, 10 bp, Artificial Sequence
  primer
ttgacccgga

SEQ ID NO: 290 - DNA, 10 bp, Artificial Sequence
  primer
tttttatggt

SEQ ID NO: 291 - DNA, 10 bp, Artificial Sequence
  primer
atgtggtgcg

SEQ ID NO: 292 - DNA, 10 bp, Artificial Sequence
  primer
aaggcgctag

SEQ ID NO: 293 - DNA, 10 bp, Artificial Sequence
  primer
tccaactttg

SEQ ID NO: 294 - DNA, 10 bp, Artificial Sequence
  primer
ccatcccatc

SEQ ID NO: 295 - DNA, 10 bp, Artificial Sequence
  primer
caatacgagg

SEQ ID NO: 296 - DNA, 10 bp, Artificial Sequence
  primer
gagtgttacc

SEQ ID NO: 297 - DNA, 10 bp, Artificial Sequence
  primer
gcctcctgta

SEQ ID NO: 298 - DNA, 10 bp, Artificial Sequence
  primer
cgaaggttgc

SEQ ID NO: 299 - DNA, 10 bp, Artificial Sequence
  primer
gaggtgctat

SEQ ID NO: 300 - DNA, 10 bp, Artificial Sequence
  primer
taggataatt

SEQ ID NO: 301 - DNA, 10 bp, Artificial Sequence
  primer
cgttgtcctc

SEQ ID NO: 302 - DNA, 10 bp, Artificial Sequence
  primer
tgagaccagc

SEQ ID NO: 303 - DNA, 10 bp, Artificial Sequence
  primer
tgcccaagct

SEQ ID NO: 304 - DNA, 10 bp, Artificial Sequence
  primer
tactgaatcg

SEQ ID NO: 305 - DNA, 10 bp, Artificial Sequence
  primer
ttacatagtc

SEQ ID NO: 306 - DNA, 10 bp, Artificial Sequence
  primer
acaaaggaaa

SEQ ID NO: 307 - DNA, 10 bp, Artificial Sequence
  primer
ctcgcttggg

SEQ ID NO: 308 - DNA, 10 bp, Artificial Sequence
  primer
ccttgcgtca

SEQ ID NO: 309 - DNA, 10 bp, Artificial Sequence
  primer
taattccgaa

SEQ ID NO: 310 - DNA, 10 bp, Artificial Sequence
  primer
gtgagcttga

SEQ ID NO: 311 - DNA, 10 bp, Artificial Sequence
  primer
atgccgattc

SEQ ID NO: 312 - DNA, 10 bp, Artificial Sequence
  primer
gcttgggctt

SEQ ID NO: 313 - DNA, 10 bp, Artificial Sequence
  primer
acaaagcgcc

SEQ ID NO: 314 - DNA, 10 bp, Artificial Sequence
  primer
gaaagctcta

SEQ ID NO: 315 - DNA, 10 bp, Artificial Sequence
  primer
taccgaccgt

SEQ ID NO: 316 - DNA, 10 bp, Artificial Sequence
  primer
tcgaagagac

SEQ ID NO: 317 - DNA, 10 bp, Artificial Sequence
  primer
gtcgcttacg

SEQ ID NO: 318 - DNA, 10 bp, Artificial Sequence
  primer
gggctctcca

SEQ ID NO: 319 - DNA, 10 bp, Artificial Sequence
  primer
gcgcccttgt

SEQ ID NO: 320 - DNA, 10 bp, Artificial Sequence
  primer
ggcaataggc

SEQ ID NO: 321 - DNA, 10 bp, Artificial Sequence
  primer
caagtcagga

SEQ ID NO: 322 - DNA, 10 bp, Artificial Sequence
  primer
gggtcgcaat

SEQ ID NO: 323 - DNA, 10 bp, Artificial Sequence
  primer
cagcaaccta

SEQ ID NO: 324 - DNA, 10 bp, Artificial Sequence
  primer
ttcccgccac

SEQ ID NO: 325 - DNA, 10 bp, Artificial Sequence
  primer
tgtgcatttt

SEQ ID NO: 326 - DNA, 10 bp, Artificial Sequence
  primer
atcaacgacg

SEQ ID NO: 327 - DNA, 10 bp, Artificial Sequence
  primer
gtgacgtcca

SEQ ID NO: 328 - DNA, 10 bp, Artificial Sequence
  primer
cgatctagtc

SEQ ID NO: 329 - DNA, 10 bp, Artificial Sequence
  primer
ttacatcctg

SEQ ID NO: 330 - DNA, 10 bp, Artificial Sequence
  primer
agccttcaat

SEQ ID NO: 331 - DNA, 10 bp, Artificial Sequence
  primer
tccatccgat

SEQ ID NO: 332 - DNA, 10 bp, Artificial Sequence
  primer
gactgggtct

SEQ ID NO: 333 - DNA, 10 bp, Artificial Sequence
  primer
ttcggtggag

SEQ ID NO: 334 - DNA, 10 bp, Artificial Sequence
  primer
gaccagcaca

SEQ ID NO: 335 - DNA, 10 bp, Artificial Sequence
  primer
cattaacgga

SEQ ID NO: 336 - DNA, 10 bp, Artificial Sequence
  primer
tttttcttga

SEQ ID NO: 337 - DNA, 10 bp, Artificial Sequence
  primer
cattgcactg

SEQ ID NO: 338 - DNA, 10 bp, Artificial Sequence
  primer
tgcggcgatc

SEQ ID NO: 339 - DNA, 10 bp, Artificial Sequence
  primer
atattgcggt

SEQ ID NO: 340 - DNA, 10 bp, Artificial Sequence
  primer
gacgtcgctc

SEQ ID NO: 341 - DNA, 10 bp, Artificial Sequence
  primer
tcgcttatcg

SEQ ID NO: 342 - DNA, 10 bp, Artificial Sequence
  primer
gcgcagacac

SEQ ID NO: 343 - DNA, 10 bp, Artificial Sequence
  primer
catgtattgt

SEQ ID NO: 344 - DNA, 10 bp, Artificial Sequence
  primer
tctataacct

SEQ ID NO: 345 - DNA, 10 bp, Artificial Sequence
  primer
gtggagacaa

SEQ ID NO: 346 - DNA, 10 bp, Artificial Sequence
  primer
cgaagattat

SEQ ID NO: 347 - DNA, 10 bp, Artificial Sequence
  primer
tagcaactgc

SEQ ID NO: 348 - DNA, 10 bp, Artificial Sequence
  primer
ataatcggta

SEQ ID NO: 349 - DNA, 10 bp, Artificial Sequence
  primer
caggatgggt

SEQ ID NO: 350 - DNA, 10 bp, Artificial Sequence
  primer
gacgattccc

SEQ ID NO: 351 - DNA, 10 bp, Artificial Sequence
  primer
cacgccttac

SEQ ID NO: 352 - DNA, 10 bp, Artificial Sequence
  primer
agttggttcc

SEQ ID NO: 353 - DNA, 10 bp, Artificial Sequence
  primer
tcttatcagg

SEQ ID NO: 354 - DNA, 10 bp, Artificial Sequence
  primer
cgagaagttc

SEQ ID NO: 355 - DNA, 10 bp, Artificial Sequence
  primer
gtggtagaat

SEQ ID NO: 356 - DNA, 10 bp, Artificial Sequence
  primer
taggcttgtg

SEQ ID NO: 357 - DNA, 10 bp, Artificial Sequence
  primer
atgcgttacg

SEQ ID NO: 358 - DNA, 10 bp, Artificial Sequence
  primer
actaccgagg

SEQ ID NO: 359 - DNA, 10 bp, Artificial Sequence
  primer
cgagttggtg

SEQ ID NO: 360 - DNA, 10 bp, Artificial Sequence
  primer
ggacgatcaa

SEQ ID NO: 361 - DNA, 10 bp, Artificial Sequence
  primer
aacagtatgc

SEQ ID NO: 362 - DNA, 10 bp, Artificial Sequence
  primer
ttggctgatc

SEQ ID NO: 363 - DNA, 10 bp, Artificial Sequence
  primer
aggattggaa

SEQ ID NO: 364 - DNA, 10 bp, Artificial Sequence
  primer
catatggaga

SEQ ID NO: 365 - DNA, 10 bp, Artificial Sequence
  primer
ctgcaggttt

SEQ ID NO: 366 - DNA, 10 bp, Artificial Sequence
  primer
ctctcttttt

SEQ ID NO: 367 - DNA, 10 bp, Artificial Sequence
  primer
agtaggggtc

SEQ ID NO: 368 - DNA, 10 bp, Artificial Sequence
  primer
acaccgcaag

SEQ ID NO: 369 - DNA, 10 bp, Artificial Sequence
  primer
gaagcgggag

SEQ ID NO: 370 - DNA, 10 bp, Artificial Sequence
  primer
gatacggact

SEQ ID NO: 371 - DNA, 10 bp, Artificial Sequence
  primer
tacgacgtgt

SEQ ID NO: 372 - DNA, 10 bp, Artificial Sequence
  primer
gtgcctcctt

SEQ ID NO: 373 - DNA, 10 bp, Artificial Sequence
  primer
ggtgactgat

SEQ ID NO: 374 - DNA, 10 bp, Artificial Sequence
  primer
atatcttacg

SEQ ID NO: 375 - DNA, 10 bp, Artificial Sequence
  primer
aatcatacgg

SEQ ID NO: 376 - DNA, 10 bp, Artificial Sequence
  primer
ctcttgggac

SEQ ID NO: 377 - DNA, 10 bp, Artificial Sequence
  primer
gacgacaaat

SEQ ID NO: 378 - DNA, 10 bp, Artificial Sequence
  primer
gttgcgaggt

SEQ ID NO: 379 - DNA, 10 bp, Artificial Sequence
  primer
aaaccgcacc

SEQ ID NO: 380 - DNA, 10 bp, Artificial Sequence
  primer
gctaacacgt

SEQ ID NO: 381 - DNA, 10 bp, Artificial Sequence
  primer
atcatgaggg

SEQ ID NO: 382 - DNA, 10 bp, Artificial Sequence
  primer
gattcacgta

SEQ ID NO: 383 - DNA, 10 bp, Artificial Sequence
  primer
tctcgaaaag

SEQ ID NO: 384 - DNA, 10 bp, Artificial Sequence
  primer
ctcgtaacca

SEQ ID NO: 385 - DNA, 10 bp, Artificial Sequence
  primer
gttacacacg

SEQ ID NO: 386 - DNA, 10 bp, Artificial Sequence
  primer
cgtgaagggt

SEQ ID NO: 387 - DNA, 10 bp, Artificial Sequence
  primer
acgagcatct

SEQ ID NO: 388 - DNA, 10 bp, Artificial Sequence
  primer
acgagggatt

SEQ ID NO: 389 - DNA, 10 bp, Artificial Sequence
  primer
gcaacgtcgg

SEQ ID NO: 390 - DNA, 10 bp, Artificial Sequence
  primer
cacggctagg

SEQ ID NO: 391 - DNA, 10 bp, Artificial Sequence
  primer
cgtgactctc

SEQ ID NO: 392 - DNA, 10 bp, Artificial Sequence
  primer
tctagacgca

SEQ ID NO: 393 - DNA, 10 bp, Artificial Sequence
  primer
ctgcgcacat

SEQ ID NO: 394 - DNA, 10 bp, Artificial Sequence
  primer
atgcttgaca

SEQ ID NO: 395 - DNA, 10 bp, Artificial Sequence
  primer
tttgtcgaca

SEQ ID NO: 396 - DNA, 10 bp, Artificial Sequence
  primer
acgtgtcagc

SEQ ID NO: 397 - DNA, 10 bp, Artificial Sequence
  primer
gaaaacatta

SEQ ID NO: 398 - DNA, 10 bp, Artificial Sequence
  primer
acattaacgg

SEQ ID NO: 399 - DNA, 10 bp, Artificial Sequence
  primer
gtacaggtcc

SEQ ID NO: 400 - DNA, 10 bp, Artificial Sequence
  primer
ctatgtgtac

SEQ ID NO: 401 - DNA, 10 bp, Artificial Sequence
  primer
gcgtacatta

SEQ ID NO: 402 - DNA, 10 bp, Artificial Sequence
  primer
gatttgtggc

SEQ ID NO: 403 - DNA, 10 bp, Artificial Sequence
  primer
tcgcgcgcta

SEQ ID NO: 404 - DNA, 10 bp, Artificial Sequence
  primer
acaagggcga

SEQ ID NO: 405 - DNA, 10 bp, Artificial Sequence
  primer
aacgcgcgat

SEQ ID NO: 406 - DNA, 10 bp, Artificial Sequence
  primer
cgtaaatgcg

SEQ ID NO: 407 - DNA, 10 bp, Artificial Sequence
  primer
taggcactac

SEQ ID NO: 408 - DNA, 10 bp, Artificial Sequence
  primer
gcgaggatcg

SEQ ID NO: 409 - DNA, 10 bp, Artificial Sequence
  primer
cacgtttact

SEQ ID NO: 410 - DNA, 10 bp, Artificial Sequence
  primer
taccaccacg

SEQ ID NO: 411 - DNA, 10 bp, Artificial Sequence
  primer
ttaacaggac

SEQ ID NO: 412 - DNA, 10 bp, Artificial Sequence
  primer
gctgtataac

SEQ ID NO: 413 - DNA, 10 bp, Artificial Sequence
  primer
gttgctggca

SEQ ID NO: 414 - DNA, 10 bp, Artificial Sequence
  primer
agtgtggcca

SEQ ID NO: 415 - DNA, 10 bp, Artificial Sequence
  primer
ctgcggttgt

SEQ ID NO: 416 - DNA, 10 bp, Artificial Sequence
  primer
tagatcagcg

SEQ ID NO: 417 - DNA, 10 bp, Artificial Sequence
  primer
ttccggttat

SEQ ID NO: 418 - DNA, 10 bp, Artificial Sequence
  primer
gataaactgt

SEQ ID NO: 419 - DNA, 10 bp, Artificial Sequence
  primer
tacagttgcc

SEQ ID NO: 420 - DNA, 10 bp, Artificial Sequence
  primer
cgatggcgaa

SEQ ID NO: 421 - DNA, 10 bp, Artificial Sequence
  primer
ccgacgtcag

SEQ ID NO: 422 - DNA, 10 bp, Artificial Sequence
  primer
tatggtgcaa

SEQ ID NO: 423 - DNA, 10 bp, Artificial Sequence
  primer
gacgacagtc

SEQ ID NO: 424 - DNA, 10 bp, Artificial Sequence
  primer
gtcaccgtcc

SEQ ID NO: 425 - DNA, 10 bp, Artificial Sequence
  primer
ggttttaaca

SEQ ID NO: 426 - DNA, 10 bp, Artificial Sequence
  primer
gaggacagta

SEQ ID NO: 427 - DNA, 10 bp, Artificial Sequence
  primer
gttacctaag

SEQ ID NO: 428 - DNA, 10 bp, Artificial Sequence
  primer
atcacgtgtt

SEQ ID NO: 429 - DNA, 10 bp, Artificial Sequence
  primer
taaggcctgg

SEQ ID NO: 430 - DNA, 10 bp, Artificial Sequence
  primer
tgttcgtagc

SEQ ID NO: 431 - DNA, 10 bp, Artificial Sequence
  primer
tgaggacgtg

SEQ ID NO: 432 - DNA, 10 bp, Artificial Sequence
  primer
gtgctgtgta

SEQ ID NO: 433 - DNA, 10 bp, Artificial Sequence
  primer
gagggtacgc

SEQ ID NO: 434 - DNA, 10 bp, Artificial Sequence
  primer
ccgtgattgt

SEQ ID NO: 435 - DNA, 10 bp, Artificial Sequence
  primer
aaaatcgcct

SEQ ID NO: 436 - DNA, 10 bp, Artificial Sequence
  primer
cgatcgcagt

SEQ ID NO: 437 - DNA, 10 bp, Artificial Sequence
  primer
acgcaataag

SEQ ID NO: 438 - DNA, 10 bp, Artificial Sequence
  primer
aaggtgcatc

SEQ ID NO: 439 - DNA, 10 bp, Artificial Sequence
  primer
cgcgtagata

SEQ ID NO: 440 - DNA, 10 bp, Artificial Sequence
  primer
cgagcagtgc

SEQ ID NO: 441 - DNA, 10 bp, Artificial Sequence
  primer
atacgtgacg

SEQ ID NO: 442 - DNA, 10 bp, Artificial Sequence
  primer
agattgcgcg

SEQ ID NO: 443 - DNA, 10 bp, Artificial Sequence
  primer
acgtgatgcc

SEQ ID NO: 444 - DNA, 10 bp, Artificial Sequence
  primer
gtacgcatcg

SEQ ID NO: 445 - DNA, 10 bp, Artificial Sequence
  primer
tcccgactta

SEQ ID NO: 446 - DNA, 10 bp, Artificial Sequence
  primer
gtttttacac

SEQ ID NO: 447 - DNA, 10 bp, Artificial Sequence
  primer
cctgagcgtg

SEQ ID NO: 448 - DNA, 10 bp, Artificial Sequence
  primer
cggcattgta

SEQ ID NO: 449 - DNA, 10 bp, Artificial Sequence
  primer
tagagtgcgt

SEQ ID NO: 450 - DNA, 10 bp, Artificial Sequence
  primer
atggccagac

SEQ ID NO: 451 - DNA, 10 bp, Artificial Sequence
  primer
cttagcatgc

SEQ ID NO: 452 - DNA, 10 bp, Artificial Sequence
  primer
acaacacctg

SEQ ID NO: 453 - DNA, 10 bp, Artificial Sequence
  primer
agtgactatc

SEQ ID NO: 454 - DNA, 10 bp, Artificial Sequence
  primer
catgctacac

SEQ ID NO: 455 - DNA, 10 bp, Artificial Sequence
  primer
aaagcgggcg

SEQ ID NO: 456 - DNA, 10 bp, Artificial Sequence
  primer
agatcgccgt

SEQ ID NO: 457 - DNA, 10 bp, Artificial Sequence
  primer
cgtagatatt

SEQ ID NO: 458 - DNA, 10 bp, Artificial Sequence
  primer
aatggcagac

SEQ ID NO: 459 - DNA, 10 bp, Artificial Sequence
  primer
gtataacgtg

SEQ ID NO: 460 - DNA, 10 bp, Artificial Sequence
  primer
atgtgcgtca

SEQ ID NO: 461 - DNA, 10 bp, Artificial Sequence
  primer
cctgccaact

SEQ ID NO: 462 - DNA, 10 bp, Artificial Sequence
  primer
tttataactc

SEQ ID NO: 463 - DNA, 10 bp, Artificial Sequence
  primer
acggttacgc

SEQ ID NO: 464 - DNA, 10 bp, Artificial Sequence
  primer
tagcctcttg

SEQ ID NO: 465 - DNA, 10 bp, Artificial Sequence
  primer
tcgcgaagtt

SEQ ID NO: 466 - DNA, 10 bp, Artificial Sequence
  primer
gtctacaacc

SEQ ID NO: 467 - DNA, 10 bp, Artificial Sequence
  primer
gtctactgcg

SEQ ID NO: 468 - DNA, 10 bp, Artificial Sequence
  primer
gttgcgtctc

SEQ ID NO: 469 - DNA, 10 bp, Artificial Sequence
  primer
gggccgctaa

SEQ ID NO: 470 - DNA, 10 bp, Artificial Sequence
  primer
gtacgtcgga

SEQ ID NO: 471 - DNA, 10 bp, Artificial Sequence
  primer
agcgagagac

SEQ ID NO: 472 - DNA, 10 bp, Artificial Sequence
  primer
tggctacggt

SEQ ID NO: 473 - DNA, 10 bp, Artificial Sequence
  primer
aggcatcacg

SEQ ID NO: 474 - DNA, 10 bp, Artificial Sequence
  primer
tagctcctcg

SEQ ID NO: 475 - DNA, 10 bp, Artificial Sequence
  primer
ggctagtcag

SEQ ID NO: 476 - DNA, 10 bp, Artificial Sequence
  primer
ctcactttat

SEQ ID NO: 477 - DNA, 10 bp, Artificial Sequence
  primer
acggccacgt

SEQ ID NO: 478 - DNA, 10 bp, Artificial Sequence
  primer
agcgtatatc

SEQ ID NO: 479 - DNA, 10 bp, Artificial Sequence
  primer
gacacgtcta

SEQ ID NO: 480 - DNA, 10 bp, Artificial Sequence
  primer
gccagcgtac

SEQ ID NO: 481 - DNA, 10 bp, Artificial Sequence
  primer
aacattagcg

SEQ ID NO: 482 - DNA, 10 bp, Artificial Sequence
  primer
agtgtgctat

SEQ ID NO: 483 - DNA, 10 bp, Artificial Sequence
  primer
cacgagcgtt

SEQ ID NO: 484 - DNA, 10 bp, Artificial Sequence
  primer
gtaacgccta

SEQ ID NO: 485 - DNA, 10 bp, Artificial Sequence
  primer
cacatagtac

SEQ ID NO: 486 - DNA, 10 bp, Artificial Sequence
  primer
cgcgatatcg

SEQ ID NO: 487 - DNA, 10 bp, Artificial Sequence
  primer
cgttctgtgc

SEQ ID NO: 488 - DNA, 10 bp, Artificial Sequence
  primer
ctgatcgcat

SEQ ID NO: 489 - DNA, 10 bp, Artificial Sequence
  primer
tggcgtgaga

SEQ ID NO: 490 - DNA, 10 bp, Artificial Sequence
  primer
ttgccaggct

SEQ ID NO: 491 - DNA, 10 bp, Artificial Sequence
  primer
gttatacaca

SEQ ID NO: 492 - DNA, 10 bp, Artificial Sequence
  primer
agtgccaact

SEQ ID NO: 493 - DNA, 10 bp, Artificial Sequence
  primer
tcacgtagca

SEQ ID NO: 494 - DNA, 10 bp, Artificial Sequence
  primer
taattcagcg

SEQ ID NO: 495 - DNA, 10 bp, Artificial Sequence
  primer
aagtatcgtc

SEQ ID NO: 496 - DNA, 10 bp, Artificial Sequence
  primer
cacagttact

SEQ ID NO: 497 - DNA, 10 bp, Artificial Sequence
  primer
ccttaccgtg

SEQ ID NO: 498 - DNA, 10 bp, Artificial Sequence
  primer
acggtgtcgt

SEQ ID NO: 499 - DNA, 10 bp, Artificial Sequence
  primer
cgcgtaagac

SEQ ID NO: 500 - DNA, 10 bp, Artificial Sequence
  primer
ttcgcaccag

SEQ ID NO: 501 - DNA, 10 bp, Artificial Sequence
  primer
cacgaacaga

SEQ ID NO: 502 - DNA, 10 bp, Artificial Sequence
  primer
gttggacatt

SEQ ID NO: 503 - DNA, 10 bp, Artificial Sequence
  primer
ggtgcttaag

SEQ ID NO: 504 - DNA, 10 bp, Artificial Sequence
  primer
tcggtctcgt

SEQ ID NO: 505 - DNA, 10 bp, Artificial Sequence
  primer
tctagtacgc

SEQ ID NO: 506 - DNA, 10 bp, Artificial Sequence
  primer
ttaggccgag

SEQ ID NO: 507 - DNA, 10 bp, Artificial Sequence
  primer
cgtcaagagc

SEQ ID NO: 508 - DNA, 10 bp, Artificial Sequence
  primer
acatgtctac

SEQ ID NO: 509 - DNA, 10 bp, Artificial Sequence
  primer
atcgttacgt

SEQ ID NO: 510 - DNA, 10 bp, Artificial Sequence
  primer
acggatcgtt

SEQ ID NO: 511 - DNA, 10 bp, Artificial Sequence
  primer
aatcttggcg

SEQ ID NO: 512 - DNA, 10 bp, Artificial Sequence
  primer
agtatctggt

SEQ ID NO: 513 - DNA, 10 bp, Artificial Sequence
  primer
caaccgacgt

SEQ ID NO: 514 - DNA, 10 bp, Artificial Sequence
  primer
tggtaacgcg

SEQ ID NO: 515 - DNA, 10 bp, Artificial Sequence
  primer
gtgcagacat

SEQ ID NO: 516 - DNA, 10 bp, Artificial Sequence
  primer
gtctagttgc

SEQ ID NO: 517 - DNA, 10 bp, Artificial Sequence
  primer
caattcgacg

SEQ ID NO: 518 - DNA, 10 bp, Artificial Sequence
  primer
cttagcacct

SEQ ID NO: 519 - DNA, 10 bp, Artificial Sequence
  primer
taatgtcgca

SEQ ID NO: 520 - DNA, 10 bp, Artificial Sequence
  primer
caatcggtac

SEQ ID NO: 521 - DNA, 10 bp, Artificial Sequence
  primer
agcacgcatt

SEQ ID NO: 522 - DNA, 10 bp, Artificial Sequence
  primer
aggtcctcgt

SEQ ID NO: 523 - DNA, 10 bp, Artificial Sequence
  primer
ttgtgcctgc

SEQ ID NO: 524 - DNA, 10 bp, Artificial Sequence
  primer
accgcctgta

SEQ ID NO: 525 - DNA, 10 bp, Artificial Sequence
  primer
gtacgtcagg

SEQ ID NO: 526 - DNA, 10 bp, Artificial Sequence
  primer
gcacacaact

SEQ ID NO: 527 - DNA, 10 bp, Artificial Sequence
  primer
tgagcactta

SEQ ID NO: 528 - DNA, 10 bp, Artificial Sequence
  primer
gtgccgcata

SEQ ID NO: 529 - DNA, 10 bp, Artificial Sequence
  primer
atgttttcgc

SEQ ID NO: 530 - DNA, 10 bp, Artificial Sequence
  primer
acacttaggt

SEQ ID NO: 531 - DNA, 10 bp, Artificial Sequence
  primer
cgtgccgtga

SEQ ID NO: 532 - DNA, 10 bp, Artificial Sequence
  primer
ttactaatca

SEQ ID NO: 533 - DNA, 10 bp, Artificial Sequence
  primer
gtggcaggta

SEQ ID NO: 534 - DNA, 10 bp, Artificial Sequence
  primer
gcgcgatatg

SEQ ID NO: 535 - DNA, 10 bp, Artificial Sequence
  primer
gaacgacgtt

SEQ ID NO: 536 - DNA, 10 bp, Artificial Sequence
  primer
atcaggagtg

SEQ ID NO: 537 - DNA, 10 bp, Artificial Sequence
  primer
gccagtaagt

SEQ ID NO: 538 - DNA, 10 bp, Artificial Sequence
  primer
gcaagaagca

SEQ ID NO: 539 - DNA, 10 bp, Artificial Sequence
  primer
aactccgcca

SEQ ID NO: 540 - DNA, 10 bp, Artificial Sequence
  primer
acttgagcct

SEQ ID NO: 541 - DNA, 10 bp, Artificial Sequence
  primer
cgtgatcgtg

SEQ ID NO: 542 - DNA, 10 bp, Artificial Sequence
  primer
aattagcgaa

SEQ ID NO: 543 - DNA, 10 bp, Artificial Sequence
  primer
acttccttag

SEQ ID NO: 544 - DNA, 10 bp, Artificial Sequence
  primer
tgtgctgata

SEQ ID NO: 545 - DNA, 10 bp, Artificial Sequence
  primer
aggcggctga

SEQ ID NO: 546 - DNA, 10 bp, Artificial Sequence
  primer
cgtttagagc

SEQ ID NO: 547 - DNA, 10 bp, Artificial Sequence
  primer
acgcgtctaa

SEQ ID NO: 548 - DNA, 10 bp, Artificial Sequence
  primer
gcgaatgtac

SEQ ID NO: 549 - DNA, 10 bp, Artificial Sequence
  primer
cgtgatccaa

SEQ ID NO: 550 - DNA, 10 bp, Artificial Sequence
  primer
caaccagatg

SEQ ID NO: 551 - DNA, 10 bp, Artificial Sequence
  primer
accattaacc

SEQ ID NO: 552 - DNA, 10 bp, Artificial Sequence
  primer
cgattcacgt

SEQ ID NO: 553 - DNA, 10 bp, Artificial Sequence
  primer
ctagaacctg

SEQ ID NO: 554 - DNA, 10 bp, Artificial Sequence
  primer
cctaacgaca

SEQ ID NO: 555 - DNA, 10 bp, Artificial Sequence
  primer
gacgtgcatg

SEQ ID NO: 556 - DNA, 10 bp, Artificial Sequence
  primer
atgtaacctt

SEQ ID NO: 557 - DNA, 10 bp, Artificial Sequence
  primer
gatacagtcg

SEQ ID NO: 558 - DNA, 10 bp, Artificial Sequence
  primer
cgtatgtctc

SEQ ID NO: 559 - DNA, 10 bp, Artificial Sequence
  primer
agattatcga

SEQ ID NO: 560 - DNA, 10 bp, Artificial Sequence
  primer
atactggtaa

SEQ ID NO: 561 - DNA, 10 bp, Artificial Sequence
  primer
gttgagtagc

SEQ ID NO: 562 - DNA, 10 bp, Artificial Sequence
  primer
accattatca

SEQ ID NO: 563 - DNA, 10 bp, Artificial Sequence
  primer
cacacttcag

SEQ ID NO: 564 - DNA, 10 bp, Artificial Sequence
  primer
gactagcggt

SEQ ID NO: 565 - DNA, 10 bp, Artificial Sequence
  primer
aattgtcgag

SEQ ID NO: 566 - DNA, 10 bp, Artificial Sequence
  primer
ctaaggacgt

SEQ ID NO: 567 - DNA, 10 bp, Artificial Sequence
  primer
attacgatga

SEQ ID NO: 568 - DNA, 10 bp, Artificial Sequence
  primer
attgaagact

SEQ ID NO: 569 - DNA, 10 bp, Artificial Sequence
  primer
gcttgtacgt

SEQ ID NO: 570 - DNA, 10 bp, Artificial Sequence
  primer
cctacgtcac

SEQ ID NO: 571 - DNA, 10 bp, Artificial Sequence
  primer
cacaacttag

SEQ ID NO: 572 - DNA, 10 bp, Artificial Sequence
  primer
gcggttcatc

SEQ ID NO: 573 - DNA, 10 bp, Artificial Sequence
  primer
gtactcatct

SEQ ID NO: 574 - DNA, 10 bp, Artificial Sequence
  primer
gtgcatcagt

SEQ ID NO: 575 - DNA, 10 bp, Artificial Sequence
  primer
tcacatccta

SEQ ID NO: 576 - DNA, 10 bp, Artificial Sequence
  primer
cacgcgctat

SEQ ID NO: 577 - DNA, 8 bp, Artificial Sequence
  primer
ctatcttg

SEQ ID NO: 578 - DNA, 8 bp, Artificial Sequence
  primer
aagtgcgt

SEQ ID NO: 579 - DNA, 8 bp, Artificial Sequence
  primer
acatgcga

SEQ ID NO: 580 - DNA, 8 bp, Artificial Sequence
  primer
accaatgg

SEQ ID NO: 581 - DNA, 8 bp, Artificial Sequence
  primer
tgcgttga

SEQ ID NO: 582 - DNA, 8 bp, Artificial Sequence
  primer
gacatgtc

SEQ ID NO: 583 - DNA, 8 bp, Artificial Sequence
  primer
ttgtgcgt

SEQ ID NO: 584 - DNA, 8 bp, Artificial Sequence
  primer
acatcgca

SEQ ID NO: 585 - DNA, 8 bp, Artificial Sequence
  primer
gaagacga

SEQ ID NO: 586 - DNA, 8 bp, Artificial Sequence
  primer
tcgataga

SEQ ID NO: 587 - DNA, 8 bp, Artificial Sequence
  primer
tcttgcaa

SEQ ID NO: 588 - DNA, 8 bp, Artificial Sequence
  primer
agcaagtt

SEQ ID NO: 589 - DNA, 8 bp, Artificial Sequence
  primer
ttcatgga

SEQ ID NO: 590 - DNA, 8 bp, Artificial Sequence
  primer
tcaattcg

SEQ ID NO: 591 - DNA, 8 bp, Artificial Sequence
  primer
cggtatgt

SEQ ID NO: 592 - DNA, 8 bp, Artificial Sequence
  primer
accactac

SEQ ID NO: 593 - DNA, 8 bp, Artificial Sequence
  primer
tcgcttat

SEQ ID NO: 594 - DNA, 8 bp, Artificial Sequence
  primer
tctcgact

SEQ ID NO: 595 - DNA, 8 bp, Artificial Sequence
  primer
gaatcggt

SEQ ID NO: 596 - DNA, 8 bp, Artificial Sequence
  primer
gttacaag

SEQ ID NO: 597 - DNA, 8 bp, Artificial Sequence
  primer
ctgtgtag

SEQ ID NO: 598 - DNA, 8 bp, Artificial Sequence
  primer
tggtagaa

SEQ ID NO: 599 - DNA, 8 bp, Artificial Sequence
  primer
atactgcg

SEQ ID NO: 600 - DNA, 8 bp, Artificial Sequence
  primer
aactcgtc

SEQ ID NO: 601 - DNA, 8 bp, Artificial Sequence
  primer
atatgtgc

SEQ ID NO: 602 - DNA, 8 bp, Artificial Sequence
  primer
aagttgcg

SEQ ID NO: 603 - DNA, 8 bp, Artificial Sequence
  primer
gatcatgt

SEQ ID NO: 604 - DNA, 8 bp, Artificial Sequence
  primer
ttgttgct

SEQ ID NO: 605 - DNA, 8 bp, Artificial Sequence
  primer
cctcttag

SEQ ID NO: 606 - DNA, 8 bp, Artificial Sequence
  primer
tcacagct

SEQ ID NO: 607 - DNA, 8 bp, Artificial Sequence
  primer
agattgac

SEQ ID NO: 608 - DNA, 8 bp, Artificial Sequence
  primer
agcctgat

SEQ ID NO: 609 - DNA, 8 bp, Artificial Sequence
  primer
cgtcaagt

SEQ ID NO: 610 - DNA, 8 bp, Artificial Sequence
  primer
aagtagac

SEQ ID NO: 611 - DNA, 8 bp, Artificial Sequence
  primer
tcagacaa

SEQ ID NO: 612 - DNA, 8 bp, Artificial Sequence
  primer
tccttgac

SEQ ID NO: 613 - DNA, 8 bp, Artificial Sequence
  primer
gtagctgt

SEQ ID NO: 614 - DNA, 8 bp, Artificial Sequence
  primer
cgtcgtaa

SEQ ID NO: 615 - DNA, 8 bp, Artificial Sequence
  primer
ccaatgga

SEQ ID NO: 616 - DNA, 8 bp, Artificial Sequence
  primer
ttgagaga

SEQ ID NO: 617 - DNA, 8 bp, Artificial Sequence
  primer
acaacacc

SEQ ID NO: 618 - DNA, 8 bp, Artificial Sequence
  primer
tctagtac

SEQ ID NO: 619 - DNA, 8 bp, Artificial Sequence
  primer
gaggaagt

SEQ ID NO: 620 - DNA, 8 bp, Artificial Sequence
  primer
gcgtattg

SEQ ID NO: 621 - DNA, 8 bp, Artificial Sequence
  primer
aagtagct

SEQ ID NO: 622 - DNA, 8 bp, Artificial Sequence
  primer
tgaacctt

SEQ ID NO: 623 - DNA, 8 bp, Artificial Sequence
  primer
tgtgttac

SEQ ID NO: 624 - DNA, 8 bp, Artificial Sequence
  primer
taacctga

SEQ ID NO: 625 - DNA, 8 bp, Artificial Sequence
  primer
gctattcc

SEQ ID NO: 626 - DNA, 8 bp, Artificial Sequence
  primer
gttagatg

SEQ ID NO: 627 - DNA, 8 bp, Artificial Sequence
  primer
caggataa

SEQ ID NO: 628 - DNA, 8 bp, Artificial Sequence
  primer
accgtagt

SEQ ID NO: 629 - DNA, 8 bp, Artificial Sequence
  primer
ccgtgtat

SEQ ID NO: 630 - DNA, 8 bp, Artificial Sequence
  primer
tccactct

SEQ ID NO: 631 - DNA, 8 bp, Artificial Sequence
  primer
tagctcat

SEQ ID NO: 632 - DNA, 8 bp, Artificial Sequence
  primer
cgctaata

SEQ ID NO: 633 - DNA, 8 bp, Artificial Sequence
  primer
tacctctg

SEQ ID NO: 634 - DNA, 8 bp, Artificial Sequence
  primer
tgcactac

SEQ ID NO: 635 - DNA, 8 bp, Artificial Sequence
  primer
cttggaag

SEQ ID NO: 636 - DNA, 8 bp, Artificial Sequence
  primer
aatgcacg

SEQ ID NO: 637 - DNA, 8 bp, Artificial Sequence
  primer
cactgtta

SEQ ID NO: 638 - DNA, 8 bp, Artificial Sequence
  primer
tcgactag

SEQ ID NO: 639 - DNA, 8 bp, Artificial Sequence
  primer
ctaggtta

SEQ ID NO: 640 - DNA, 8 bp, Artificial Sequence
  primer
gcagatgt

SEQ ID NO: 641 - DNA, 8 bp, Artificial Sequence
  primer
agttcaga

SEQ ID NO: 642 - DNA, 8 bp, Artificial Sequence
  primer
ctccatca

SEQ ID NO: 643 - DNA, 8 bp, Artificial Sequence
  primer
tggttacg

SEQ ID NO: 644 - DNA, 8 bp, Artificial Sequence
  primer
acgtagca

SEQ ID NO: 645 - DNA, 8 bp, Artificial Sequence
  primer
ctcttcca

SEQ ID NO: 646 - DNA, 8 bp, Artificial Sequence
  primer
cgtcagat

SEQ ID NO: 647 - DNA, 8 bp, Artificial Sequence
  primer
tggatcat

SEQ ID NO: 648 - DNA, 8 bp, Artificial Sequence
  primer
atatcgac

SEQ ID NO: 649 - DNA, 8 bp, Artificial Sequence
  primer
ttgtggag

SEQ ID NO: 650 - DNA, 8 bp, Artificial Sequence
  primer
ttagagca

SEQ ID NO: 651 - DNA, 8 bp, Artificial Sequence
  primer
taactacc

SEQ ID NO: 652 - DNA, 8 bp, Artificial Sequence
  primer
ctatgagg

SEQ ID NO: 653 - DNA, 8 bp, Artificial Sequence
  primer
cttctcac

SEQ ID NO: 654 - DNA, 8 bp, Artificial Sequence
  primer
cgttctct

SEQ ID NO: 655 - DNA, 8 bp, Artificial Sequence
  primer
gtcactat

SEQ ID NO: 656 - DNA, 8 bp, Artificial Sequence
  primer
tcgttagc

SEQ ID NO: 657 - DNA, 8 bp, Artificial Sequence
  primer
atcgtgta

SEQ ID NO: 658 - DNA, 8 bp, Artificial Sequence
  primer
gagagcaa

SEQ ID NO: 659 - DNA, 8 bp, Artificial Sequence
  primer
agacgcaa

SEQ ID NO: 660 - DNA, 8 bp, Artificial Sequence
  primer
tccagtta

SEQ ID NO: 661 - DNA, 8 bp, Artificial Sequence
  primer
aatgccac

SEQ ID NO: 662 - DNA, 8 bp, Artificial Sequence
  primer
atcacgtg

SEQ ID NO: 663 - DNA, 8 bp, Artificial Sequence
  primer
actgtgca

SEQ ID NO: 664 - DNA, 8 bp, Artificial Sequence
  primer
tcactgca

SEQ ID NO: 665 - DNA, 8 bp, Artificial Sequence
  primer
gcatccaa

SEQ ID NO: 666 - DNA, 8 bp, Artificial Sequence
  primer
agcactat

SEQ ID NO: 667 - DNA, 8 bp, Artificial Sequence
  primer
cgaaggat

SEQ ID NO: 668 - DNA, 8 bp, Artificial Sequence
  primer
ccttgtgt

SEQ ID NO: 669 - DNA, 8 bp, Artificial Sequence
  primer
tgcggata

SEQ ID NO: 670 - DNA, 8 bp, Artificial Sequence
  primer
aggaatgg

SEQ ID NO: 671 - DNA, 8 bp, Artificial Sequence
  primer
atcgtaac

SEQ ID NO: 672 - DNA, 8 bp, Artificial Sequence
  primer
gaatgtct

SEQ ID NO: 673 - DNA, 9 bp, Artificial Sequence
  primer
ttgctacat

SEQ ID NO: 674 - DNA, 9 bp, Artificial Sequence
  primer
taacgtatg

SEQ ID NO: 675 - DNA, 9 bp, Artificial Sequence
  primer
cagtatgta

SEQ ID NO: 676 - DNA, 9 bp, Artificial Sequence
  primer
tcaataacg

SEQ ID NO: 677 - DNA, 9 bp, Artificial Sequence
  primer
cacacttat

SEQ ID NO: 678 - DNA, 9 bp, Artificial Sequence
  primer
gactgtaat

SEQ ID NO: 679 - DNA, 9 bp, Artificial Sequence
  primer
tatacactg

SEQ ID NO: 680 - DNA, 9 bp, Artificial Sequence
  primer
actgcatta

SEQ ID NO: 681 - DNA, 9 bp, Artificial Sequence
  primer
acattaagc

SEQ ID NO: 682 - DNA, 9 bp, Artificial Sequence
  primer
catattacg

SEQ ID NO: 683 - DNA, 9 bp, Artificial Sequence
  primer
atatctacg

SEQ ID NO: 684 - DNA, 9 bp, Artificial Sequence
  primer
agtaactgt

SEQ ID NO: 685 - DNA, 9 bp, Artificial Sequence
  primer
atgacgtta

SEQ ID NO: 686 - DNA, 9 bp, Artificial Sequence
  primer
attatgcga

SEQ ID NO: 687 - DNA, 9 bp, Artificial Sequence
  primer
agtatacac

SEQ ID NO: 688 - DNA, 9 bp, Artificial Sequence
  primer
ttagcgtta

SEQ ID NO: 689 - DNA, 9 bp, Artificial Sequence
  primer
tatgacact

SEQ ID NO: 690 - DNA, 9 bp, Artificial Sequence
  primer
attaacgct

SEQ ID NO: 691 - DNA, 9 bp, Artificial Sequence
  primer
taggacaat

SEQ ID NO: 692 - DNA, 9 bp, Artificial Sequence
  primer
aagacgtta

SEQ ID NO: 693 - DNA, 9 bp, Artificial Sequence
  primer
tataagcgt

SEQ ID NO: 694 - DNA, 9 bp, Artificial Sequence
  primer
atacctggc

SEQ ID NO: 695 - DNA, 9 bp, Artificial Sequence
  primer
ctcgagatc

SEQ ID NO: 696 - DNA, 9 bp, Artificial Sequence
  primer
atggtgagg

SEQ ID NO: 697 - DNA, 9 bp, Artificial Sequence
  primer
atgtcgacg

SEQ ID NO: 698 - DNA, 9 bp, Artificial Sequence
  primer
gacgtctga

SEQ ID NO: 699 - DNA, 9 bp, Artificial Sequence
  primer
tacactgcg

SEQ ID NO: 700 - DNA, 9 bp, Artificial Sequence
  primer
atcgtcagg

SEQ ID NO: 701 - DNA, 9 bp, Artificial Sequence
  primer
tgcacgtac

SEQ ID NO: 702 - DNA, 9 bp, Artificial Sequence
  primer
gtcgtgcat

SEQ ID NO: 703 - DNA, 9 bp, Artificial Sequence
  primer
gagtgttac

SEQ ID NO: 704 - DNA, 9 bp, Artificial Sequence
  primer
agactgtac

SEQ ID NO: 705 - DNA, 9 bp, Artificial Sequence
  primer
tgcgactta

SEQ ID NO: 706 - DNA, 9 bp, Artificial Sequence
  primer
tgtccgtaa

SEQ ID NO: 707 - DNA, 9 bp, Artificial Sequence
  primer
gtaatcgag

SEQ ID NO: 708 - DNA, 9 bp, Artificial Sequence
  primer
gtaccttag

SEQ ID NO: 709 - DNA, 9 bp, Artificial Sequence
  primer
atcacgtgt

SEQ ID NO: 710 - DNA, 9 bp, Artificial Sequence
  primer
acttagcgt

SEQ ID NO: 711 - DNA, 9 bp, Artificial Sequence
  primer
gtaatcgtg

SEQ ID NO: 712 - DNA, 9 bp, Artificial Sequence
  primer
atgccgtta

SEQ ID NO: 713 - DNA, 9 bp, Artificial Sequence
  primer
ataacgtgc

SEQ ID NO: 714 - DNA, 9 bp, Artificial Sequence
  primer
ctacgttgt

SEQ ID NO: 715 - DNA, 9 bp, Artificial Sequence
  primer
tatgacgca

SEQ ID NO: 716 - DNA, 9 bp, Artificial Sequence
  primer
ccgataaca

SEQ ID NO: 717 - DNA, 9 bp, Artificial Sequence
  primer
atgcgcata

SEQ ID NO: 718 - DNA, 9 bp, Artificial Sequence
  primer
gataagcgt

SEQ ID NO: 719 - DNA, 9 bp, Artificial Sequence
  primer
atatctgcg

SEQ ID NO: 720 - DNA, 9 bp, Artificial Sequence
  primer
acttagacg

SEQ ID NO: 721 - DNA, 9 bp, Artificial Sequence
  primer
atcaccgta

SEQ ID NO: 722 - DNA, 9 bp, Artificial Sequence
  primer
taagacacg

SEQ ID NO: 723 - DNA, 9 bp, Artificial Sequence
  primer
aatgccgta

SEQ ID NO: 724 - DNA, 9 bp, Artificial Sequence
  primer
aatcacgtg

SEQ ID NO: 725 - DNA, 9 bp, Artificial Sequence
  primer
tcgttagtc

SEQ ID NO: 726 - DNA, 9 bp, Artificial Sequence
  primer
catcatgtc

SEQ ID NO: 727 - DNA, 9 bp, Artificial Sequence
  primer
taagacggt

SEQ ID NO: 728 - DNA, 9 bp, Artificial Sequence
  primer
tgcatagtg

SEQ ID NO: 729 - DNA, 9 bp, Artificial Sequence
  primer
gagcgttat

SEQ ID NO: 730 - DNA, 9 bp, Artificial Sequence
  primer
tgccttaca

SEQ ID NO: 731 - DNA, 9 bp, Artificial Sequence
  primer
ttcgcgtta

SEQ ID NO: 732 - DNA, 9 bp, Artificial Sequence
  primer
gtgttaacg

SEQ ID NO: 733 - DNA, 9 bp, Artificial Sequence
  primer
gacactgaa

SEQ ID NO: 734 - DNA, 9 bp, Artificial Sequence
  primer
ctgttatcg

SEQ ID NO: 735 - DNA, 9 bp, Artificial Sequence
  primer
ggtcgttat

SEQ ID NO: 736 - DNA, 9 bp, Artificial Sequence
  primer
cgagagtat

SEQ ID NO: 737 - DNA, 9 bp, Artificial Sequence
  primer
atacagtcc

SEQ ID NO: 738 - DNA, 9 bp, Artificial Sequence
  primer
aattcacgc

SEQ ID NO: 739 - DNA, 9 bp, Artificial Sequence
  primer
tatgtgcac

SEQ ID NO: 740 - DNA, 9 bp, Artificial Sequence
  primer
gatgacgta

SEQ ID NO: 741 - DNA, 9 bp, Artificial Sequence
  primer
gatgcgata

SEQ ID NO: 742 - DNA, 9 bp, Artificial Sequence
  primer
gagcgatta

SEQ ID NO: 743 - DNA, 9 bp, Artificial Sequence
  primer
tgtcacaga

SEQ ID NO: 744 - DNA, 9 bp, Artificial Sequence
  primer
tactaaccg

SEQ ID NO: 745 - DNA, 9 bp, Artificial Sequence
  primer
cataacgag

SEQ ID NO: 746 - DNA, 9 bp, Artificial Sequence
  primer
cgtatacct

SEQ ID NO: 747 - DNA, 9 bp, Artificial Sequence
  primer
tatcacgtg

SEQ ID NO: 748 - DNA, 9 bp, Artificial Sequence
  primer
gaacgttac

SEQ ID NO: 749 - DNA, 9 bp, Artificial Sequence
  primer
gtcgtatac

SEQ ID NO: 750 - DNA, 9 bp, Artificial Sequence
  primer
atgtcgaca

SEQ ID NO: 751 - DNA, 9 bp, Artificial Sequence
  primer
atacagcac

SEQ ID NO: 752 - DNA, 9 bp, Artificial Sequence
  primer
tacttacgc

SEQ ID NO: 753 - DNA, 9 bp, Artificial Sequence
  primer
aactacggt

SEQ ID NO: 754 - DNA, 9 bp, Artificial Sequence
  primer
tagaacggt

SEQ ID NO: 755 - DNA, 9 bp, Artificial Sequence
  primer
gaatgtcac

SEQ ID NO: 756 - DNA, 9 bp, Artificial Sequence
  primer
tgtacgtct

SEQ ID NO: 757 - DNA, 9 bp, Artificial Sequence
  primer
aacattgcg

SEQ ID NO: 758 - DNA, 9 bp, Artificial Sequence
  primer
ttgaacgct

SEQ ID NO: 759 - DNA, 9 bp, Artificial Sequence
  primer
aatcaggac

SEQ ID NO: 760 - DNA, 9 bp, Artificial Sequence
  primer
attcgcaca

SEQ ID NO: 761 - DNA, 9 bp, Artificial Sequence
  primer
ccatgtact

SEQ ID NO: 762 - DNA, 9 bp, Artificial Sequence
  primer
tgtcctgtt

SEQ ID NO: 763 - DNA, 9 bp, Artificial Sequence
  primer
taattgcgc

SEQ ID NO: 764 - DNA, 9 bp, Artificial Sequence
  primer
gatagtgtg

SEQ ID NO: 765 - DNA, 9 bp, Artificial Sequence
  primer
atagacgca

SEQ ID NO: 766 - DNA, 9 bp, Artificial Sequence
  primer
tgtaccgtt

SEQ ID NO: 767 - DNA, 9 bp, Artificial Sequence
  primer
attgtcgca

SEQ ID NO: 768 - DNA, 9 bp, Artificial Sequence
  primer
gtcacgtaa

SEQ ID NO: 769 - DNA, 11 bp, Artificial Sequence
  primer
ttacactatgc

SEQ ID NO: 770 - DNA, 11 bp, Artificial Sequence
  primer
gcgatagtcgt

SEQ ID NO: 771 - DNA, 11 bp, Artificial Sequence
  primer
ctattcacagt

SEQ ID NO: 772 - DNA, 11 bp, Artificial Sequence
  primer
agagtcactgt

SEQ ID NO: 773 - DNA, 11 bp, Artificial Sequence
  primer
agagtcgaagc

SEQ ID NO: 774 - DNA, 11 bp, Artificial Sequence
  primer
ctgaatatgtg

SEQ ID NO: 775 - DNA, 11 bp, Artificial Sequence
  primer
actccacagga

SEQ ID NO: 776 - DNA, 11 bp, Artificial Sequence
  primer
atcctcgtaag

SEQ ID NO: 777 - DNA, 11 bp, Artificial Sequence
  primer
taccatcgcct

SEQ ID NO: 778 - DNA, 11 bp, Artificial Sequence
  primer
aacgcctataa

SEQ ID NO: 779 - DNA, 11 bp, Artificial Sequence
  primer
ctgtcgaactt

SEQ ID NO: 780 - DNA, 11 bp, Artificial Sequence
  primer
tcagatgtccg

SEQ ID NO: 781 - DNA, 11 bp, Artificial Sequence
  primer
ctgcttatcgt

SEQ ID NO: 782 - DNA, 11 bp, Artificial Sequence
  primer
acattcgcaca

SEQ ID NO: 783 - DNA, 11 bp, Artificial Sequence
  primer
ccttaatgcat

SEQ ID NO: 784 - DNA, 11 bp, Artificial Sequence
  primer
ggctagctact

SEQ ID NO: 785 - DNA, 11 bp, Artificial Sequence
  primer
ttccagttggc

SEQ ID NO: 786 - DNA, 11 bp, Artificial Sequence
  primer
gagtcacaagg

SEQ ID NO: 787 - DNA, 11 bp, Artificial Sequence
  primer
cagaaggttca

SEQ ID NO: 788 - DNA, 11 bp, Artificial Sequence
  primer
tcaacgtgcag

SEQ ID NO: 789 - DNA, 11 bp, Artificial Sequence
  primer
caagcttacta

SEQ ID NO: 790 - DNA, 11 bp, Artificial Sequence
  primer
agaactcgttg

SEQ ID NO: 791 - DNA, 11 bp, Artificial Sequence
  primer
ccgatacagag

SEQ ID NO: 792 - DNA, 11 bp, Artificial Sequence
  primer
gtacgctgatc

SEQ ID NO: 793 - DNA, 11 bp, Artificial Sequence
  primer
tcctcagtgaa

SEQ ID NO: 794 - DNA, 11 bp, Artificial Sequence
  primer
gagccaacatt

SEQ ID NO: 795 - DNA, 11 bp, Artificial Sequence
  primer
gagatcgatgg

SEQ ID NO: 796 - DNA, 11 bp, Artificial Sequence
  primer
atcgtcagctg

SEQ ID NO: 797 - DNA, 11 bp, Artificial Sequence
  primer
gaagcacacgt

SEQ ID NO: 798 - DNA, 11 bp, Artificial Sequence
  primer
atcacgcaacc

SEQ ID NO: 799 - DNA, 11 bp, Artificial Sequence
  primer
tcgaatagtcg

SEQ ID NO: 800 - DNA, 11 bp, Artificial Sequence
  primer
tattaccgtct

SEQ ID NO: 801 - DNA, 11 bp, Artificial Sequence
  primer
cagtcacgaca

SEQ ID NO: 802 - DNA, 11 bp, Artificial Sequence
  primer
ttactcgacgt

SEQ ID NO: 803 - DNA, 11 bp, Artificial Sequence
  primer
gcaatgttgaa

SEQ ID NO: 804 - DNA, 11 bp, Artificial Sequence
  primer
gacacgagcaa

SEQ ID NO: 805 - DNA, 11 bp, Artificial Sequence
  primer
cgagattacaa

SEQ ID NO: 806 - DNA, 11 bp, Artificial Sequence
  primer
taccgactaca

SEQ ID NO: 807 - DNA, 11 bp, Artificial Sequence
  primer
accgttgccat

SEQ ID NO: 808 - DNA, 11 bp, Artificial Sequence
  primer
atgtaatcgcc

SEQ ID NO: 809 - DNA, 11 bp, Artificial Sequence
  primer
aagcctgatgt

SEQ ID NO: 810 - DNA, 11 bp, Artificial Sequence
  primer
aagtaacgtgg

SEQ ID NO: 811 - DNA, 11 bp, Artificial Sequence
  primer
gtagaggttgg

SEQ ID NO: 812 - DNA, 11 bp, Artificial Sequence
  primer
ctcttgcctca

SEQ ID NO: 813 - DNA, 11 bp, Artificial Sequence
  primer
atcgtgaagtg

SEQ ID NO: 814 - DNA, 11 bp, Artificial Sequence
  primer
accagcactat

SEQ ID NO: 815 - DNA, 11 bp, Artificial Sequence
  primer
caccagaatgt

SEQ ID NO: 816 - DNA, 11 bp, Artificial Sequence
  primer
gagtgaacaac

SEQ ID NO: 817 - DNA, 11 bp, Artificial Sequence
  primer
taacgttacgc

SEQ ID NO: 818 - DNA, 11 bp, Artificial Sequence
  primer
cttggatcttg

SEQ ID NO: 819 - DNA, 11 bp, Artificial Sequence
  primer
gttccaacgtt

SEQ ID NO: 820 - DNA, 11 bp, Artificial Sequence
  primer
caaggaccgta

SEQ ID NO: 821 - DNA, 11 bp, Artificial Sequence
  primer
gacttcacgca

SEQ ID NO: 822 - DNA, 11 bp, Artificial Sequence
  primer
cacactactgg

SEQ ID NO: 823 - DNA, 11 bp, Artificial Sequence
  primer
tcagatgaatc

SEQ ID NO: 824 - DNA, 11 bp, Artificial Sequence
  primer
tatggatctgg

SEQ ID NO: 825 - DNA, 11 bp, Artificial Sequence
  primer
tcttaggtgtg

SEQ ID NO: 826 - DNA, 11 bp, Artificial Sequence
  primer
tgtcagcgtca

SEQ ID NO: 827 - DNA, 11 bp, Artificial Sequence
  primer
gtctaggacag

SEQ ID NO: 828 - DNA, 11 bp, Artificial Sequence
  primer
gcctcttcata

SEQ ID NO: 829 - DNA, 11 bp, Artificial Sequence
  primer
agaagtgttac

SEQ ID NO: 830 - DNA, 11 bp, Artificial Sequence
  primer
catgaggcttg

SEQ ID NO: 831 - DNA, 11 bp, Artificial Sequence
  primer
tggattgctca

SEQ ID NO: 832 - DNA, 11 bp, Artificial Sequence
  primer
atctacctaag

SEQ ID NO: 833 - DNA, 11 bp, Artificial Sequence
  primer
atgagcagtga

SEQ ID NO: 834 - DNA, 11 bp, Artificial Sequence
  primer
ccaggagatac

SEQ ID NO: 835 - DNA, 11 bp, Artificial Sequence
  primer
ccgttatactt

SEQ ID NO: 836 - DNA, 11 bp, Artificial Sequence
  primer
ctcagtacaag

SEQ ID NO: 837 - DNA, 11 bp, Artificial Sequence
  primer
ggtgatcgtag

SEQ ID NO: 838 - DNA, 11 bp, Artificial Sequence
  primer
cgaacgagaca

SEQ ID NO: 839 - DNA, 11 bp, Artificial Sequence
  primer
actacgagctt

SEQ ID NO: 840 - DNA, 11 bp, Artificial Sequence
  primer
ttgccacagca

SEQ ID NO: 841 - DNA, 11 bp, Artificial Sequence
  primer
gtcaactctac

SEQ ID NO: 842 - DNA, 11 bp, Artificial Sequence
  primer
tggactgtgtc

SEQ ID NO: 843 - DNA, 11 bp, Artificial Sequence
  primer
ggaatggactt

SEQ ID NO: 844 - DNA, 11 bp, Artificial Sequence
  primer
cgagaacataa

SEQ ID NO: 845 - DNA, 11 bp, Artificial Sequence
  primer
acctggtcagt

SEQ ID NO: 846 - DNA, 11 bp, Artificial Sequence
  primer
cgaacgacaca

SEQ ID NO: 847 - DNA, 11 bp, Artificial Sequence
  primer
agtctagccat

SEQ ID NO: 848 - DNA, 11 bp, Artificial Sequence
  primer
aggcctagatg

SEQ ID NO: 849 - DNA, 11 bp, Artificial Sequence
  primer
ggtgcgttagt

SEQ ID NO: 850 - DNA, 11 bp, Artificial Sequence
  primer
attgtgtccga

SEQ ID NO: 851 - DNA, 11 bp, Artificial Sequence
  primer
gcagacattaa

SEQ ID NO: 852 - DNA, 11 bp, Artificial Sequence
  primer
attggctcatg

SEQ ID NO: 853 - DNA, 11 bp, Artificial Sequence
  primer
gaggttacatg

SEQ ID NO: 854 - DNA, 11 bp, Artificial Sequence
  primer
cctataggacc

SEQ ID NO: 855 - DNA, 11 bp, Artificial Sequence
  primer
ttagacggtct

SEQ ID NO: 856 - DNA, 11 bp, Artificial Sequence
  primer
gattgacgcac

SEQ ID NO: 857 - DNA, 11 bp, Artificial Sequence
  primer
aagacacctcg

SEQ ID NO: 858 - DNA, 11 bp, Artificial Sequence
  primer
tcgaataatcg

SEQ ID NO: 859 - DNA, 11 bp, Artificial Sequence
  primer
tctatgtcgga

SEQ ID NO: 860 - DNA, 11 bp, Artificial Sequence
  primer
tcgcatgaacc

SEQ ID NO: 861 - DNA, 11 bp, Artificial Sequence
  primer
tgttatgtctc

SEQ ID NO: 862 - DNA, 11 bp, Artificial Sequence
  primer
tggatcctaca

SEQ ID NO: 863 - DNA, 11 bp, Artificial Sequence
  primer
atcgttcagcc

SEQ ID NO: 864 - DNA, 11 bp, Artificial Sequence
  primer
taccgcaagca

SEQ ID NO: 865 - DNA, 12 bp, Artificial Sequence
  primer
gctgttgaaccg

SEQ ID NO: 866 - DNA, 12 bp, Artificial Sequence
  primer
atactccgagat

SEQ ID NO: 867 - DNA, 12 bp, Artificial Sequence
  primer
cttaaggagcgc

SEQ ID NO: 868 - DNA, 12 bp, Artificial Sequence
  primer
tatactacaagc

SEQ ID NO: 869 - DNA, 12 bp, Artificial Sequence
  primer
tagtggtcgtca

SEQ ID NO: 870 - DNA, 12 bp, Artificial Sequence
  primer
gtgcttcaggag

SEQ ID NO: 871 - DNA, 12 bp, Artificial Sequence
  primer
gacgcatacctc

SEQ ID NO: 872 - DNA, 12 bp, Artificial Sequence
  primer
cctacctgtgga

SEQ ID NO: 873 - DNA, 12 bp, Artificial Sequence
  primer
gcggtcacatat

SEQ ID NO: 874 - DNA, 12 bp, Artificial Sequence
  primer
ctgcattcacga

SEQ ID NO: 875 - DNA, 12 bp, Artificial Sequence
  primer
tggatccttcat

SEQ ID NO: 876 - DNA, 12 bp, Artificial Sequence
  primer
ttgtgctggact

SEQ ID NO: 877 - DNA, 12 bp, Artificial Sequence
  primer
attgagagctat

SEQ ID NO: 878 - DNA, 12 bp, Artificial Sequence
  primer
tcgctaatgtag

SEQ ID NO: 879 - DNA, 12 bp, Artificial Sequence
  primer
ctactggcacaa

SEQ ID NO: 880 - DNA, 12 bp, Artificial Sequence
  primer
agagccagtcgt

SEQ ID NO: 881 - DNA, 12 bp, Artificial Sequence
  primer
aatactggctaa

SEQ ID NO: 882 - DNA, 12 bp, Artificial Sequence
  primer
ctgcatgcataa

SEQ ID NO: 883 - DNA, 12 bp, Artificial Sequence
  primer
ttgtcacaactc

SEQ ID NO: 884 - DNA, 12 bp, Artificial Sequence
  primer
tgctaactctcc

SEQ ID NO: 885 - DNA, 12 bp, Artificial Sequence
  primer
tctctagttcgg

SEQ ID NO: 886 - DNA, 12 bp, Artificial Sequence
  primer
ttacgtccgcaa

SEQ ID NO: 887 - DNA, 12 bp, Artificial Sequence
  primer
gtgttgctacca

SEQ ID NO: 888 - DNA, 12 bp, Artificial Sequence
  primer
cgcatgtatgcc

SEQ ID NO: 889 - DNA, 12 bp, Artificial Sequence
  primer
cctgttctgatt

SEQ ID NO: 890 - DNA, 12 bp, Artificial Sequence
  primer
taagatgcttga

SEQ ID NO: 891 - DNA, 12 bp, Artificial Sequence
  primer
atatatctcagc

SEQ ID NO: 892 - DNA, 12 bp, Artificial Sequence
  primer
ttcctcgtggtt

SEQ ID NO: 893 - DNA, 12 bp, Artificial Sequence
  primer
atgtcgatctag

SEQ ID NO: 894 - DNA, 12 bp, Artificial Sequence
  primer
catccactaatc

SEQ ID NO: 895 - DNA, 12 bp, Artificial Sequence
  primer
gcctctggtaac

SEQ ID NO: 896 - DNA, 12 bp, Artificial Sequence
  primer
agtcaagagatt

SEQ ID NO: 897 - DNA, 12 bp, Artificial Sequence
  primer
actgaggcgttc

SEQ ID NO: 898 - DNA, 12 bp, Artificial Sequence
  primer
taaggctgacat

SEQ ID NO: 899 - DNA, 12 bp, Artificial Sequence
  primer
agttcgcataca

SEQ ID NO: 900 - DNA, 12 bp, Artificial Sequence
  primer
gcagaattgcga

SEQ ID NO: 901 - DNA, 12 bp, Artificial Sequence
  primer
ggttatgaagaa

SEQ ID NO: 902 - DNA, 12 bp, Artificial Sequence
  primer
agaagtcgcctc

SEQ ID NO: 903 - DNA, 12 bp, Artificial Sequence
  primer
ttcgcgttattg

SEQ ID NO: 904 - DNA, 12 bp, Artificial Sequence
  primer
tacctggtcggt

SEQ ID NO: 905 - DNA, 12 bp, Artificial Sequence
  primer
ggttaccgagga

SEQ ID NO: 906 - DNA, 12 bp, Artificial Sequence
  primer
acacacttctag

SEQ ID NO: 907 - DNA, 12 bp, Artificial Sequence
  primer
ggaagtgattaa

SEQ ID NO: 908 - DNA, 12 bp, Artificial Sequence
  primer
tccatcagataa

SEQ ID NO: 909 - DNA, 12 bp, Artificial Sequence
  primer
tgtctgtatcat

SEQ ID NO: 910 - DNA, 12 bp, Artificial Sequence
  primer
aattggctatag

SEQ ID NO: 911 - DNA, 12 bp, Artificial Sequence
  primer
acgtcggaaggt

SEQ ID NO: 912 - DNA, 12 bp, Artificial Sequence
  primer
aggcatccgttg

SEQ ID NO: 913 - DNA, 12 bp, Artificial Sequence
  primer
accgtcgcttga

SEQ ID NO: 914 - DNA, 12 bp, Artificial Sequence
  primer
taccgtcaagtg

SEQ ID NO: 915 - DNA, 12 bp, Artificial Sequence
  primer
ctcgatatagtt

SEQ ID NO: 916 - DNA, 12 bp, Artificial Sequence
  primer
cgtcaacgtggt

SEQ ID NO: 917 - DNA, 12 bp, Artificial Sequence
  primer
tagtcaacgtag

SEQ ID NO: 918 - DNA, 12 bp, Artificial Sequence
  primer
tgagtaggtcag

SEQ ID NO: 919 - DNA, 12 bp, Artificial Sequence
  primer
cttggcatgtac

SEQ ID NO: 920 - DNA, 12 bp, Artificial Sequence
  primer
tgccgagacttc

SEQ ID NO: 921 - DNA, 12 bp, Artificial Sequence
  primer
ctaagacttaag

SEQ ID NO: 922 - DNA, 12 bp, Artificial Sequence
  primer
ttctcgtgtgcg

SEQ ID NO: 923 - DNA, 12 bp, Artificial Sequence
  primer
cacctgcacgat

SEQ ID NO: 924 - DNA, 12 bp, Artificial Sequence
  primer
attaagcctaag

SEQ ID NO: 925 - DNA, 12 bp, Artificial Sequence
  primer
ggtggaaccatg

SEQ ID NO: 926 - DNA, 12 bp, Artificial Sequence
  primer
actaacgcgact

SEQ ID NO: 927 - DNA, 12 bp, Artificial Sequence
  primer
cagttgtgctat

SEQ ID NO: 928 - DNA, 12 bp, Artificial Sequence
  primer
acgctgttagca

SEQ ID NO: 929 - DNA, 12 bp, Artificial Sequence
  primer
gtcaacgctaag

SEQ ID NO: 930 - DNA, 12 bp, Artificial Sequence
  primer
agcttaggtatg

SEQ ID NO: 931 - DNA, 12 bp, Artificial Sequence
  primer
cgcaggacgatt

SEQ ID NO: 932 - DNA, 12 bp, Artificial Sequence
  primer
aaccggctgtct

SEQ ID NO: 933 - DNA, 12 bp, Artificial Sequence
  primer
gttgctcacgtg

SEQ ID NO: 934 - DNA, 12 bp, Artificial Sequence
  primer
gaatcttccgcg

SEQ ID NO: 935 - DNA, 12 bp, Artificial Sequence
  primer
agagcgtacacg

SEQ ID NO: 936 - DNA, 12 bp, Artificial Sequence
  primer
aaggctaatgtc

SEQ ID NO: 937 - DNA, 12 bp, Artificial Sequence
  primer
tctatgtagacg

SEQ ID NO: 938 - DNA, 12 bp, Artificial Sequence
  primer
agacggtctagt

SEQ ID NO: 939 - DNA, 12 bp, Artificial Sequence
  primer
ttggtcacacgc

SEQ ID NO: 940 - DNA, 12 bp, Artificial Sequence
  primer
gtcgatatatgg

SEQ ID NO: 941 - DNA, 12 bp, Artificial Sequence
  primer
aacatggatacg

SEQ ID NO: 942 - DNA, 12 bp, Artificial Sequence
  primer
ttcgcagttcct

SEQ ID NO: 943 - DNA, 12 bp, Artificial Sequence
  primer
cgcatgttgtgc

SEQ ID NO: 944 - DNA, 12 bp, Artificial Sequence
  primer
tgttaagttgga

SEQ ID NO: 945 - DNA, 12 bp, Artificial Sequence
  primer
caagtgtgatga

SEQ ID NO: 946 - DNA, 12 bp, Artificial Sequence
  primer
ctggtaccacgt

SEQ ID NO: 947 - DNA, 12 bp, Artificial Sequence
  primer
cgctaggatcac

SEQ ID NO: 948 - DNA, 12 bp, Artificial Sequence
  primer
tgctcattacgg

SEQ ID NO: 949 - DNA, 12 bp, Artificial Sequence
  primer
tgctcagtaaca

SEQ ID NO: 950 - DNA, 12 bp, Artificial Sequence
  primer
acgatcatagcc

SEQ ID NO: 951 - DNA, 12 bp, Artificial Sequence
  primer
acgatacgtgga

SEQ ID NO: 952 - DNA, 12 bp, Artificial Sequence
  primer
gttcgatgatgg

SEQ ID NO: 953 - DNA, 12 bp, Artificial Sequence
  primer
aagagctgtgcc

SEQ ID NO: 954 - DNA, 12 bp, Artificial Sequence
  primer
ggttggatcaac

SEQ ID NO: 955 - DNA, 12 bp, Artificial Sequence
  primer
gcgcgcttatga

SEQ ID NO: 956 - DNA, 12 bp, Artificial Sequence
  primer
cgtcgatcatca

SEQ ID NO: 957 - DNA, 12 bp, Artificial Sequence
  primer
gagactgcactc

SEQ ID NO: 958 - DNA, 12 bp, Artificial Sequence
  primer
gatagatcgcat

SEQ ID NO: 959 - DNA, 12 bp, Artificial Sequence
  primer
ggccatcatcag

SEQ ID NO: 960 - DNA, 12 bp, Artificial Sequence
  primer
ggtgttccactg

SEQ ID NO: 961 - DNA, 14 bp, Artificial Sequence
  primer
agctatacagaggt

SEQ ID NO: 962 - DNA, 14 bp, Artificial Sequence
  primer
aggccgttctgtct

SEQ ID NO: 963 - DNA, 14 bp, Artificial Sequence
  primer
cattggtctgctat

SEQ ID NO: 964 - DNA, 14 bp, Artificial Sequence
  primer
ctacatacgcgcca

SEQ ID NO: 965 - DNA, 14 bp, Artificial Sequence
  primer
gcttaacggcgctt

SEQ ID NO: 966 - DNA, 14 bp, Artificial Sequence
  primer
tacgatactccacc

SEQ ID NO: 967 - DNA, 14 bp, Artificial Sequence
  primer
accggcataagaag

SEQ ID NO: 968 - DNA, 14 bp, Artificial Sequence
  primer
ggatgcttcgataa

SEQ ID NO: 969 - DNA, 14 bp, Artificial Sequence
  primer
gtgtacctgaatgt

SEQ ID NO: 970 - DNA, 14 bp, Artificial Sequence
  primer
cgcggatacacaga

SEQ ID NO: 971 - DNA, 14 bp, Artificial Sequence
  primer
ttccacggcactgt

SEQ ID NO: 972 - DNA, 14 bp, Artificial Sequence
  primer
tagccaggcaacaa

SEQ ID NO: 973 - DNA, 14 bp, Artificial Sequence
  primer
agcgtcaacacgta

SEQ ID NO: 974 - DNA, 14 bp, Artificial Sequence
  primer
taacgctactcgcg

SEQ ID NO: 975 - DNA, 14 bp, Artificial Sequence
  primer
tagatagacgatct

SEQ ID NO: 976 - DNA, 14 bp, Artificial Sequence
  primer
actcttgcaatgct

SEQ ID NO: 977 - DNA, 14 bp, Artificial Sequence
  primer
actcggttaggtcg

SEQ ID NO: 978 - DNA, 14 bp, Artificial Sequence
  primer
cattatctacgcat

SEQ ID NO: 979 - DNA, 14 bp, Artificial Sequence
  primer
cacaccggcgatta

SEQ ID NO: 980 - DNA, 14 bp, Artificial Sequence
  primer
tacgcagtactgtg

SEQ ID NO: 981 - DNA, 14 bp, Artificial Sequence
  primer
caagcgcgtgaatg

SEQ ID NO: 982 - DNA, 14 bp, Artificial Sequence
  primer
gaatggactgacga

SEQ ID NO: 983 - DNA, 14 bp, Artificial Sequence
  primer
ctagcgctgaagtt

SEQ ID NO: 984 - DNA, 14 bp, Artificial Sequence
  primer
tgcggcagaccaat

SEQ ID NO: 985 - DNA, 14 bp, Artificial Sequence
  primer
aaggcatagagatt

SEQ ID NO: 986 - DNA, 14 bp, Artificial Sequence
  primer
ttctcctcgccatg

SEQ ID NO: 987 - DNA, 14 bp, Artificial Sequence
  primer
tcattggtcgtgaa

SEQ ID NO: 988 - DNA, 14 bp, Artificial Sequence
  primer
attacgctatacga

SEQ ID NO: 989 - DNA, 14 bp, Artificial Sequence
  primer
atgatcctccacgg

SEQ ID NO: 990 - DNA, 14 bp, Artificial Sequence
  primer
cgtcgttagtaatc

SEQ ID NO: 991 - DNA, 14 bp, Artificial Sequence
  primer
tgcacatagtctca

SEQ ID NO: 992 - DNA, 14 bp, Artificial Sequence
  primer
gtcaaggagtcacg

SEQ ID NO: 993 - DNA, 14 bp, Artificial Sequence
  primer
ggttggaatcttgc

SEQ ID NO: 994 - DNA, 14 bp, Artificial Sequence
  primer
catcggtgcactca

SEQ ID NO: 995 - DNA, 14 bp, Artificial Sequence
  primer
aatgcactagacgt

SEQ ID NO: 996 - DNA, 14 bp, Artificial Sequence
  primer
tacagtcaggctcg

SEQ ID NO: 997 - DNA, 14 bp, Artificial Sequence
  primer
agagaagcttagcc

SEQ ID NO: 998 - DNA, 14 bp, Artificial Sequence
  primer
ccataggatcgtat

SEQ ID NO: 999 - DNA, 14 bp, Artificial Sequence
  primer
ttgtgctacacctg

SEQ ID NO: 1000 - DNA, 14 bp, Artificial Sequence
  primer
ctccagtaatacta

SEQ ID NO: 1001 - DNA, 14 bp, Artificial Sequence
  primer
tgatgccgatgtgg

SEQ ID NO: 1002 - DNA, 14 bp, Artificial Sequence
  primer
gtcataccgcttaa

SEQ ID NO: 1003 - DNA, 14 bp, Artificial Sequence
  primer
acgttctcttgaga

SEQ ID NO: 1004 - DNA, 14 bp, Artificial Sequence
  primer
cagccatatcgtgt

SEQ ID NO: 1005 - DNA, 14 bp, Artificial Sequence
  primer
ttgaacgtagcaat

SEQ ID NO: 1006 - DNA, 14 bp, Artificial Sequence
  primer
acaatcgcggtaat

SEQ ID NO: 1007 - DNA, 14 bp, Artificial Sequence
  primer
gttcctgtagatcc

SEQ ID NO: 1008 - DNA, 14 bp, Artificial Sequence
  primer
agagccttacggca

SEQ ID NO: 1009 - DNA, 14 bp, Artificial Sequence
  primer
aatatggcgccacc

SEQ ID NO: 1010 - DNA, 14 bp, Artificial Sequence
  primer
accatataggttcg

SEQ ID NO: 1011 - DNA, 14 bp, Artificial Sequence
  primer
atgcaccacagctg

SEQ ID NO: 1012 - DNA, 14 bp, Artificial Sequence
  primer
ctactattgaacag

SEQ ID NO: 1013 - DNA, 14 bp, Artificial Sequence
  primer
tgccatcactctag

SEQ ID NO: 1014 - DNA, 14 bp, Artificial Sequence
  primer
gcgaacgagaatcg

SEQ ID NO: 1015 - DNA, 14 bp, Artificial Sequence
  primer
gaatcaaggagacc

SEQ ID NO: 1016 - DNA, 14 bp, Artificial Sequence
  primer
caacatctatgcag

SEQ ID NO: 1017 - DNA, 14 bp, Artificial Sequence
  primer
caatccgtcatgga

SEQ ID NO: 1018 - DNA, 14 bp, Artificial Sequence
  primer
agctcttagccata

SEQ ID NO: 1019 - DNA, 14 bp, Artificial Sequence
  primer
aacaaggcaactgg

SEQ ID NO: 1020 - DNA, 14 bp, Artificial Sequence
  primer
gtcgtcgctcctat

SEQ ID NO: 1021 - DNA, 14 bp, Artificial Sequence
  primer
gtcatcattagatg

SEQ ID NO: 1022 - DNA, 14 bp, Artificial Sequence
  primer
gcactaagtagcag

SEQ ID NO: 1023 - DNA, 14 bp, Artificial Sequence
  primer
accttaccggacct

SEQ ID NO: 1024 - DNA, 14 bp, Artificial Sequence
  primer
gctcaggtatgtca

SEQ ID NO: 1025 - DNA, 14 bp, Artificial Sequence
  primer
tgtcacgagttagt

SEQ ID NO: 1026 - DNA, 14 bp, Artificial Sequence
  primer
cagatgacttacgt

SEQ ID NO: 1027 - DNA, 14 bp, Artificial Sequence
  primer
gaagtagcgattga

SEQ ID NO: 1028 - DNA, 14 bp, Artificial Sequence
  primer
gcaggcaatctgta

SEQ ID NO: 1029 - DNA, 14 bp, Artificial Sequence
  primer
ccttatacaacaag

SEQ ID NO: 1030 - DNA, 14 bp, Artificial Sequence
  primer
ccttagattgattg

SEQ ID NO: 1031 - DNA, 14 bp, Artificial Sequence
  primer
agccacgagtgata

SEQ ID NO: 1032 - DNA, 14 bp, Artificial Sequence
  primer
ggatgactcgtgac

SEQ ID NO: 1033 - DNA, 14 bp, Artificial Sequence
  primer
cttcgttcgccatt

SEQ ID NO: 1034 - DNA, 14 bp, Artificial Sequence
  primer
tcttgcgtattgat

SEQ ID NO: 1035 - DNA, 14 bp, Artificial Sequence
  primer
cttaacgtggtggc

SEQ ID NO: 1036 - DNA, 14 bp, Artificial Sequence
  primer
tgctgttacggaag

SEQ ID NO: 1037 - DNA, 14 bp, Artificial Sequence
  primer
ctgaattagttctc

SEQ ID NO: 1038 - DNA, 14 bp, Artificial Sequence
  primer
cctccaagtacaga

SEQ ID NO: 1039 - DNA, 14 bp, Artificial Sequence
  primer
ctggtaattcgcgg

SEQ ID NO: 1040 - DNA, 14 bp, Artificial Sequence
  primer
cgactgcaatctgg

SEQ ID NO: 1041 - DNA, 14 bp, Artificial Sequence
  primer
tggatcgcgattgg

SEQ ID NO: 1042 - DNA, 14 bp, Artificial Sequence
  primer
cgactattcctgcg

SEQ ID NO: 1043 - DNA, 14 bp, Artificial Sequence
  primer
caagtaggtccgtc

SEQ ID NO: 1044 - DNA, 14 bp, Artificial Sequence
  primer
agtaatcagtgttc

SEQ ID NO: 1045 - DNA, 14 bp, Artificial Sequence
  primer
ttattctcactacg

SEQ ID NO: 1046 - DNA, 14 bp, Artificial Sequence
  primer
catgtcttcttcgt

SEQ ID NO: 1047 - DNA, 14 bp, Artificial Sequence
  primer
aggcacataccatc

SEQ ID NO: 1048 - DNA, 14 bp, Artificial Sequence
  primer
aggttagaggatgt

SEQ ID NO: 1049 - DNA, 14 bp, Artificial Sequence
  primer
caactggcaagtgc

SEQ ID NO: 1050 - DNA, 14 bp, Artificial Sequence
  primer
cgctcacatagagg

SEQ ID NO: 1051 - DNA, 14 bp, Artificial Sequence
  primer
gcaatgtcgagatc

SEQ ID NO: 1052 - DNA, 14 bp, Artificial Sequence
  primer
gttctgtggtgctc

SEQ ID NO: 1053 - DNA, 14 bp, Artificial Sequence
  primer
aagtgatcagacta

SEQ ID NO: 1054 - DNA, 14 bp, Artificial Sequence
  primer
attgaaggattcca

SEQ ID NO: 1055 - DNA, 14 bp, Artificial Sequence
  primer
acgccatgctacta

SEQ ID NO: 1056 - DNA, 14 bp, Artificial Sequence
  primer
ctgaagatgtctgc

SEQ ID NO: 1057 - DNA, 16 bp, Artificial Sequence
  primer
gacaatctctgccgat

SEQ ID NO: 1058 - DNA, 16 bp, Artificial Sequence
  primer
ggtccgcctaatgtaa

SEQ ID NO: 1059 - DNA, 16 bp, Artificial Sequence
  primer
agccacaggcaattcc

SEQ ID NO: 1060 - DNA, 16 bp, Artificial Sequence
  primer
atctcaagttctcaac

SEQ ID NO: 1061 - DNA, 16 bp, Artificial Sequence
  primer
tgtaacgcatacgacg

SEQ ID NO: 1062 - DNA, 16 bp, Artificial Sequence
  primer
tatctcgaataccagc

SEQ ID NO: 1063 - DNA, 16 bp, Artificial Sequence
  primer
accgcaacacaggcaa

SEQ ID NO: 1064 - DNA, 16 bp, Artificial Sequence
  primer
ggccagtaacatgact

SEQ ID NO: 1065 - DNA, 16 bp, Artificial Sequence
  primer
gtgaacagttaaggtg

SEQ ID NO: 1066 - DNA, 16 bp, Artificial Sequence
  primer
ccaggatccgtattgc

SEQ ID NO: 1067 - DNA, 16 bp, Artificial Sequence
  primer
gacctagcactagacc

SEQ ID NO: 1068 - DNA, 16 bp, Artificial Sequence
  primer
cgccatcctattcacg

SEQ ID NO: 1069 - DNA, 16 bp, Artificial Sequence
  primer
aagtgcagtaatggaa

SEQ ID NO: 1070 - DNA, 16 bp, Artificial Sequence
  primer
tcaacgcgttcgtcta

SEQ ID NO: 1071 - DNA, 16 bp, Artificial Sequence
  primer
agcggccactatctaa

SEQ ID NO: 1072 - DNA, 16 bp, Artificial Sequence
  primer
ctcggcgccatataga

SEQ ID NO: 1073 - DNA, 16 bp, Artificial Sequence
  primer
cgataacttagaagaa

SEQ ID NO: 1074 - DNA, 16 bp, Artificial Sequence
  primer
cataggatgtgacgcc

SEQ ID NO: 1075 - DNA, 16 bp, Artificial Sequence
  primer
ggcttgtcgtcgtatc

SEQ ID NO: 1076 - DNA, 16 bp, Artificial Sequence
  primer
cttgtctgaatattag

SEQ ID NO: 1077 - DNA, 16 bp, Artificial Sequence
  primer
acagttcgagtgtcgg

SEQ ID NO: 1078 - DNA, 16 bp, Artificial Sequence
  primer
ctctaacctgtgacgt

SEQ ID NO: 1079 - DNA, 16 bp, Artificial Sequence
  primer
cgcgctaattcaacaa

SEQ ID NO: 1080 - DNA, 16 bp, Artificial Sequence
  primer
actcacgaatgcggca

SEQ ID NO: 1081 - DNA, 16 bp, Artificial Sequence
  primer
aatcttcggcattcat

SEQ ID NO: 1082 - DNA, 16 bp, Artificial Sequence
  primer
aagtatcaggatcgcg

SEQ ID NO: 1083 - DNA, 16 bp, Artificial Sequence
  primer
agtaactctgcagaca

SEQ ID NO: 1084 - DNA, 16 bp, Artificial Sequence
  primer
ggattgaacattgtgc

SEQ ID NO: 1085 - DNA, 16 bp, Artificial Sequence
  primer
gtgatgctcacgcatc

SEQ ID NO: 1086 - DNA, 16 bp, Artificial Sequence
  primer
cgtagcgtaacggata

SEQ ID NO: 1087 - DNA, 16 bp, Artificial Sequence
  primer
tgcgatgcaccgttag

SEQ ID NO: 1088 - DNA, 16 bp, Artificial Sequence
  primer
ccagtatgctctcagg

SEQ ID NO: 1089 - DNA, 16 bp, Artificial Sequence
  primer
aatgacgttgaagcct

SEQ ID NO: 1090 - DNA, 16 bp, Artificial Sequence
  primer
tcgattctataggagt

SEQ ID NO: 1091 - DNA, 16 bp, Artificial Sequence
  primer
cgataggttcagctat

SEQ ID NO: 1092 - DNA, 16 bp, Artificial Sequence
  primer
ccatgttgatagaata

SEQ ID NO: 1093 - DNA, 16 bp, Artificial Sequence
  primer
gagccacttctacagg

SEQ ID NO: 1094 - DNA, 16 bp, Artificial Sequence
  primer
gcgaactctcggtaat

SEQ ID NO: 1095 - DNA, 16 bp, Artificial Sequence
  primer
gacctgagtagctggt

SEQ ID NO: 1096 - DNA, 16 bp, Artificial Sequence
  primer
cgagtctattagcctg

SEQ ID NO: 1097 - DNA, 16 bp, Artificial Sequence
  primer
gtagtgccatacacct

SEQ ID NO: 1098 - DNA, 16 bp, Artificial Sequence
  primer
ccagtggtctatagca

SEQ ID NO: 1099 - DNA, 16 bp, Artificial Sequence
  primer
gtcagtgcgttattgc

SEQ ID NO: 1100 - DNA, 16 bp, Artificial Sequence
  primer
agtgtcggagtgacga

SEQ ID NO: 1101 - DNA, 16 bp, Artificial Sequence
  primer
aatctccgctatagtt

SEQ ID NO: 1102 - DNA, 16 bp, Artificial Sequence
  primer
cgagtaggtctgactt

SEQ ID NO: 1103 - DNA, 16 bp, Artificial Sequence
  primer
ctgtcgctctaataac

SEQ ID NO: 1104 - DNA, 16 bp, Artificial Sequence
  primer
gctgtcaatataactg

SEQ ID NO: 1105 - DNA, 16 bp, Artificial Sequence
  primer
agctcaagttgaatcc

SEQ ID NO: 1106 - DNA, 16 bp, Artificial Sequence
  primer
aattcatgctcctaac

SEQ ID NO: 1107 - DNA, 16 bp, Artificial Sequence
  primer
ccaaggtctggtgata

SEQ ID NO: 1108 - DNA, 16 bp, Artificial Sequence
  primer
ctccacgtatcttgaa

SEQ ID NO: 1109 - DNA, 16 bp, Artificial Sequence
  primer
tagccgaacaacactt

SEQ ID NO: 1110 - DNA, 16 bp, Artificial Sequence
  primer
agtacacgacatatgc

SEQ ID NO: 1111 - DNA, 16 bp, Artificial Sequence
  primer
acgttctagactcctg

SEQ ID NO: 1112 - DNA, 16 bp, Artificial Sequence
  primer
cgactcaagcactgct

SEQ ID NO: 1113 - DNA, 16 bp, Artificial Sequence
  primer
tgaagctcacgattaa

SEQ ID NO: 1114 - DNA, 16 bp, Artificial Sequence
  primer
tatctaacgtatggta

SEQ ID NO: 1115 - DNA, 16 bp, Artificial Sequence
  primer
tataccatgttccttg

SEQ ID NO: 1116 - DNA, 16 bp, Artificial Sequence
  primer
ttcctacgatgacttc

SEQ ID NO: 1117 - DNA, 16 bp, Artificial Sequence
  primer
ctctccaatatgtgcc

SEQ ID NO: 1118 - DNA, 16 bp, Artificial Sequence
  primer
gagtagagtcttgcca

SEQ ID NO: 1119 - DNA, 16 bp, Artificial Sequence
  primer
gcgagatgtggtccta

SEQ ID NO: 1120 - DNA, 16 bp, Artificial Sequence
  primer
aagctacacggaccac

SEQ ID NO: 1121 - DNA, 16 bp, Artificial Sequence
  primer
atacaactggcaaccg

SEQ ID NO: 1122 - DNA, 16 bp, Artificial Sequence
  primer
cggtagatgctatgct

SEQ ID NO: 1123 - DNA, 16 bp, Artificial Sequence
  primer
tcttgaccggtcatca

SEQ ID NO: 1124 - DNA, 16 bp, Artificial Sequence
  primer
agatcgtgcatgcgat

SEQ ID NO: 1125 - DNA, 16 bp, Artificial Sequence
  primer
tcctcgagacagcctt

SEQ ID NO: 1126 - DNA, 16 bp, Artificial Sequence
  primer
tagccggtaccactta

SEQ ID NO: 1127 - DNA, 16 bp, Artificial Sequence
  primer
gtaaggcagcgtgcaa

SEQ ID NO: 1128 - DNA, 16 bp, Artificial Sequence
  primer
tagtctgctcctggtc

SEQ ID NO: 1129 - DNA, 16 bp, Artificial Sequence
  primer
tggattatagcagcag

SEQ ID NO: 1130 - DNA, 16 bp, Artificial Sequence
  primer
aagaatgatcagacat

SEQ ID NO: 1131 - DNA, 16 bp, Artificial Sequence
  primer
cagcgctatatacctc

SEQ ID NO: 1132 - DNA, 16 bp, Artificial Sequence
  primer
gagtagtacctccacc

SEQ ID NO: 1133 - DNA, 16 bp, Artificial Sequence
  primer
gacgtgatcctctaga

SEQ ID NO: 1134 - DNA, 16 bp, Artificial Sequence
  primer
gttccgttcactacga

SEQ ID NO: 1135 - DNA, 16 bp, Artificial Sequence
  primer
tgcaagcaccaggatg

SEQ ID NO: 1136 - DNA, 16 bp, Artificial Sequence
  primer
ttagttggcggctgag

SEQ ID NO: 1137 - DNA, 16 bp, Artificial Sequence
  primer
cagatgcagacatacg

SEQ ID NO: 1138 - DNA, 16 bp, Artificial Sequence
  primer
gacgcttgatgattat

SEQ ID NO: 1139 - DNA, 16 bp, Artificial Sequence
  primer
tggatcacgactagga

SEQ ID NO: 1140 - DNA, 16 bp, Artificial Sequence
  primer
ctcgtcggtataacgc

SEQ ID NO: 1141 - DNA, 16 bp, Artificial Sequence
  primer
aagcacggatgcgatt

SEQ ID NO: 1142 - DNA, 16 bp, Artificial Sequence
  primer
agatcttccggtgaac

SEQ ID NO: 1143 - DNA, 16 bp, Artificial Sequence
  primer
ggacaatagcaacctg

SEQ ID NO: 1144 - DNA, 16 bp, Artificial Sequence
  primer
gataatcggttccaat

SEQ ID NO: 1145 - DNA, 16 bp, Artificial Sequence
  primer
ctcaagctacagttgt

SEQ ID NO: 1146 - DNA, 16 bp, Artificial Sequence
  primer
gttggcatgatgtaga

SEQ ID NO: 1147 - DNA, 16 bp, Artificial Sequence
  primer
cagcatgaggtaagtg

SEQ ID NO: 1148 - DNA, 16 bp, Artificial Sequence
  primer
gcctcatcacacgtca

SEQ ID NO: 1149 - DNA, 16 bp, Artificial Sequence
  primer
tcgatactacacatcg

SEQ ID NO: 1150 - DNA, 16 bp, Artificial Sequence
  primer
tacacgaggcttgatc

SEQ ID NO: 1151 - DNA, 16 bp, Artificial Sequence
  primer
ttctcgtgtccgcatt

SEQ ID NO: 1152 - DNA, 16 bp, Artificial Sequence
  primer
ggtgaagcaacagcat

SEQ ID NO: 1153 - DNA, 18 bp, Artificial Sequence
  primer
cgaaccgactgtacagtt

SEQ ID NO: 1154 - DNA, 18 bp, Artificial Sequence
  primer
ccgactgcggataagtta

SEQ ID NO: 1155 - DNA, 18 bp, Artificial Sequence
  primer
cgacaggtaggtaagcag

SEQ ID NO: 1156 - DNA, 18 bp, Artificial Sequence
  primer
tgatacgttggtatacag

SEQ ID NO: 1157 - DNA, 18 bp, Artificial Sequence
  primer
ctactatagaatacgtag

SEQ ID NO: 1158 - DNA, 18 bp, Artificial Sequence
  primer
agactgtggcaatggcat

SEQ ID NO: 1159 - DNA, 18 bp, Artificial Sequence
  primer
ggaagactgatacaacga

SEQ ID NO: 1160 - DNA, 18 bp, Artificial Sequence
  primer
tatgcacatatagcgctt

SEQ ID NO: 1161 - DNA, 18 bp, Artificial Sequence
  primer
catggtaatcgaccgagg

SEQ ID NO: 1162 - DNA, 18 bp, Artificial Sequence
  primer
gtcattgccgtcattgcc

SEQ ID NO: 1163 - DNA, 18 bp, Artificial Sequence
  primer
cctaagaactccgaagct

SEQ ID NO: 1164 - DNA, 18 bp, Artificial Sequence
  primer
tcgctcaccgtactagga

SEQ ID NO: 1165 - DNA, 18 bp, Artificial Sequence
  primer
tattactgtcacagcagg

SEQ ID NO: 1166 - DNA, 18 bp, Artificial Sequence
  primer
tgagacaggctacgagtc

SEQ ID NO: 1167 - DNA, 18 bp, Artificial Sequence
  primer
aagctatgcgaacacgtt

SEQ ID NO: 1168 - DNA, 18 bp, Artificial Sequence
  primer
aacggaggagtgagccaa

SEQ ID NO: 1169 - DNA, 18 bp, Artificial Sequence
  primer
ccactatggacatcatgg

SEQ ID NO: 1170 - DNA, 18 bp, Artificial Sequence
  primer
atggtggtggatagctcg

SEQ ID NO: 1171 - DNA, 18 bp, Artificial Sequence
  primer
tcaccggttacacatcgc

SEQ ID NO: 1172 - DNA, 18 bp, Artificial Sequence
  primer
aagatactgagatatgga

SEQ ID NO: 1173 - DNA, 18 bp, Artificial Sequence
  primer
gacctgttcttgaactag

SEQ ID NO: 1174 - DNA, 18 bp, Artificial Sequence
  primer
aagtagagctctcggtta

SEQ ID NO: 1175 - DNA, 18 bp, Artificial Sequence
  primer
ctatgttcttactctctt

SEQ ID NO: 1176 - DNA, 18 bp, Artificial Sequence
  primer
caaggctataagcggtta

SEQ ID NO: 1177 - DNA, 18 bp, Artificial Sequence
  primer
gaagctaattaaccgata

SEQ ID NO: 1178 - DNA, 18 bp, Artificial Sequence
  primer
ttcacgtctgccaagcac

SEQ ID NO: 1179 - DNA, 18 bp, Artificial Sequence
  primer
atcgtatagatcgagaca

SEQ ID NO: 1180 - DNA, 18 bp, Artificial Sequence
  primer
gtcacagattcacatcat

SEQ ID NO: 1181 - DNA, 18 bp, Artificial Sequence
  primer
gtgcctgtgaactatcag

SEQ ID NO: 1182 - DNA, 18 bp, Artificial Sequence
  primer
cagcgtacaagatagtcg

SEQ ID NO: 1183 - DNA, 18 bp, Artificial Sequence
  primer
gcatggcatggtagacct

SEQ ID NO: 1184 - DNA, 18 bp, Artificial Sequence
  primer
ggtatgctactcttcgca

SEQ ID NO: 1185 - DNA, 18 bp, Artificial Sequence
  primer
atgttcagtcacaagcga

SEQ ID NO: 1186 - DNA, 18 bp, Artificial Sequence
  primer
taggaagtgtgtaatagc

SEQ ID NO: 1187 - DNA, 18 bp, Artificial Sequence
  primer
aatccatgtagctgtacg

SEQ ID NO: 1188 - DNA, 18 bp, Artificial Sequence
  primer
ccagattcactggcatag

SEQ ID NO: 1189 - DNA, 18 bp, Artificial Sequence
  primer
ttgtctctacgtaatatc

SEQ ID NO: 1190 - DNA, 18 bp, Artificial Sequence
  primer
gtggtgcttgtgacaatt

SEQ ID NO: 1191 - DNA, 18 bp, Artificial Sequence
  primer
cagcctacttggctgaga

SEQ ID NO: 1192 - DNA, 18 bp, Artificial Sequence
  primer
tactcaatgcatctgtgt

SEQ ID NO: 1193 - DNA, 18 bp, Artificial Sequence
  primer
tgtagagagacgaatata

SEQ ID NO: 1194 - DNA, 18 bp, Artificial Sequence
  primer
gcctacaaccatcctact

SEQ ID NO: 1195 - DNA, 18 bp, Artificial Sequence
  primer
gcgtggcattgagattca

SEQ ID NO: 1196 - DNA, 18 bp, Artificial Sequence
  primer
gcatgccagctaactgag

SEQ ID NO: 1197 - DNA, 18 bp, Artificial Sequence
  primer
gcgagtaatccggttgga

SEQ ID NO: 1198 - DNA, 18 bp, Artificial Sequence
  primer
gcctctaccagaacgtca

SEQ ID NO: 1199 - DNA, 18 bp, Artificial Sequence
  primer
gtcagcagaagactgacc

SEQ ID NO: 1200 - DNA, 18 bp, Artificial Sequence
  primer
gataacagacgtagcagg

SEQ ID NO: 1201 - DNA, 18 bp, Artificial Sequence
  primer
caggagatcgcatgtcgt

SEQ ID NO: 1202 - DNA, 18 bp, Artificial Sequence
  primer
ctggaaggaatggagcca

SEQ ID NO: 1203 - DNA, 18 bp, Artificial Sequence
  primer
attggttctctaccacaa

SEQ ID NO: 1204 - DNA, 18 bp, Artificial Sequence
  primer
ctcattgttgacggctca

SEQ ID NO: 1205 - DNA, 18 bp, Artificial Sequence
  primer
ttcaggactgtagttcat

SEQ ID NO: 1206 - DNA, 18 bp, Artificial Sequence
  primer
agaccgcactaactcaag

SEQ ID NO: 1207 - DNA, 18 bp, Artificial Sequence
  primer
ggaatattgtgcagaccg

SEQ ID NO: 1208 - DNA, 18 bp, Artificial Sequence
  primer
cctattactaatagctca

SEQ ID NO: 1209 - DNA, 18 bp, Artificial Sequence
  primer
atggcatgagtacttcgg

SEQ ID NO: 1210 - DNA, 18 bp, Artificial Sequence
  primer
gacacgtatgcgtctagc

SEQ ID NO: 1211 - DNA, 18 bp, Artificial Sequence
  primer
gaaggtacggaatctgtt

SEQ ID NO: 1212 - DNA, 18 bp, Artificial Sequence
  primer
tataacgtccgacactgt

SEQ ID NO: 1213 - DNA, 18 bp, Artificial Sequence
  primer
gctaatacattaccgccg

SEQ ID NO: 1214 - DNA, 18 bp, Artificial Sequence
  primer
gaagccaacactcctgac

SEQ ID NO: 1215 - DNA, 18 bp, Artificial Sequence
  primer
cgaataacgagctgtgat

SEQ ID NO: 1216 - DNA, 18 bp, Artificial Sequence
  primer
gcctaccgatcgcactta

SEQ ID NO: 1217 - DNA, 18 bp, Artificial Sequence
  primer
ctgaggagaatagcctgc

SEQ ID NO: 1218 - DNA, 18 bp, Artificial Sequence
  primer
cagcatggacagtacttc

SEQ ID NO: 1219 - DNA, 18 bp, Artificial Sequence
  primer
ggtatagagccttcctta

SEQ ID NO: 1220 - DNA, 18 bp, Artificial Sequence
  primer
cgctctgcatatatagca

SEQ ID NO: 1221 - DNA, 18 bp, Artificial Sequence
  primer
cggctctactatgctcgt

SEQ ID NO: 1222 - DNA, 18 bp, Artificial Sequence
  primer
cctaatgcgaagctcacc

SEQ ID NO: 1223 - DNA, 18 bp, Artificial Sequence
  primer
acaaccggtgaggcagta

SEQ ID NO: 1224 - DNA, 18 bp, Artificial Sequence
  primer
ttggttcgaaccaaccgc

SEQ ID NO: 1225 - DNA, 18 bp, Artificial Sequence
  primer
atactaggttgaactaag

SEQ ID NO: 1226 - DNA, 18 bp, Artificial Sequence
  primer
gcgttgagagtaacatat

SEQ ID NO: 1227 - DNA, 18 bp, Artificial Sequence
  primer
agttgtataataagcgtc

SEQ ID NO: 1228 - DNA, 18 bp, Artificial Sequence
  primer
gtatgatgccgtccaatt

SEQ ID NO: 1229 - DNA, 18 bp, Artificial Sequence
  primer
ggactctctgaagagtct

SEQ ID NO: 1230 - DNA, 18 bp, Artificial Sequence
  primer
ggactctcttgacttgaa

SEQ ID NO: 1231 - DNA, 18 bp, Artificial Sequence
  primer
gataacagtgcttcgtcc

SEQ ID NO: 1232 - DNA, 18 bp, Artificial Sequence
  primer
ggccattatagatgaact

SEQ ID NO: 1233 - DNA, 18 bp, Artificial Sequence
  primer
atagagagcacagagcag

SEQ ID NO: 1234 - DNA, 18 bp, Artificial Sequence
  primer
gtgtgagtgtatcataac

SEQ ID NO: 1235 - DNA, 18 bp, Artificial Sequence
  primer
ataaccttagtgcgcgtc

SEQ ID NO: 1236 - DNA, 18 bp, Artificial Sequence
  primer
ccgactgatatgcatgga

SEQ ID NO: 1237 - DNA, 18 bp, Artificial Sequence
  primer
ggatatctgatcgcatca

SEQ ID NO: 1238 - DNA, 18 bp, Artificial Sequence
  primer
cagcattaacgaggcgaa

SEQ ID NO: 1239 - DNA, 18 bp, Artificial Sequence
  primer
gcgaggcctacatattcg

SEQ ID NO: 1240 - DNA, 18 bp, Artificial Sequence
  primer
cgataagtggtaaggtct

SEQ ID NO: 1241 - DNA, 18 bp, Artificial Sequence
  primer
agatcctgagtcgagcaa

SEQ ID NO: 1242 - DNA, 18 bp, Artificial Sequence
  primer
aagatataacgagaccga

SEQ ID NO: 1243 - DNA, 18 bp, Artificial Sequence
  primer
ccgactgattgagaacgt

SEQ ID NO: 1244 - DNA, 18 bp, Artificial Sequence
  primer
tcggcttatatgacacgt

SEQ ID NO: 1245 - DNA, 18 bp, Artificial Sequence
  primer
aataacgtacgccggagg

SEQ ID NO: 1246 - DNA, 18 bp, Artificial Sequence
  primer
aacacagcattgcgcacg

SEQ ID NO: 1247 - DNA, 18 bp, Artificial Sequence
  primer
gtagtctgacagcaacaa

SEQ ID NO: 1248 - DNA, 18 bp, Artificial Sequence
  primer
agaatgacttgagctgct

SEQ ID NO: 1249 - DNA, 20 bp, Artificial Sequence
  primer
actggtagtaacgtccacct

SEQ ID NO: 1250 - DNA, 20 bp, Artificial Sequence
  primer
agactggttgttattcgcct

SEQ ID NO: 1251 - DNA, 20 bp, Artificial Sequence
  primer
tatcattgacagcgagctca

SEQ ID NO: 1252 - DNA, 20 bp, Artificial Sequence
  primer
tggagtctgaagaaggactc

SEQ ID NO: 1253 - DNA, 20 bp, Artificial Sequence
  primer
catctggactacggcaacga

SEQ ID NO: 1254 - DNA, 20 bp, Artificial Sequence
  primer
aactgtcataagacagacaa

SEQ ID NO: 1255 - DNA, 20 bp, Artificial Sequence
  primer
cctcaacatgacatacaccg

SEQ ID NO: 1256 - DNA, 20 bp, Artificial Sequence
  primer
caataccgttcgcgattcta

SEQ ID NO: 1257 - DNA, 20 bp, Artificial Sequence
  primer
gcgtctacgttgattcggcc

SEQ ID NO: 1258 - DNA, 20 bp, Artificial Sequence
  primer
tgaacagaggcacttgcagg

SEQ ID NO: 1259 - DNA, 20 bp, Artificial Sequence
  primer
cgactagaacctactactgc

SEQ ID NO: 1260 - DNA, 20 bp, Artificial Sequence
  primer
gcaccgcacgtggagagata

SEQ ID NO: 1261 - DNA, 20 bp, Artificial Sequence
  primer
ctgagagaccgactgatgcg

SEQ ID NO: 1262 - DNA, 20 bp, Artificial Sequence
  primer
tcgtccttctacttaatgat

SEQ ID NO: 1263 - DNA, 20 bp, Artificial Sequence
  primer
caagctataccatccgaatt

SEQ ID NO: 1264 - DNA, 20 bp, Artificial Sequence
  primer
caatacgtatagtcttagat

SEQ ID NO: 1265 - DNA, 20 bp, Artificial Sequence
  primer
ccatccacagtgacctatgt

SEQ ID NO: 1266 - DNA, 20 bp, Artificial Sequence
  primer
tatccgttggagaaggttca

SEQ ID NO: 1267 - DNA, 20 bp, Artificial Sequence
  primer
cgcctaggtacctgagtacg

SEQ ID NO: 1268 - DNA, 20 bp, Artificial Sequence
  primer
cagagtgctcgtgttcgcga

SEQ ID NO: 1269 - DNA, 20 bp, Artificial Sequence
  primer
cgcttggacatccttaagaa

SEQ ID NO: 1270 - DNA, 20 bp, Artificial Sequence
  primer
gaccgcatgattagtcttac

SEQ ID NO: 1271 - DNA, 20 bp, Artificial Sequence
  primer
cttggccgtagtcactcagt

SEQ ID NO: 1272 - DNA, 20 bp, Artificial Sequence
  primer
gatagcgatattcagttcgc

SEQ ID NO: 1273 - DNA, 20 bp, Artificial Sequence
  primer
atccaacactaagacaacca

SEQ ID NO: 1274 - DNA, 20 bp, Artificial Sequence
  primer
ccattctgttgcgtgtcctc

SEQ ID NO: 1275 - DNA, 20 bp, Artificial Sequence
  primer
acattctgtacgcttgcagc

SEQ ID NO: 1276 - DNA, 20 bp, Artificial Sequence
  primer
tgctgaacgccaatcgctta

SEQ ID NO: 1277 - DNA, 20 bp, Artificial Sequence
  primer
tcctctacaagaatattgcg

SEQ ID NO: 1278 - DNA, 20 bp, Artificial Sequence
  primer
cgaccaacgcagcctgattc

SEQ ID NO: 1279 - DNA, 20 bp, Artificial Sequence
  primer
attgcgagcttgagtagcgc

SEQ ID NO: 1280 - DNA, 20 bp, Artificial Sequence
  primer
aaggtgcgagcataggaatc

SEQ ID NO: 1281 - DNA, 20 bp, Artificial Sequence
  primer
cacttaagtgtgatatagat

SEQ ID NO: 1282 - DNA, 20 bp, Artificial Sequence
  primer
atcggtatgctgacctagac

SEQ ID NO: 1283 - DNA, 20 bp, Artificial Sequence
  primer
tacaatctcgaatgcaggat

SEQ ID NO: 1284 - DNA, 20 bp, Artificial Sequence
  primer
ccatatgaagcgcagccgtc

SEQ ID NO: 1285 - DNA, 20 bp, Artificial Sequence
  primer
cgtctcgtggacattcgagg

SEQ ID NO: 1286 - DNA, 20 bp, Artificial Sequence
  primer
ccgagtacagaagcgtggaa

SEQ ID NO: 1287 - DNA, 20 bp, Artificial Sequence
  primer
ttacgtggtcgacaggcagt

SEQ ID NO: 1288 - DNA, 20 bp, Artificial Sequence
  primer
agctgcaatctgcatgatta

SEQ ID NO: 1289 - DNA, 20 bp, Artificial Sequence
  primer
acctgccgaagcagcctaca

SEQ ID NO: 1290 - DNA, 20 bp, Artificial Sequence
  primer
aacatgataaccacatggtt

SEQ ID NO: 1291 - DNA, 20 bp, Artificial Sequence
  primer
atccgactgattgaattacc

SEQ ID NO: 1292 - DNA, 20 bp, Artificial Sequence
  primer
tcacgctgactcttatcagg

SEQ ID NO: 1293 - DNA, 20 bp, Artificial Sequence
  primer
gcgcgctcgaagtacaacat

SEQ ID NO: 1294 - DNA, 20 bp, Artificial Sequence
  primer
acagccagatgcgttgttcc

SEQ ID NO: 1295 - DNA, 20 bp, Artificial Sequence
  primer
ggagctctgacctgcaagaa

SEQ ID NO: 1296 - DNA, 20 bp, Artificial Sequence
  primer
aacattagcctcaagtaaga

SEQ ID NO: 1297 - DNA, 20 bp, Artificial Sequence
  primer
tgtgattatgccgaatgagg

SEQ ID NO: 1298 - DNA, 20 bp, Artificial Sequence
  primer
gagtaataatccaatcagta

SEQ ID NO: 1299 - DNA, 20 bp, Artificial Sequence
  primer
ctccttggcgacagctgaac

SEQ ID NO: 1300 - DNA, 20 bp, Artificial Sequence
  primer
ttacgcacacatacacagac

SEQ ID NO: 1301 - DNA, 20 bp, Artificial Sequence
  primer
acgccgtatggcgacttagg

SEQ ID NO: 1302 - DNA, 20 bp, Artificial Sequence
  primer
agaacgacaattacgatggc

SEQ ID NO: 1303 - DNA, 20 bp, Artificial Sequence
  primer
tgctaacgtaccactgccac

SEQ ID NO: 1304 - DNA, 20 bp, Artificial Sequence
  primer
catccagaatgtctatcata

SEQ ID NO: 1305 - DNA, 20 bp, Artificial Sequence
  primer
ggagaacgcctatagcactc

SEQ ID NO: 1306 - DNA, 20 bp, Artificial Sequence
  primer
acctcttgtgacggccagtc

SEQ ID NO: 1307 - DNA, 20 bp, Artificial Sequence
  primer
tgccataacttggcataaga

SEQ ID NO: 1308 - DNA, 20 bp, Artificial Sequence
  primer
acaattgtctgaccacgctc

SEQ ID NO: 1309 - DNA, 20 bp, Artificial Sequence
  primer
tcgtcaccttcacagaacga

SEQ ID NO: 1310 - DNA, 20 bp, Artificial Sequence
  primer
agcagcagatgatgatccaa

SEQ ID NO: 1311 - DNA, 20 bp, Artificial Sequence
  primer
tcgtgccttggattccagga

SEQ ID NO: 1312 - DNA, 20 bp, Artificial Sequence
  primer
tgttatagccacgatactat

SEQ ID NO: 1313 - DNA, 20 bp, Artificial Sequence
  primer
aatctcacctgtaccttccg

SEQ ID NO: 1314 - DNA, 20 bp, Artificial Sequence
  primer
gagtagcggaagcgttagcg

SEQ ID NO: 1315 - DNA, 20 bp, Artificial Sequence
  primer
aatactccggcgaggtatac

SEQ ID NO: 1316 - DNA, 20 bp, Artificial Sequence
  primer
ttcgcatccttgcacgaaca

SEQ ID NO: 1317 - DNA, 20 bp, Artificial Sequence
  primer
aaccggctaatactactggc

SEQ ID NO: 1318 - DNA, 20 bp, Artificial Sequence
  primer
ctagcatcttagacaccaga

SEQ ID NO: 1319 - DNA, 20 bp, Artificial Sequence
  primer
tagttgcgtgatacaagata

SEQ ID NO: 1320 - DNA, 20 bp, Artificial Sequence
  primer
tcgtctcgacacagttggtc

SEQ ID NO: 1321 - DNA, 20 bp, Artificial Sequence
  primer
tccgttcgcgtgcgaactga

SEQ ID NO: 1322 - DNA, 20 bp, Artificial Sequence
  primer
tctgactctggtgtacagtc

SEQ ID NO: 1323 - DNA, 20 bp, Artificial Sequence
  primer
acagcgcaattatatcctgt

SEQ ID NO: 1324 - DNA, 20 bp, Artificial Sequence
  primer
agatccgtacgtgagactag

SEQ ID NO: 1325 - DNA, 20 bp, Artificial Sequence
  primer
tacattgaagcatccgaaca

SEQ ID NO: 1326 - DNA, 20 bp, Artificial Sequence
  primer
ctcctgagagatcaacgcca

SEQ ID NO: 1327 - DNA, 20 bp, Artificial Sequence
  primer
tcacctcgaatgagttcgtt

SEQ ID NO: 1328 - DNA, 20 bp, Artificial Sequence
  primer
tagcgacttaaggtccaagc

SEQ ID NO: 1329 - DNA, 20 bp, Artificial Sequence
  primer
agtacgtattgccgtgcaag

SEQ ID NO: 1330 - DNA, 20 bp, Artificial Sequence
  primer
agccacgaaccgacgtcata

SEQ ID NO: 1331 - DNA, 20 bp, Artificial Sequence
  primer
tgatgtgtacgctactacta

SEQ ID NO: 1332 - DNA, 20 bp, Artificial Sequence
  primer
ccactgtgtgcagcagacga

SEQ ID NO: 1333 - DNA, 20 bp, Artificial Sequence
  primer
ctattgtacagcgaacgctg

SEQ ID NO: 1334 - DNA, 20 bp, Artificial Sequence
  primer
ctccgatatcgcacggatcg

SEQ ID NO: 1335 - DNA, 20 bp, Artificial Sequence
  primer
aacttatcgtcggacgcatg

SEQ ID NO: 1336 - DNA, 20 bp, Artificial Sequence
  primer
tatcctaattcgtgccggtc

SEQ ID NO: 1337 - DNA, 20 bp, Artificial Sequence
  primer
acagccttcctgtgtggact

SEQ ID NO: 1338 - DNA, 20 bp, Artificial Sequence
  primer
cctccgtgaggatcgtacca

SEQ ID NO: 1339 - DNA, 20 bp, Artificial Sequence
  primer
gctctaagtaacagaactaa

SEQ ID NO: 1340 - DNA, 20 bp, Artificial Sequence
  primer
gacttaccgcgcgttctggt

SEQ ID NO: 1341 - DNA, 20 bp, Artificial Sequence
  primer
tctgaggatacacatgtgga

SEQ ID NO: 1342 - DNA, 20 bp, Artificial Sequence
  primer
tgtaatcacactggtgtcgg

SEQ ID NO: 1343 - DNA, 20 bp, Artificial Sequence
  primer
cactaggcggcagacataca

SEQ ID NO: 1344 - DNA, 20 bp, Artificial Sequence
  primer
ctagagcacagtaccacgtt

SEQ ID NO: 1345 - DNA, 22 bp, Artificial Sequence
  primer
ttcagaggtctacgcttccggt

SEQ ID NO: 1346 - DNA, 22 bp, Artificial Sequence
  primer
aacacagactgcgttatgccaa

SEQ ID NO: 1347 - DNA, 22 bp, Artificial Sequence
  primer
tgctgagttctatacagcagtg

SEQ ID NO: 1348 - DNA, 22 bp, Artificial Sequence
  primer
acctattatatgatagcgtcat

SEQ ID NO: 1349 - DNA, 22 bp, Artificial Sequence
  primer
atcgtgagctacagtgaatgca

SEQ ID NO: 1350 - DNA, 22 bp, Artificial Sequence
  primer
cgtgatgtatccggccttgcag

SEQ ID NO: 1351 - DNA, 22 bp, Artificial Sequence
  primer
tcttctggtcctagagttgtgc

SEQ ID NO: 1352 - DNA, 22 bp, Artificial Sequence
  primer
tgatgtcggcggcggatcagat

SEQ ID NO: 1353 - DNA, 22 bp, Artificial Sequence
  primer
tcggccttagcgttcagcatcc

SEQ ID NO: 1354 - DNA, 22 bp, Artificial Sequence
  primer
ttaagtaggtcagccactgcac

SEQ ID NO: 1355 - DNA, 22 bp, Artificial Sequence
  primer
ccaggtgagttgatctgacacc

SEQ ID NO: 1356 - DNA, 22 bp, Artificial Sequence
  primer
tatactattactgtgttcgatc

SEQ ID NO: 1357 - DNA, 22 bp, Artificial Sequence
  primer
ccgcagtatgtctagtgttgtc

SEQ ID NO: 1358 - DNA, 22 bp, Artificial Sequence
  primer
gtctaccgcgtacgaagctctc

SEQ ID NO: 1359 - DNA, 22 bp, Artificial Sequence
  primer
atgcgagtccgtggtcgatcct

SEQ ID NO: 1360 - DNA, 22 bp, Artificial Sequence
  primer
tggtagattggtgtgagaacta

SEQ ID NO: 1361 - DNA, 22 bp, Artificial Sequence
  primer
aggttcgtcgatcaactgctaa

SEQ ID NO: 1362 - DNA, 22 bp, Artificial Sequence
  primer
acgacaagcatcctgcgatatc

SEQ ID NO: 1363 - DNA, 22 bp, Artificial Sequence
  primer
ttgaatcacagagagcgtgatt

SEQ ID NO: 1364 - DNA, 22 bp, Artificial Sequence
  primer
gtacttagtgcttacgtcagct

SEQ ID NO: 1365 - DNA, 22 bp, Artificial Sequence
  primer
gattattaaggccaagctcata

SEQ ID NO: 1366 - DNA, 22 bp, Artificial Sequence
  primer
gcatgcagagacgtactcatcg

SEQ ID NO: 1367 - DNA, 22 bp, Artificial Sequence
  primer
tagcggatggtgtcctggcact

SEQ ID NO: 1368 - DNA, 22 bp, Artificial Sequence
  primer
tacggctgccaacttaataact

SEQ ID NO: 1369 - DNA, 22 bp, Artificial Sequence
  primer
ctcatatgacaacttctatagt

SEQ ID NO: 1370 - DNA, 22 bp, Artificial Sequence
  primer
caagcaatagttgtcggccacc

SEQ ID NO: 1371 - DNA, 22 bp, Artificial Sequence
  primer
ttcagcaatccgtactgctaga

SEQ ID NO: 1372 - DNA, 22 bp, Artificial Sequence
  primer
tgagacgttgctgacattctcc

SEQ ID NO: 1373 - DNA, 22 bp, Artificial Sequence
  primer
gttccgatgagttagatgtata

SEQ ID NO: 1374 - DNA, 22 bp, Artificial Sequence
  primer
ttgacgcttggaggagtacaag

SEQ ID NO: 1375 - DNA, 22 bp, Artificial Sequence
  primer
ttcatgttacctccacattgtg

SEQ ID NO: 1376 - DNA, 22 bp, Artificial Sequence
  primer
gagcacgtgccagattgcaacc

SEQ ID NO: 1377 - DNA, 22 bp, Artificial Sequence
  primer
ggtcgacaagcacaagccttct

SEQ ID NO: 1378 - DNA, 22 bp, Artificial Sequence
  primer
taggcaggtaagatgaccgact

SEQ ID NO: 1379 - DNA, 22 bp, Artificial Sequence
  primer
cgaggcatgccaagtcgccaat

SEQ ID NO: 1380 - DNA, 22 bp, Artificial Sequence
  primer
agtgttgataggcggatgagag

SEQ ID NO: 1381 - DNA, 22 bp, Artificial Sequence
  primer
ttcggtctagacctctcacaat

SEQ ID NO: 1382 - DNA, 22 bp, Artificial Sequence
  primer
gtgacgctcatatcttgccacc

SEQ ID NO: 1383 - DNA, 22 bp, Artificial Sequence
  primer
gatgtaattctacgcgcggact

SEQ ID NO: 1384 - DNA, 22 bp, Artificial Sequence
  primer
gatggcgatgttgcattacatg

SEQ ID NO: 1385 - DNA, 22 bp, Artificial Sequence
  primer
tatgctctgaattaacgtagaa

SEQ ID NO: 1386 - DNA, 22 bp, Artificial Sequence
  primer
aggcaatatggtgatccgtagc

SEQ ID NO: 1387 - DNA, 22 bp, Artificial Sequence
  primer
tgacagcgatgcatacagtagt

SEQ ID NO: 1388 - DNA, 22 bp, Artificial Sequence
  primer
ttctgctaacggtatccaatac

SEQ ID NO: 1389 - DNA, 22 bp, Artificial Sequence
  primer
gagtcgtccatacgatctagga

SEQ ID NO: 1390 - DNA, 22 bp, Artificial Sequence
  primer
agacggactcaacgccaattcc

SEQ ID NO: 1391 - DNA, 22 bp, Artificial Sequence
  primer
gtagtgttgagcggaccgagct

SEQ ID NO: 1392 - DNA, 22 bp, Artificial Sequence
  primer
aatataactagatcatagccag

SEQ ID NO: 1393 - DNA, 22 bp, Artificial Sequence
  primer
tcaatcggagaatacagaacgt

SEQ ID NO: 1394 - DNA, 22 bp, Artificial Sequence
  primer
atctccgtcgtccgaaccaaca

SEQ ID NO: 1395 - DNA, 22 bp, Artificial Sequence
  primer
taggcgttcagcggtatgctta

SEQ ID NO: 1396 - DNA, 22 bp, Artificial Sequence
  primer
tgcgtgctatacaacctatacg

SEQ ID NO: 1397 - DNA, 22 bp, Artificial Sequence
  primer
atggccggcatacatctgtatg

SEQ ID NO: 1398 - DNA, 22 bp, Artificial Sequence
  primer
tgatgctgacataacactgaat

SEQ ID NO: 1399 - DNA, 22 bp, Artificial Sequence
  primer
atccaaggtacctgaacatcct

SEQ ID NO: 1400 - DNA, 22 bp, Artificial Sequence
  primer
tagtgacgaccaggtgagcctc

SEQ ID NO: 1401 - DNA, 22 bp, Artificial Sequence
  primer
aggaggatccgtcaagtcgacc

SEQ ID NO: 1402 - DNA, 22 bp, Artificial Sequence
  primer
agagtatgccagatcgtgaggc

SEQ ID NO: 1403 - DNA, 22 bp, Artificial Sequence
  primer
ccactcactaggatggctgcgt

SEQ ID NO: 1404 - DNA, 22 bp, Artificial Sequence
  primer
tatccaacctgttatagcgatt

SEQ ID NO: 1405 - DNA, 22 bp, Artificial Sequence
  primer
tcttgcagtgagttgagtctgc

SEQ ID NO: 1406 - DNA, 22 bp, Artificial Sequence
  primer
ccactgttgtacatacacctgg

SEQ ID NO: 1407 - DNA, 22 bp, Artificial Sequence
  primer
atgcgcgtaggccactaagtcc

SEQ ID NO: 1408 - DNA, 22 bp, Artificial Sequence
  primer
acagcggtctacaaccgactgc

SEQ ID NO: 1409 - DNA, 22 bp, Artificial Sequence
  primer
tcgcgctccagacaattgcagc

SEQ ID NO: 1410 - DNA, 22 bp, Artificial Sequence
  primer
ccggtagaccaggagtggtcat

SEQ ID NO: 1411 - DNA, 22 bp, Artificial Sequence
  primer
atctcctaacctagagccatct

SEQ ID NO: 1412 - DNA, 22 bp, Artificial Sequence
  primer
ccacatcgaatctaacaactac

SEQ ID NO: 1413 - DNA, 22 bp, Artificial Sequence
  primer
tagtcttattgaatacgtccta

SEQ ID NO: 1414 - DNA, 22 bp, Artificial Sequence
  primer
tccttaagccttggaactggcg

SEQ ID NO: 1415 - DNA, 22 bp, Artificial Sequence
  primer
ccgtgatggattgacgtagagg

SEQ ID NO: 1416 - DNA, 22 bp, Artificial Sequence
  primer
gcctggataacagatgtcttag

SEQ ID NO: 1417 - DNA, 22 bp, Artificial Sequence
  primer
ctcgacctataatcttctgcca

SEQ ID NO: 1418 - DNA, 22 bp, Artificial Sequence
  primer
agctacttctccttcctaatca

SEQ ID NO: 1419 - DNA, 22 bp, Artificial Sequence
  primer
acacgctattgccttccagtta

SEQ ID NO: 1420 - DNA, 22 bp, Artificial Sequence
  primer
aagcctgtgcatgcaatgagaa

SEQ ID NO: 1421 - DNA, 22 bp, Artificial Sequence
  primer
tcgttggttatagcacaacttc

SEQ ID NO: 1422 - DNA, 22 bp, Artificial Sequence
  primer
gcgatgccttccaacataccaa

SEQ ID NO: 1423 - DNA, 22 bp, Artificial Sequence
  primer
ccaccgttagcacgtgctacgt

SEQ ID NO: 1424 - DNA, 22 bp, Artificial Sequence
  primer
gttaccacaatgccgccatcaa

SEQ ID NO: 1425 - DNA, 22 bp, Artificial Sequence
  primer
ggtgcattaagaacgaactacc

SEQ ID NO: 1426 - DNA, 22 bp, Artificial Sequence
  primer
tccttccggataatgccgattc

SEQ ID NO: 1427 - DNA, 22 bp, Artificial Sequence
  primer
aaccgcaacttctagcggaaga

SEQ ID NO: 1428 - DNA, 22 bp, Artificial Sequence
  primer
tccttaagcagttgaacctagg

SEQ ID NO: 1429 - DNA, 22 bp, Artificial Sequence
  primer
tactaagtcagataagatcaga

SEQ ID NO: 1430 - DNA, 22 bp, Artificial Sequence
  primer
ttcgccataactagatgaatgc

SEQ ID NO: 1431 - DNA, 22 bp, Artificial Sequence
  primer
aagaagttagacgcggtggctg

SEQ ID NO: 1432 - DNA, 22 bp, Artificial Sequence
  primer
gtatctgatcgaagagcggtgg

SEQ ID NO: 1433 - DNA, 22 bp, Artificial Sequence
  primer
tcaagagctacgaagtaagtcc

SEQ ID NO: 1434 - DNA, 22 bp, Artificial Sequence
  primer
cgagtacacagcagcataccta

SEQ ID NO: 1435 - DNA, 22 bp, Artificial Sequence
  primer
ctcgataagttactctgctaga

SEQ ID NO: 1436 - DNA, 22 bp, Artificial Sequence
  primer
atggtgctggttctccgtctgt

SEQ ID NO: 1437 - DNA, 22 bp, Artificial Sequence
  primer
tcaagcggtccaaggctgagac

SEQ ID NO: 1438 - DNA, 22 bp, Artificial Sequence
  primer
tgtcctgctctgttgctaccgt

SEQ ID NO: 1439 - DNA, 22 bp, Artificial Sequence
  primer
agtcatatcgcgtcacacgttg

SEQ ID NO: 1440 - DNA, 22 bp, Artificial Sequence
  primer
ggtgaataaggacatgagaagc

SEQ ID NO: 1441 - DNA, 24 bp, Artificial Sequence
  primer
cctgatcttatctagtagagactc

SEQ ID NO: 1442 - DNA, 24 bp, Artificial Sequence
  primer
ttctgtgtaggtgtgccaatcacc

SEQ ID NO: 1443 - DNA, 24 bp, Artificial Sequence
  primer
gacttccagatgcttaagacgaca

SEQ ID NO: 1444 - DNA, 24 bp, Artificial Sequence
  primer
gtccttcgacggagaacatccgag

SEQ ID NO: 1445 - DNA, 24 bp, Artificial Sequence
  primer
cttggttagtgtaccgtcaacgtc

SEQ ID NO: 1446 - DNA, 24 bp, Artificial Sequence
  primer
aagcggcatgtgcctaatcgacgt

SEQ ID NO: 1447 - DNA, 24 bp, Artificial Sequence
  primer
cgaccgtcgttacacggaatccga

SEQ ID NO: 1448 - DNA, 24 bp, Artificial Sequence
  primer
tcgcaagtgtgccgttctgttcat

SEQ ID NO: 1449 - DNA, 24 bp, Artificial Sequence
  primer
cgtactgaagttcggagtcgccgt

SEQ ID NO: 1450 - DNA, 24 bp, Artificial Sequence
  primer
ccactacagaatggtagcagatca

SEQ ID NO: 1451 - DNA, 24 bp, Artificial Sequence
  primer
agtaggagagaggcctacacaaca

SEQ ID NO: 1452 - DNA, 24 bp, Artificial Sequence
  primer
agccaagatactcgttcggtatgg

SEQ ID NO: 1453 - DNA, 24 bp, Artificial Sequence
  primer
gttccgagtacattgaatcctggc

SEQ ID NO: 1454 - DNA, 24 bp, Artificial Sequence
  primer
aggcgtacgagttattgccagagg

SEQ ID NO: 1455 - DNA, 24 bp, Artificial Sequence
  primer
gtggcatcacacatatctcagcat

SEQ ID NO: 1456 - DNA, 24 bp, Artificial Sequence
  primer
gagaccgatatgttgatgccagaa

SEQ ID NO: 1457 - DNA, 24 bp, Artificial Sequence
  primer
caactgtagccagtcgattgctat

SEQ ID NO: 1458 - DNA, 24 bp, Artificial Sequence
  primer
tatcaatgcaatgagaggatgcag

SEQ ID NO: 1459 - DNA, 24 bp, Artificial Sequence
  primer
gtatgctcggctccaagtactgtt

SEQ ID NO: 1460 - DNA, 24 bp, Artificial Sequence
  primer
agagactcttataggcttgacgga

SEQ ID NO: 1461 - DNA, 24 bp, Artificial Sequence
  primer
acttaacagatatggatcatcgcc

SEQ ID NO: 1462 - DNA, 24 bp, Artificial Sequence
  primer
aatcagagcgagtctcgcttcagg

SEQ ID NO: 1463 - DNA, 24 bp, Artificial Sequence
  primer
accaccgaggaacaggtgcgacaa

SEQ ID NO: 1464 - DNA, 24 bp, Artificial Sequence
  primer
tggtacatgtcaaccgtaagcctg

SEQ ID NO: 1465 - DNA, 24 bp, Artificial Sequence
  primer
cgtgccgcggtgttcttgtatatg

SEQ ID NO: 1466 - DNA, 24 bp, Artificial Sequence
  primer
gacaagcgcgcgtgagacatatca

SEQ ID NO: 1467 - DNA, 24 bp, Artificial Sequence
  primer
agtgcactccgaacaagagttagt

SEQ ID NO: 1468 - DNA, 24 bp, Artificial Sequence
  primer
cctcattaccgcgttaggagtccg

SEQ ID NO: 1469 - DNA, 24 bp, Artificial Sequence
  primer
tgcttattgcttagttgctatctc

SEQ ID NO: 1470 - DNA, 24 bp, Artificial Sequence
  primer
gcgtgatcctgttctattcgttag

SEQ ID NO: 1471 - DNA, 24 bp, Artificial Sequence
  primer
ggccagaactatgacgagtataag

SEQ ID NO: 1472 - DNA, 24 bp, Artificial Sequence
  primer
gatggcgactatctaattgcaatg

SEQ ID NO: 1473 - DNA, 24 bp, Artificial Sequence
  primer
tagtaaccatagctctgtacaact

SEQ ID NO: 1474 - DNA, 24 bp, Artificial Sequence
  primer
cgtgatcgccaatacacatgtcgc

SEQ ID NO: 1475 - DNA, 24 bp, Artificial Sequence
  primer
taataacggatcgatatgcacgcg

SEQ ID NO: 1476 - DNA, 24 bp, Artificial Sequence
  primer
atcatcgcgctaatactatctgaa

SEQ ID NO: 1477 - DNA, 24 bp, Artificial Sequence
  primer
cacgtgcgtgcaggtcactagtat

SEQ ID NO: 1478 - DNA, 24 bp, Artificial Sequence
  primer
aggtccaatgccgagcgatcagaa

SEQ ID NO: 1479 - DNA, 24 bp, Artificial Sequence
  primer
cagcataacaacgagccaggtcag

SEQ ID NO: 1480 - DNA, 24 bp, Artificial Sequence
  primer
atggcgtccaatactccgacctat

SEQ ID NO: 1481 - DNA, 24 bp, Artificial Sequence
  primer
aggaacatcgtgaataatgaagac

SEQ ID NO: 1482 - DNA, 24 bp, Artificial Sequence
  primer
tctcgacgttcatgtaattaagga

SEQ ID NO: 1483 - DNA, 24 bp, Artificial Sequence
  primer
tcgcggttaaccttacttagacga

SEQ ID NO: 1484 - DNA, 24 bp, Artificial Sequence
  primer
atcatatctacggctctggcgccg

SEQ ID NO: 1485 - DNA, 24 bp, Artificial Sequence
  primer
gcagatggagaccagaggtacagg

SEQ ID NO: 1486 - DNA, 24 bp, Artificial Sequence
  primer
agacagaagattaccacgtgctat

SEQ ID NO: 1487 - DNA, 24 bp, Artificial Sequence
  primer
ccacggacaacatgccgcttaact

SEQ ID NO: 1488 - DNA, 24 bp, Artificial Sequence
  primer
cttgaagtctcaagctatgagaga

SEQ ID NO: 1489 - DNA, 24 bp, Artificial Sequence
  primer
acagcagtcgtgcttaggtcactg

SEQ ID NO: 1490 - DNA, 24 bp, Artificial Sequence
  primer
aggtgttaatgaacgtaggtgaga

SEQ ID NO: 1491 - DNA, 24 bp, Artificial Sequence
  primer
agccactatgttcaaggctgagcc

SEQ ID NO: 1492 - DNA, 24 bp, Artificial Sequence
  primer
gcaggcggtgtcgtgtgacaatga

SEQ ID NO: 1493 - DNA, 24 bp, Artificial Sequence
  primer
agccattgctacagaggttactta

SEQ ID NO: 1494 - DNA, 24 bp, Artificial Sequence
  primer
acaatcgaacctacactgagtccg

SEQ ID NO: 1495 - DNA, 24 bp, Artificial Sequence
  primer
ccgatctcaataggtaccacgaac

SEQ ID NO: 1496 - DNA, 24 bp, Artificial Sequence
  primer
gatacgtggcgctatgctaattaa

SEQ ID NO: 1497 - DNA, 24 bp, Artificial Sequence
  primer
agagagatggcacacattgacgtc

SEQ ID NO: 1498 - DNA, 24 bp, Artificial Sequence
  primer
ctcaactcatccttgtagccgatg

SEQ ID NO: 1499 - DNA, 24 bp, Artificial Sequence
  primer
gtggaataacgcgatacgactctt

SEQ ID NO: 1500 - DNA, 24 bp, Artificial Sequence
  primer
atctaccatgcgaatgctctctag

SEQ ID NO: 1501 - DNA, 24 bp, Artificial Sequence
  primer
atacgcacgcctgacacaaggacc

SEQ ID NO: 1502 - DNA, 24 bp, Artificial Sequence
  primer
gtccactctcagtgtgtagagtcc

SEQ ID NO: 1503 - DNA, 24 bp, Artificial Sequence
  primer
aatatatccagattctctgtgcag

SEQ ID NO: 1504 - DNA, 24 bp, Artificial Sequence
  primer
ccttccgccacatgttcgacaagg

SEQ ID NO: 1505 - DNA, 24 bp, Artificial Sequence
  primer
actgtgccatcatccgaggagcca

SEQ ID NO: 1506 - DNA, 24 bp, Artificial Sequence
  primer
tctatgccgctatggcgtcgtgta

SEQ ID NO: 1507 - DNA, 24 bp, Artificial Sequence
  primer
cgtaacctaaggtaatatgtctgc

SEQ ID NO: 1508 - DNA, 24 bp, Artificial Sequence
  primer
tactgaccgtatcaagattactaa

SEQ ID NO: 1509 - DNA, 24 bp, Artificial Sequence
  primer
tcatcggagcgccatacggtacgt

SEQ ID NO: 1510 - DNA, 24 bp, Artificial Sequence
  primer
gcaagaggaatgaacgaagtgatt

SEQ ID NO: 1511 - DNA, 24 bp, Artificial Sequence
  primer
ggctgattgacatcctgacttagt

SEQ ID NO: 1512 - DNA, 24 bp, Artificial Sequence
  primer
aaggcgctagattggattaacgta

SEQ ID NO: 1513 - DNA, 24 bp, Artificial Sequence
  primer
gctagctagaagaataggattcgt

SEQ ID NO: 1514 - DNA, 24 bp, Artificial Sequence
  primer
caggtgacggcctctataactcat

SEQ ID NO: 1515 - DNA, 24 bp, Artificial Sequence
  primer
caggttacacataccactatcttc

SEQ ID NO: 1516 - DNA, 24 bp, Artificial Sequence
  primer
ttgctacgtaccgtcttaatccgt

SEQ ID NO: 1517 - DNA, 24 bp, Artificial Sequence
  primer
ctcaacatgtcttgcaagcttcga

SEQ ID NO: 1518 - DNA, 24 bp, Artificial Sequence
  primer
ggtgcggtacgtagaaccagatca

SEQ ID NO: 1519 - DNA, 24 bp, Artificial Sequence
  primer
aatgctctccaagatcctgaccta

SEQ ID NO: 1520 - DNA, 24 bp, Artificial Sequence
  primer
gcttcgcaggtctggatgatggag

SEQ ID NO: 1521 - DNA, 24 bp, Artificial Sequence
  primer
acattgaccagacagcaccttgcg

SEQ ID NO: 1522 - DNA, 24 bp, Artificial Sequence
  primer
aggtatcaatgtgcttaataggcg

SEQ ID NO: 1523 - DNA, 24 bp, Artificial Sequence
  primer
tccggacacacgattagtaacgga

SEQ ID NO: 1524 - DNA, 24 bp, Artificial Sequence
  primer
tacgaagtactacagatcggtcag

SEQ ID NO: 1525 - DNA, 24 bp, Artificial Sequence
  primer
aattgtcagacgaatactgctgga

SEQ ID NO: 1526 - DNA, 24 bp, Artificial Sequence
  primer
tgaatcatgagccagaggttatgc

SEQ ID NO: 1527 - DNA, 24 bp, Artificial Sequence
  primer
cacaagacacgtcattaacatcaa

SEQ ID NO: 1528 - DNA, 24 bp, Artificial Sequence
  primer
gaatgactacattactccgccagg

SEQ ID NO: 1529 - DNA, 24 bp, Artificial Sequence
  primer
agccagagatactggaacttgact

SEQ ID NO: 1530 - DNA, 24 bp, Artificial Sequence
  primer
tatcagacacatcacaatggatac

SEQ ID NO: 1531 - DNA, 24 bp, Artificial Sequence
  primer
ctaggacaccgctagtcggttgaa

SEQ ID NO: 1532 - DNA, 24 bp, Artificial Sequence
  primer
gtataactgcgtgtcctggtgtat

SEQ ID NO: 1533 - DNA, 24 bp, Artificial Sequence
  primer
atgcaatactaaggtggacctccg

SEQ ID NO: 1534 - DNA, 24 bp, Artificial Sequence
  primer
atgcagacgcttgcgataagtcat

SEQ ID NO: 1535 - DNA, 24 bp, Artificial Sequence
  primer
ttgctcgatacacgtagaccagtg

SEQ ID NO: 1536 - DNA, 24 bp, Artificial Sequence
  primer
tactggaggacgattgtctatcat

SEQ ID NO: 1537 - DNA, 26 bp, Artificial Sequence
  primer
actaaggcacgctgattcgagcatta

SEQ ID NO: 1538 - DNA, 26 bp, Artificial Sequence
  primer
cggattctggcacgtacaagtagcag

SEQ ID NO: 1539 - DNA, 26 bp, Artificial Sequence
  primer
ttatggctccagatctagtcaccagc

SEQ ID NO: 1540 - DNA, 26 bp, Artificial Sequence
  primer
catacactccaggcatgtatgatagg

SEQ ID NO: 1541 - DNA, 26 bp, Artificial Sequence
  primer
agttgtaagccaacgagtgtagcgta

SEQ ID NO: 1542 - DNA, 26 bp, Artificial Sequence
  primer
gtatcagctccttcctctgattccgg

SEQ ID NO: 1543 - DNA, 26 bp, Artificial Sequence
  primer
aacatacagaatgtctatggtcagct

SEQ ID NO: 1544 - DNA, 26 bp, Artificial Sequence
  primer
gactcatattcatgttcagtatagag

SEQ ID NO: 1545 - DNA, 26 bp, Artificial Sequence
  primer
agagtgaacgaacgtgaccgacgctc

SEQ ID NO: 1546 - DNA, 26 bp, Artificial Sequence
  primer
aattggcgtccttgccacaacatctt

SEQ ID NO: 1547 - DNA, 26 bp, Artificial Sequence
  primer
tcgtagacgcctcgtacatccgagat

SEQ ID NO: 1548 - DNA, 26 bp, Artificial Sequence
  primer
ccggctcgtgaggcgataatcatata

SEQ ID NO: 1549 - DNA, 26 bp, Artificial Sequence
  primer
agtcctgatcacgaccacgactcacg

SEQ ID NO: 1550 - DNA, 26 bp, Artificial Sequence
  primer
ggcactcaatcctccatggagaagct

SEQ ID NO: 1551 - DNA, 26 bp, Artificial Sequence
  primer
tcatcattcctcacgttcaccggtga

SEQ ID NO: 1552 - DNA, 26 bp, Artificial Sequence
  primer
tcaactctgtgctaaccggtcgtaca

SEQ ID NO: 1553 - DNA, 26 bp, Artificial Sequence
  primer
tgttcttatgcattaatgccaggctt

SEQ ID NO: 1554 - DNA, 26 bp, Artificial Sequence
  primer
gattcacgacctcaacagcatcactc

SEQ ID NO: 1555 - DNA, 26 bp, Artificial Sequence
  primer
ggcgagttcgaccagaatgctggaca

SEQ ID NO: 1556 - DNA, 26 bp, Artificial Sequence
  primer
ttccgtatacaatgcgattaagatct

SEQ ID NO: 1557 - DNA, 26 bp, Artificial Sequence
  primer
gagtaatccgtaaccggccaacgttg

SEQ ID NO: 1558 - DNA, 26 bp, Artificial Sequence
  primer
cgcttccatcatggtacggtacgtat

SEQ ID NO: 1559 - DNA, 26 bp, Artificial Sequence
  primer
ccgtcgtggtgtgttgactggtcaac

SEQ ID NO: 1560 - DNA, 26 bp, Artificial Sequence
  primer
tattcgcatctccgtattagttgtag

SEQ ID NO: 1561 - DNA, 26 bp, Artificial Sequence
  primer
tattattgtattctaggcggtgcaac

SEQ ID NO: 1562 - DNA, 26 bp, Artificial Sequence
  primer
aggctgcctacttcctcgtcatctcg

SEQ ID NO: 1563 - DNA, 26 bp, Artificial Sequence
  primer
gtaacatacggctcatcgaatgcatc

SEQ ID NO: 1564 - DNA, 26 bp, Artificial Sequence
  primer
ttatggcacggatattaccgtacgcc

SEQ ID NO: 1565 - DNA, 26 bp, Artificial Sequence
  primer
atagcacttcctctaatgctctgctg

SEQ ID NO: 1566 - DNA, 26 bp, Artificial Sequence
  primer
tcacaggcaatagcctaatattatat

SEQ ID NO: 1567 - DNA, 26 bp, Artificial Sequence
  primer
ggcggatgttcgttaatattataagg

SEQ ID NO: 1568 - DNA, 26 bp, Artificial Sequence
  primer
tgcaatagccgttgtctctgccagcg

SEQ ID NO: 1569 - DNA, 26 bp, Artificial Sequence
  primer
tacagcgcgttggcgagtactgatag

SEQ ID NO: 1570 - DNA, 26 bp, Artificial Sequence
  primer
tgcagttagtaccttctcacgccaac

SEQ ID NO: 1571 - DNA, 26 bp, Artificial Sequence
  primer
ccattggctacctagcagactctacc

SEQ ID NO: 1572 - DNA, 26 bp, Artificial Sequence
  primer
aacagtagctcgcgtcttgctctcgt

SEQ ID NO: 1573 - DNA, 26 bp, Artificial Sequence
  primer
gcagtccatcagctctcgcttataga

SEQ ID NO: 1574 - DNA, 26 bp, Artificial Sequence
  primer
tatctctctgtcgccagcttgaccaa

SEQ ID NO: 1575 - DNA, 26 bp, Artificial Sequence
  primer
cagactgttcaagcttgctgtaggag

SEQ ID NO: 1576 - DNA, 26 bp, Artificial Sequence
  primer
taaccggaactcgttcagcaacattc

SEQ ID NO: 1577 - DNA, 26 bp, Artificial Sequence
  primer
tcaattatgcatgtcgtccgatctct

SEQ ID NO: 1578 - DNA, 26 bp, Artificial Sequence
  primer
ttgtctaagtcaacctgtggataatc

SEQ ID NO: 1579 - DNA, 26 bp, Artificial Sequence
  primer
tctaagagtggtatgaccaggagtcc

SEQ ID NO: 1580 - DNA, 26 bp, Artificial Sequence
  primer
tcgtagtactactggaacaggtaatc

SEQ ID NO: 1581 - DNA, 26 bp, Artificial Sequence
  primer
atgtcaacattctaatcatctctcgg

SEQ ID NO: 1582 - DNA, 26 bp, Artificial Sequence
  primer
agcgcgcaactgttacggtgatccga

SEQ ID NO: 1583 - DNA, 26 bp, Artificial Sequence
  primer
gcgatagaataatggtgtcacacacg

SEQ ID NO: 1584 - DNA, 26 bp, Artificial Sequence
  primer
aaggctgcgatgagaggcgtacatcg

SEQ ID NO: 1585 - DNA, 26 bp, Artificial Sequence
  primer
ggttcatggtctcagtcgtgatcgcg

SEQ ID NO: 1586 - DNA, 26 bp, Artificial Sequence
  primer
tagtgactctatgtcacctcggagcc

SEQ ID NO: 1587 - DNA, 26 bp, Artificial Sequence
  primer
atgtgatagcaatggcacctctagtc

SEQ ID NO: 1588 - DNA, 26 bp, Artificial Sequence
  primer
tcgcgaagtgtaatgcatcatccgct

SEQ ID NO: 1589 - DNA, 26 bp, Artificial Sequence
  primer
atgtggcgacgatccaagttcaacgc

SEQ ID NO: 1590 - DNA, 26 bp, Artificial Sequence
  primer
accttgtatgagtcggagtgtccggc

SEQ ID NO: 1591 - DNA, 26 bp, Artificial Sequence
  primer
acctcaagagagtagacagttgagtt

SEQ ID NO: 1592 - DNA, 26 bp, Artificial Sequence
  primer
ggtgtaatcctgtgtgcgaagctggt

SEQ ID NO: 1593 - DNA, 26 bp, Artificial Sequence
  primer
atagcggaactgtacgacgctccagt

SEQ ID NO: 1594 - DNA, 26 bp, Artificial Sequence
  primer
aagcacgagtcgaccattagcctgga

SEQ ID NO: 1595 - DNA, 26 bp, Artificial Sequence
  primer
attccggtaacatcagaaggtacaat

SEQ ID NO: 1596 - DNA, 26 bp, Artificial Sequence
  primer
gtgcaacggcagtccagtatcctggt

SEQ ID NO: 1597 - DNA, 26 bp, Artificial Sequence
  primer
ccatcttatacacggtgaccgaagat

SEQ ID NO: 1598 - DNA, 26 bp, Artificial Sequence
  primer
gcacttaatcaagcttgagtgatgct

SEQ ID NO: 1599 - DNA, 26 bp, Artificial Sequence
  primer
agtattacgtgagtacgaagatagca

SEQ ID NO: 1600 - DNA, 26 bp, Artificial Sequence
  primer
ttcttaggttaagttccttctggacc

SEQ ID NO: 1601 - DNA, 26 bp, Artificial Sequence
  primer
gtccttgctagacactgaccgttgct

SEQ ID NO: 1602 - DNA, 26 bp, Artificial Sequence
  primer
gccgctatgtgtgctgcatcctaagc

SEQ ID NO: 1603 - DNA, 26 bp, Artificial Sequence
  primer
ccatcaataacagacttatgttgtga

SEQ ID NO: 1604 - DNA, 26 bp, Artificial Sequence
  primer
cgcgtgtgcttacaagtgctaacaag

SEQ ID NO: 1605 - DNA, 26 bp, Artificial Sequence
  primer
cgatatgtgttcgcaataagagagcc

SEQ ID NO: 1606 - DNA, 26 bp, Artificial Sequence
  primer
cgcggatgtgagcggctcaattagca

SEQ ID NO: 1607 - DNA, 26 bp, Artificial Sequence
  primer
gctgcatgactatcggatggaggcat

SEQ ID NO: 1608 - DNA, 26 bp, Artificial Sequence
  primer
ctatgccgtgtatggtacgagtggcg

SEQ ID NO: 1609 - DNA, 26 bp, Artificial Sequence
  primer
ccggctggagttcattacgtaggctg

SEQ ID NO: 1610 - DNA, 26 bp, Artificial Sequence
  primer
tgtaggcctactgagctagtattaga

SEQ ID NO: 1611 - DNA, 26 bp, Artificial Sequence
  primer
ccgtcaagtgactattcttctaatct

SEQ ID NO: 1612 - DNA, 26 bp, Artificial Sequence
  primer
ggtcttacgccagagactgcgcttct

SEQ ID NO: 1613 - DNA, 26 bp, Artificial Sequence
  primer
cgaagtgtgattattaactgtaatct

SEQ ID NO: 1614 - DNA, 26 bp, Artificial Sequence
  primer
gcacgcgtggccgtaagcatcgatta

SEQ ID NO: 1615 - DNA, 26 bp, Artificial Sequence
  primer
atcctgcgtcggaacgtactatagct

SEQ ID NO: 1616 - DNA, 26 bp, Artificial Sequence
  primer
agtatcatcatatccattcgcagtac

SEQ ID NO: 1617 - DNA, 26 bp, Artificial Sequence
  primer
agtcctgacgttcatatatagactcc

SEQ ID NO: 1618 - DNA, 26 bp, Artificial Sequence
  primer
cttgcagtaatctgaatctgaaggtt

SEQ ID NO: 1619 - DNA, 26 bp, Artificial Sequence
  primer
ataacttggttccagtaacgcatagt

SEQ ID NO: 1620 - DNA, 26 bp, Artificial Sequence
  primer
gataaggatatggctgtagcgaagtg

SEQ ID NO: 1621 - DNA, 26 bp, Artificial Sequence
  primer
gtggagcgttacagacatgctgaaca

SEQ ID NO: 1622 - DNA, 26 bp, Artificial Sequence
  primer
cgcttccggcaggcgtcatataagtc

SEQ ID NO: 1623 - DNA, 26 bp, Artificial Sequence
  primer
ataacattctaacctctataagccga

SEQ ID NO: 1624 - DNA, 26 bp, Artificial Sequence
  primer
acgatctatgatccatatggacttcc

SEQ ID NO: 1625 - DNA, 26 bp, Artificial Sequence
  primer
tgaagctcagatatcatgcctcgagc

SEQ ID NO: 1626 - DNA, 26 bp, Artificial Sequence
  primer
agacttcaccgcaataactcgtagat

SEQ ID NO: 1627 - DNA, 26 bp, Artificial Sequence
  primer
agactaagacatacgccatcaccgct

SEQ ID NO: 1628 - DNA, 26 bp, Artificial Sequence
  primer
tgtagcgtgatgtatcgtaattctgt

SEQ ID NO: 1629 - DNA, 26 bp, Artificial Sequence
  primer
tgtgctattggcacctcacgctgacc

SEQ ID NO: 1630 - DNA, 26 bp, Artificial Sequence
  primer
tgtagataagtatccagcgactctct

SEQ ID NO: 1631 - DNA, 26 bp, Artificial Sequence
  primer
aattcgccaattgtgtgtaggcgcaa

SEQ ID NO: 1632 - DNA, 26 bp, Artificial Sequence
  primer
cgattatgagtacttgtagaccagct

SEQ ID NO: 1633 - DNA, 28 bp, Artificial Sequence
  primer
ttgcaagaacaacgtatctcatatgaac

SEQ ID NO: 1634 - DNA, 28 bp, Artificial Sequence
  primer
caccgtgctgttattacttggtattcgg

SEQ ID NO: 1635 - DNA, 28 bp, Artificial Sequence
  primer
cacgtgtattgttgcaccagaacgacaa

SEQ ID NO: 1636 - DNA, 28 bp, Artificial Sequence
  primer
atgcacgtaattacttccggagaagacg

SEQ ID NO: 1637 - DNA, 28 bp, Artificial Sequence
  primer
tatgttgtctgatatggttcatgtggca

SEQ ID NO: 1638 - DNA, 28 bp, Artificial Sequence
  primer
agcgcgactagttgatgccaacattgta

SEQ ID NO: 1639 - DNA, 28 bp, Artificial Sequence
  primer
ataggcaggtccaggctcggaacaagtc

SEQ ID NO: 1640 - DNA, 28 bp, Artificial Sequence
  primer
gcggtagtcggtcaagaactagaaccgt

SEQ ID NO: 1641 - DNA, 28 bp, Artificial Sequence
  primer
actatacactctagctattaggaagcat

SEQ ID NO: 1642 - DNA, 28 bp, Artificial Sequence
  primer
gatcatcttgcttctcctgtggagataa

SEQ ID NO: 1643 - DNA, 28 bp, Artificial Sequence
  primer
ctactacgagtccataactgatagcctc

SEQ ID NO: 1644 - DNA, 28 bp, Artificial Sequence
  primer
gcacagacacctgtcctatctagcagga

SEQ ID NO: 1645 - DNA, 28 bp, Artificial Sequence
  primer
aagcgaggcgcgaaggagatggaaggat

SEQ ID NO: 1646 - DNA, 28 bp, Artificial Sequence
  primer
ctgaagacgccagtctggataggtgcct

SEQ ID NO: 1647 - DNA, 28 bp, Artificial Sequence
  primer
gtaagctctgtccttcgagattgataag

SEQ ID NO: 1648 - DNA, 28 bp, Artificial Sequence
  primer
ggttagagagattattgtgcgcatccat

SEQ ID NO: 1649 - DNA, 28 bp, Artificial Sequence
  primer
ccaggaggacctatgatcttgccgccat

SEQ ID NO: 1650 - DNA, 28 bp, Artificial Sequence
  primer
actattcgagctactgtatgtgtatccg

SEQ ID NO: 1651 - DNA, 28 bp, Artificial Sequence
  primer
gacatcgcgatacgtaactccggagtgt

SEQ ID NO: 1652 - DNA, 28 bp, Artificial Sequence
  primer
ccgcaattcgtctatatattctagcata

SEQ ID NO: 1653 - DNA, 28 bp, Artificial Sequence
  primer
ctacacttgaggttgatgctcaagatca

SEQ ID NO: 1654 - DNA, 28 bp, Artificial Sequence
  primer
cgatcagttctagttcaccgcggacaat

SEQ ID NO: 1655 - DNA, 28 bp, Artificial Sequence
  primer
aagaatgatgattggccgcgaaccaagc

SEQ ID NO: 1656 - DNA, 28 bp, Artificial Sequence
  primer
cacgaccggaactagactcctaccaatt

SEQ ID NO: 1657 - DNA, 28 bp, Artificial Sequence
  primer
agttgcctgtgagtgaggctactatctc

SEQ ID NO: 1658 - DNA, 28 bp, Artificial Sequence
  primer
gattcttccgatgatcatgccactacaa

SEQ ID NO: 1659 - DNA, 28 bp, Artificial Sequence
  primer
cgctgaagtgaactatgcaagcaccgca

SEQ ID NO: 1660 - DNA, 28 bp, Artificial Sequence
  primer
attatcgtgatggtgagactgagctcgt

SEQ ID NO: 1661 - DNA, 28 bp, Artificial Sequence
  primer
cgaggccactctgagccaggtaagtatc

SEQ ID NO: 1662 - DNA, 28 bp, Artificial Sequence
  primer
tgccgaggacagccgatcacatcttcgt

SEQ ID NO: 1663 - DNA, 28 bp, Artificial Sequence
  primer
gttgacatgaaggttatcgtcgatattc

SEQ ID NO: 1664 - DNA, 28 bp, Artificial Sequence
  primer
gtggtccaggtcaagctctgatcgaatg

SEQ ID NO: 1665 - DNA, 28 bp, Artificial Sequence
  primer
ccagtccggtgtactcagacctaataac

SEQ ID NO: 1666 - DNA, 28 bp, Artificial Sequence
  primer
cgagacactgcatgagcgtagtcttatt

SEQ ID NO: 1667 - DNA, 28 bp, Artificial Sequence
  primer
gacggcttgtatacttctctacggtctg

SEQ ID NO: 1668 - DNA, 28 bp, Artificial Sequence
  primer
ttagctggatggaagccatattccgtag

SEQ ID NO: 1669 - DNA, 28 bp, Artificial Sequence
  primer
cagcctacacttgattactcaacaactc

SEQ ID NO: 1670 - DNA, 28 bp, Artificial Sequence
  primer
gtacgtagtgtcacgcgcctacgttcgt

SEQ ID NO: 1671 - DNA, 28 bp, Artificial Sequence
  primer
ctacaacttctcaatcatgcctctgttg

SEQ ID NO: 1672 - DNA, 28 bp, Artificial Sequence
  primer
cgaggacagaattcgacataaggagaga

SEQ ID NO: 1673 - DNA, 28 bp, Artificial Sequence
  primer
gccgaacgacacagtgagttgataggta

SEQ ID NO: 1674 - DNA, 28 bp, Artificial Sequence
  primer
gaacactatatgctgtcgctgtctgagg

SEQ ID NO: 1675 - DNA, 28 bp, Artificial Sequence
  primer
gttaagttcttcggcggtcatgctcatt

SEQ ID NO: 1676 - DNA, 28 bp, Artificial Sequence
  primer
ttgcttacagatcgcgtatccatagtat

SEQ ID NO: 1677 - DNA, 28 bp, Artificial Sequence
  primer
gaggaccacctctgcgaagttcactgtg

SEQ ID NO: 1678 - DNA, 28 bp, Artificial Sequence
  primer
aatcctagcatatcgagaacgacactga

SEQ ID NO: 1679 - DNA, 28 bp, Artificial Sequence
  primer
tgaatactatagccatagtcgacttccg

SEQ ID NO: 1680 - DNA, 28 bp, Artificial Sequence
  primer
gacatccacgaagctggtaatcggaacc

SEQ ID NO: 1681 - DNA, 28 bp, Artificial Sequence
  primer
ttagccgtcttagaagtgtctgaccggc

SEQ ID NO: 1682 - DNA, 28 bp, Artificial Sequence
  primer
ctattctgccgtaattgattccttcgtt

SEQ ID NO: 1683 - DNA, 28 bp, Artificial Sequence
  primer
acgcctctggtcgaaggtagattagctc

SEQ ID NO: 1684 - DNA, 28 bp, Artificial Sequence
  primer
cagcctattgatcgtaagtagatggtcc

SEQ ID NO: 1685 - DNA, 28 bp, Artificial Sequence
  primer
ttaagtgaggtggacaaccatcaacttc

SEQ ID NO: 1686 - DNA, 28 bp, Artificial Sequence
  primer
aaggccttgcggctaagtagtattcatc

SEQ ID NO: 1687 - DNA, 28 bp, Artificial Sequence
  primer
ttgtgatactaattcttctcaagagtca

SEQ ID NO: 1688 - DNA, 28 bp, Artificial Sequence
  primer
gcattaggtgacgaccttagtccatcac

SEQ ID NO: 1689 - DNA, 28 bp, Artificial Sequence
  primer
gcggatggacgtatacagtgagtcgtgc

SEQ ID NO: 1690 - DNA, 28 bp, Artificial Sequence
  primer
gaacatgccagcctcaactaggctaaga

SEQ ID NO: 1691 - DNA, 28 bp, Artificial Sequence
  primer
tccgtcattagagtatgagtgactacta

SEQ ID NO: 1692 - DNA, 28 bp, Artificial Sequence
  primer
aacacttagtaaccagttcggactggac

SEQ ID NO: 1693 - DNA, 28 bp, Artificial Sequence
  primer
cgctaactattgcgtatattcgcggctt

SEQ ID NO: 1694 - DNA, 28 bp, Artificial Sequence
  primer
gccatctacgatcttcggcttatcctag

SEQ ID NO: 1695 - DNA, 28 bp, Artificial Sequence
  primer
cctgagaatgttgactaagatcttgtga

SEQ ID NO: 1696 - DNA, 28 bp, Artificial Sequence
  primer
tcggttagtctaatcatcacgcaacgga

SEQ ID NO: 1697 - DNA, 28 bp, Artificial Sequence
  primer
attatctattgaagcagtgacagcgatc

SEQ ID NO: 1698 - DNA, 28 bp, Artificial Sequence
  primer
gaggagaatcacggaacacggtcacatg

SEQ ID NO: 1699 - DNA, 28 bp, Artificial Sequence
  primer
gctgcaagcattatgaccatggcatctg

SEQ ID NO: 1700 - DNA, 28 bp, Artificial Sequence
  primer
gaacaacctataacgacgttgtggacaa

SEQ ID NO: 1701 - DNA, 28 bp, Artificial Sequence
  primer
ttaatcatcgatagacgacatggaatca

SEQ ID NO: 1702 - DNA, 28 bp, Artificial Sequence
  primer
tcgagtgtaagcacactacgatctggaa

SEQ ID NO: 1703 - DNA, 28 bp, Artificial Sequence
  primer
gctacgcacagtctctgcacagctacac

SEQ ID NO: 1704 - DNA, 28 bp, Artificial Sequence
  primer
cctgtatgtacgttctggctaatacctt

SEQ ID NO: 1705 - DNA, 28 bp, Artificial Sequence
  primer
tgaagcaccggtacatggtgtatccgga

SEQ ID NO: 1706 - DNA, 28 bp, Artificial Sequence
  primer
tgctggaacctaactcggtgatgacgat

SEQ ID NO: 1707 - DNA, 28 bp, Artificial Sequence
  primer
cgctatcttactgccaagttctcatata

SEQ ID NO: 1708 - DNA, 28 bp, Artificial Sequence
  primer
aacgcgcgcgtatcggcaataatctcaa

SEQ ID NO: 1709 - DNA, 28 bp, Artificial Sequence
  primer
ccattaggatgaccatcgactattagag

SEQ ID NO: 1710 - DNA, 28 bp, Artificial Sequence
  primer
tactgctagactgcgtgcattcatggcg

SEQ ID NO: 1711 - DNA, 28 bp, Artificial Sequence
  primer
cattgcgcgctccacgaactctattgtc

SEQ ID NO: 1712 - DNA, 28 bp, Artificial Sequence
  primer
gacgcgcctagaactgtatagctctacg

SEQ ID NO: 1713 - DNA, 28 bp, Artificial Sequence
  primer
cattgcaacttgtcggtgatggcaatcc

SEQ ID NO: 1714 - DNA, 28 bp, Artificial Sequence
  primer
ttaatgcacatgcagtacggcaccacag

SEQ ID NO: 1715 - DNA, 28 bp, Artificial Sequence
  primer
agcggtacgtggacgagtggtaattaat

SEQ ID NO: 1716 - DNA, 28 bp, Artificial Sequence
  primer
gacgtattgctatgcattggaagatgct

SEQ ID NO: 1717 - DNA, 28 bp, Artificial Sequence
  primer
aacacttcgaccattgcgcctcaatggt

SEQ ID NO: 1718 - DNA, 28 bp, Artificial Sequence
  primer
cggtacgctctagcggtcataagatgca

SEQ ID NO: 1719 - DNA, 28 bp, Artificial Sequence
  primer
cctgaataacagccgcgcctaattagat

SEQ ID NO: 1720 - DNA, 28 bp, Artificial Sequence
  primer
aagcgtctaatgtgccttaagtcacatg

SEQ ID NO: 1721 - DNA, 28 bp, Artificial Sequence
  primer
gctctccaagaaccagaagtaagcatcg

SEQ ID NO: 1722 - DNA, 28 bp, Artificial Sequence
  primer
gaggagagttgtccgagtggtgtgatgt

SEQ ID NO: 1723 - DNA, 28 bp, Artificial Sequence
  primer
taacgagtggtgcgtctaagcaattgag

SEQ ID NO: 1724 - DNA, 28 bp, Artificial Sequence
  primer
ccaacagtatgctgacataactatgata

SEQ ID NO: 1725 - DNA, 28 bp, Artificial Sequence
  primer
gatccttgccacgcctatgagatatcgc

SEQ ID NO: 1726 - DNA, 28 bp, Artificial Sequence
  primer
aacgcgctaccgtccttgtgcatagagg

SEQ ID NO: 1727 - DNA, 28 bp, Artificial Sequence
  primer
ctacatgtgccttatagtacagaggaac

SEQ ID NO: 1728 - DNA, 28 bp, Artificial Sequence
  primer
cagcctcgtagttagcgtgattcatgcg

SEQ ID NO: 1729 - DNA, 29 bp, Artificial Sequence
  primer
ctcctcgccgattgaagtgcgtagaacta

SEQ ID NO: 1730 - DNA, 29 bp, Artificial Sequence
  primer
cagcaggcctcaataggataagccaacta

SEQ ID NO: 1731 - DNA, 29 bp, Artificial Sequence
  primer
gaccatcaatctcgaagactacgctctgt

SEQ ID NO: 1732 - DNA, 29 bp, Artificial Sequence
  primer
ggttgctccgtctgttcagcacactgtta

SEQ ID NO: 1733 - DNA, 29 bp, Artificial Sequence
  primer
aatgtcgactggccattatcgccaagtgt

SEQ ID NO: 1734 - DNA, 29 bp, Artificial Sequence
  primer
gatagcttgccatgcgaatggatctccag

SEQ ID NO: 1735 - DNA, 29 bp, Artificial Sequence
  primer
ccagaccggagccaattggctgccaatat

SEQ ID NO: 1736 - DNA, 29 bp, Artificial Sequence
  primer
aacgtcgctccatacgttacctaatgcag

SEQ ID NO: 1737 - DNA, 29 bp, Artificial Sequence
  primer
gaatatgacgcgaacagtctattcggatc

SEQ ID NO: 1738 - DNA, 29 bp, Artificial Sequence
  primer
gacgagaatgtattaaggataagcaaggt

SEQ ID NO: 1739 - DNA, 29 bp, Artificial Sequence
  primer
aagtcgtatgaatcgctatcacatgagtc

SEQ ID NO: 1740 - DNA, 29 bp, Artificial Sequence
  primer
gtcgtggagactacaattctcctcacgtt

SEQ ID NO: 1741 - DNA, 29 bp, Artificial Sequence
  primer
gttgccaccgttacacgactatcgacagt

SEQ ID NO: 1742 - DNA, 29 bp, Artificial Sequence
  primer
aggataggctacgccttactctcctaagc

SEQ ID NO: 1743 - DNA, 29 bp, Artificial Sequence
  primer
taatcatcctgttcgcctcgaggttgtta

SEQ ID NO: 1744 - DNA, 29 bp, Artificial Sequence
  primer
gacaagcagtaataattactgagtggacg

SEQ ID NO: 1745 - DNA, 29 bp, Artificial Sequence
  primer
tacagcgttacgcaggtatatcaaggtag

SEQ ID NO: 1746 - DNA, 29 bp, Artificial Sequence
  primer
ctaacatcacttactattagcggtctcgt

SEQ ID NO: 1747 - DNA, 29 bp, Artificial Sequence
  primer
ccgcgcttcttgacacgttctccactagg

SEQ ID NO: 1748 - DNA, 29 bp, Artificial Sequence
  primer
caagtaacatgagatgctatcggtacatt

SEQ ID NO: 1749 - DNA, 29 bp, Artificial Sequence
  primer
cgaccactaggctgtgaccacgatacgct

SEQ ID NO: 1750 - DNA, 29 bp, Artificial Sequence
  primer
caggtcatgtgacgcagtcggcagtcaac

SEQ ID NO: 1751 - DNA, 29 bp, Artificial Sequence
  primer
actccatcgttagttcttccgccgtgctg

SEQ ID NO: 1752 - DNA, 29 bp, Artificial Sequence
  primer
ctcaccacgtatgcgtcactcggttacgt

SEQ ID NO: 1753 - DNA, 29 bp, Artificial Sequence
  primer
tgcctatgctatggaccttgcgcgactct

SEQ ID NO: 1754 - DNA, 29 bp, Artificial Sequence
  primer
aatgaaggtcaacgctctgtagttacgcg

SEQ ID NO: 1755 - DNA, 29 bp, Artificial Sequence
  primer
caccattgattcatggcttccatcactgc

SEQ ID NO: 1756 - DNA, 29 bp, Artificial Sequence
  primer
gacacgcaaggtaattcgagattgcagca

SEQ ID NO: 1757 - DNA, 29 bp, Artificial Sequence
  primer
caccgagaggaaggttcgatcgcttctcg

SEQ ID NO: 1758 - DNA, 29 bp, Artificial Sequence
  primer
cagttatcggattgtgatattcactcctg

SEQ ID NO: 1759 - DNA, 29 bp, Artificial Sequence
  primer
atactgtaacgcctcaacctatgctgact

SEQ ID NO: 1760 - DNA, 29 bp, Artificial Sequence
  primer
atctgtcttattctggcacactcagactt

SEQ ID NO: 1761 - DNA, 29 bp, Artificial Sequence
  primer
tccaaccggtgacgtgctcttgatccaac

SEQ ID NO: 1762 - DNA, 29 bp, Artificial Sequence
  primer
cacactcagttcggctatctctgcgatag

SEQ ID NO: 1763 - DNA, 29 bp, Artificial Sequence
  primer
agctgtaagtcaggtctacgactcgtact

SEQ ID NO: 1764 - DNA, 29 bp, Artificial Sequence
  primer
gtcggcggcacgcacagctaacattcgta

SEQ ID NO: 1765 - DNA, 29 bp, Artificial Sequence
  primer
atatggtagccagccacgtatactgaaca

SEQ ID NO: 1766 - DNA, 29 bp, Artificial Sequence
  primer
tggacaatccgactctaacacagaggtag

SEQ ID NO: 1767 - DNA, 29 bp, Artificial Sequence
  primer
tccgccgctgacagttcaatctatcaatt

SEQ ID NO: 1768 - DNA, 29 bp, Artificial Sequence
  primer
ggttccttagaatatgcacctatcagcga

SEQ ID NO: 1769 - DNA, 29 bp, Artificial Sequence
  primer
cggctgtacgacatggatcataagagtgt

SEQ ID NO: 1770 - DNA, 29 bp, Artificial Sequence
  primer
tgcagatgtacgctgtggccagtggagag

SEQ ID NO: 1771 - DNA, 29 bp, Artificial Sequence
  primer
cctactcacttaacaataatcggttcggt

SEQ ID NO: 1772 - DNA, 29 bp, Artificial Sequence
  primer
cgcttcctactgcctgtgccgcgacataa

SEQ ID NO: 1773 - DNA, 29 bp, Artificial Sequence
  primer
ctagaccgaccggttatgcgctattgttc

SEQ ID NO: 1774 - DNA, 29 bp, Artificial Sequence
  primer
ttgtgagcacgtctgcggcaagcctatgg

SEQ ID NO: 1775 - DNA, 29 bp, Artificial Sequence
  primer
tcatcggccggcgctgttgttgttaccat

SEQ ID NO: 1776 - DNA, 29 bp, Artificial Sequence
  primer
gcggttaggtgcagttaggaagactatca

SEQ ID NO: 1777 - DNA, 29 bp, Artificial Sequence
  primer
tatgcggtcgtgaggcgtagcattctaga

SEQ ID NO: 1778 - DNA, 29 bp, Artificial Sequence
  primer
ccatctattcgtcgaactctcagctcgta

SEQ ID NO: 1779 - DNA, 29 bp, Artificial Sequence
  primer
atcagatctactgatcgcggtagagtatc

SEQ ID NO: 1780 - DNA, 29 bp, Artificial Sequence
  primer
tacacataggcggcgcagccttctaatta

SEQ ID NO: 1781 - DNA, 29 bp, Artificial Sequence
  primer
ttaaccgtagttcttagcttacgccgctc

SEQ ID NO: 1782 - DNA, 29 bp, Artificial Sequence
  primer
actatagaggacatggcactcctcttcta

SEQ ID NO: 1783 - DNA, 29 bp, Artificial Sequence
  primer
cagttcgtattaagattgaatgtagcggt

SEQ ID NO: 1784 - DNA, 29 bp, Artificial Sequence
  primer
agttatcggtatccgcttatccgtacgta

SEQ ID NO: 1785 - DNA, 29 bp, Artificial Sequence
  primer
agcttattcatacactgcaccacagcaag

SEQ ID NO: 1786 - DNA, 29 bp, Artificial Sequence
  primer
ccgtcggctagtctatcctctaattagaa

SEQ ID NO: 1787 - DNA, 29 bp, Artificial Sequence
  primer
gtccgcttccatgcctgctgtacgaacac

SEQ ID NO: 1788 - DNA, 29 bp, Artificial Sequence
  primer
tctcttcctccttcattgttcgctagctc

SEQ ID NO: 1789 - DNA, 29 bp, Artificial Sequence
  primer
tctcttgagcggtcctcatacaggtctgc

SEQ ID NO: 1790 - DNA, 29 bp, Artificial Sequence
  primer
gaccaagtgtaggtgatatcaccggtact

SEQ ID NO: 1791 - DNA, 29 bp, Artificial Sequence
  primer
aagattgtgataggttggtagttaccaca

SEQ ID NO: 1792 - DNA, 29 bp, Artificial Sequence
  primer
tcgcctccgaagagtatagcatcggcaga

SEQ ID NO: 1793 - DNA, 29 bp, Artificial Sequence
  primer
gaggtagttatgagcatcgaggtcctgtt

SEQ ID NO: 1794 - DNA, 29 bp, Artificial Sequence
  primer
ggacgcaagatcgcaggtacttgtaagct

SEQ ID NO: 1795 - DNA, 29 bp, Artificial Sequence
  primer
actcgtacacgtcatcgtgcaggtctcag

SEQ ID NO: 1796 - DNA, 29 bp, Artificial Sequence
  primer
taatccgtcaggagtgagatggctcgaca

SEQ ID NO: 1797 - DNA, 29 bp, Artificial Sequence
  primer
aagatggttccgcgcattgactagcaagt

SEQ ID NO: 1798 - DNA, 29 bp, Artificial Sequence
  primer
tccgcgatctgcggatcttgaatgctcac

SEQ ID NO: 1799 - DNA, 29 bp, Artificial Sequence
  primer
ttcacgagagtcaactgctagtatcctag

SEQ ID NO: 1800 - DNA, 29 bp, Artificial Sequence
  primer
ttccaactggattcttccaactcctcgaa

SEQ ID NO: 1801 - DNA, 29 bp, Artificial Sequence
  primer
cactactactcaagttatacggtgttgac

SEQ ID NO: 1802 - DNA, 29 bp, Artificial Sequence
  primer
caactggattctcaggatgcgtctctagc

SEQ ID NO: 1803 - DNA, 29 bp, Artificial Sequence
  primer
tggactagagtggagcgattacgtaatat

SEQ ID NO: 1804 - DNA, 29 bp, Artificial Sequence
  primer
gaggtcattcaactggactcgccacggac

SEQ ID NO: 1805 - DNA, 29 bp, Artificial Sequence
  primer
caggtgtgtaacgctgcaatcacatgaat

SEQ ID NO: 1806 - DNA, 29 bp, Artificial Sequence
  primer
tatgctgaggtattagttctaactatgcg

SEQ ID NO: 1807 - DNA, 29 bp, Artificial Sequence
  primer
cgtctgagtcggataaggaaggttaccgc

SEQ ID NO: 1808 - DNA, 29 bp, Artificial Sequence
  primer
gtactatcgtcgcaggcactatctctgcc

SEQ ID NO: 1809 - DNA, 29 bp, Artificial Sequence
  primer
gcttcctccttgcaacttcattgcttcga

SEQ ID NO: 1810 - DNA, 29 bp, Artificial Sequence
  primer
tgtctacgaagtagaagacacgaataatg

SEQ ID NO: 1811 - DNA, 29 bp, Artificial Sequence
  primer
ccgtcatctaaggcagagtacatccgcga

SEQ ID NO: 1812 - DNA, 29 bp, Artificial Sequence
  primer
ccggaggcgtactaactgaccacaacacc

SEQ ID NO: 1813 - DNA, 29 bp, Artificial Sequence
  primer
aactcgtcgctgcctgaataggtcagagt

SEQ ID NO: 1814 - DNA, 29 bp, Artificial Sequence
  primer
ttataagattaatgtcggtcagtgtcgga

SEQ ID NO: 1815 - DNA, 29 bp, Artificial Sequence
  primer
cgtctcgatggatccacacgaacctgttg

SEQ ID NO: 1816 - DNA, 29 bp, Artificial Sequence
  primer
atgccatcatggtcgtcctatcttaaggc

SEQ ID NO: 1817 - DNA, 29 bp, Artificial Sequence
  primer
gcgcttcagcgattcgtcatgcaaggcac

SEQ ID NO: 1818 - DNA, 29 bp, Artificial Sequence
  primer
ccaagcgataccgaggtacggttaacgag

SEQ ID NO: 1819 - DNA, 29 bp, Artificial Sequence
  primer
atatgacagacaggtggacctaagcaagc

SEQ ID NO: 1820 - DNA, 29 bp, Artificial Sequence
  primer
cactacatcgtcaggcctggaagcctcag

SEQ ID NO: 1821 - DNA, 29 bp, Artificial Sequence
  primer
gccgtgtagacgaggacattatgtcgtat

SEQ ID NO: 1822 - DNA, 29 bp, Artificial Sequence
  primer
caacgtatatacacaccttgtgaagagaa

SEQ ID NO: 1823 - DNA, 29 bp, Artificial Sequence
  primer
tccaacgtaattccgccgtctgtcgagac

SEQ ID NO: 1824 - DNA, 29 bp, Artificial Sequence
  primer
aattcgtgcttcgatcaccgtagactcag

SEQ ID NO: 1825 - DNA, 30 bp, Artificial Sequence
  primer
actatattgtattcacgtccgacgactcgc

SEQ ID NO: 1826 - DNA, 30 bp, Artificial Sequence
  primer
gacgagcttgtggtacactatacctatgag

SEQ ID NO: 1827 - DNA, 30 bp, Artificial Sequence
  primer
tgattcaagcaccaggcatgcttaagctag

SEQ ID NO: 1828 - DNA, 30 bp, Artificial Sequence
  primer
cggtctcctataggaaggctcattctgacg

SEQ ID NO: 1829 - DNA, 30 bp, Artificial Sequence
  primer
agtcagtgtcgaatcaatcaaggcgtcctt

SEQ ID NO: 1830 - DNA, 30 bp, Artificial Sequence
  primer
cgaacgtaatggccatcacgcgctggccta

SEQ ID NO: 1831 - DNA, 30 bp, Artificial Sequence
  primer
cgaacctggaccacctggcattaccattac

SEQ ID NO: 1832 - DNA, 30 bp, Artificial Sequence
  primer
acattaggttcctgtaatgtcttatcaacg

SEQ ID NO: 1833 - DNA, 30 bp, Artificial Sequence
  primer
cgtctaatgcaccgtatcgtcttcgcgcat

SEQ ID NO: 1834 - DNA, 30 bp, Artificial Sequence
  primer
tctatgacttacaacggaatcttacttcgt

SEQ ID NO: 1835 - DNA, 30 bp, Artificial Sequence
  primer
gtaaccgatcggtaccgtctgctattgttc

SEQ ID NO: 1836 - DNA, 30 bp, Artificial Sequence
  primer
ggtgattgataagcaacacatattaggagg

SEQ ID NO: 1837 - DNA, 30 bp, Artificial Sequence
  primer
aattatcgacgctaataggcgagctgttca

SEQ ID NO: 1838 - DNA, 30 bp, Artificial Sequence
  primer
ggaggtacatgacgagtggacagacagacc

SEQ ID NO: 1839 - DNA, 30 bp, Artificial Sequence
  primer
ctctaatccgttatgcggtgatgtaatccg

SEQ ID NO: 1840 - DNA, 30 bp, Artificial Sequence
  primer
gcaagcacgcggcttggcgaacttctatgc

SEQ ID NO: 1841 - DNA, 30 bp, Artificial Sequence
  primer
tagatgtaggcctggtaggcagaggagtaa

SEQ ID NO: 1842 - DNA, 30 bp, Artificial Sequence
  primer
ccgagtggcgaccacacaggtacgcattaa

SEQ ID NO: 1843 - DNA, 30 bp, Artificial Sequence
  primer
gtcctggctcagattagtgcacttagttat

SEQ ID NO: 1844 - DNA, 30 bp, Artificial Sequence
  primer
gcggtacctacatgttatgactcagacgac

SEQ ID NO: 1845 - DNA, 30 bp, Artificial Sequence
  primer
tctctgccaatgctggtctcatcgaatcca

SEQ ID NO: 1846 - DNA, 30 bp, Artificial Sequence
  primer
tctctacacagctacatactatactgtaac

SEQ ID NO: 1847 - DNA, 30 bp, Artificial Sequence
  primer
tacgacggacgctggtggtgtaagagaagg

SEQ ID NO: 1848 - DNA, 30 bp, Artificial Sequence
  primer
gcctcgatatatctacgtatagttcaagtt

SEQ ID NO: 1849 - DNA, 30 bp, Artificial Sequence
  primer
ggctcctgcattcattgaaggtcggccttg

SEQ ID NO: 1850 - DNA, 30 bp, Artificial Sequence
  primer
cagttcggtgattcaagagaacaatggtgg

SEQ ID NO: 1851 - DNA, 30 bp, Artificial Sequence
  primer
tataacgaagccggctggaacggtaactca

SEQ ID NO: 1852 - DNA, 30 bp, Artificial Sequence
  primer
ctgtatcaattcaagtgacagtggcacgtc

SEQ ID NO: 1853 - DNA, 30 bp, Artificial Sequence
  primer
agcaattgcggttcataggcgtaattatat

SEQ ID NO: 1854 - DNA, 30 bp, Artificial Sequence
  primer
catatggacctggagatcaccgttcagtcc

SEQ ID NO: 1855 - DNA, 30 bp, Artificial Sequence
  primer
gaaggccgttggtctatctcttactggagc

SEQ ID NO: 1856 - DNA, 30 bp, Artificial Sequence
  primer
gtgcgttcatctagcctaagacgctgacct

SEQ ID NO: 1857 - DNA, 30 bp, Artificial Sequence
  primer
gagtaacttatatcctctctacgacatcga

SEQ ID NO: 1858 - DNA, 30 bp, Artificial Sequence
  primer
attctacgctgatgtctccgctgaacagga

SEQ ID NO: 1859 - DNA, 30 bp, Artificial Sequence
  primer
tcatcaacgttactcactagtaccacggct

SEQ ID NO: 1860 - DNA, 30 bp, Artificial Sequence
  primer
aaccattcttgaacgttgagaacctggtgg

SEQ ID NO: 1861 - DNA, 30 bp, Artificial Sequence
  primer
acgacacctccgcggaacatacctgattag

SEQ ID NO: 1862 - DNA, 30 bp, Artificial Sequence
  primer
gcgcacttattgaagtaatctcatggccaa

SEQ ID NO: 1863 - DNA, 30 bp, Artificial Sequence
  primer
gcgccaattcagccagttagcgtctccgtg

SEQ ID NO: 1864 - DNA, 30 bp, Artificial Sequence
  primer
agcaacaagtcgctgtatatcgactggccg

SEQ ID NO: 1865 - DNA, 30 bp, Artificial Sequence
  primer
ccttacaatagacctcgcggcgttcatgcc

SEQ ID NO: 1866 - DNA, 30 bp, Artificial Sequence
  primer
ggatccaacttcagcgaagcaccaacgtcg

SEQ ID NO: 1867 - DNA, 30 bp, Artificial Sequence
  primer
gcgccagttctcgtactctcgagaagcgac

SEQ ID NO: 1868 - DNA, 30 bp, Artificial Sequence
  primer
gagtgcggccaatctggaactcatgacgtt

SEQ ID NO: 1869 - DNA, 30 bp, Artificial Sequence
  primer
cctgagagtgattcgtgtctgcgaagatgc

SEQ ID NO: 1870 - DNA, 30 bp, Artificial Sequence
  primer
gtgactggttaaggcaatattggtcgaccg

SEQ ID NO: 1871 - DNA, 30 bp, Artificial Sequence
  primer
ctatcaagccttacaaggtcacgtccacta

SEQ ID NO: 1872 - DNA, 30 bp, Artificial Sequence
  primer
actgcgtccttgcgtcggaactccttgtgt

SEQ ID NO: 1873 - DNA, 30 bp, Artificial Sequence
  primer
tgcaactcagtggcggcgacaccaagagct

SEQ ID NO: 1874 - DNA, 30 bp, Artificial Sequence
  primer
ttcggttctactaggatctctatctgagct

SEQ ID NO: 1875 - DNA, 30 bp, Artificial Sequence
  primer
agctaatctattaagacagattagacagga

SEQ ID NO: 1876 - DNA, 30 bp, Artificial Sequence
  primer
ggaccgctcttaggttatgcacctgcgtat

SEQ ID NO: 1877 - DNA, 30 bp, Artificial Sequence
  primer
ctctaatactagtccacaggttagtacgaa

SEQ ID NO: 1878 - DNA, 30 bp, Artificial Sequence
  primer
atccatatatgctcgtcgtcagccagtgtt

SEQ ID NO: 1879 - DNA, 30 bp, Artificial Sequence
  primer
gctattactgtgttgatgtccacaggagaa

SEQ ID NO: 1880 - DNA, 30 bp, Artificial Sequence
  primer
gctacggcgcagatctagacaactggaagt

SEQ ID NO: 1881 - DNA, 30 bp, Artificial Sequence
  primer
gcctcttgtgttagccgaataccaatgacc

SEQ ID NO: 1882 - DNA, 30 bp, Artificial Sequence
  primer
tgaggacgataacattacctctcgagtcgc

SEQ ID NO: 1883 - DNA, 30 bp, Artificial Sequence
  primer
cgattaccaatccgacgacttcgcagcagc

SEQ ID NO: 1884 - DNA, 30 bp, Artificial Sequence
  primer
atgacacgagtccagtacatatgcgaagac

SEQ ID NO: 1885 - DNA, 30 bp, Artificial Sequence
  primer
gcgctcgcatgcactagtgtagactgacga

SEQ ID NO: 1886 - DNA, 30 bp, Artificial Sequence
  primer
gcacatctcagaattgatggtctatgtcgc

SEQ ID NO: 1887 - DNA, 30 bp, Artificial Sequence
  primer
ttcttcgacgccgcgtactaataggtcaat

SEQ ID NO: 1888 - DNA, 30 bp, Artificial Sequence
  primer
ggaagcgcctctaacaaccgatgcttgtgg

SEQ ID NO: 1889 - DNA, 30 bp, Artificial Sequence
  primer
ctctagacgcgtcgtgactccaatctgttg

SEQ ID NO: 1890 - DNA, 30 bp, Artificial Sequence
  primer
gtagttcgtcggagtgacctcgtactcact

SEQ ID NO: 1891 - DNA, 30 bp, Artificial Sequence
  primer
atgctgtcgagtgtccggcatagagcacac

SEQ ID NO: 1892 - DNA, 30 bp, Artificial Sequence
  primer
gcgcatcttgcagcgtcctgtagttctgaa

SEQ ID NO: 1893 - DNA, 30 bp, Artificial Sequence
  primer
gcgattgttgaggaaccacagcggcaccta

SEQ ID NO: 1894 - DNA, 30 bp, Artificial Sequence
  primer
cacgcgtactctgcttgctgtgtggtcggt

SEQ ID NO: 1895 - DNA, 30 bp, Artificial Sequence
  primer
catccaacgcaggacctagtagtcatgctt

SEQ ID NO: 1896 - DNA, 30 bp, Artificial Sequence
  primer
ttctagttgtgatgagaatcgctagcgtgc

SEQ ID NO: 1897 - DNA, 30 bp, Artificial Sequence
  primer
cattctgaatctggtctctctcgatcatcc

SEQ ID NO: 1898 - DNA, 30 bp, Artificial Sequence
  primer
attaatgtagaggatagttccgttctctcc

SEQ ID NO: 1899 - DNA, 30 bp, Artificial Sequence
  primer
gtatcgcgcttacgaatgaggtgtggcttc

SEQ ID NO: 1900 - DNA, 30 bp, Artificial Sequence
  primer
gctggtgagagagccagattatcggtggag

SEQ ID NO: 1901 - DNA, 30 bp, Artificial Sequence
  primer
ggcacgagcaggtagaactagaacctagat

SEQ ID NO: 1902 - DNA, 30 bp, Artificial Sequence
  primer
tgtattatctcgaagcggtgcgttagagtc

SEQ ID NO: 1903 - DNA, 30 bp, Artificial Sequence
  primer
cacgtgttctagctactaatggcgtcaatt

SEQ ID NO: 1904 - DNA, 30 bp, Artificial Sequence
  primer
cgcgctacattacttcctacaccatgcgta

SEQ ID NO: 1905 - DNA, 30 bp, Artificial Sequence
  primer
tgaggcaactagtgttcgcaagatgacgga

SEQ ID NO: 1906 - DNA, 30 bp, Artificial Sequence
  primer
ttattattgtctgtggaacgcacgccagtc

SEQ ID NO: 1907 - DNA, 30 bp, Artificial Sequence
  primer
gctatagtattatccatgaattccgtcggc

SEQ ID NO: 1908 - DNA, 30 bp, Artificial Sequence
  primer
gtatcaatagctcaattcgtcagagttgtg

SEQ ID NO: 1909 - DNA, 30 bp, Artificial Sequence
  primer
tagtccatgcgtggatatattgagagctga

SEQ ID NO: 1910 - DNA, 30 bp, Artificial Sequence
  primer
gcacagtacgacttataacaggtctagatc

SEQ ID NO: 1911 - DNA, 30 bp, Artificial Sequence
  primer
actcaatggtggcacgctcggcgcagcata

SEQ ID NO: 1912 - DNA, 30 bp, Artificial Sequence
  primer
gtagtaccactccgccttaggcagcttaag

SEQ ID NO: 1913 - DNA, 30 bp, Artificial Sequence
  primer
cgctcaactgatgcgtgcaaccaatgttat

SEQ ID NO: 1914 - DNA, 30 bp, Artificial Sequence
  primer
gcagcttgactgcctagacagcagttacag

SEQ ID NO: 1915 - DNA, 30 bp, Artificial Sequence
  primer
gcaacttcttagtacgaattcatcgtccaa

SEQ ID NO: 1916 - DNA, 30 bp, Artificial Sequence
  primer
atccgtatgctgcggcagtggaggtggctt

SEQ ID NO: 1917 - DNA, 30 bp, Artificial Sequence
  primer
tgcggatcaatccagttctgtgtactgtga

SEQ ID NO: 1918 - DNA, 30 bp, Artificial Sequence
  primer
ttatgattatcaccggcgtaacattccgaa

SEQ ID NO: 1919 - DNA, 30 bp, Artificial Sequence
  primer
gctacctagattcttcaactcatcgctacc

SEQ ID NO: 1920 - DNA, 30 bp, Artificial Sequence
  primer
cagtgttagaatggcggtgtgtagccgcta

SEQ ID NO: 1921 - DNA, 35 bp, Artificial Sequence
  primer
gcttatagactacagctgcgaggtataaggtcact

SEQ ID NO: 1922 - DNA, 35 bp, Artificial Sequence
  primer
cgctcagcaggatgctatcctaagttaatgtggtg

SEQ ID NO: 1923 - DNA, 35 bp, Artificial Sequence
  primer
gaactgagcggacatcagctaggcctacaatacat

SEQ ID NO: 1924 - DNA, 35 bp, Artificial Sequence
  primer
tcgtgaacttctgcgttggtctctaccaaggcggt

SEQ ID NO: 1925 - DNA, 35 bp, Artificial Sequence
  primer
taagtcaggtatcttatcagtggtacacggtacga

SEQ ID NO: 1926 - DNA, 35 bp, Artificial Sequence
  primer
taataatgttgcgcgtgaccgaggaggaatccact

SEQ ID NO: 1927 - DNA, 35 bp, Artificial Sequence
  primer
ctaggagttctcgtaagctggagtaccgtaacgtg

SEQ ID NO: 1928 - DNA, 35 bp, Artificial Sequence
  primer
ggactctcctcagaggatccttcttgcgcaggcat

SEQ ID NO: 1929 - DNA, 35 bp, Artificial Sequence
  primer
gctagaggcctgagtacaccttctcgcatcaggat

SEQ ID NO: 1930 - DNA, 35 bp, Artificial Sequence
  primer
atatcgcgagcactaacgtcgttgtcgttctagga

SEQ ID NO: 1931 - DNA, 35 bp, Artificial Sequence
  primer
agcggttactatacctggcggctgacgttgttagt

SEQ ID NO: 1932 - DNA, 35 bp, Artificial Sequence
  primer
gagctaggtagatctccaagtgtagctaagaagag

SEQ ID NO: 1933 - DNA, 35 bp, Artificial Sequence
  primer
ggagtcgctggtgacgtatgccgaggatgagcttc

SEQ ID NO: 1934 - DNA, 35 bp, Artificial Sequence
  primer
cgccgacctcctgttcacgaagccgcctgatgtaa

SEQ ID NO: 1935 - DNA, 35 bp, Artificial Sequence
  primer
agtaggcacttagttatcgattacgttagttagtc

SEQ ID NO: 1936 - DNA, 35 bp, Artificial Sequence
  primer
ggatgacgtctcagtctacctcgcagtgtcgtcta

SEQ ID NO: 1937 - DNA, 35 bp, Artificial Sequence
  primer
ctggttcgcgttagcaatactaaggcagtcaggag

SEQ ID NO: 1938 - DNA, 35 bp, Artificial Sequence
  primer
atatggtcatattggcctcttcgaacacagactgt

SEQ ID NO: 1939 - DNA, 35 bp, Artificial Sequence
  primer
tatcagaggatagcaggtctgagttgcaaggctaa

SEQ ID NO: 1940 - DNA, 35 bp, Artificial Sequence
  primer
ggtggtctgaccatagctgttcttctcacagagac

SEQ ID NO: 1941 - DNA, 35 bp, Artificial Sequence
  primer
gcaataccaacgagatgagtattcgttgaagctct

SEQ ID NO: 1942 - DNA, 35 bp, Artificial Sequence
  primer
ccaagtcgacgctgcatgaatgagcgctattcact

SEQ ID NO: 1943 - DNA, 35 bp, Artificial Sequence
  primer
ccattagatcgcttcgagacaattaggagacatga

SEQ ID NO: 1944 - DNA, 35 bp, Artificial Sequence
  primer
gatgactgtacctcctatcattgagtgtggaccaa

SEQ ID NO: 1945 - DNA, 35 bp, Artificial Sequence
  primer
atatctggatgaatagtggttaggtaagcaagtaa

SEQ ID NO: 1946 - DNA, 35 bp, Artificial Sequence
  primer
accgactatgttaattcgtgtctggatggcagaat

SEQ ID NO: 1947 - DNA, 35 bp, Artificial Sequence
  primer
gtggcagtcttgctagtatcttagaccatcaccaa

SEQ ID NO: 1948 - DNA, 35 bp, Artificial Sequence
  primer
cgctatcttagtcgagcacaatgtcttcgtatagg

SEQ ID NO: 1949 - DNA, 35 bp, Artificial Sequence
  primer
attagtacggcacgaaccggccattcatggcagct

SEQ ID NO: 1950 - DNA, 35 bp, Artificial Sequence
  primer
agtacgactatcaagactccagcgctctccttgga

SEQ ID NO: 1951 - DNA, 35 bp, Artificial Sequence
  primer
atgagcctcggagcgaacgttatcgatcaggctgt

SEQ ID NO: 1952 - DNA, 35 bp, Artificial Sequence
  primer
ttgcgtgcagtagcaccgatacacagcgcttgtat

SEQ ID NO: 1953 - DNA, 35 bp, Artificial Sequence
  primer
aacggctgcatcacctacactatactcaacatcta

SEQ ID NO: 1954 - DNA, 35 bp, Artificial Sequence
  primer
gtcgctatgcgagaagtggcgtggaatgctatggt

SEQ ID NO: 1955 - DNA, 35 bp, Artificial Sequence
  primer
catggatacctactgacttgacttctagaggaccg

SEQ ID NO: 1956 - DNA, 35 bp, Artificial Sequence
  primer
gagtgacgcagacaccgtaacgtcgaatcttctag

SEQ ID NO: 1957 - DNA, 35 bp, Artificial Sequence
  primer
agtaccgtctgtgtgaatattgttcctacgttaca

SEQ ID NO: 1958 - DNA, 35 bp, Artificial Sequence
  primer
ggctaatcgatagtgacgagttctgcacgcctgaa

SEQ ID NO: 1959 - DNA, 35 bp, Artificial Sequence
  primer
ggcgagcgctcgtggttctgagtcgctgttagatg

SEQ ID NO: 1960 - DNA, 35 bp, Artificial Sequence
  primer
tatctccagcgttataagctactggagccgctcgg

SEQ ID NO: 1961 - DNA, 35 bp, Artificial Sequence
  primer
ccttctgcgcaagtcaaggattcgcttagatggac

SEQ ID NO: 1962 - DNA, 35 bp, Artificial Sequence
  primer
gttgctgacagccgttgcgtacttgccttaagaac

SEQ ID NO: 1963 - DNA, 35 bp, Artificial Sequence
  primer
gtggcctaatcactcgcgcttcataggccgatagg

SEQ ID NO: 1964 - DNA, 35 bp, Artificial Sequence
  primer
tgcatctagcctacatcggaccttgttatggtaat

SEQ ID NO: 1965 - DNA, 35 bp, Artificial Sequence
  primer
ggacagctactggacaccaccgaactggtagtgtc

SEQ ID NO: 1966 - DNA, 35 bp, Artificial Sequence
  primer
aactggcgatggacggccgctcttccgctacatag

SEQ ID NO: 1967 - DNA, 35 bp, Artificial Sequence
  primer
ggagcagttagctatggagcaggccgataacctga

SEQ ID NO: 1968 - DNA, 35 bp, Artificial Sequence
  primer
actctacggtgcacctcagccttcatgcaataggc

SEQ ID NO: 1969 - DNA, 35 bp, Artificial Sequence
  primer
cttgtagcacaatacattactctccacgtgatagc

SEQ ID NO: 1970 - DNA, 35 bp, Artificial Sequence
  primer
ggacgctatcgataccgttattcctactctgtcgg

SEQ ID NO: 1971 - DNA, 35 bp, Artificial Sequence
  primer
ggatgatcgtcaacgatcaactgacagttagtcga

SEQ ID NO: 1972 - DNA, 35 bp, Artificial Sequence
  primer
tgacagtagcaatgtctcacgtctgcacaacggaa

SEQ ID NO: 1973 - DNA, 35 bp, Artificial Sequence
  primer
gtcgcaggacctcacggatagtagtgcgaggtcta

SEQ ID NO: 1974 - DNA, 35 bp, Artificial Sequence
  primer
atatcggcggacgcaatgacagttgttggctgatg

SEQ ID NO: 1975 - DNA, 35 bp, Artificial Sequence
  primer
aagcaccaaggaggtatgttccatcgaggcgctcg

SEQ ID NO: 1976 - DNA, 35 bp, Artificial Sequence
  primer
gaccgcaccttatagctatatcctggtctagtact

SEQ ID NO: 1977 - DNA, 35 bp, Artificial Sequence
  primer
tctcagaggaaggttgagcgtctgaccaggttggc

SEQ ID NO: 1978 - DNA, 35 bp, Artificial Sequence
  primer
tggacctagagacctagctcgtctcttcgcgatcg

SEQ ID NO: 1979 - DNA, 35 bp, Artificial Sequence
  primer
cggagtggttccacgcgacctcgcaactaatcctt

SEQ ID NO: 1980 - DNA, 35 bp, Artificial Sequence
  primer
ggagccgcgcgcagactgaccttgcttgatctact

SEQ ID NO: 1981 - DNA, 35 bp, Artificial Sequence
  primer
actctaagtatatgcgcagttagtatactgaacca

SEQ ID NO: 1982 - DNA, 35 bp, Artificial Sequence
  primer
gagcattgcttcgcttcgatgtctattctgatcag

SEQ ID NO: 1983 - DNA, 35 bp, Artificial Sequence
  primer
gcttgtattgccactcgagtaggtcgtggcagtag

SEQ ID NO: 1984 - DNA, 35 bp, Artificial Sequence
  primer
atctggacattgcattcggtgtgtatacagaaggc

SEQ ID NO: 1985 - DNA, 35 bp, Artificial Sequence
  primer
ggttgcgatcagcttgatagcaggtcatatcctca

SEQ ID NO: 1986 - DNA, 35 bp, Artificial Sequence
  primer
gcaggtactaacctgagatgcgtagctaacacagg

SEQ ID NO: 1987 - DNA, 35 bp, Artificial Sequence
  primer
atctgcaaggacgtaacgtcctcggaaggtgaggt

SEQ ID NO: 1988 - DNA, 35 bp, Artificial Sequence
  primer
ataatcttacgagcctccagtgaataatgcaagca

SEQ ID NO: 1989 - DNA, 35 bp, Artificial Sequence
  primer
caatctccgcacagtcttgttcaggtacagactta

SEQ ID NO: 1990 - DNA, 35 bp, Artificial Sequence
  primer
atgtgcgcaattcagcgtaagtgcctattcataat

SEQ ID NO: 1991 - DNA, 35 bp, Artificial Sequence
  primer
tcggacgcacacatcctgttgtcgagaagaggaag

SEQ ID NO: 1992 - DNA, 35 bp, Artificial Sequence
  primer
tcggaagcatcacatgagcatcaggagttcattgc

SEQ ID NO: 1993 - DNA, 35 bp, Artificial Sequence
  primer
atctggttgtggacttctatacagtaccagagtgg

SEQ ID NO: 1994 - DNA, 35 bp, Artificial Sequence
  primer
cgtctgaatatagttagctagtagtgtaatccagg

SEQ ID NO: 1995 - DNA, 35 bp, Artificial Sequence
  primer
taatatctgatccgacctattatctaggactactc

SEQ ID NO: 1996 - DNA, 35 bp, Artificial Sequence
  primer
tatgcggccgtccgtacctcgtctgcttcagttgg

SEQ ID NO: 1997 - DNA, 35 bp, Artificial Sequence
  primer
tggctcaagttccatattgccaagacgacctggag

SEQ ID NO: 1998 - DNA, 35 bp, Artificial Sequence
  primer
gcagttctgctaggcggtccgaggcaattgaagag

SEQ ID NO: 1999 - DNA, 35 bp, Artificial Sequence
  primer
catggcacagacgaagtatgcaccacgctcattaa

SEQ ID NO: 2000 - DNA, 35 bp, Artificial Sequence
  primer
ggagcgtactacgaccattcaaccgaatatgttac

SEQ ID NO: 2001 - DNA, 35 bp, Artificial Sequence
  primer
gcgtagatctcgcgacagagacaaggtgcgaatgg

SEQ ID NO: 2002 - DNA, 35 bp, Artificial Sequence
  primer
tggactgaggttctccggtctatactcctgtagga

SEQ ID NO: 2003 - DNA, 35 bp, Artificial Sequence
  primer
tggctatagcaacggcttcttgtgatcgcattgca

SEQ ID NO: 2004 - DNA, 35 bp, Artificial Sequence
  primer
ggcgaagaatcatgcgagacggagtagacggacgt

SEQ ID NO: 2005 - DNA, 35 bp, Artificial Sequence
  primer
gagcattgcgagttgcacacgtgatatcagactgt

SEQ ID NO: 2006 - DNA, 35 bp, Artificial Sequence
  primer
ctgttgacctatgccagaatcaatacctcagatta

SEQ ID NO: 2007 - DNA, 35 bp, Artificial Sequence
  primer
gttaacaagtagatgccaagatacaacgagagacc

SEQ ID NO: 2008 - DNA, 35 bp, Artificial Sequence
  primer
gagcaagattatagttaggaagatagttaactcgc

SEQ ID NO: 2009 - DNA, 35 bp, Artificial Sequence
  primer
tccggagtcgagcatatgtgaccaactctcaacgc

SEQ ID NO: 2010 - DNA, 35 bp, Artificial Sequence
  primer
ggagctgcgatgccgttaccgacgtcatcttcaag

SEQ ID NO: 2011 - DNA, 35 bp, Artificial Sequence
  primer
gctctatcttacacattggcgtactggactcgcga

SEQ ID NO: 2012 - DNA, 35 bp, Artificial Sequence
  primer
ttctacatattcatcgcctaccgagttgcgcgaag

SEQ ID NO: 2013 - DNA, 35 bp, Artificial Sequence
  primer
tggacgtctgacctgtgtctacatcggtggtgcta

SEQ ID NO: 2014 - DNA, 35 bp, Artificial Sequence
  primer
ggcaggacagctccgtgttctactcgaaccgcact

SEQ ID NO: 2015 - DNA, 35 bp, Artificial Sequence
  primer
tgacaacctcatgtctccgaccgcaggcatacaat

SEQ ID NO: 2016 - DNA, 35 bp, Artificial Sequence
  primer
gcaggcctaacaagtggtcacgaggagtccttatt

SEQ ID NO: 2017 - DNA, 90 bp, Artificial Sequence
  primer
cccatacacacaccatgaagcttgaactaattaacattctcaaactaattaacaagcatgcaagcatgtttttacacaatgacaatatat

SEQ ID NO: 2018 - DNA, 90 bp, Artificial Sequence
  primer
atgggtgagggcgcagaggcaaagacatggaggtccggaagggtagaagctcacatcaagtcgagtatgttgaatgcaatcccatatata

SEQ ID NO: 2019 - DNA, 24 bp, Artificial Sequence
  primer
cccatacacacaccatgaagcttg

SEQ ID NO: 2020 - DNA, 24 bp, Artificial Sequence
  primer
ggtagaagctcacatcaagtcgag

SEQ ID NO: 2021 - DNA, 90 bp, Artificial Sequence
  primer
aatcacagaacgaggtctggacgagaacagagctggacatctacacgcaccgcatggtagtagagcatgtactgcaaaagcttgaagcgc

SEQ ID NO: 2022 - DNA, 90 bp, Artificial Sequence
  primer
gatgctgagggcgaagttgtcagccaagtcctcaatgtcataggcgagatcgcagtagttctgtaaccattccctgctaaactggtccat

SEQ ID NO: 2023 - DNA, 24 bp, Artificial Sequence
  primer
acgagaacagagctggacatctac

SEQ ID NO: 2024 - DNA, 24 bp, Artificial Sequence
  primer
tcaatgtcataggcgagatcgcag

SEQ ID NO: 2025 - DNA, 90 bp, Artificial Sequence
  primer
agaccaacaagcagcaagtagtcagagaagtacaagagaaggagagcaagaaggatagtaagttgcaagcttaccgttacaaagatgata

SEQ ID NO: 2026 - DNA, 90 bp, Artificial Sequence
  primer
ggaggagcacaactaggcgtttatcaagatgggtcatcgagctcttggtgtcttcaaccttcttgacatcaacttctccaatcttcgtct

SEQ ID NO: 2027 - DNA, 26 bp, Artificial Sequence
  primer
ggagagcaagaaggatagtaagttgc

SEQ ID NO: 2028 - DNA, 25 bp, Artificial Sequence
  primer
cgagctcttggtgtcttcaaccttc

SEQ ID NO: 2029 - DNA, 90 bp, Artificial Sequence
  primer
tggggtagtcctgaagctctaggtatgcctcttcatctccctgcacctctggtgctagcacctcctgctcttcgggcacctctaccgggg

SEQ ID NO: 2030 - DNA, 90 bp, Artificial Sequence
  primer
ggatactgatgtagctttcacccgggagtattccaaggtatcgattttccacggggaacgcgaagtgcactagttgaggtttagattgcc

SEQ ID NO: 2031 - DNA, 25 bp, Artificial Sequence
  primer
gaagctctaggtatgcctcttcatc

SEQ ID NO: 2032 - DNA, 25 bp, Artificial Sequence
  primer
gtgcactagttgaggtttagattgc

SEQ ID NO: 2033 - DNA, 90 bp, Artificial Sequence
  primer
tcgggaaaacgaacgggcgaactacagatgtcagtacgaagtagtctatggcaggaaatacgtagtccatacgtggtgccagcccaagcc

SEQ ID NO: 2034 - DNA, 90 bp, Artificial Sequence
  primer
agcaggagggagaaaggaaacgtggcattcatcggctgtctgccattgccatgtgagacaaggaaatctacttcacccccatctatcgag

SEQ ID NO: 2035 - DNA, 24 bp, Artificial Sequence
  primer
gggcgaactacagatgtcagtacg

SEQ ID NO: 2036 - DNA, 24 bp, Artificial Sequence
  primer
ctgtctgccattgccatgtgagac

SEQ ID NO: 2037 - DNA, 90 bp, Artificial Sequence
  primer
agacataagattaactatgaacaaattcacgggtccgattcctttgggatttgcagcttgcaagaaccttcaaatactcattatatcttc

SEQ ID NO: 2038 - DNA, 90 bp, Artificial Sequence
  primer
ttaagttgcagaatttgatacgaagaacttgaagcatggtgaggttgccgagctcattggggatggttccagaaaggctattgtagctta

SEQ ID NO: 2039 - DNA, 24 bp, Artificial Sequence
  primer
gaacaaattcacgggtccgattcc

SEQ ID NO: 2040 - DNA, 24 bp, Artificial Sequence
  primer
cgaagaacttgaagcatggtgagg

SEQ ID NO: 2041 - DNA, 10 bp, Artificial Sequence
  primer
gttacacacg

SEQ ID NO: 2042 - DNA, 70 bp, Artificial Sequence
  primer
aatgatacggcgaccaccgagatctacacctctctattcgtcggcagcgtcagatgtgtataagagacag

SEQ ID NO: 2043 - DNA, 66 bp, Artificial Sequence
  primer
caagcagaagacggcatacgagattaaggcgagtctcgtgggctcggagatgtgtataagagacag

SEQ ID NO: 2044 - DNA, 12 bp, Artificial Sequence
  primer
taagagacagaa

SEQ ID NO: 2045 - DNA, 12 bp, Artificial Sequence
  primer
taagagacagat

SEQ ID NO: 2046 - DNA, 12 bp, Artificial Sequence
  primer
taagagacagac

SEQ ID NO: 2047 - DNA, 12 bp, Artificial Sequence
  primer
taagagacagag

SEQ ID NO: 2048 - DNA, 12 bp, Artificial Sequence
  primer
taagagacagta

SEQ ID NO: 2049 - DNA, 12 bp, Artificial Sequence
  primer
taagagacagtt

SEQ ID NO: 2050 - DNA, 12 bp, Artificial Sequence
  primer
taagagacagtc

SEQ ID NO: 2051 - DNA, 12 bp, Artificial Sequence
  primer
taagagacagtg

SEQ ID NO: 2052 - DNA, 12 bp, Artificial Sequence
  primer
taagagacagca

SEQ ID NO: 2053 - DNA, 12 bp, Artificial Sequence
  primer
taagagacagct

SEQ ID NO: 2054 - DNA, 12 bp, Artificial Sequence
  primer
taagagacagcc

SEQ ID NO: 2055 - DNA, 12 bp, Artificial Sequence
  primer
taagagacagcg

SEQ ID NO: 2056 - DNA, 12 bp, Artificial Sequence
  primer
taagagacagga

SEQ ID NO: 2057 - DNA, 12 bp, Artificial Sequence
  primer
taagagacaggt

SEQ ID NO: 2058 - DNA, 12 bp, Artificial Sequence
  primer
taagagacaggc

SEQ ID NO: 2059 - DNA, 12 bp, Artificial Sequence
  primer
taagagacaggg

(19) United States
(12) Patent Application Publication
(10) Pub. No.: US 2019/0233889 A1
(43) Pub. Date: Aug. 1, 2019

(54) METHOD FOR PRODUCING DNA LIBRARY AND METHOD FOR ANALYZING GENOMIC DNA USING THE DNA LIBRARY

(71) Applicant: TOYOTA JIDOSHA KABUSHIKI KAISHA, Toyota-shi, Aichi-ken (JP)

(72) Inventors: Hiroyuki ENOKI, Hamamatsu-shi (JP); Yoshie TAKEUCHI, Hamamatsu-shi (JP)

(73) Assignee: TOYOTA JIDOSHA KABUSHIKI KAISHA, Toyota-shi, Aichi-ken (JP)

(21) Appl. No.: 16/313,706

(22) PCT Filed: Apr. 3, 2017
(86) PCT No.: PCT/JP2017/013965

§ 371 Date: Dec. 27, 2018

(30) Foreign Application Priority Data
Jun. 29, 2016 (JP) 2016-129048
Sep. 13, 2016 (JP) 2016-178528
Mar. 31, 2017 (JP) 2017-071020

(51) Int. Cl. C12Q 1/6874; C12N 15/10

(52) U.S. Cl. CPC C12Q 1/6874; C12Q 2600/16; C12N 15/1093

(57) ABSTRACT

A DNA library with excellent reproducibility is readily produced. A nucleic acid amplification reaction is conducted in a reaction solution containing genomic DNA and a random primer at a high concentration to obtain a DNA fragment by the nucleic acid amplification reaction using the genomic DNA as a template.

### TECHNICAL FIELD

The present invention relates to a method for producing a DNA library that can be used for analyzing a DNA marker, for example, and a method for genomic DNA analysis using such DNA library.

### BACKGROUND ART

In general, genomic analysis is performed to conduct comprehensive analysis of genetic information contained in the genome, such as nucleotide sequence information. However, an analysis aimed at determination of the nucleotide sequence for whole genome is disadvantageous in terms of the number of processes and the cost. In cases of organisms with large genomic sizes, in addition, genomic analysis based on nucleotide sequence analysis has limitations because of genome complexity.

Patent Literature 1 discloses an amplified fragment length polymorphism (AFLP) marker technique wherein a sample-specific index is incorporated into a restriction-enzyme-treated fragment that had been ligated to an adapter and only a part of the sequence of the restriction-enzyme-treated fragment is to be determined. According to the technique disclosed in Patent Literature 1, the complexity of genomic DNA is reduced by treating genomic DNA with a restriction enzyme, the nucleotide sequence of a target part of the restriction-enzyme-treated fragment is determined, and the target restriction-enzyme-treated fragment is thus determined sufficiently. The technique disclosed in Patent Literature 1, however, requires processes such as treatment of genomic DNA with a restriction enzyme and ligation reaction with the use of an adapter. Thus, it is difficult to achieve a cost reduction.

Meanwhile, Patent Literature 2 discloses as follows. That is, a DNA marker for identification that is highly correlated with the results of taste evaluation was found from among DNA bands obtained by amplifying DNAs extracted from a rice sample via PCR in the presence of adequate primers by the so-called RAPD (randomly amplified polymorphic DNA) technique. The method disclosed in Patent Literature 2 involves the use of a plurality of sequence-tagged sites (STSs, which are primers) identified by particular sequences. According to the method disclosed in Patent Literature 2, a DNA marker for identification amplified with the use of an STS primer is detected via electrophoresis. However, the RAPD technique disclosed in Patent Literature 2 yields significantly poor reproducibility of PCR amplification, and, accordingly, such technique cannot be generally adopted as a DNA marker technique.

Patent Literature 3 discloses a method for producing a genomic library wherein PCR is carried out with the use of a single type of primer designed on the basis of a sequence that appears relatively frequently in the target genome, the entire genomic region is substantially uniformly amplified, and a genomic library can be thus produced. While Patent Literature 3 describes that a genomic library can be produced by conducting PCR with the use of a random primer containing a random sequence, it does not describe any actual procedures or results of experimentation. Accordingly, the method described in Patent Literature 3 is deduced to require nucleotide sequence information of the genome so as to identify the genome appearing frequency, which would increase the number of procedures and the cost. According to the method described in Patent Literature 3, in addition, the entire genome is to be amplified, and complexity of genomic DNA cannot be reduced, disadvantageously.

### CITATION LIST

### Patent Literature

Patent Literature 1: JP Patent No. 5389638
Patent Literature 2: JP Patent Publication (Kokai) No. 2003-79375 A
Patent Literature 3: JP Patent No. 3972106

### SUMMARY OF INVENTION

### Technical Problem

For a technique for genome information analysis, such as genetic linkage analysis conducted with the use of a DNA marker, production of a DNA library in a more convenient and highly reproducible manner is desired. As described above, a wide variety of techniques for producing a DNA library are known. To date, however, there have been no techniques known to be sufficient in terms of convenience and/or reproducibility. Under the above circumstances, it is an object of the present invention to provide a method for producing a DNA library with more convenience and higher reproducibility, and it is another object to provide a method for analyzing genomic DNA with the use of such DNA library.

### Solution to Problem

The present inventors have conducted concentrated studies in order to attain the above objects. As a result, they discovered that high reproducibility could be achieved by conducting PCR with the use of a random primer while designating the concentration of such random primer within a designated range in a reaction solution. This has led to the completion of the present invention.

The present invention includes the following.

(1) A method for producing a DNA library, comprising conducting a nucleic acid amplification reaction in a reaction solution containing genomic DNA and a random primer at a high concentration using genomic DNA as a template to obtain DNA fragments.
(2) The method for producing a DNA library according to (1), wherein the reaction solution comprises the random primer at a concentration of 4 to 200 μM.
(3) The method for producing a DNA library according to (1), wherein the reaction solution comprises the random primer at a concentration of 4 to 100 μM.
(4) The method for producing a DNA library according to (1), wherein the random primer comprises 9 to 30 nucleotides.
(5) The method for producing a DNA library according to (1), wherein the DNA fragments each comprise 100 to 500 nucleotides.
(6) A method for analyzing genomic DNA, comprising using a DNA library produced by the method for producing a DNA library according to any one of (1) to (5) as a DNA marker.
(7) The method for analyzing genomic DNA according to (6), which comprises determining the nucleotide sequence of the DNA library produced by the method for producing a DNA library according to any one of (1) to (5) and confirming the presence or absence of the DNA marker based on the nucleotide sequence.
(8) The method for analyzing genomic DNA according to (7), wherein the presence or absence of the DNA marker is confirmed based on the number of reads of the nucleotide sequence of the DNA library in the step of confirming the presence or absence of the DNA marker.
(9) The method for analyzing genomic DNA according to (7), wherein the nucleotide sequence of the DNA library is compared with known sequence information or with the nucleotide sequence of a DNA library produced using genomic DNA from a different organism or tissue, and the presence or absence of the DNA marker is confirmed based on differences in the nucleotide sequences.
(10) The method for analyzing genomic DNA according to (6), which comprises:

a step of preparing a pair of primers for specifically amplifying the DNA marker based on the nucleotide sequence of the DNA marker;

a step of conducting a nucleic acid amplification reaction using genomic DNA extracted from a target organism as a template and the pair of primers; and

a step of confirming the presence or absence of the DNA marker in the genomic DNA based on the results of the nucleic acid amplification reaction.

(11) A method for producing a DNA library, comprising:

a step of conducting a nucleic acid amplification reaction in a first reaction solution comprising genomic DNA and a random primer at a high concentration to obtain first DNA fragments by the nucleic acid amplification reaction using the genomic DNA as a template; and

a step of conducting a nucleic acid amplification reaction in a second reaction solution comprising the obtained first DNA fragments and a nucleotide, as a primer, which has a 3′-end nucleotide sequence having 70% identity to at least a 5′-end nucleotide sequence of the random primer to ligate the nucleotides to the first DNA fragments, thereby obtaining second DNA fragments.

(12) The method for producing a DNA library according to (11), wherein the first reaction solution comprises the random primer at a concentration of 4 to 100 μM.
(13) The method for producing a DNA library according to (11), wherein the first reaction solution comprises the random primer at a concentration of 4 to 100 μM.
(14) The method for producing a DNA library according to (11), wherein the random primer comprises 9 to 30 nucleotides.
(15) The method for producing a DNA library according to (11), wherein the first DNA fragments each comprise 100 to 500 nucleotides.
(16) The method for producing a DNA library according to (11), wherein the primer for amplifying the second DNA fragments comprises a region used for a nucleotide sequencing reaction, or the primer used for a nucleic acid amplification reaction using the second DNA fragments as templates or a nucleic acid amplification reaction to be conducted repeatedly comprises a region used for a nucleotide sequencing reaction.
(17) A method for analyzing a DNA library, comprising a step of determining a nucleotide sequence for a second DNA fragment obtained by the method for producing a DNA library according to any one of (11) to (15) or a DNA fragment obtained using a primer comprising a region complementary to a sequencer primer to be used in a nucleotide sequencing reaction in the method for producing a DNA library according to (16).
(18) A method for analyzing genomic DNA, comprising using a DNA library produced by the method for producing a DNA library according to any one of (11) to (17) as a DNA marker.
(19) The method for analyzing genomic DNA according to (18), which comprises determining the nucleotide sequence of the DNA library produced by the method for producing a DNA library according to any one of ((11) to (17) and confirming the presence or absence of the DNA marker based on the nucleotide sequence.
(20) The method for analyzing genomic DNA according to (19), wherein the presence or absence of the DNA marker is confirmed based on the number of reads of the nucleotide sequence of the DNA library in the step of confirming the presence or absence of the DNA marker.
(21) The method for analyzing genomic DNA according to (19), wherein the nucleotide sequence of the DNA library is compared with known sequence information or with the nucleotide sequence of a DNA library produced using genomic DNA from a different organism or tissue, and the presence or absence of the DNA marker is confirmed based on differences in the nucleotide sequences.
(22) The method for analyzing genomic DNA according to (18), which comprises: a step of preparing a pair of primers for specifically amplifying the DNA marker based on the nucleotide sequence of the DNA marker; a step of conducting a nucleic acid amplification reaction using genomic DNA extracted from a target organism as a template and the pair of primers; and a step of confirming the presence or absence of the DNA marker in the genomic DNA based on the results of the nucleic acid amplification reaction.
(23) A DNA library, which is produced by the method for producing a DNA library according to any one of (1) to (5) and (11) to (16).

The present description includes part or all of the contents as disclosed in the descriptions and/or drawings of Japanese Patent Application Nos. 2016-129048, 2016-178528, and 2017-071020, which are priority documents of the present application.

### Advantageous Effects of Invention

A DNA library can be produced in a very convenient manner by the method for producing a DNA library according to the present invention because the method is based on a nucleic acid amplification method using random primers. In addition, reproducibility of a nucleic acid fragment to be amplified is excellent in the method for producing a DNA library according to the present invention even though the method is a nucleic acid amplification method using random primers. Therefore, according to the method for producing a DNA library of the present invention, the produced DNA library can be used as a DNA marker and thus can be used for genomic DNA analysis such as genetic linkage analysis.

The method for analyzing genomic DNA with the use of a DNA library according to the present invention involves the use of a DNA library produced in a simple manner with excellent reproducibility. Accordingly, genomic DNA can be analyzed in a cost-effective manner with high accuracy.

### BRIEF DESCRIPTION OF DRAWINGS

FIG. 1 shows a flow chart demonstrating the method for producing a DNA library and the method for genomic DNA analysis with the use of the DNA library according to the present invention.

FIG. 2 shows a characteristic diagram demonstrating a correlation between an amplified fragment length and a fluorescence unit (FU) in which the amplified fragment length is determined based on an electrophoretic pattern of the DNA library amplified via PCR using DNA of the sugarcane variety NiF8 as a template under general conditions.

FIG. 3 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template at an annealing temperature of 45° C.

FIG. 4 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template at an annealing temperature of 40° C.

FIG. 5 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template at an annealing temperature of 37° C.

FIG. 6 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and 2.5 units of an enzyme.

FIG. 7 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and 12.5 units of an enzyme.

FIG. 8 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and MgCl₂ at the concentration doubled from the original level.

FIG. 9 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and MgCl₂ at the concentration tripled from the original level.

FIG. 10 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and MgCl₂ at the concentration quadrupled from the original level.

FIG. 11 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 8 nucleotides.

FIG. 12 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 9 nucleotides.

FIG. 13 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 11 nucleotides.

FIG. 14 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 12 nucleotides.

FIG. 15 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 14 nucleotides.

FIG. 16 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 16 nucleotides.

FIG. 17 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 18 nucleotides.

FIG. 18 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 20 nucleotides.

FIG. 19 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 2 μM.

FIG. 20 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 4 μM.

FIG. 21 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 6 μM.

FIG. 22 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 6 μM.

FIG. 23 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 8 μM.

FIG. 24 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 8 μM.

FIG. 25 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 10 μM.

FIG. 26 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 10 μM.

FIG. 27 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 20 μM.

FIG. 28 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 20 μM.

FIG. 29 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 40 μM.

FIG. 30 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 40 μM.

FIG. 31 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 60 μM.

FIG. 32 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 60 μM.

FIG. 33 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 100 μM.

FIG. 34 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 100 μM.

FIG. 35 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 200 μM.

FIG. 36 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 200 μM.

FIG. 37 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 300 μM.

FIG. 38 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 300 μM.

FIG. 39 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 400 μM.

FIG. 40 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 400 μM.

FIG. 41 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 500 μM.

FIG. 42 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 500 μM.

FIG. 43 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 600 μM.

FIG. 44 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 700 M.

FIG. 45 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 800 μM.

FIG. 46 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 900 μM.

FIG. 47 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 1000 μM.

FIG. 48 shows a characteristic diagram demonstrating the results of MiSeq analysis of a DNA library amplified using DNA of the sugarcane variety NiF8 as a template and a random primer.

FIG. 49 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the rice variety Nipponbare as a template and a random primer.

FIG. 50 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the rice variety Nipponbare as a template and a random primer.

FIG. 51 shows a characteristic diagram demonstrating the results of MiSeq analysis of a DNA library amplified using DNA of the rice variety Nipponbare as a template and a random primer.

FIG. 52 shows a characteristic diagram demonstrating positions of MiSeq read patterns in the genome information of the rice variety Nipponbare.

FIG. 53 shows a characteristic diagram demonstrating the frequency distribution of the number of mismatched nucleotides between the random primer and the rice genome.

FIG. 54 shows a characteristic diagram demonstrating the number of reads of the sugarcane varieties NiF8 and Ni9 and hybrid progeny lines thereof at the marker N80521152.

FIG. 55 shows a photograph demonstrating electrophoretic patterns of the sugarcane varieties NiF8 and Ni9 and hybrid progeny lines thereof at the PCR marker N80521152.

FIG. 56 shows a characteristic diagram demonstrating the number of reads of the sugarcane varieties NiF8 and Ni9 and hybrid progeny lines thereof at the marker N80997192.

FIG. 57 shows a photograph demonstrating electrophoretic patterns of the sugarcane varieties NiF8 and Ni9 and hybrid progeny lines thereof at the PCR marker N80997192.

FIG. 58 shows a characteristic diagram demonstrating the number of reads of the sugarcane varieties NiF8 and Ni9 and hybrid progeny lines thereof at the marker N80533142.

FIG. 59 shows a photograph demonstrating electrophoretic patterns of the sugarcane varieties NiF8 and Ni9 and hybrid progeny lines thereof at the PCR marker N80533142.

FIG. 60 shows a characteristic diagram demonstrating the number of reads of the sugarcane varieties NiF8 and Ni9 and hybrid progeny lines thereof at the marker N91552391.

FIG. 61 shows a photograph demonstrating electrophoretic patterns of the sugarcane varieties NiF8 and Ni9 and hybrid progeny lines thereof at the PCR marker N91552391.

FIG. 62 shows a characteristic diagram demonstrating the number of reads of the sugarcane varieties NiF8 and Ni9 and hybrid progeny lines thereof at the marker N91653962.

FIG. 63 shows a photograph demonstrating electrophoretic patterns of the sugarcane varieties NiF8 and Ni9 and hybrid progeny lines thereof at the PCR marker N91653962.

FIG. 64 shows a characteristic diagram demonstrating the number of reads of the sugarcane varieties NiF8 and Ni9 and hybrid progeny lines thereof at the marker N91124801.

FIG. 65 shows a photograph demonstrating electrophoretic patterns of the sugarcane varieties NiF8 and Ni9 and hybrid progeny lines thereof at the PCR marker N91124801.

FIG. 66 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 9 nucleotides.

FIG. 67 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 9 nucleotides.

FIG. 68 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 10 nucleotides.

FIG. 69 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 10 nucleotides.

FIG. 70 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 11 nucleotides.

FIG. 71 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 11 nucleotides.

FIG. 72 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 12 nucleotides.

FIG. 73 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 12 nucleotides.

FIG. 74 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 14 nucleotides.

FIG. 75 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 14 nucleotides.

FIG. 76 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 16 nucleotides.

FIG. 77 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 16 nucleotides.

FIG. 78 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 18 nucleotides.

FIG. 79 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 18 nucleotides.

FIG. 80 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 20 nucleotides.

FIG. 81 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 20 nucleotides.

FIG. 82 shows a characteristic diagram demonstrating the results of investigating the reproducibility of the DNA library amplified using DNA of the sugarcane variety NiF8 as a template and random primers each comprising 8 to 35 nucleotides used at a concentration of 0.6 to 300 μM.

FIG. 83 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 1 type of random primer.

FIG. 84 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 1 type of random primer.

FIG. 85 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 2 types of random primers.

FIG. 86 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 2 types of random primers.

FIG. 87 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 3 types of random primers.

FIG. 88 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 3 types of random primers.

FIG. 89 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 12 types of random primers.

FIG. 90 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 12 types of random primers.

FIG. 91 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 24 types of random primers.

FIG. 92 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 24 types of random primers.

FIG. 93 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 48 types of random primers.

FIG. 94 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 48 types of random primers.

FIG. 95 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer B comprising 10 nucleotides.

FIG. 96 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer B comprising 10 nucleotides.

FIG. 97 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer C comprising 10 nucleotides.

FIG. 98 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer C comprising 10 nucleotides.

FIG. 99 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer D comprising 10 nucleotides.

FIG. 100 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer D comprising 10 nucleotides.

FIG. 101 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer E comprising 10 nucleotides.

FIG. 102 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer E comprising 10 nucleotides.

FIG. 103 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer F comprising 10 nucleotides.

FIG. 104 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer F comprising 10 nucleotides.

FIG. 105 shows a characteristic diagram demonstrating a correlation between an amplified fragment length and a fluorescence unit (FU) in which the amplified fragment length is determined based on an electrophoretic pattern (appeared for the first time) of the DNA library amplified using human genomic DNA as a template and a random primer A comprising 10 nucleotides.

FIG. 106 shows a characteristic diagram demonstrating a correlation between an amplified fragment length and a fluorescence unit (FU) in which the amplified fragment length is determined based on an electrophoretic pattern (appeared for the second time) of the DNA library amplified using human genomic DNA as a template and a random primer A comprising 10 nucleotides.

FIG. 107 schematically shows a characteristic diagram of a method for producing a DNA library applied to a next-generation sequencer.

FIG. 108 schematically shows a characteristic diagram of a method for producing a DNA library applied to a next-generation sequencer.

FIG. 109 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer G comprising 10 nucleotides.

FIG. 110 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer G comprising 10 nucleotides.

FIG. 111 shows a characteristic diagram demonstrating a correlation between an amplified fragment length and a fluorescence unit (FU) in which the amplified fragment length is determined based on an electrophoretic pattern (appeared for the first time) of the DNA library amplified using a DNA library of the sugarcane variety NiF8 produced using a random primer G comprising 10 nucleotides as a template and a next-generation sequencer.

FIG. 112 shows a characteristic diagram demonstrating a correlation between an amplified fragment length and a fluorescence unit (FU) in which the amplified fragment length is determined based on an electrophoretic pattern (appeared for the second time) of the DNA library amplified using a DNA library of the sugarcane variety NiF8 produced using a random primer G comprising 10 nucleotides as a template and a next-generation sequencer.

FIG. 113 shows a characteristic diagram demonstrating the results of MiSeq analysis of a DNA library amplified using DNA of the sugarcane variety NiF8 as a template and a random primer G comprising 10 nucleotides.

FIG. 114 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the rice variety Nipponbare as a template and a random primer B comprising 12 nucleotides.

FIG. 115 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the rice variety Nipponbare as a template and a random primer B comprising 12 nucleotides.

FIG. 116 shows a characteristic diagram demonstrating a correlation between an amplified fragment length and a fluorescence unit (FU) in which the amplified fragment length is determined based on an electrophoretic pattern (appeared for the first time) of the DNA library amplified using a DNA library of the rice variety Nipponbare produced using a random primer B comprising 12 nucleotides as a template and a next-generation sequencer.

FIG. 117 shows a characteristic diagram demonstrating a correlation between an amplified fragment length and a fluorescence unit (FU) in which the amplified fragment length is determined based on an electrophoretic pattern (appeared for the second time) of the DNA library amplified using a DNA library of the rice variety Nipponbare produced using a random primer B comprising 12 nucleotides as a template and a next-generation sequencer.

FIG. 118 shows a characteristic diagram demonstrating a distribution of the read pattern obtained by MiSeq analysis of a DNA library amplified using DNA of the rice variety Nipponbare as a template and a random primer B comprising 12 nucleotides and the degree of consistency between the random primer sequence and the reference sequence of rice variety Nipponbare.

FIG. 119 shows a characteristic diagram demonstrating the results of MiSeq analysis of a DNA library amplified using DNA of the rice variety Nipponbare as a template and a random primer B comprising 12 nucleotides.

### DESCRIPTION OF EMBODIMENTS

Hereafter, the present invention is described in detail.

According to the method for producing a DNA library of the present invention, a nucleic acid amplification reaction is conducted in a reaction solution, which is prepared to contain a primer having an arbitrary nucleotide sequence (hereafter, referred to as “random primer”) at a high concentration, and the amplified nucleic acid fragment is determined to be a DNA library. The expression “high concentration” used herein means that the concentration is higher than the primer concentration in a general nucleic acid amplification reaction. Specifically, the method for producing a DNA library of the present invention is characterized in that a random primer is used at a higher concentration than a primer used in a general nucleic acid amplification reaction. As a template contained in a reaction solution, genomic DNA prepared from a target organism for which a DNA library is produced can be used.

In the method for producing a DNA library of the present invention, a target organism species is not particularly limited, and a target organism species can be any organism species such as an animal including a human, a plant, a microorganism, or a virus. In other words, according to the method for producing a DNA library of the present invention, a DNA library can be produced from any organism species.

In the method for producing a DNA library of the present invention, the concentration of a random primer is specified as described above. Thus, a nucleic acid fragment (or nucleic acid fragments) can be amplified with high reproducibility. The term “reproducibility” used herein means an extent of concordance among nucleic acid fragments amplified by a plurality of nucleic acid amplification reactions carried out with the use of the same template and the same random primer. That is, the term “high reproducibility (or the expression “reproducibility is high”)” means that the extent of concordance among nucleic acid fragments amplified by a plurality of nucleic acid amplification reactions carried out with the use of the same template and the same random primer is high.

The extent of reproducibility can be evaluated by, for example, conducting a plurality of nucleic acid amplification reactions with the use of the same template and the same random primer, calculating the Spearman's rank correlation coefficient for the fluorescence unit (FU) obtained as a result of electrophoresis of the resulting amplified fragments, and evaluating the extent of reproducibility on the basis of such coefficient. The Spearman's rank correlation coefficient is generally represented by the symbol p. When p is greater than 0.9, for example, the reproducibility of the amplification reaction of interest can be evaluated to be sufficient.

[Random Primer]

A sequence constituting a random primer that can be used in the method for producing a DNA library according to the present invention is not particularly limited. For example, a random primer comprising nucleotides comprising 9 to 30 nucleotides can be used. In particular, a random primer may be composed of any nucleotide sequence comprising 9 to 30 nucleotides, a nucleotide type (i.e., a sequence type) is not particularly limited, and a random primer may be composed of 1 or more types of nucleotide sequences, preferably 1 to 10,000 types of nucleotide sequences, more preferably 1 to 1,000 types of nucleotide sequences, further preferably 1 to 100 types of nucleotide sequences, and most preferably 1 to 96 types of nucleotide sequences. With the use of nucleotides (or a group of nucleotides) within the range mentioned above for a random primer, an amplified nucleic acid fragment can be obtained with higher reproducibility. When a random primer comprises a plurality of nucleotide sequences, it is not necessary that all nucleotide sequences comprise the same number of nucleotides (9 to 30 nucleotides). A random primer may comprise a plurality of nucleotide sequences composed of a different number of nucleotides.

In general, in order to obtain a specific amplicon by a nucleic acid amplification reaction, the nucleotide sequence of a primer corresponding to the amplicon is designed. For example, a pair of primers are designed such that the primers sandwich a site corresponding to an amplicon of a template DNA of genomic DNA or the like. In such case, as the primers are designed to be hybridized to a specific region included in a template, they may be referred to as “specific primers.”

Meanwhile, a random primer is different from a primer that is designed to obtain a specific amplicon, and it is designed to obtain a random amplicon but not to be hybridized to a specific region of a template DNA. A random primer may have any nucleotide sequence and can contribute to random amplicon amplification when it is incidentally hybridized to a region included in template DNA.

In other words, a random primer can be regarded as nucleotides involved in random amplicon amplification comprising an arbitrary sequence as described above. Here, such arbitrary sequence is not particularly limited. However, it may be designed as, for example, a nucleotide sequence randomly selected from the group consisting of adenine, guanine, cytosine, and thymine or a specific nucleotide sequence. Examples of a specific nucleotide sequence include a nucleotide sequence including a restriction enzyme recognition sequence or a nucleotide sequence having an adapter sequence used for a next-generation sequencer.

When designing plural types of nucleotides for random primers, it is possible to use a method for designing a plurality of nucleotide sequences having certain lengths by randomly selecting from the group consisting of adenine, guanine, cytosine, and thymine. In addition, when designing different types of nucleotides for random primers, it is also possible to use a method for designing a plurality of nucleotide sequences each comprising a common part consisting of a specific nucleotide sequence and a non-common part consisting of an arbitrary nucleotide sequence. Here, the non-common part may consist of a nucleotide sequence randomly selected from the group consisting of adenine, guanine, cytosine, and thymine or all or one of combinations of four types of nucleotides which are adenine, guanine, cytosine, and thymine. The common part is not particularly limited, and it may consist of any nucleotide sequence. It may consist of, for example, a nucleotide sequence including a restriction enzyme recognition sequence, a nucleotide sequence having an adapter sequence used for a next-generation sequencer, or a nucleotide sequence common in a specific gene family.

When designing plural types of nucleotide sequences having certain lengths by randomly selecting nucleotides from four types of nucleotides for a plurality of random primers, 30% or more, preferably 50% or more, more preferably 70% or more, and further preferably 90% or more of the entire such sequences exhibit 70% or less, preferably 60% or less, more preferably 50% or less, and most preferably 40% or less identity. By designing different types of nucleotide sequences having certain lengths by randomly selecting nucleotides from different types of nucleotides for a plurality of random primers exhibiting the identity within such range, an amplified fragment can be obtained over the entire genomic DNA of the target organism species. Thus, uniformity of the amplified fragment can be enhanced.

When designing a plurality of nucleotide sequences each comprising a common part consisting of a specific nucleotide sequence and a non-common part consisting of an arbitrary nucleotide sequence for a plurality of random primers, it is possible to design, for example, a nucleotide sequence comprising a non-common part consisting of several nucleotides on the 3′ end side and a common part consisting of the remaining nucleotides on the 5′ end side. By allowing a non-common part to consist of n number of nucleotides on the 3′ end side, it is possible to design 4ⁿ types of random primers. Here, the expression “n number” may refer to 1 to 5, preferably 2 to 4, and more preferably 2 to 3.

For example, it is possible to design, as a random primer comprising a common part and a non-common part, 16 types of random primers in total, each of which has an adapter sequence (common part) used for a next-generation sequencer on the 5′ end side and two nucleotides (non-common part) on the 3′ end side in total. It is possible to design 64 types of random primers in total by setting the number of nucleotides on the 3′ end side to 3 nucleotides (non-common part). The more types of random primers, the more comprehensively the amplified fragments can be obtained throughout the genomic DNA of the target organism species. Therefore, when designing a random primer consisting of a common part and a non-common part, it is preferable that 3 nucleotides exist on the 3′ end side.

However, for example, after designing 64 types of nucleotide sequences each comprising a common part and a non-common part consisting of 3 nucleotides, not more than 63 types of random primers selected from these 64 types of nucleotide sequences may be used. In other words, as compared with the case of using all 64 types of random primers, in the case of using not more than 63 types of random primers, excellent results can be obtained in a nucleic acid amplification reaction or analysis using a next generation sequencer. Specifically, when 64 types of random primers are used, the number of reads of a specific nucleic acid amplification fragment might become remarkably large. In such case, favorable analysis results can be obtained by using the remaining 63 random primers excluding one or more random primers involved in the amplification of the specific nucleic acid amplification fragment from 64 types of random primers.

Similarly, in the case of designing 16 types of random primers each comprising a common part and a non-common part of 2 nucleotides, when not more than 15 types of random primers selected from 16 types of random primers are used, favorable analysis results may be obtained in a nucleic acid amplification reaction or analysis using a next generation sequencer.

Nucleotides constituting a random primer are preferably designed such that the G-C content is 5% to 95%, more preferably 10% to 906, further preferably 15% to 80%, and most preferably 20% to 70%. With the use of a set of nucleotides having a G-C content within the above range as a random primer, amplified nucleic acid fragments can be obtained with enhanced reproducibility. The G-C content is the percentage of guanine and cytosine contained in the whole nucleotide chain.

Further, nucleotides constituting a random primer are designed such that consecutive nucleotides account for preferably 80% or less, more preferably 70% or less, further preferably 60% or less, and most preferably 50% or less with respect to the entire sequence length. Alternatively, nucleotides constituting a random primer are designed such that the number of consecutive nucleotides is preferably 8 or less, more preferably 7 or less, further preferably 6 or less, and most preferably 5 or less. An amplified nucleic acid fragment can be obtained with enhanced reproducibility with the use of a set of nucleotides constituting a random primer, for which the number of consecutive nucleotides falls within the above range.

In addition, it is preferable that nucleotides constituting a random primer be designed not to constitute a complementary region of 6 or more, more preferably 5 or more, and further preferably 4 or more nucleotides in a molecule. When the nucleotides designed not to constitute a complementary region within the above range, double strand formation occurring in a molecule can be prevented, and amplified nucleic acid fragments can be obtained with enhanced reproducibility.

Further, when plural types of nucleotides are designed for a random primer, in particular, it is preferable that a plurality of nucleotides be designed not to constitute a complementary region of 6 or more, more preferably 5 or more, and further preferably 4 or more nucleotides while forming a plurality of nucleotide sequences. When different types of nucleotide sequences are designed Thus, double strand formation occurring between nucleotide sequences can be prevented, and amplified nucleic acid fragments can be obtained with enhanced reproducibility.

When plural types of nucleotides are designed for random primers, it is preferable that the nucleotides be designed not to constitute a complementary sequence of 6 or more, more preferably 5 or more, and further preferably 4 or more nucleotides at the 3′ end side. When they are designed not to form a complementary sequence within the above range at the 3′ end side, double strand formation occurring between nucleotide sequences can be prevented, and amplified nucleic acid fragments can be obtained with enhanced reproducibility.

The terms “complementary region” and “complementary sequence” refer to, for example, a region and a sequence exhibiting 80% to 100% identity (e.g., a region and a sequence each comprising 5 nucleotides in which 4 or 5 nucleotides are complementary to each other) or a region and a sequence exhibiting 90% to 100% identity (e.g., a region and a sequence each comprising 5 nucleotides in which 5 nucleotides are complementary to each other).

Further, nucleotides constituting a random primer are preferably designed to have a Tm value suitable for thermal cycle conditions (in particular, an annealing temperature) in a nucleic acid amplification reaction. A Tm value can be calculated by a conventional method, such as the nearest neighbor base pair approach, the Wallace method, or the GC % method, although a method of calculation is not particularly limited thereto. Specifically, nucleotides used for a random primer are preferably designed to have a Tm value of 10° C. to 85° C., more preferably 12° C. to 75° C., further preferably 14° C. to 70° C., and most preferably 16° C. to 65° C. By designing Tm values for nucleotides within the above range, amplified nucleic acid fragments can be obtained with enhanced reproducibility under given thermal cycle conditions (in particular, at a given annealing temperature) in a nucleic acid amplification reaction.

Furthermore, when different types of nucleotides constituting a random primer are designed, in particular, a variation for Tm among a plurality of nucleotides is preferably 50° C. or less, more preferably 45° C. or less, further preferably 40° C. or less, and most preferably 35° C. or less. When the nucleotides are designed such that a variation for Tm among a plurality of nucleotides falls within the above range, amplified nucleic acid fragments can be obtained with enhanced reproducibility under given thermal cycle conditions (in particular, at a given annealing temperature) in a nucleic acid amplification reaction.

[Nucleic Acid Amplification Reaction]

According to the method for producing a DNA library of the present invention, many amplification fragments are obtained via a nucleic acid amplification reaction conducted with the use of the random primer and genomic DNA as a template described above. In particular, in such a nucleic acid amplification reaction, the concentration of a random prime in a reaction solution is set higher than the primer concentration in a usual nucleic acid amplification reaction. Thus, many amplification fragments can be obtained using genomic DNA as a template while achieving high reproducibility. The thus obtained many amplification fragments can be used for a DNA library that can be applied to genotyping and the like.

A nucleic acid amplification reaction is a reaction for synthesizing amplification fragments in a reaction solution containing genomic DNA as a template, the above-mentioned random primers. DNA polymerase, deoxynucleoside triphosphate as a substrate (i.e., dNTP, which is a mixture of dATP, dCTP, dTITP, and dGTP), and a buffer under given thermal cycle conditions. As it is necessary to add Mg²⁺ at a given concentration to a reaction solution in a nucleic acid amplification reaction, the buffer of the above composition contains MgCl₂. When the buffer does not contain MgCl₂, MgCl₂ is further added to the above composition.

In particular, in a nucleic acid amplification reaction, it is preferable to adequately set the concentration of a random primer in accordance with the nucleotide length of the random primer. When different types of nucleotides constitute random primers with different nucleotide lengths, the average of nucleotide lengths of random primers may be set as the nucleotide length (the average may be a simple average or the weight average taking the amount of nucleotides into account).

Specifically, a nucleic acid amplification reaction is conducted using a random primer comprising 9 to 30 nucleotides at a random primer concentration of 4 to 200 μM and preferably at 4 to 100 μM. Under such conditions, many amplified fragments, and in particular, many amplified fragments comprising 100 to 500 nucleotides via a nucleic acid amplification reaction can be obtained while achieving high reproducibility.

More specifically, when a random primer comprises 9 to 10 nucleotides, the random primer concentration is preferably 40 to 60 μM. When a random primer comprises 10 to 14 nucleotides, it is preferable that the random primer concentration satisfy 100 μM or less and y>3E+08x^{-6.974}, provided that the nucleotide length of the random primer is represented by “y” and the concentration of the random primer is represented by “x.” When a random primer comprises 14 to 18 nucleotides, the random primer concentration is preferably 4 to 100 μM. When a random primer comprises 18 to 28 nucleotides, the random primer concentration satisfies preferably 4 μM or more and y<8E+08x^{-5.533}. When a random primer comprises 28 to 29 nucleotides, the random primer concentration is preferably 6 to 10 μM. By setting the random primer concentration in accordance with the nucleotide length of a random primer as described above, many amplified fragments can be obtained with improved certainty while achieving high reproducibility.

As described in the Examples below, the above inequations (y>3E+08x^{-6.94} and y<8E+08x^{-5.533}) are developed to be able to represent the random primer concentration at which many DNA fragments comprising 100 to 500 nucleotides can be obtained with favorable reproducibility as a result of thorough inspection of the correlation between the random primer length and the random primer concentration.

The amount of genomic DNA as a template in a nucleic acid amplification reaction is not particularly limited. However, it is preferably 0.1 to 1000 ng, more preferably 1 to 500 ng, further preferably 5 to 200 ng, and most preferably 10 to 100 ng, when the amount of the reaction solution is 50 μl. By setting the amount of genomic DNA as a template within the above range, many amplified fragments can be obtained without inhibiting the amplification reaction with a random primer, while achieving high reproducibility.

Genomic DNA can be prepared in accordance with a conventional technique without particular limitations. With the use of a commercially available kit, genomic DNA can be easily prepared from a target organism species. Genomic DNA extracted from an organism in accordance with a conventional technique or with the use of a commercially available kit may be used as is, genomic DNA extracted from an organism and purified may be used, or genomic DNA subjected to restriction enzyme treatment or ultrasonic treatment may be used.

DNA polymerase used in a nucleic acid amplification reaction is not particularly limited, and an enzyme having DNA polymerase activity under thermal cycle conditions for a nucleic acid amplification reaction can be used. Specifically, heat-stable DNA polymerase used for a general nucleic acid amplification reaction can be used. Examples of DNA polymerase include thermophilic bacteria-derived DNA polymerase such as Taq DNA polymerase, and hyperthermophilic Archaea-derived DNA polymerase such as KOD DNA polymerase or Pfu DNA polymerase. In a nucleic acid amplification reaction, it is particularly preferable to use Pfu DNA polymerase as DNA polymerase in combination with the random primer described above. With the use of such DNA polymerases, many amplified fragments can be obtained with improved certainty while achieving high reproducibility.

In a nucleic acid amplification reaction, the concentration of deoxynucleoside triphosphate as a substrate (i.e., dNTP, which is a mixture of dATP, dCTP, dTTP, and dGTP) is not particularly limited, and it can be 5 μM to 0.6 mM, preferably 10 μM to 0.4 mM, and more preferably 20 μM to 0.2 mM. By setting the concentration of dNTP as a substrate within such range, errors caused by incorrect incorporation by DNA polymerase can be prevented, and many amplified fragments can be obtained while achieving high reproducibility.

A buffer used in a nucleic acid amplification reaction is not particularly limited. For example, a solution comprising MgCl₂ as described above, Tris-HCl (pH 8.3), and KCl can be used. The concentration of Mg²⁺ is not particularly limited. For example, it can be 0.1 to 4.0 mM, preferably 0.2 to 3.0 mM, more preferably 0.3 to 2.0 mM, and further preferably 0.5 to 1.5 mM. By designating the concentration of Mg²⁺ in the reaction solution within such range, many amplified fragments can be obtained while achieving high reproducibility.

Thermal cycling conditions of a nucleic acid amplification reaction are not particularly limited, and a common thermal cycle can be adopted. A specific example of a thermal cycle comprises a first step of thermal denaturation in which genomic DNA as a template is dissociated into single strands, a cycle comprising thermal denaturation, annealing, and extension repeated a plurality of times (e.g., 20 to 40 times), a step of extension for a given period of time according to need, and the final step of storage.

Thermal denaturation can be performed at, for example, 93° C. to 99° C., preferably 95° C. to 98° C., and more preferably 97° C. to 98° C. Annealing can be performed at, for example, 30° C. to 70° C., preferably 35° C. to 68° C., and more preferably 37° C. to 65° C., although it varies depending on the Tm value of a random primer. Extension can be performed at, for example, 70° C. to 76° C., preferably 71° C. to 75° C., and more preferably 72° C. to 74° C. Storage can be performed at, for example, 4° C.

The first step of thermal denaturation can be performed within the temperature range described above for a period of, for example, 5 seconds to 10 minutes, preferably 10 seconds to 5 minutes, and more preferably 30 seconds to 2 minutes. In the cycle comprising “thermal denaturation, annealing, and extension,” thermal denaturation can be carried out within the temperature range described above for a period of, for example, 2 seconds to 5 minutes, preferably 5 seconds to 2 minutes, and more preferably 10 seconds to 1 minute. In the cycle comprising “thermal denaturation, annealing, and extension,” annealing can be carried out within the temperature range described above for a period of, for example, 1 second to 3 minutes, preferably 3 seconds to 2 minutes, and more preferably 5 seconds to 1 minute. In the cycle comprising “thermal denaturation, annealing, and extension,” extension can be carried out within the temperature range described above for a period of, for example, 1 second to 3 minutes, preferably 3 seconds to 2 minutes, and more preferably 5 seconds to 1 minute.

According to the method for producing a DNA library of the present invention, amplified fragments may be obtained by a nucleic acid amplification reaction that employs a hot start method. The hot start method is intended to prevent mis-priming or non-specific amplification caused by primer-dimer formation prior the cycle comprising “thermal denaturation, annealing, and extension.” The hot start method involves the use of an enzyme in which DNA polymerase activity has been suppressed by binding an anti-DNA polymerase antibody thereto or chemical modification thereof. Thus, DNA polymerase activity can be suppressed and a non-specific reaction prior to the thermal cycle can be prevented. According to the hot start method, a temperature is set high in the first thermal cycle, DNA polymerase activity is thus recovered, and the subsequent nucleic acid amplification reaction is then allowed to proceed.

As described above, many amplified fragments can be obtained with the use of genomic DNA as a template and a random primer by conducting a nucleic acid amplification reaction with the use of a random primer comprising 9 to 30 nucleotides and setting the concentration thereof to 4 to 200 μM in a reaction solution. With the use of the random primer comprising 9 to 30 nucleotides while setting the concentration thereof to 4 to 200 μM in a reaction solution, a nucleic acid amplification reaction can be performed with very high reproducibility. According to the nucleic acid amplification reaction, specifically, many amplified fragments can be obtained while achieving very high reproducibility. Therefore, the thus obtained many amplified fragments can be used for a DNA library in genetic analysis targeting genomic DNA.

By performing a nucleic acid amplification reaction with the use of the random primer comprising 9 to 30 nucleotides and setting the concentration thereof in a reaction solution to 4 to 200 μM, in particular, many amplified fragments comprising about 100 to 500 nucleotides can be obtained with the use of genomic DNA as a template. Such many amplified fragments comprising about 100 to 500 nucleotides are suitable for mass analysis of nucleotide sequences with the use of, for example, a next-generation sequencer, and highly accurate sequence information can thus be obtained. According to the present invention, a DNA library including DNA fragments comprising about 100 to 500 nucleotides can be produced.

By performing a nucleic acid amplification reaction with the use of the random primer comprising 9 to 30 nucleotides and setting the concentration thereof to 4 to 200 μM in a reaction solution, in particular, amplified fragments can be obtained uniformly across genomic DNA. In other words, DNA fragments are amplified in a distributed manner across the genome but not in a localized manner in a specific region of genomic DNA in a nucleic acid amplification reaction with the use of such random primer. That is, according to the present invention, a DNA library can be produced uniformly across the entire genome.

After performing the nucleic acid amplification reaction using the above-mentioned random primer, restriction enzyme treatment, size selection treatment, sequence capture treatment, and the like can be performed on the obtained amplified fragments. By carrying out restriction enzyme treatment, size selection treatment, and sequence capture treatment on the amplified fragments, specific amplified fragments (a fragment having a specific restriction enzyme site, an amplified fragment with a specific size range, and an amplified fragment having a specific sequence) can be obtained from among the obtained amplified fragments. Then, specific amplified fragments obtained by these treatments can be used for a DNA library.

[Method of Genomic DNA Analysis]

With the use of the DNA library produced in the manner described above, genomic DNA analysis such as genotyping can be performed. Such DNA library has very high reproducibility, the size thereof is suitable for a next-generation sequencer, and it has uniformity across the entire genome. Accordingly, the DNA library can be used as a DNA marker (also referred to as “genetic marker” or “gene marker”). The term “DNA marker” refers to a wide range of characteristic nucleotide sequences present in genomic DNA. In addition, a DNA marker may be especially a nucleotide sequence on the genome serving as a marker associated with genetic traits. A DNA marker can be used for, for example, genotype identification, linkage mapping, gene mapping, breeding comprising a step of selection with the use of a marker, back crossing using a marker, quantitative trait locus mapping, bulked segregant analysis, variety identification, or discontinuous imbalance mapping.

For example, the nucleotide sequence of a DNA library prepared as described above is determined using a next generation sequencer or the like, and the presence or absence of a DNA marker can be confirmed based on the obtained nucleotide sequence.

As an example, the presence or absence of a DNA marker can be confirmed from the number of reads of the obtained nucleotide sequence. While a next-generation sequencer is not particularly limited, such sequencer is also referred to as a “second-generation sequencer,” and such sequencer is an apparatus for nucleotide sequencing that allows simultaneous determination of nucleotide sequences of several tens of millions of DNA fragments. The sequencing principle of a next-generation sequencer is not particularly limited. For example, sequencing can be carried out in accordance with a method in which sequencing is carried out while amplifying and synthesizing target DNA on flow cells by bridge PCR method and the sequencing-by-synthesis method, or in accordance with a method in which sequencing is carried out by emulsion PCR and the pyrosequencing method for assaying the amount of pyrophosphoric acids released upon DNA synthesis. More specific examples of next-generation sequencers include MiniSeq, MiSeq, NextSeq, HiSeq, and HiSeq X Series (Illumina, Inc.) and Roche 454 GS FLX sequencers (Roche).

In another example, the presence or absence of a DNA marker can be confirmed by comparing the nucleotide sequence obtained for the DNA library prepared as described above with the reference nucleotide sequence. Here, the reference nucleotide sequence means a known sequence as a reference, and it can be, for example, a known sequence stored in a database. That is, a DNA library is prepared as described above for a given organism, its nucleotide sequence is determined, and the nucleotide sequence of the DNA library is compared with the reference nucleotide sequence. A nucleotide sequence that differs from the reference nucleotide sequence can be designated as a DNA marker (a characteristic nucleotide sequence existing in the genomic DNA) related to the organism. For each specified DNA marker, the relevance to the genetic trait (phenotype) can be determined by further analysis according to a conventional method. In other words, a DNA marker related to a phenotype (sometimes referred to as a “selective marker”) can be identified from among the DNA markers identified as described above.

Furthermore, in another example, the presence or absence of a DNA marker can be confirmed by comparing the nucleotide sequence obtained for the DNA library prepared as described above with the nucleotide sequence of a DNA library prepared as described above using genomic DNA from a different organism or tissue. In other words, a DNA library is prepared as described above for each of two or more organisms or two different tissues, the nucleotide sequences thereof are determined, and the nucleotide sequences of the DNA libraries are compared with each other. Then, a nucleotide sequence that differs between the DNA libraries can be designated as a DNA marker (a characteristic nucleotide sequence existing in the genomic DNA) related to the sampled organism or tissue. For each specified DNA marker, the relevance to the genetic trait (phenotype) can be determined by further analysis according to a conventional method. In other words, a DNA marker related to a phenotype (sometimes referred to as a “selective marker”) can be identified from among the DNA markers identified as described above.

As an aside, it is also possible to design a pair of primers which specifically amplify the DNA marker based on the obtained nucleotide sequence. It is also possible to confirm the presence or absence of the DNA marker in the extracted genomic DNA by performing a nucleic acid amplification reaction using a pair of designed primers and genomic DNA extracted from a target organism as a template.

Alternatively, DNA libraries prepared as described above can be used for metagenomic analysis for examining a wide variety of microorganisms and the like, genome mutation analysis of somatic cells of tumor tissue or the like, genotyping using microarrays, determination and analysis of ploidy, calculation and analysis of the number of chromosomes, analysis of the increase and decrease of chromosomes, analysis of partial insertion/deletion/replication/translocation of chromosomes, analysis of contamination with foreign genome, parentage discrimination analysis, and testing and analysis of crossed seed purity.

[Application to Next Generation Sequencing Technology]

As described above, by conducting a nucleic acid amplification reaction with a random primer contained at a high concentration in a reaction solution, it is possible to obtain many amplified fragments with favorable reproducibility using genomic DNA as a template. Since each obtained amplified fragment has nucleotide sequence at both ends thereof which are the same as those of the random primer, it can be easily applied to the next generation sequence technology by utilizing the nucleotide sequence.

Specifically, as described above, a nucleic acid amplification reaction is conducted in a reaction solution (first reaction solution) containing genomic DNA and a random primer at a high concentration to obtain many amplified fragments (first DNA fragments) using the genomic DNA as a template. Next, a nucleic acid amplification reaction is conducted in a reaction solution (second reaction solution) containing the obtained many amplified fragments (first DNA fragments) and a primer designed based on the nucleotide sequence of the random primer (referred to as “next generation sequencer primer”). A next generation sequencer primer to be used herein is a nucleotide sequence including a region used for a nucleotide sequencing reaction. More specifically, for example, the next-generation sequencer primer may be a nucleotide sequence having a region necessary for a nucleotide sequencing reaction (sequence reaction) by a next-generation sequencer, in which the nucleotide sequence at the 3′ end of the primer is a nucleotide sequence having 70% or more identity, preferably 80% or more identity, more preferably 90% or more identity, still more preferably 95% or more identity, further preferably 97% or more identity, and most preferably 100% identity to the nucleotide sequence on the 5′ end side of the first DNA fragment.

Here, the “region used for a nucleotide sequencing reaction” included in a next-generation sequencer primer is not particularly limited because it varies depending on type of the next-generation sequencer. However, in the case of conducting a nucleotide sequencing reaction using a next-generation sequencer with a sequence primer, such region may be, for example, a nucleotide sequence complementary to the nucleotide sequence of the sequence primer. In a case in which a sequencing reaction is conducted by a next-generation sequencer using capture beads bound to given DNA, the “region used for a nucleotide sequencing reaction” refers to a nucleotide sequence complementary to the nucleotide sequence of the DNA bound to capture beads. Further, in a case in which a next-generation sequencer reads a sequence based on a current change when a DNA chain having a terminal hairpin loop passes through a protein having nano-sized pores, the “region used for a nucleotide sequencing reaction” may be a nucleotide sequence complementary to the nucleotide sequence forming the hairpin loop.

By designing the nucleotide sequence at the 3′ end of a next-generation sequencer primer as described above, the next-generation sequencer primer can be hybridized to the 3′ end of the first DNA fragment under stringent conditions, and the second DNA fragment can be amplified using the first DNA fragment as a template. Stringent conditions mean conditions under which a so-called specific hybrid is formed while a nonspecific hybrid is not formed. For example, such conditions can be appropriately determined with reference to Molecular Cloning: A Laboratory Manual (Third Edition). Specifically, stringency can be determined by setting the temperature and the salt concentration in a solution upon Southern hybridization, and the temperature and the salt concentration in a solution in the washing step of Southern hybridization. More specifically, for example, the sodium concentration is set to 25 to 500 mM and preferably 25 to 300 mM and the temperature is set to 42° C. to 68° C. and preferably 42° C. to 65° C. under stringent conditions. More specifically, the sodium concentration is 5×SSC (83 mM NaCl, 83 mM sodium citrate) and the temperature is 42° C.

In particular, when different types of random primers are used to obtain a first DNA fragment, next-generation sequencer primers may be prepared to correspond to all or some of random primers.

For example, in a case in which a set of different types of random primers (each having an arbitrary 3′-end sequence of several nucleotides) each comprising a common nucleotide sequence except several nucleotides (e.g., about 1 to 3 nucleotides) at the 3′ end is used, all of the obtained many first DNA fragments have a common 5′-end sequence. Accordingly, the 3′-end nucleotide sequence of a next generation sequencer primer is designated to be a nucleotide sequence having 70% or more identity to the 5′-end nucleotide sequence common to the first DNA fragments. By designing next-generation sequencer primers as described above, it is possible to obtain next generation sequencer primers corresponding to all random primers. By using such next generation sequencer primers, it is possible to amplify second DNA fragments using all of the first DNA fragments as templates.

Similarly, even in a case in which a set of different types of random primers (each having an arbitrary 3′-end sequence of several nucleotides) each comprising a common nucleotide sequence except several nucleotides (e.g., about 1 to 3 nucleotides) at the 3′ end is used, it is also possible to obtain second DNA fragments using some of the obtained many first DNA fragments as templates. Specifically, the 3′-end nucleotide sequence of a next generation sequencer primer is designated to be a nucleotide sequence having 70% or more identity to the 5′-end nucleotide sequence common to the first DNA fragments and the sequence comprising several nucleotides following the nucleotide sequence (corresponding to several nucleotides (arbitrary sequence) at the 3′ end of the random primer) such that second DNA fragments can be amplified using some of the first DNA fragments as templates.

Meanwhile, in a case in which first DNA fragments are obtained using different types of random primers each consisting of an arbitrary nucleotide sequence, it is possible to obtain second DNA fragments using different types of next-generation sequencer primers such that the second DNA fragments correspond to all of the first DNA fragments, or it is also possible to obtain second DNA fragments using different types of next-generation sequencer primers such that the second DNA fragments correspond to some of the first DNA fragments.

As described above, the second DNA fragments amplified using next-generation sequencer primers have a region necessary for a nucleotide sequencing reaction (sequence reaction) by a next-generation sequencer, which is included in the next-generation sequencer primers. The region necessary for a sequence reaction is not particularly limited as it varies depending on a next generation sequencer. For example, when a next-generation sequencer primer is used in a next-generation sequencer based on the principle that sequencing is carried out while amplifying and synthesizing target DNA on flow cells by bridge PCR method and the sequencing-by-synthesis method, the next-generation sequencer primer needs to contain a region necessary for bridge PCR and a region necessary for the sequencing-by-synthesis method. The region necessary for bridge PCR is a region that is hybridized to an oligonucleotide immobilized on flow cells and has a length of 9 nucleotides including the 5′ end of the next generation sequencer primer. In addition, a region necessary for the sequencing-by-synthesis method is a region to which a sequence primer used in a sequence reaction is hybridized, and is a region in the middle of the next generation sequencer primer.

In addition, a next-generation sequencer may be an Ion Torrent sequencer. In the case of using the Ion Torrent sequencer, a next-generation sequencer primer has a so-called ion adapter on the 5′ end side and binds to a particle for conducting emulsion PCR. In addition, in the Ion Torrent sequencer, particles coated with a template amplified by emulsion PCR are placed on an ion chip and subjected to a sequence reaction.

Here, a nucleic acid amplification reaction using a next-generation sequencer primer and a second reaction solution containing the first DNA is not particularly limited, and conventional conditions for nucleic acid amplification reaction can be applied. That is, the conditions in [Nucleic acid amplification reaction] described above can be used. For example, the second reaction solution contains first DNA fragments as templates, the above-described next-generation sequencer primer, DNA polymerase, deoxynucleoside triphosphate as a substrate (i.e., dNTP, which is a mixture of dATP, dCTP, dTTP, and dGTP), and a buffer.

In particular, the concentration of the next-generation sequencer primer can be set to 0.01 to 5.0 μM, preferably 0.1 to 2.5 μM, and most preferably 0.3 to 0.7 μM.

While the amount of the first DNA fragments serving as templates in a nucleic acid amplification reaction is not particularly limited, it is preferably 0.1 to 1000 ng, more preferably 1 to 500 ng, further preferably 5 to 200 ng, and most preferably 10 to 100 ng when the amount of the reaction solution is 50 μl.

A method for preparing first DNA fragments as templates is not particularly limited. In the method, the reaction solution obtained after the completion of the nucleic acid amplification reaction using the above-described random primers may be used as is, or the reaction solution may be used after purifying the first DNA fragments therefrom.

Regarding the type of DNA polymerase, the concentration of deoxynucleoside triphosphate as a substrate (dNTP, i.e., a mixture of dATP, dCTP, dTTP and dGTP), the buffer composition, and temperature cycle conditions used for the nucleic acid amplification reaction, the conditions in [Nucleic acid amplification reaction] described above can be used. In addition, in a nucleic acid amplification reaction using next-generation sequencer primers, a hot start method may be employed, or amplified fragments may be obtained by a nucleic acid amplification reaction.

As described above, by using the first DNA fragments obtained using random primers as templates and using the second DNA fragments amplified using next-generation sequencer primers, it is possible to readily prepare a DNA library that can be applied to a next-generation sequencer.

In the above examples, a DNA library is prepared using the first DNA fragments obtained using random primers as templates and amplifying the second DNA fragments using next-generation sequencer primers. However, the scope of the present invention is not limited to Such examples. For example, the DNA library according to the present invention may be prepared by amplifying second DNA fragments using first DNA fragments obtained using random primers as templates and further obtaining third DNA fragments using the second DNA fragments as templates and next-generation sequencer primers, thereby obtaining a DNA library of the third DNA fragments applicable to a next generation sequencer.

Similarly, in order to prepare a DNA library applicable to a next-generation sequencer, after a nucleic acid amplification reaction using second DNA fragments as templates, a nucleic acid amplification reaction is repeatedly conducted using the obtained DNA fragments as templates, and next-generation sequencer primers are used for the final nucleic acid amplification reaction. In such case, the number of nucleic acid amplification reactions to be repeated is not particularly limited, but it is 2 to 10 times, preferably 2 to 5 times, and more preferably 2 to 3 times.

### EXAMPLES

Hereafter, the present invention is described in greater detail with reference to the Examples below, although the scope of the present invention is not limited to these Examples.

### Example 1

### 1. Flowchart

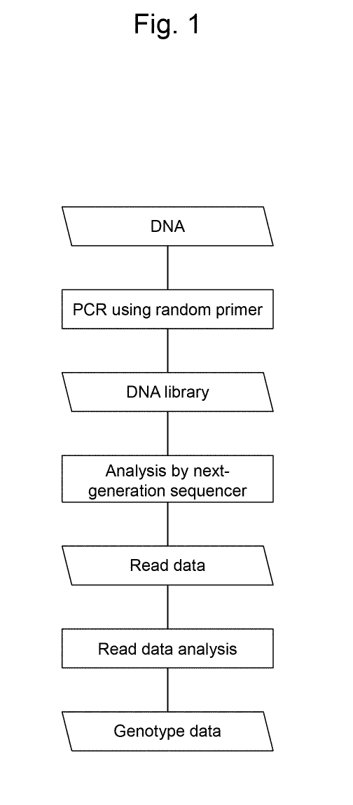

In this Example, a DNA library was prepared via PCR using genomic DNAs extracted from various types of organism species as templates and various sets of random primers in accordance with the flow chart shown in FIG. 1. In addition, with the use of the prepared DNA library, sequence analysis was performed by a so-called next-generation sequencer, and the genotype was analyzed based on the obtained read data.

### 2. Materials

In this Example, genomic DNAs were extracted from the sugarcane varieties NiF8 and Ni9, 22 hybrid progeny lines thereof, and the rice variety Nipponbare using the DNeasy Plant Mini Kit (QIAGEN), and the extracted genomic DNAs were purified. The purified genomic DNAs were used as NiF8-derived genomic DNA, Ni9-derived genomic DNA, genomic DNAs from 22 hybrid progeny lines, and Nipponbare-derived genomic DNA, respectively. In this Example, Human Genomic DNA was purchased as human DNA from TakaraBio and used as human-derived genomic DNA.

### 3. Method

### 3.1 Correlation Between PCR Conditions and DNA Fragment Sizes

### 3.1.1 Random Primer Designing

In order to design random primers, the GC content was set between 20% and 70%, and the number of consecutive nucleotides was adjusted to 5 or less. The nucleotide length was set at 16 levels (i.e., 8, 9, 10, 11, 12, 14, 16, 18, 20, 22, 24, 26, 28, 29, 30, and 35 nucleotides). For each nucleotide length, 96 types of nucleotide sequences were designed, and a set of 96 types of random primers was prepared for each nucleotide length. Concerning 10-nucleotide primers, 6 sets (each comprising 96 types of random primers) were designed (these 6 sets are referred to as 10-nucleotide primer A to 10-nucleotide primer F). In this Example, specifically, 21 different sets of random primers were prepared.

Tables 1 to 21 show nucleotide sequences of random primers contained in these 21 different sets of random primers.

| Table 1-1 | | |
|---|---|---|
| Table 1 | | |
| Random primer list (10-nucleotide A) | | |
| | Primer | SEQ ID |
| No. | sequence | NO: |
| 1 | AGACGTCGTT | 1 |
| 2 | GAGGCGATAT | 2 |
| 3 | GTGCGAACGT | 3 |
| 4 | TTATACTGCC | 4 |
| 5 | CAAGTTCGCA | 5 |
| 6 | ACAAGGTAGT | 6 |
| 7 | ACACAGCGAC | 7 |
| 8 | TTACCGATGT | 8 |
| 9 | CACAGAGTCG | 9 |
| 10 | TTCAGCGCGT | 10 |
| 11 | AGGACCGTGA | 11 |
| 12 | GTCTGTTCGC | 12 |
| 13 | ACCTGTCCAC | 13 |
| 14 | CCGCAATGAC | 14 |
| 15 | CTGCCGATCA | 15 |
| 16 | TACACGGAGC | 16 |
| 17 | CCGCATTCAT | 17 |
| 18 | GACTCTAGAC | 18 |
| 19 | GGAGAACTTA | 19 |
| 20 | TCCGGTATGC | 20 |
| 21 | GGTCAGGAGT | 21 |
| 22 | ACATTGGCAG | 22 |
| 23 | CGTAGACTGC | 23 |
| 24 | AGACTGTACT | 24 |
| 25 | TAGACGCAGT | 25 |
| 26 | CCGATAATCT | 26 |
| 27 | GAGAGCTAGT | 27 |
| 28 | GTACCGCGTT | 28 |
| 29 | GACTTGCGCA | 29 |
| 30 | CGTGATTGCG | 30 |
| 31 | ATCGTCTCTG | 31 |
| 32 | CGTAGCTACG | 32 |
| 33 | GCCGAATAGT | 33 |
| 34 | GTACCTAGGC | 34 |
| 35 | GCTTACATGA | 35 |
| 36 | TCCACGTAGT | 36 |
| 37 | AGAGGCCATC | 37 |
| 38 | CGGTGATGCT | 38 |
| 39 | CACTGTGCTT | 39 |
| 40 | CATGATGGCT | 40 |
| 41 | GCCACACATG | 41 |
| 42 | CACACACTGT | 42 |
| 43 | CAGAATCATA | 43 |
| 44 | ATCGTCTACG | 44 |
| 45 | CGAGCAATAC | 45 |
| 46 | ACAAGCGCAC | 46 |
| 47 | GCTTAGATGT | 47 |
| 48 | TGCATTCTGG | 48 |
| 49 | TGTCGGACCA | 49 |
| 50 | AGGCACTCGT | 50 |
| 51 | CTGCATGTGA | 51 |
| 52 | ACCACGCCTA | 52 |
| 53 | GAGGTCGTAC | 53 |
| 54 | AATACTCTGT | 54 |
| 55 | TGCCAACTGA | 55 |
| 56 | CGTGTTCGGT | 56 |
| 57 | GTAGAGAGTT | 57 |
| 58 | TACAGCGTAA | 58 |
| 59 | TGACGTGATG | 59 |
| 60 | AGACGTCGGT | 60 |
| 61 | CGCTAGGTTC | 61 |
| 62 | GCCTTATAGC | 62 |
| 63 | CCTTCGATCT | 63 |
| 64 | AGGCAACGTG | 64 |

Table 1-2

| No. | Primer sequence | SEQ ID NO: |
|---|---|---|
| 65 | TGAGCGGTGT | 65 |
| 66 | GTGTCGAACG | 66 |
| 67 | CGATGTTGCG | 67 |
| 68 | AACAAGACAC | 68 |
| 69 | GATGCTGGTT | 69 |
| 70 | ACCGGTAGTC | 70 |
| 71 | GTGACTAGCA | 71 |
| 72 | AGCCTATATT | 72 |
| 73 | TCGTGAGCTT | 73 |
| 74 | ACACTATGGC | 74 |
| 75 | GACTCTGTCG | 75 |
| 76 | TCGATGATGC | 76 |
| 77 | CTTGGACACT | 77 |
| 78 | GGCTGATCGT | 78 |
| 79 | ACTCACAGGC | 79 |
| 80 | ATGTGCGTAC | 80 |
| 81 | CACCATCGAT | 81 |
| 82 | AGCCATTAAC | 82 |
| 83 | AATCGACTGT | 83 |
| 84 | AATACTAGCG | 84 |
| 85 | TCGTCACTGA | 85 |
| 86 | CAGGCTCTTA | 86 |
| 87 | GGTCGGTGAT | 87 |
| 88 | CATTAGGCGT | 88 |
| 89 | ACTCGCGAGT | 89 |
| 90 | TTCCGAATAA | 90 |
| 91 | TGAGCATCGT | 91 |
| 92 | GCCACGTAAC | 92 |
| 93 | GAACTACATG | 93 |
| 94 | TCGTGAGGAC | 94 |
| 95 | GCGGCCTTAA | 95 |
| 96 | GCTAAGGACC | 96 |

TABLE 2-1
Table 2
Random primer list (10-nucleotide B)

| No. | Primer sequence | SEQ ID NO: |
|---|---|---|
| 1 | ATAGCCATTA | 97 |
| 2 | CAGTAATCAT | 98 |
| 3 | ACTCCTTAAT | 99 |
| 4 | TCGAACATTA | 100 |
| 5 | ATTATGAGGT | 101 |
| 6 | AATCTTAGAG | 102 |
| 7 | TTAGATGATG | 103 |
| 8 | TACATATCTG | 104 |
| 9 | TCCTTAATCA | 105 |
| 10 | GTTGAGATTA | 106 |
| 11 | TGTTAACGTA | 107 |
| 12 | CATACAGTAA | 108 |
| 13 | CTTATACGAA | 109 |
| 14 | AGATCTATGT | 110 |
| 15 | AAGACTTAGT | 111 |
| 16 | TGCGCAATAA | 112 |
| 17 | TTGGCCATAT | 113 |
| 18 | TATTACGAGG | 114 |
| 19 | TTATGATCGC | 115 |
| 20 | AACTTAGGAG | 116 |
| 21 | TCACAATCGT | 117 |
| 22 | GAGTATATGG | 118 |
| 23 | ATCAGGACAA | 119 |
| 24 | GTACTGATAG | 120 |
| 25 | CTTATACTCG | 121 |
| 26 | TAACGGACTA | 122 |
| 27 | GCGTTGTATA | 123 |
| 28 | CTTAAGTGCT | 124 |
| 29 | ATACGACTGT | 125 |
| 30 | ACTGTTATCG | 126 |
| 31 | AATCTTGACG | 127 |
| 32 | ACATCACCTT | 128 |
| 33 | GGTATAGTAC | 129 |
| 34 | CTAATCCACA | 130 |
| 35 | GCACCTTATT | 131 |
| 36 | ATTGACGGTA | 132 |
| 37 | GACATATGGT | 133 |
| 38 | GATAGTCGTA | 134 |
| 39 | CAATTATCGC | 135 |
| 40 | CTTAGGTGAT | 136 |
| 41 | CATACTACTG | 137 |
| 42 | TAACGCGAAT | 138 |
| 43 | CAAGTTACGA | 139 |
| 44 | AATCTCAAGG | 140 |
| 45 | GCAATCATCA | 141 |
| 46 | TGTAACGTTC | 142 |
| 47 | TATCGTTGGT | 143 |
| 48 | CGCTTAAGAT | 144 |
| 49 | TTAGAACTGG | 145 |
| 50 | GTCATAACGT | 146 |
| 51 | AGAGCAGTAT | 147 |
| 52 | CAACATCACT | 148 |
| 53 | CAGAAGCTTA | 149 |
| 54 | AACTAACGTG | 150 |
| 55 | TTATACCGCT | 151 |
| 56 | GAATTCGAGA | 152 |
| 57 | TTACGTAACC | 153 |
| 58 | GCATGGTTAA | 154 |
| 59 | GCACCTAATT | 155 |
| 60 | TGTAGGTTGT | 156 |
| 61 | CCATCTGGAA | 157 |
| 62 | TTCGCGTTGA | 158 |
| 63 | AACCGAGGTT | 159 |
| 64 | GTACGCTGTT | 160 |

Table 2-2

| No. | Primer sequence | SEQ ID NO: |
|---|---|---|
| 65 | AGTATCCTGG | 161 |
| 66 | GGTTGTACAG | 162 |
| 67 | ACGTACACCA | 163 |
| 68 | TGTCGAGCAA | 164 |
| 69 | GTCGTGTTAC | 165 |
| 70 | GTGCAATAGG | 166 |
| 71 | ACTCGATGCT | 167 |
| 72 | GAATCGCGTA | 168 |
| 73 | CGGTCATTGT | 169 |
| 74 | ATCAGGCGAT | 170 |
| 75 | GTAAGATGCG | 171 |
| 76 | GGTCTCTTGA | 172 |
| 77 | TCCTCGCTAA | 173 |
| 78 | CTGCGTGATA | 174 |
| 79 | CATACTCGTC | 175 |
| 80 | ATCTGAGCTC | 176 |
| 81 | ACGGATAGTG | 177 |
| 82 | ACTGCAATGC | 178 |
| 83 | TAACGACGTG | 179 |
| 84 | TAGACTGTCG | 180 |
| 85 | CAGCACTTCA | 181 |
| 86 | AACATTCGCC | 182 |
| 87 | ACTAGTGCGT | 183 |
| 88 | ACGCTGTTCT | 184 |
| 89 | CGTCGAATGC | 185 |
| 90 | CTCTGACGGT | 186 |
| 91 | GTCGCCATGT | 187 |
| 92 | GGTCCACGTT | 188 |
| 93 | CGAGCGACTT | 189 |
| 94 | TTGACGCGTG | 190 |
| 95 | CTGAGAGCCT | 191 |
| 96 | CGCGCTAACT | 192 |

TABLE 3-1
Table 3
Random primer list (10-nucleotide C)

| No. | Primer sequence | SEQ ID NO: |
|---|---|---|
| 1 | GGTCGTGAAG | 193 |
| 2 | AGGTTGACCA | 194 |
| 3 | TAACGGCAAC | 195 |
| 4 | GAGGCTGGAT | 196 |
| 5 | GTGCACACCT | 197 |
| 6 | TGAGGACCAG | 198 |
| 7 | TACTTGCGAG | 199 |
| 8 | AACTGTGAGA | 200 |
| 9 | CTCCATCAAC | 201 |
| 10 | CGGACTGTTA | 902 |
| 11 | TAGGACAGTC | 203 |
| 12 | AGAGGACACA | 204 |
| 13 | ACATTCGCGG | 205 |
| 14 | GCTTACTGCA | 206 |
| 15 | CAATACGTAA | 207 |
| 16 | AGACTTGCGC | 208 |
| 17 | GAGCGGTGTT | 209 |
| 18 | CGTGAGAGGT | 210 |
| 19 | AATCCGTCAG | 211 |
| 20 | ATACGTACCG | 212 |
| 21 | AACTGATTCC | 913 |
| 22 | CTGAGCGTAC | 214 |
| 23 | GTCGGATTCG | 215 |
| 24 | GCCGACCATA | 216 |
| 25 | GCAGAACTAA | 217 |
| 26 | CTAACGACCG | 218 |
| 27 | GCTGGACCAT | 219 |
| 28 | GACGCGGTTA | 220 |
| 29 | AGTGGTGAGC | 221 |
| 30 | CAGGCAGTCA | 222 |
| 31 | TCTGACGTCA | 223 |
| 32 | TACATGACGT | 224 |
| 33 | TGAGGCAACC | 225 |
| 34 | CAACTGCAGT | 226 |
| 35 | CGGAGATACG | 227 |
| 36 | CTTCGCAAGT | 228 |
| 37 | CTGGCATACG | 229 |
| 38 | TAACGTTCGC | 230 |
| 39 | CCGGCGTTAA | 231 |
| 40 | ACAAGACGCC | 232 |
| 41 | CCATTAGACT | 233 |
| 42 | GTCTGTGACA | 234 |
| 43 | GGCATTGGAC | 235 |
| 44 | TCTTCGCAGG | 236 |
| 45 | TAGCCTGTGC | 237 |
| 46 | CACTGACCTA | 238 |
| 47 | CCGCACGATT | 239 |
| 48 | ATAGCACACG | 240 |
| 49 | GCACGTCATA | 241 |
| 50 | AAGCCGTTGG | 242 |
| 51 | CGGACCGTTA | 243 |
| 52 | TACACAGCGT | 244 |
| 53 | CGGAGTTCAG | 245 |
| 54 | TAGAACGTCA | 246 |
| 55 | GGCATTGGAG | 247 |
| 56 | GGCACTCGTT | 248 |
| 57 | GTACCGTTAA | 249 |
| 58 | AATACGTGTC | 250 |
| 59 | CCATTGACGT | 251 |
| 60 | CGTGAATCGC | 252 |
| 61 | ATCAACGCGG | 253 |
| 62 | CGCCAAGGTA | 254 |
| 63 | AGAAGACGCC | 255 |
| 64 | CCGCATAGTC | 256 |

Table 3-2

| No. | Primer sequence | SEQ ID NO: |
|---|---|---|
| 65 | CTTATATGTG | 257 |
| 66 | GGTCTCATCG | 258 |
| 67 | CCACCATGTC | 259 |
| 68 | ACGAATGTGT | 260 |
| 69 | GGTAGTAACA | 261 |
| 70 | GCCACTTAAT | 962 |
| 71 | ATATTGCGCC | 263 |
| 72 | GACCAATAGT | 264 |
| 73 | AACAACACGG | 265 |
| 74 | ATAGCCGATG | 266 |
| 75 | CGAGAGCATA | 267 |
| 76 | CGAGACATGA | 268 |
| 77 | CGCCAAGTTA | 269 |
| 78 | TTATAATCGC | 270 |
| 79 | TAGAAGTGCA | 271 |
| 80 | GGAGGCATGT | 272 |
| 81 | GCCACTTCGA | 273 |
| 82 | TCCACGGTAC | 274 |
| 83 | CAACTATGCA | 275 |
| 84 | CAAGGAGGAC | 976 |
| 85 | GAGGTACCTA | 277 |
| 86 | GAGCGCATAA | 278 |
| 87 | TCGTCACGTG | 279 |
| 88 | AACTGTGACA | 280 |
| 89 | TCCACGTGAG | 281 |
| 90 | ACACTGCTCT | 282 |
| 91 | TACGGTGAGC | 283 |
| 92 | CGGACTAAGT | 284 |
| 93 | AAGCCACGTT | 285 |
| 94 | CAATTACTCG | 286 |
| 95 | TCTGGCCATA | 287 |
| 96 | TCAGGCTAGT | 288 |

| Table 4-1 | | |
|---|---|---|
| Table 4 | | |
| Random primer list (10-nucleotide D) | | |
| No. | Primer sequence | SEQ ID NO: |
| 1 | TTGACCCGGA | 289 |
| 2 | TTTTTATGGT | 990 |
| 3 | ATGTGGTGCG | 291 |
| 4 | AAGGCGCTAG | 292 |
| 5 | TCCAACTTTG | 293 |
| 6 | CCATCCCATC | 294 |
| 7 | CAATACGAGG | 295 |
| 8 | GAGTGTTACC | 296 |
| 9 | GCCTCCTGTA | 297 |
| 10 | CGAAGGTTGC | 298 |
| 11 | GAGGTGCTAT | 299 |
| 12 | TAGGATAATT | 300 |
| 13 | CGTTGTCCTC | 301 |
| 14 | TGAGACCAGC | 302 |
| 15 | TGCCCAAGCT | 303 |
| 16 | TACTGAATCG | 304 |
| 17 | TTACATAGTC | 305 |
| 18 | ACAAAGGAAA | 306 |
| 19 | CTCGCTTGGG | 307 |
| 20 | CCTTGCGTCA | 308 |
| 21 | TAATTCCGAA | 309 |
| 22 | GTGAGCTTGA | 310 |
| 23 | ATGCCGATTC | 311 |
| 24 | GCTTGGGCTT | 312 |
| 25 | ACAAAGCGCC | 313 |
| 26 | GAAAGCTCTA | 314 |
| 27 | TACCGACCGT | 315 |
| 28 | TCGAAGAGAC | 316 |
| 29 | GTCGCTTACG | 317 |
| 30 | GGGCTCTCCA | 318 |
| 31 | GCGCCCTTGT | 319 |
| 32 | GGCAATAGGC | 320 |
| 33 | CAAGTCAGGA | 321 |
| 34 | GGGTCGCAAT | 322 |
| 35 | CAGCAACCTA | 323 |
| 36 | TTCCCGCCAC | 324 |
| 37 | TGTGCATTTT | 325 |
| 38 | ATCAACGACG | 326 |
| 39 | GTGACGTCCA | 327 |
| 40 | CGATCTAGTC | 328 |
| 41 | TTACATCCTG | 329 |
| 42 | AGCCTTCAAT | 330 |
| 43 | TCCATCCGAT | 331 |
| 44 | GACTGGGTCT | 332 |
| 45 | TTCGGTGGAG | 333 |
| 46 | GACCAGCACA | 334 |
| 47 | CATTAACGGA | 335 |
| 48 | TTTTTCTTGA | 336 |
| 49 | CATTGCACTG | 337 |
| 50 | TGCGGCGATC | 338 |
| 51 | ATATTGCGGT | 339 |
| 52 | GACGTCGCTC | 340 |
| 53 | TCGCTTATCG | 341 |
| 54 | GCGCAGACAC | 342 |
| 55 | CATGTATTGT | 343 |
| 56 | TCTATAACCT | 344 |
| 57 | GTGGAGACAA | 345 |
| 58 | CGAAGATTAT | 346 |
| 59 | TAGCAACTGC | 347 |
| 60 | ATAATCGGTA | 348 |
| 61 | CAGGATGGGT | 349 |
| 62 | GACGATTCCC | 350 |
| 63 | CACGCCTTAC | 351 |
| 64 | AGTTGGTTCC | 352 |

Table 4-2

| No. | Primer sequence | SEQ ID NO: |
|---|---|---|
| 65 | TCTTATCAGG | 353 |
| 66 | CGAGAAGTTC | 354 |
| 67 | GTGGTAGAAT | 355 |
| 68 | TAGGCTTGTG | 356 |
| 69 | ATGCGTTACG | 357 |
| 70 | ACTACCGAGG | 358 |
| 71 | CGAGTTGGTG | 359 |
| 72 | GGACGATCAA | 360 |
| 73 | AACAGTATGC | 361 |
| 74 | TTGGCTGATC | 362 |
| 75 | AGGATTGGAA | 363 |
| 76 | CATATGGAGA | 364 |
| 77 | CTGCAGGTTT | 365 |
| 78 | CTCTCTTTTT | 366 |
| 79 | AGTAGGGGTC | 367 |
| 80 | ACACCGCAAG | 368 |
| 81 | GAAGCGGGAG | 369 |
| 82 | GATACGGACT | 370 |
| 83 | TACGACGTGT | 371 |
| 84 | GTGCCTCCTT | 372 |
| 85 | GGTGACTGAT | 373 |
| 86 | ATATCTTACG | 374 |
| 87 | AATCATACGG | 375 |
| 88 | GTCTTGGGAC | 376 |
| 89 | GAGGACAAAT | 377 |
| 90 | GTTGCGAGGT | 378 |
| 91 | AAACCGCACC | 379 |
| 92 | GCTAACACGT | 380 |
| 93 | ATCATGAGGG | 381 |
| 94 | GATTCACGTA | 382 |
| 95 | TCTCGAAAAG | 383 |
| 96 | CTCGTAACCA | 384 |

| Table 5-1 | | |
|---|---|---|
| Table 5 | | |
| Random primer list (10-nucleotide E | | |
| No. | Primer sequence | SEQ ID NO: |
| 1 | GTTACACACG | 385 |
| 2 | CGTGAAGGGT | 386 |
| 3 | ACGAGCATCT | 387 |
| 4 | ACGAGGGATT | 388 |
| 5 | GCAACGTCGG | 389 |
| 6 | CACGGCTAGG | 390 |
| 7 | CGTGACTCTC | 391 |
| 8 | TCTAGACGCA | 392 |
| 9 | CTGCGCACAT | 393 |
| 10 | ATGCTTGACA | 394 |
| 11 | TTTGTCGACA | 395 |
| 12 | ACGTGTCAGC | 396 |
| 13 | GAAAACATTA | 397 |
| 14 | ACATTAACGG | 398 |
| 15 | GTACAGGTCC | 399 |
| 16 | CTATGTGTAC | 400 |
| 17 | GCGTACATTA | 401 |
| 18 | GATTTGTGGC | 402 |
| 19 | TCGCGCGCTA | 403 |
| 20 | ACAAGGGCGA | 404 |
| 21 | AACGCGCGAT | 405 |
| 22 | CGTAAATGCG | 406 |
| 23 | TAGGCACTAC | 407 |
| 24 | GCGAGGATCG | 408 |
| 25 | CACGTTTACT | 409 |
| 26 | TACCACCACG | 410 |
| 27 | TTAACAGGAC | 411 |
| 28 | GCTGTATAAC | 412 |
| 29 | GTTGCTGGCA | 413 |
| 30 | AGTGTGGCCA | 414 |
| 31 | CTGCGGTTGT | 415 |
| 32 | TAGATCAGCG | 416 |
| 33 | TTCCGGTTAT | 417 |
| 34 | GATAAACTGT | 418 |
| 35 | TACAGTTGCC | 419 |
| 36 | CGATGGCGAA | 420 |
| 37 | CCGACGTCAG | 421 |
| 38 | TATGGTGCAA | 422 |
| 39 | GACGACAGTC | 423 |
| 40 | GTCACCGTCC | 424 |
| 41 | GGTTTTAACA | 425 |
| 42 | GAGGACAGTA | 426 |
| 43 | GTTACCTAAG | 427 |
| 44 | ATCACGTGTT | 428 |
| 45 | TAAGGCCTGG | 429 |
| 46 | TGTTCGTAGC | 430 |
| 47 | TGAGGACGTG | 431 |
| 48 | GTGCTGTGTA | 432 |
| 49 | GAGGGTACGC | 433 |
| 50 | CCGTGATTGT | 434 |
| 51 | AAAATCGCCT | 435 |
| 52 | CGATCGCAGT | 436 |
| 53 | ACGCAATAAG | 437 |
| 54 | AAGGTGCATC | 438 |
| 55 | CGCGTAGATA | 439 |
| 56 | CGAGCAGTGC | 440 |
| 57 | ATACGTGACG | 441 |
| 58 | AGATTGCGCG | 442 |
| 59 | ACGTGATGCC | 443 |
| 60 | GTACGCATCG | 444 |
| 61 | TCCCGACTTA | 445 |
| 62 | GTTTTTACAC | 446 |
| 63 | CCTGAGCGTG | 447 |
| 64 | CGGCATTGTA | 448 |

Table 5-2

| No. | Primer sequence | SEQ ID NO: |
|---|---|---|
| 65 | TAGAGTGCGT | 449 |
| 66 | ATGGCCAGAC | 450 |
| 67 | CTTAGCATGC | 451 |
| 68 | ACAACACCTG | 452 |
| 69 | AGTGACTATC | 453 |
| 70 | CATGCTACAC | 454 |
| 71 | AAAGCGGGCG | 455 |
| 72 | AGATCGCCGT | 456 |
| 73 | CGTAGATATT | 457 |
| 74 | AATGGCAGAC | 458 |
| 75 | GTATAACGTG | 459 |
| 76 | ATGTGCGTCA | 460 |
| 77 | CCTGCCAACT | 461 |
| 78 | TTTATAACTC | 462 |
| 79 | ACGGTTACGC | 463 |
| 80 | TAGCCTCTTG | 464 |
| 81 | TCGCGAAGTT | 465 |
| 82 | GTCTACAACC | 466 |
| 83 | GTCTACTGCG | 467 |
| 84 | GTTGCGTCTC | 468 |
| 85 | GGGCCGCTAA | 469 |
| 86 | GTACGTCGGA | 470 |
| 87 | AGCGAGAGAC | 471 |
| 88 | TGGCTACGGT | 472 |
| 89 | AGGCATCACG | 473 |
| 90 | TAGCTCCTCG | 474 |
| 91 | GGCTAGTCAG | 475 |
| 92 | CTCACTTTAT | 476 |
| 93 | ACGGCCACGT | 477 |
| 94 | AGCGTATATC | 478 |
| 95 | GACACGTCTA | 479 |
| 96 | GCCAGCGTAC | 480 |

| Table 6-1 | | |
|---|---|---|
| Table 6 | | |
| Random primer list (10-nucleotide F) | | |
| No. | Primer sequence | SEQ ID NO: |
| 1 | AACATTAGCG | 481 |
| 2 | AGTGTGCTAT | 482 |
| 3 | CACGAGCGTT | 483 |
| 4 | GTAACGCCTA | 484 |
| 5 | CACATAGTAC | 485 |
| 6 | CGCGATATCG | 486 |
| 7 | CGTTCTGTGC | 487 |
| 8 | CTGATCGCAT | 488 |
| 9 | TGGCGTGAGA | 489 |
| 10 | TTGCCAGGCT | 490 |
| 11 | GTTATACACA | 491 |
| 12 | AGTGCCAACT | 492 |
| 13 | TCACGTAGCA | 493 |
| 14 | TAATTCAGCG | 494 |
| 15 | AAGTATCGTC | 495 |
| 16 | CACAGTTACT | 496 |
| 17 | CCTTACCGTG | 497 |
| 18 | ACGGTGTCGT | 498 |
| 19 | CGCGTAAGAC | 499 |
| 20 | TTCGCACCAG | 500 |
| 21 | CACGAACAGA | 501 |
| 22 | GTTGGACATT | 502 |
| 23 | GGTGCTTAAG | 503 |
| 24 | TCGGTCTCGT | 504 |
| 25 | TCTAGTACGC | 505 |
| 26 | TTAGGCCGAG | 506 |
| 27 | CGTCAAGAGC | 507 |
| 28 | ACATGTCTAC | 508 |
| 29 | ATCGTTACGT | 509 |
| 30 | ACGGATCGTT | 510 |
| 31 | AATCTTGGCG | 511 |
| 32 | AGTATCTGGT | 512 |
| 33 | CAACCGACGT | 513 |
| 34 | TGGTAACGCG | 514 |
| 35 | GTGCAGACAT | 515 |
| 36 | GTCTAGTTGC | 516 |
| 37 | CAATTCGACG | 517 |
| 38 | CTTAGCACCT | 518 |
| 39 | TAATGTCGCA | 519 |
| 40 | CAATCGGTAC | 520 |
| 41 | AGCACGCATT | 521 |
| 42 | AGGTCCTCGT | 522 |
| 43 | TTGTGCCTGC | 523 |
| 44 | ACCGCCTGTA | 524 |
| 45 | GTACGTCAGG | 525 |
| 46 | GCACACAACT | 526 |
| 47 | TGAGCACTTA | 527 |
| 48 | GTGCCGCATA | 528 |
| 49 | ATGTTTTCGC | 599 |
| 50 | ACACTTAGGT | 530 |
| 51 | CGTGCCGTGA | 531 |
| 52 | TTACTAATCA | 532 |
| 53 | GTGGCAGGTA | 533 |
| 54 | GCGCGATATG | 534 |
| 55 | GAACGACGTT | 535 |
| 56 | ATCAGGAGTG | 536 |
| 57 | GuCAGTAAGT | 537 |
| 58 | GCAAGAAGCA | 538 |
| 59 | AACTCCGCCA | 539 |
| 60 | ACTTGAGCCT | 540 |
| 61 | CGTGATCGTG | 541 |
| 62 | AATTAGCGAA | 542 |
| 63 | ACTTCCTTAG | 543 |
| 64 | TGTGCTGATA | 544 |

Table 6-2

| No. | Primer sequence | SEQ ID NO: |
|---|---|---|
| 65 | AGGCGGCTGA | 545 |
| 66 | CCTTTAGAGC | 546 |
| 67 | ACGCGTCTAA | 547 |
| 68 | GCGAATGTAC | 548 |
| 69 | CGTGATCCAA | 549 |
| 70 | CAACCAGATG | 550 |
| 71 | ACCATTAACC | 551 |
| 72 | CGATTCACGT | 552 |
| 73 | CTAGAACCTG | 553 |
| 74 | CCTAACGACA | 554 |
| 75 | GACGTGCATG | 555 |
| 76 | ATGTAACCTT | 556 |
| 77 | GATACAGTCG | 557 |
| 78 | CGTATGTCTC | 558 |
| 79 | AGATTATCGA | 559 |
| 80 | ATACTGGTAA | 560 |
| 81 | GTTGAGTAGC | 561 |
| 82 | ACCATTATCA | 562 |
| 83 | CACACTTCAG | 563 |
| 84 | GACTAGCGGT | 564 |
| 85 | AATTGTCGAG | 565 |
| 86 | CTAAGGACGT | 566 |
| 87 | ATTACGATGA | 567 |
| 88 | ATTGAAGACT | 568 |
| 89 | GCTTGTACGT | 569 |
| 90 | CCTACGTCAC | 570 |
| 91 | CACAACTTAG | 571 |
| 92 | GCGGTTCATC | 572 |
| 93 | GTACTCATCT | 573 |
| 94 | GTGCATCAGT | 574 |
| 95 | TCACATCCTA | 575 |
| 96 | CACGCGCTAT | 576 |

| Table 7-1 | | |
|---|---|---|
| Table 7 | | |
| Random primer list (8-nucleotide) | | |
| No. | Primer sequence | SEQ ID NO: |
| 1 | CTATCTTG | 577 |
| 2 | AAGTGCGT | 578 |
| 3 | ACATGCGA | 579 |
| 4 | ACCAATGG | 580 |
| 5 | TGCGTTGA | 581 |
| 6 | GACATGTC | 582 |
| 7 | TTGTGCGT | 583 |
| 8 | ACATCGCA | 584 |
| 9 | GAAGACGA | 585 |
| 10 | TCGATAGA | 586 |
| 11 | TCTTGCAA | 587 |
| 12 | AGCAAGTT | 588 |
| 13 | TTCATGGA | 589 |
| 14 | TCAATTCG | 590 |
| 15 | CGGTATGT | 591 |
| 16 | ACCACTAC | 592 |
| 17 | TCGCTTAT | 593 |
| 18 | TCTCGACT | 594 |
| 19 | GAATCGGT | 595 |
| 20 | GTTACAAG | 596 |
| 21 | CTGTGTAG | 597 |
| 22 | TGGTAGAA | 598 |
| 23 | ATACTGCG | 599 |
| 24 | AACTCGTC | 600 |
| 25 | ATATGTGC | 601 |
| 26 | AAGTTGCG | 602 |
| 27 | GATCATGT | 603 |
| 28 | TTGTTGCT | 604 |
| 29 | CCTCTTAG | 605 |
| 30 | TCACAGCT | 606 |
| 31 | AGATTGAC | 607 |
| 32 | AGCCTGAT | 608 |
| 33 | CGTCAAGT | 609 |
| 34 | AAGTAGAC | 610 |
| 35 | TCAGACAA | 611 |
| 36 | TCCTTGAC | 612 |
| 37 | GTAGCTGT | 613 |
| 38 | CGTCGTAA | 614 |
| 39 | CCAATGGA | 615 |
| 40 | TTGAGAGA | 616 |
| 41 | ACAACACC | 617 |
| 42 | TCTAGTAC | 618 |
| 43 | GAGGAAGT | 619 |
| 44 | GCGTATTG | 620 |
| 45 | AAGTAGCT | 621 |
| 46 | TGAACCTT | 629 |
| 47 | TGTGTTAC | 623 |
| 48 | TAACCTGA | 624 |
| 49 | GCTATTCC | 695 |
| 50 | GTTAGATG | 626 |
| 51 | CAGGATAA | 627 |
| 52 | ACCGTAGT | 628 |
| 53 | CCGTGTAT | 629 |
| 54 | TCCACTCT | 630 |
| 55 | TAGCTCAT | 631 |
| 56 | CGCTAATA | 632 |
| 57 | TACCTCTG | 633 |
| 58 | TGCACTAC | 634 |
| 59 | CTTGGAAG | 635 |
| 60 | AATGCACG | 636 |
| 61 | CACTGTTA | 637 |
| 62 | TCGACTAG | 638 |
| 63 | CTAGGTTA | 639 |
| 64 | GCAGATGT | 640 |

Table 7-2

| No. | Primer sequence | SEQ ID NO: |
|---|---|---|
| 65 | AGTTCAGA | 641 |
| 66 | CTCCATCA | 642 |
| 67 | TGGTTACG | 643 |
| 68 | ACGTAGCA | 644 |
| 69 | CTCTTCCA | 645 |
| 70 | CGTCAGAT | 646 |
| 71 | TGGATCAT | 647 |
| 72 | ATATCGAC | 648 |
| 73 | TTGTGGAG | 649 |
| 74 | TTAGAGCA | 650 |
| 75 | TAACTACC | 651 |
| 76 | CTATGAGG | 652 |
| 77 | CTTCTCAC | 653 |
| 78 | CGTTCTCT | 654 |
| 79 | GTCACTAT | 655 |
| 80 | TCGTTAGC | 656 |
| 81 | ATCGTGTA | 657 |
| 82 | GAGAGCAA | 658 |
| 83 | AGACGCAA | 659 |
| 84 | TCCAGTTA | 660 |
| 85 | AATGCCAC | 661 |
| 86 | ATCACGTG | 662 |
| 87 | ACTGTGCA | 663 |
| 88 | TCACTGCA | 664 |
| 89 | GCATCCAA | 665 |
| 90 | AGCACTAT | 666 |
| 91 | CGAAGGAT | 667 |
| 92 | CCTTGTGT | 668 |
| 93 | TGCGGATA | 669 |
| 94 | AGGAATGG | 670 |
| 95 | ATCGTAAC | 671 |
| 96 | GAATGTCT | 672 |

| Table 8-1 | | |
|---|---|---|
| Table 8 | | |
| Random primer list (9-nucleotide) | | |
| No. | Primer sequence | SEQ ID NO: |
| 1 | TTGCTACAT | 673 |
| 2 | TAACGTATG | 674 |
| 3 | CAGTATGTA | 675 |
| 4 | TCAATAACG | 676 |
| 5 | CACACTTAT | 677 |
| 6 | GACTGTAAT | 678 |
| 7 | TATACACTG | 679 |
| 8 | ACTGCATTA | 680 |
| 9 | ACATTAAGC | 681 |
| 10 | CATATTACG | 682 |
| 11 | ATATCTACG | 683 |
| 12 | AGTAACTGT | 684 |
| 13 | ATGACGTTA | 685 |
| 14 | ATTATGCGA | 686 |
| 15 | AGTATACAC | 687 |
| 16 | TTAGCGTTA | 688 |
| 17 | TATGACACT | 689 |
| 18 | ATTAACGCT | 690 |
| 19 | TAGGACAAT | 691 |
| 20 | AAGACGTTA | 692 |
| 21 | TATAAGCGT | 693 |
| 22 | ATACCTGGC | 694 |
| 23 | CTCGAGATC | 695 |
| 24 | ATGGTGAGG | 696 |
| 25 | ATGTCGACG | 697 |
| 26 | GACGTCTGA | 698 |
| 27 | TACACTGCG | 699 |
| 28 | ATCGTCAGG | 700 |
| 29 | TGCACGTAC | 701 |
| 30 | GTCGTGCAT | 702 |
| 31 | GAGTGTTAC | 703 |
| 32 | AGACTGTAC | 704 |
| 33 | TGCGACTTA | 705 |
| 34 | TGTCCGTAA | 706 |
| 35 | GTAATCGAG | 707 |
| 36 | GTACCTTAG | 708 |
| 37 | ATCACGTGT | 709 |
| 38 | ACTTAGCGT | 710 |
| 39 | GTAATCGTG | 711 |
| 40 | ATGCCGTTA | 712 |
| 41 | ATAACGTGC | 713 |
| 42 | CTACGTTGT | 714 |
| 43 | TATGACGCA | 715 |
| 44 | CCGATAACA | 716 |
| 45 | ATGCGCATA | 717 |
| 46 | GATAAGCGT | 718 |
| 47 | ATATCTGCG | 719 |
| 48 | ACTTAGACG | 720 |
| 49 | ATCACCGTA | 721 |
| 50 | TAAGACACG | 722 |
| 51 | AATGCCGTA | 723 |
| 52 | AATCACGTG | 724 |
| 53 | TCGTTAGTC | 725 |
| 54 | CATCATGTC | 726 |
| 55 | TAAGACGGT | 727 |
| 56 | TGCATAGTG | 728 |
| 57 | GAGCGTTAT | 799 |
| 58 | TGCCTTACA | 730 |
| 59 | TTCGCGTTA | 731 |
| 60 | GTGTTAACG | 732 |
| 61 | GACACTGAA | 733 |
| 62 | CTGTTATCG | 734 |
| 63 | GGTCGTTAT | 735 |
| 64 | CGAGAGTAT | 736 |

Table 8-2

| No. | Primer sequence | SEQ ID NO: |
|---|---|---|
| 65 | ATACAGTCC | 737 |
| 66 | AATTCACGC | 738 |
| 67 | TATGTGCAC | 739 |
| 68 | GATGACGTA | 740 |
| 69 | GATGCGATA | 741 |
| 70 | GAGCGATTA | 742 |
| 71 | TGTCACAGA | 743 |
| 72 | TACTAACCG | 744 |
| 73 | CATAACGAG | 745 |
| 74 | CGTATACCT | 748 |
| 75 | TATCACGTG | 747 |
| 76 | GAACGTTAC | 748 |
| 77 | GTcGTATAC | 749 |
| 78 | ATGTCGACA | 750 |
| 79 | ATACAGCAC | 751 |
| 80 | TACTTACGC | 752 |
| 81 | AACTACGGT | 753 |
| 82 | TAGAACGGT | 754 |
| 83 | GAATGTCAC | 755 |
| 84 | TGTACGTCT | 756 |
| 85 | AACATTGCG | 757 |
| 86 | TTGAACGCT | 758 |
| 87 | AATCAGGAC | 759 |
| 88 | ATTCGCACA | 760 |
| 89 | CCATGTACT | 761 |
| 90 | TGTCCTGTT | 762 |
| 91 | TAATTGCGC | 763 |
| 92 | GATAGTGTG | 764 |
| 93 | ATAGACGCA | 765 |
| 94 | TGTACCGTT | 766 |
| 95 | ATTGTCGCA | 767 |
| 96 | GTCACGTAA | 768 |

TABLE 9
Random primer list (11-nucleotide)

| No. | Primer sequence | SEQ ID NO: |
|---|---|---|
| 1 | TTACACTATGC | 769 |
| 2 | GCGATAGTCGT | 770 |
| 3 | CTATTCACAGT | 771 |
| 4 | AGAGTCACTGT | 772 |
| 5 | AGAGTCGAAGC | 773 |
| 6 | CTGAATATGTG | 774 |
| 7 | ACTCCACAGGA | 775 |
| 8 | ATCCTCGTAAG | 776 |
| 9 | TACCATCGCCT | 777 |
| 10 | AACGCCTATAA | 778 |
| 11 | CTGTCGAACTT | 779 |
| 12 | TCAGATGTCCG | 780 |
| 13 | CTGCTTATCGT | 781 |
| 14 | ACATTCGCACA | 782 |
| 15 | CCTTAATGCAT | 783 |
| 16 | GGCTAGCTACT | 784 |
| 17 | TTCCAGTTGGC | 785 |
| 18 | GAGTCACAAGG | 786 |
| 19 | CAGAAGGTTCA | 787 |
| 20 | TCAACGTGCAG | 788 |
| 21 | CAAGCTTACTA | 789 |
| 22 | AGAACTCGTTG | 790 |
| 23 | CCGATACAGAG | 791 |
| 24 | GTACGCTGATC | 792 |
| 25 | TCCTCAGTGAA | 793 |
| 26 | GAGCCAACATT | 794 |
| 27 | GAGATCGATGG | 795 |
| 28 | ATCGTCAGCTG | 796 |
| 29 | GAAGCACACGT | 797 |
| 30 | ATCACGCAACC | 798 |
| 31 | TCGAATAGTCG | 799 |
| 32 | TATTACCGTCT | 800 |
| 33 | CAGTCACGACA | 801 |
| 34 | TTACTCGACGT | 802 |
| 35 | GCAATGTTGAA | 803 |
| 36 | GACACGAGCAA | 804 |
| 37 | CGAGATTACAA | 805 |
| 38 | TACCGACTACA | 806 |
| 39 | ACCGTTGCCAT | 807 |
| 40 | ATGTAATCGCC | 808 |
| 41 | AAGCCTGATGT | 809 |
| 42 | AAGTAACGTGG | 810 |
| 43 | GTAGAGGTTGG | 811 |
| 44 | CTCTTGCCTCA | 812 |
| 45 | ATCGTGAAGTG | 813 |
| 46 | ACCAGCACTAT | 814 |
| 47 | CACCAGAATGT | 815 |
| 48 | GAGTGAACAAC | 816 |
| 49 | TAACGTTACGC | 817 |
| 50 | CTTGGATCTTG | 818 |
| 51 | GTTCCAACGTT | 819 |
| 52 | CAAGGACCGTA | 820 |
| 53 | GACTTCACGCA | 821 |
| 54 | CACACTACTGG | 822 |
| 55 | TCAGATGAATC | 823 |
| 56 | TATGGATCTGG | 824 |
| 57 | TCTTAGGTGTG | 825 |
| 58 | TGTCAGCGTCA | 826 |
| 59 | GTCTAGGACAG | 827 |
| 60 | GCCTCTTCATA | 828 |
| 61 | AGAAGTGTTAC | 829 |
| 62 | CATGAGGCTTG | 830 |
| 63 | TGGATTGCTCA | 831 |
| 64 | ATCTACCTAAG | 832 |
| 65 | ATGAGCAGTGA | 833 |
| 66 | CCAGGAGATAC | 834 |
| 67 | CCGTTATACTT | 835 |
| 68 | CTCAGTACAAG | 836 |
| 69 | GGTGATCGTAG | 837 |
| 70 | CGAACGAGACA | 838 |
| 71 | ACTACGAGCTT | 839 |
| 72 | TTGCCACAGCA | 840 |
| 73 | GTCAACTCTAC | 841 |
| 74 | TGGACTGTGTC | 842 |
| 75 | GGAATGGACTT | 843 |
| 76 | CGAGAACATAA | 844 |
| 77 | ACCTGGTCAGT | 845 |
| 78 | CGAACGACACA | 846 |
| 79 | AGTCTAGCCAT | 847 |
| 80 | AGGCCTAGATG | 848 |
| 81 | GGTGCGTTAGT | 849 |
| 82 | ATTGTGTCCGA | 850 |
| 83 | GCAGACATTAA | 851 |
| 84 | ATTGGCTCATG | 852 |
| 85 | GAGGTTACATG | 853 |
| 86 | CCTATAGGACC | 854 |
| 87 | TTAGACGGTCT | 855 |
| 88 | GATTGACGCAC | 856 |
| 89 | AAGACACCTCG | 857 |
| 90 | TCGAATAATCG | 858 |
| 91 | TCTATGTCGGA | 859 |
| 92 | TCGCATGAACC | 860 |
| 93 | TGTTATGTCTC | 861 |
| 94 | TGGATCCTACA | 862 |
| 95 | ATCGTTCAGCC | 863 |
| 96 | TACCGCAAGCA | 864 |

TABLE 10
Random primer list (12-nucleotide)

| No. | Primer sequence | SEQ ID NO: |
|---|---|---|
| 1 | GCTGTTGAACCG | 865 |
| 2 | ATACTCCGAGAT | 866 |
| 3 | CTTAAGGAGCGC | 867 |
| 4 | TATACTACAAGC | 868 |
| 5 | TAGTGGTCGTCA | 869 |
| 6 | GTGCTTCAGGAG | 870 |
| 7 | GACGCATACCTC | 871 |
| 8 | CCTACCTGTGGA | 872 |
| 9 | GCGGTCACATAT | 873 |
| 10 | CTGCATTCACGA | 874 |
| 11 | TGGATCCTTCAT | 875 |
| 12 | TTGTGCTGGACT | 876 |
| 13 | ATTGAGAGCTAT | 877 |
| 14 | TCGCTAATGTAG | 878 |
| 15 | CTACTGGCACAA | 879 |
| 16 | AGAGCCAGTCGT | 880 |
| 17 | AATACTGGCTAA | 881 |
| 18 | CTGCATGCATAA | 882 |
| 19 | TTGTCACAACTC | 883 |
| 20 | TGCTAACTCTCC | 884 |
| 21 | TCTCTAGTTCGG | 885 |
| 22 | TTACGTCCGCAA | 886 |
| 23 | GTGTTGCTACCA | 887 |
| 24 | CGCATGTATGCC | 888 |
| 25 | CCTGTTCTGATT | 889 |
| 26 | TAAGATGCTTGA | 890 |
| 27 | ATATATCTCAGC | 891 |
| 28 | TTCCTCGTGGTT | 892 |
| 29 | ATGTCGATCTAG | 893 |
| 30 | CATCCACTAATC | 894 |
| 31 | GCCTCTGGTAAC | 895 |
| 32 | AGTCAAGAGATT | 896 |
| 33 | ACTGAGGCGTTC | 897 |
| 34 | TAAGGCTGACAT | 898 |
| 35 | AGTTCGCATACA | 899 |
| 36 | GCAGAATTGCGA | 900 |
| 37 | GGTTATGAAGAA | 901 |
| 38 | AGAAGTCGCCTC | 902 |
| 39 | TTCGCGTTATTG | 903 |
| 40 | TACCTGGTCGGT | 904 |
| 41 | GGTTACCGAGGA | 905 |
| 42 | ACACACTTCTAG | 906 |
| 43 | GGAAGTGATTAA | 907 |
| 44 | TCCATCAGATAA | 908 |
| 45 | TGTCTGTATCAT | 909 |
| 46 | AATTGGCTATAG | 910 |
| 47 | ACGTCGGAAGGT | 911 |
| 48 | AGGCATCCGTTG | 912 |
| 49 | ACCGTCGCTTGA | 913 |
| 50 | TACCGTCAAGTG | 914 |
| 51 | CTCGATATAGTT | 915 |
| 52 | CGTCAACGTGGT | 916 |
| 53 | TAGTCAACGTAG | 917 |
| 54 | TGAGTAGGTCAG | 918 |
| 55 | CTTGGCATGTAC | 919 |
| 56 | TGCCGAGACTTC | 920 |
| 57 | CTAAGACTTAAG | 921 |
| 58 | TTCTCGTGTGCG | 922 |
| 59 | CACCTGCACGAT | 923 |
| 60 | ATTAAGCCTAAG | 924 |
| 61 | GGTGGAACCATG | 925 |
| 62 | ACTAACGCGACT | 926 |
| 63 | CAGTTGTGCTAT | 927 |
| 64 | ACGCTGTTAGCA | 928 |
| 65 | GTCAACGCTAAG | 929 |
| 66 | AGCTTAGGTATG | 930 |
| 67 | CGCAGGACGATT | 931 |
| 68 | AACCGGCTGTCT | 932 |
| 69 | GTTGCTCACGTG | 933 |
| 70 | GAATCTTCCGCG | 934 |
| 71 | AGAGCGTACACG | 935 |
| 72 | AAGGCTAATGTC | 936 |
| 73 | TCTATGTAGACG | 937 |
| 74 | AGACGGTCTAGT | 938 |
| 75 | TTGGTCACACGC | 939 |
| 76 | GTCGATATATGG | 940 |
| 77 | AACATGGATACG | 941 |
| 78 | TTCGCAGTTCCT | 942 |
| 79 | CGCATGTTGTGC | 943 |
| 80 | TGTTAAGTTGGA | 944 |
| 81 | CAAGTGTGATGA | 945 |
| 82 | CTGGTACCACGT | 946 |
| 83 | CGCTAGGATCAC | 947 |
| 84 | TGCTCATTACGG | 948 |
| 85 | TGCTCAGTAACA | 949 |
| 86 | ACGATCATAGCC | 950 |
| 87 | ACGATACGTGGA | 951 |
| 88 | GTTCGATGATGG | 952 |
| 89 | AAGAGCTGTGCC | 953 |
| 90 | GGTTGGATCAAC | 954 |
| 91 | GCGCGCTTATGA | 955 |
| 92 | CGTCGATCATCA | 956 |
| 93 | GAGACTGCACTC | 957 |
| 94 | GATAGATCGCAT | 958 |
| 95 | GGCCATCATCAG | 959 |
| 96 | GGTGTTCCACTG | 960 |

TABLE 11
Random primer list (14-nucleotide)

| No. | Primer sequence | SEQ ID NO: |
|---|---|---|
| 1 | AGCTATACAGAGGT | 961 |
| 2 | AGGCCGTTCTGTCT | 962 |
| 3 | CATTGGTCTGCTAT | 963 |
| 4 | CTACATACGCGCCA | 964 |
| 5 | GCTTAACGGCGCTT | 965 |
| 6 | TACGATACTCCACC | 966 |
| 7 | ACCGGCATAAGAAG | 967 |
| 8 | GGATGCTTCGATAA | 968 |
| 9 | GTGTACCTGAATGT | 969 |
| 10 | CGCGGATACACAGA | 970 |
| 11 | TTCCACGGCACTGT | 971 |
| 12 | TAGCCAGGCAACAA | 972 |
| 13 | AGCGTCAACACGTA | 973 |
| 14 | TAACGCTACTCGCG | 974 |
| 15 | TAGATAGACGATCT | 975 |
| 16 | ACTCTTGCAATGCT | 976 |
| 17 | ACTCGGTTAGGTCG | 977 |
| 18 | CATTATCTACGCAT | 978 |
| 19 | CACACCGGCGATTA | 979 |
| 20 | TACGCAGTACTGTG | 980 |
| 21 | CAAGCGCGTGAATG | 981 |
| 22 | GAATGGACTGACGA | 982 |
| 23 | CTAGCGCTGAAGTT | 983 |
| 24 | TGCGGCAGACCAAT | 984 |
| 25 | AAGGCATAGAGATT | 985 |
| 26 | TTCTCCTCGCCATG | 986 |
| 27 | TCATTGGTCGTGAA | 987 |
| 28 | ATTACGCTATACGA | 988 |
| 29 | ATGATCCTCCACGG | 989 |
| 30 | CGTCGTTAGTAATC | 990 |
| 31 | TGCACATAGTCTCA | 991 |
| 32 | GTCAAGGAGTCACG | 992 |
| 33 | GGTTGGAATCTTGC | 993 |
| 34 | CATCGGTGCACTCA | 994 |
| 35 | AATGCACTAGACGT | 995 |
| 36 | TACAGTCAGGCTCG | 996 |
| 37 | AGAGAAGCTTAGCC | 997 |
| 38 | CCATAGGATCGTAT | 998 |
| 39 | TTGTGCTACACCTG | 999 |
| 40 | CTCCAGTAATACTA | 1000 |
| 41 | TGATGCCGATGTGG | 1001 |
| 42 | GTCATACCGCTTAA | 1002 |
| 43 | ACGTTCTCTTGAGA | 1003 |
| 44 | CAGCCATATCGTGT | 1004 |
| 45 | TTGAACGTAGCAAT | 1005 |
| 46 | ACAATCGCGGTAAT | 1006 |
| 47 | GTTCCTGTAGATCC | 1007 |
| 48 | AGAGCCTTACGGCA | 1008 |
| 49 | AATATGGCGCCACC | 1009 |
| 50 | ACCATATAGGTTCG | 1010 |
| 51 | ATGCACCACAGCTG | 1011 |
| 52 | CTACTATTGAACAG | 1012 |
| 53 | TGCCATCACTCTAG | 1013 |
| 54 | GCGAACGAGAATCG | 1014 |
| 55 | GAATCAAGGAGACC | 1015 |
| 56 | CAACATCTATGCAG | 1016 |
| 57 | CAATCCGTCATGGA | 1017 |
| 58 | AGCTCTTAGCCATA | 1018 |
| 59 | AACAAGGCAACTGG | 1019 |
| 60 | GTCGTCGCTCCTAT | 1020 |
| 61 | GTCATCATTAGATG | 1021 |
| 62 | GCACTAAGTAGCAG | 1022 |
| 63 | ACCTTACCGGACCT | 1023 |
| 64 | GCTCAGGTATGTCA | 1024 |
| 65 | TGTCACGAGTTAGT | 1025 |
| 66 | CAGATGACTTACGT | 1026 |
| 67 | GAAGTAGCGATTGA | 1027 |
| 68 | GCAGGCAATCTGTA | 1028 |
| 69 | CCTTATACAACAAG | 1029 |
| 70 | CCTTAGATTGATTG | 1030 |
| 71 | AGCCACGAGTGATA | 1031 |
| 72 | GGATGACTCGTGAC | 1032 |
| 73 | CTTCGTTCGCCATT | 1033 |
| 74 | TCTTGCGTATTGAT | 1034 |
| 75 | CTTAACGTGGTGGC | 1035 |
| 76 | TGCTGTTACGGAAG | 1036 |
| 77 | CTGAATTAGTTCTC | 1037 |
| 78 | CCTCCAAGTACAGA | 1038 |
| 79 | CTGGTAATTCGCGG | 1039 |
| 80 | CGACTGCAATCTGG | 1040 |
| 81 | TGGATCGCGATTGG | 1041 |
| 82 | CGACTATTCCTGCG | 1042 |
| 83 | CAAGTAGGTCCGTC | 1043 |
| 84 | AGTAATCAGTGTTC | 1044 |
| 85 | TTATTCTCACTACG | 1045 |
| 86 | CATGTCTTCTTCGT | 1046 |
| 87 | AGGCACATACCATC | 1047 |
| 88 | AGGTTAGAGGATGT | 1048 |
| 89 | CAACTGGCAAGTGC | 1049 |
| 90 | CGCTCACATAGAGG | 1050 |
| 91 | GCAATGTCGAGATC | 1051 |
| 92 | GTTCTGTGGTGCTC | 1052 |
| 93 | AAGTGATCAGACTA | 1053 |
| 94 | ATTGAAGGATTCCA | 1054 |
| 95 | ACGCCATGCTACTA | 1055 |
| 96 | CTGAAGATGTCTGC | 1056 |

TABLE 12
Random primer list (16-nucleotide)

| No. | Primer sequence | SEQ ID NO: |
|---|---|---|
| 1 | GACAATCTCTGCCGAT | 1057 |
| 2 | GGTCCGCCTAATGTAA | 1058 |
| 3 | AGCCACAGGCAATTCC | 1059 |
| 4 | ATCTCAAGTTCTCAAC | 1060 |
| 5 | TGTAACGCATACGACG | 1061 |
| 6 | TATCTCGAATACCAGC | 1062 |
| 7 | ACCGCAACACAGGCAA | 1063 |
| 8 | GGCCAGTAACATGACT | 1064 |
| 9 | GTGAACAGTTAAGGTG | 1065 |
| 10 | CCAGGATCCGTATTGC | 1066 |
| 11 | GACCTAGCACTAGACC | 1067 |
| 12 | CGCCATCCTATTCACG | 1068 |
| 13 | AAGTGCAGTAATGGAA | 1069 |
| 14 | TCAACGCGTTCGTCTA | 1070 |
| 15 | AGCGGCCACTATCTAA | 1071 |
| 16 | CTCGGCGCCATATAGA | 1072 |
| 17 | CGATAACTTAGAAGAA | 1073 |
| 18 | CATAGGATGTGACGCC | 1074 |
| 19 | GGCTTGTCGTCGTATC | 1075 |
| 20 | CTTGTCTGAATATTAG | 1076 |
| 21 | ACAGTTCGAGTGTCGG | 1077 |
| 22 | CTCTAACCTGTGACGT | 1078 |
| 23 | CGCGCTAATTCAACAA | 1079 |
| 24 | ACTCACGAATGCGGCA | 1080 |
| 25 | AATCTTCGGCATTCAT | 1081 |
| 26 | AAGTATCAGGATCGCG | 1082 |
| 27 | AGTAACTCTGCAGACA | 1083 |
| 28 | GGATTGAACATTGTGC | 1084 |
| 29 | GTGATGCTCACGCATC | 1085 |
| 30 | CGTAGCGTAACGGATA | 1086 |
| 31 | TGCGATGCACCGTTAG | 1087 |
| 32 | CCAGTATGCTCTCAGG | 1088 |
| 33 | AATGACGTTGAAGCCT | 1089 |
| 34 | TCGATTCTATAGGAGT | 1090 |
| 35 | CGATAGGTTCAGCTAT | 1091 |
| 36 | CCATGTTGATAGAATA | 1092 |
| 37 | GAGCCACTTCTACAGG | 1093 |
| 38 | GCGAACTCTCGGTAAT | 1094 |
| 39 | GACCTGAGTAGCTGGT | 1095 |
| 40 | CGAGTCTATTAGCCTG | 1096 |
| 41 | GTAGTGCCATACACCT | 1097 |
| 42 | CCAGTGGTCTATAGCA | 1098 |
| 43 | GTCAGTGCGTTATTGC | 1099 |
| 44 | AGTGTCGGAGTGACGA | 1100 |
| 45 | AATCTCCGCTATAGTT | 1101 |
| 46 | CGAGTAGGTCTGACTT | 1102 |
| 47 | CTGTCGCTCTAATAAC | 1103 |
| 48 | GCTGTCAATATAACTG | 1104 |
| 49 | AGCTCAAGTTGAATCC | 1105 |
| 50 | AATTCATGCTCCTAAC | 1106 |
| 51 | CCAAGGTCTGGTGATA | 1107 |
| 52 | CTCCACGTATCTTGAA | 1108 |
| 53 | TAGCCGAACAACACTT | 1109 |
| 54 | AGTACACGACATATGC | 1110 |
| 55 | ACGTTCTAGACTCCTG | 1111 |
| 56 | CGACTCAAGCACTGCT | 1112 |
| 57 | TGAAGCTCACGATTAA | 1113 |
| 58 | TATCTAACGTATGGTA | 1114 |
| 59 | TATACCATGTTCCTTG | 1115 |
| 60 | TTCCTACGATGACTTC | 1116 |
| 61 | CTCTCCAATATGTGCC | 1117 |
| 62 | GAGTAGAGTCTTGCCA | 1113 |
| 63 | GCGAGATGTGGTCCTA | 1119 |
| 64 | AAGCTACACGGACCAC | 1120 |
| 65 | ATACAACTGGCAACCG | 1121 |
| 66 | CGGTAGATGCTATGCT | 1122 |
| 67 | TCTTGACCGGTCATCA | 1123 |
| 68 | AGATCGTGCATGCGAT | 1124 |
| 69 | TCCTCGAGACAGCCTT | 1125 |
| 70 | TAGCCGGTACCACTTA | 1126 |
| 71 | GTAAGGCAGCGTGCAA | 1127 |
| 72 | TAGTCTGCTCCTGGTC | 1128 |
| 73 | TGGATTATAGCAGCAG | 1129 |
| 74 | AAGAATGATCAGACAT | 1130 |
| 75 | CAGCGCTATATACCTC | 1131 |
| 76 | GAGTAGTACCTCCACC | 1132 |
| 77 | GACGTGATCCTCTAGA | 1133 |
| 78 | GTTCCGTTCACTACGA | 1134 |
| 79 | TGCAAGCACCAGGATG | 1135 |
| 80 | TTAGTTGGCGGCTGAG | 1136 |
| 81 | CAGATGCAGACATACG | 1137 |
| 82 | GACGCTTGATGATTAT | 1138 |
| 83 | TGGATCACGACTAGGA | 1139 |
| 84 | CTCGTCGGTATAACGC | 1140 |
| 85 | AAGCACGGATGCGATT | 1141 |
| 86 | AGATCTTCCGGTGAAC | 1142 |
| 87 | GGACAATAGCAACCTG | 1143 |
| 88 | GATAATCGGTTCCAAT | 1144 |
| 89 | CTCAAGCTACAGTTGT | 1145 |
| 90 | GTTGGCATGATGTAGA | 1146 |
| 91 | CAGCATGAGGTAAGTG | 1147 |
| 92 | GCCTCATCACACGTCA | 1148 |
| 93 | TCGATACTACACATCG | 1149 |
| 94 | TACACGAGGCTTGATC | 1150 |
| 95 | TTCTCGTGTCCGCATT | 1151 |
| 96 | GGTGAAGCAACAGCAT | 1152 |

TABLE 13
Random primer list (18-nucleotide)

| No. | Primer sequence | SEQ ID NO: |
|---|---|---|
| 1 | CGAACCGACTGTACAGTT | 1153 |
| 2 | CCGACTGCGGATAAGTTA | 1154 |
| 3 | CGACAGGTAGGTAAGCAG | 1155 |
| 4 | TGATACGTTGGTATACAG | 1156 |
| 5 | CTACTATAGAATACGTAG | 1157 |
| 6 | AGACTGTGGCAATGGCAT | 1158 |
| 7 | GGAAGACTGATACAACGA | 1159 |
| 8 | TATGCACATATAGCGCTT | 1160 |
| 9 | CATGGTAATCGACCGAGG | 1161 |
| 10 | GTCATTGCCGTCATTGCC | 1162 |
| 11 | CCTAAGAACTCCGAAGCT | 1163 |
| 12 | TCGCTCACCGTACTAGGA | 1164 |
| 13 | TATTACTGTCACAGCAGG | 1165 |
| 14 | TGAGACAGGCTACGAGTC | 1166 |
| 15 | AAGCTATGCGAACACGTT | 1167 |
| 16 | AACGGAGGAGTGAGCCAA | 1168 |
| 17 | CCACTATGGACATCATGG | 1169 |
| 18 | ATGGTGGTGGATAGCTCG | 1170 |
| 19 | TCACCGGTTACACATCGC | 1171 |
| 20 | AAGATACTGAGATATGGA | 1172 |
| 21 | GACCTGTTCTTGAACTAG | 1173 |
| 22 | AAGTAGAGCTCTCGGTTA | 1174 |
| 23 | CTATGTTCTTACTCTCTT | 1175 |
| 24 | CAAGGCTATAAGCGGTTA | 1176 |
| 25 | GAAGCTAATTAACCGATA | 1177 |
| 26 | TTCACGTCTGCCAAGCAC | 1178 |
| 27 | ATCGTATAGATCGAGACA | 1179 |
| 28 | GTCACAGATTCACATCAT | 1180 |
| 29 | GTGCCTGTGAACTATCAG | 1181 |
| 30 | CAGCGTACAAGATAGTCG | 1182 |
| 31 | GCATGGCATGGTAGACCT | 1183 |
| 32 | GGTATGCTACTCTTCGCA | 1184 |
| 33 | ATGTTCAGTCACAAGCGA | 1185 |
| 34 | TAGGAAGTGTGTAATAGC | 1186 |
| 35 | AATCCATGTAGCTGTACG | 1187 |
| 36 | CCAGATTCACTGGCATAG | 1188 |
| 37 | TTGTCTCTACGTAATATC | 1189 |
| 38 | GTGGTGCTTGTGACAATT | 1190 |
| 39 | CAGCCTACTTGGCTGAGA | 1191 |
| 40 | TACTCAATGCATCTGTGT | 1192 |
| 41 | TGTAGAGAGACGAATATA | 1193 |
| 42 | GCCTACAACCATCCTACT | 1194 |
| 43 | GCGTGGCATTGAGATTCA | 1195 |
| 44 | GCATGCCAGCTAACTGAG | 1196 |
| 45 | GCGAGTAATCCGGTTGGA | 1197 |
| 46 | GCCTCTACCAGAACGTCA | 1198 |
| 47 | GTCAGCAGAAGACTGACC | 1199 |
| 48 | GATAACAGACGTAGCAGG | 1200 |
| 49 | CAGGAGATCGCATGTCGT | 1201 |
| 50 | CTGGAAGGAATGGAGCCA | 1202 |
| 51 | ATTGGTTCTCTACCACAA | 1203 |
| 52 | CTCATTGTTGACGGCTCA | 1204 |
| 53 | TTCAGGACTGTAGTTCAT | 1205 |
| 54 | AGACCGCACTAACTCAAG | 1206 |
| 55 | GGAATATTGTGCAGACCG | 1207 |
| 56 | CCTATTACTAATAGCTCA | 1208 |
| 57 | ATGGCATGAGTACTTCGG | 1209 |
| 58 | GACACGTATGCGTCTAGC | 1210 |
| 59 | GAAGGTACGGAATCTGTT | 1211 |
| 60 | TATAACGTCCGACACTGT | 1212 |
| 61 | GCTAATACATTACCGCCG | 1213 |
| 62 | GAAGCCAACACTCCTGAC | 1214 |
| 63 | CGAATAACGAGCTGTGAT | 1215 |
| 64 | GCCTACCGATCGCACTTA | 1216 |
| 65 | CTGAGGAGAATAGCCTGC | 1217 |
| 66 | CAGCATGGACAGTACTTC | 1218 |
| 67 | GGTATAGAGCCTTCCTTA | 1219 |
| 68 | CGCTCTGCATATATAGCA | 1220 |
| 69 | CGGCTCTACTATGCTCGT | 1221 |
| 70 | CCTAATGCGAAGCTCACC | 1222 |
| 71 | ACAACCGGTGAGGCAGTA | 1223 |
| 72 | TTGGTTCGAACCAACCGC | 1224 |
| 73 | ATACTAGGTTGAACTAAG | 1225 |
| 74 | GCGTTGAGAGTAACATAT | 1226 |
| 75 | AGTTGTATAATAAGCGTC | 1227 |
| 76 | GTATGATGCCGTCCAATT | 1228 |
| 77 | GGACTCTCTGAAGAGTCT | 1229 |
| 78 | GGACTCTCTTGACTTGAA | 1230 |
| 79 | GATAACAGTGCTTCGTCC | 1231 |
| 80 | GGCCATTATAGATGAACT | 1232 |
| 81 | ATAGAGAGCACAGAGCAG | 1233 |
| 82 | GTGTGAGTGTATCATAAC | 1234 |
| 83 | ATAACCTTAGTGCGCGTC | 1235 |
| 84 | CCGACTGATATGCATGGA | 1236 |
| 85 | GGATATCTGATCGCATCA | 1237 |
| 86 | CAGCATTAACGAGGCGAA | 1238 |
| 87 | GCGAGGCCTACATATTCG | 1239 |
| 88 | CGATAAGTGGTAAGGTCT | 1240 |
| 89 | AGATCCTGAGTCGAGCAA | 1241 |
| 90 | AAGATATAACGAGACCGA | 1242 |
| 91 | CCGACTGATTGAGAACGT | 1243 |
| 92 | TCGGCTTATATGACACGT | 1244 |
| 93 | AATAACGTACGCCGGAGG | 1245 |
| 94 | AACACAGCATTGCGCACG | 1246 |
| 95 | GTAGTCTGACAGCAACAA | 1247 |
| 96 | AGAATGACTTGAGCTGCT | 1248 |

TABLE 14
Random primer list (20-nucleotide)

| No. | Primer sequence | SEQ ID NO: |
|---|---|---|
| 1 | ACTGGTAGTAACGTCCACCT | 1249 |
| 2 | AGACTGGTTGTTATTCGCCT | 1250 |
| 3 | TATCATTGACAGCGAGCTCA | 1251 |
| 4 | TGGAGTCTGAAGAAGGACTC | 1252 |
| 5 | CATCTGGACTACGGCAACGA | 1253 |
| 6 | AACTGTCATAAGACAGACAA | 1254 |
| 7 | CCTCAACATGACATACACCG | 1255 |
| 8 | CAATACCGTTCGCGATTCTA | 1256 |
| 9 | GCGTCTACGTTGATTCGGCC | 1257 |
| 10 | TGAACAGAGGCACTTGCAGG | 1258 |
| 11 | CGACTAGAACCTACTACTGC | 1259 |
| 12 | GCACCGCACGTGGAGAGATA | 1260 |
| 13 | CTGAGAGACCGACTGATGCG | 1261 |
| 14 | TCGTCCTTCTACTTAATGAT | 1262 |
| 15 | CAAGCTATACCATCCGAATT | 1263 |
| 16 | CAATACGTATAGTCTTAGAT | 1264 |
| 17 | CCATCCACAGTGACCTATGT | 1265 |
| 18 | TATCCGTTGGAGAAGGTTCA | 1266 |
| 19 | CGCCTAGGTACCTGAGTACG | 1267 |
| 20 | CAGAGTGCTCGTGTTCGCGA | 1268 |
| 21 | CGCTTGGACATCCTTAAGAA | 1269 |
| 22 | GACCGCATGATTAGTCTTAC | 1270 |
| 23 | CTTGGCCGTAGTCACTCAGT | 1271 |
| 24 | GATAGCGATATTCAGTTCGC | 1272 |
| 25 | ATCCAACACTAAGACAACCA | 1273 |
| 26 | CCATTCTGTTGCGTGTCCTC | 1274 |
| 27 | ACATTCTGTACGCTTGCAGC | 1275 |
| 28 | TGCTGAACGCCAATCGCTTA | 1276 |
| 29 | TCCTCTACAAGAATATTGCG | 1277 |
| 30 | CGACCAACGCAGCCTGATTC | 1278 |
| 31 | ATTGCGAGCTTGAGTAGCGC | 1279 |
| 32 | AAGGTGCGAGCATAGGAATC | 1280 |
| 33 | CACTTAAGTGTGATATAGAT | 1281 |
| 34 | ATCGGTATGCTGACCTAGAC | 1282 |
| 35 | TACAATCTCGAATGCAGGAT | 1283 |
| 36 | CCATATGAAGCGCAGCCGTC | 1284 |
| 37 | CGTCTCGTGGACATTCGAGG | 1285 |
| 38 | CCGAGTACAGAAGCGTGGAA | 1286 |
| 39 | TTACGTGGTCGACAGGCAGT | 1287 |
| 40 | AGCTGCAATCTGCATGATTA | 1288 |
| 41 | ACCTGCCGAAGCAGCCTACA | 1289 |
| 42 | AACATGATAACCACATGGTT | 1290 |
| 43 | ATCCGACTGATTGAATTACC | 1291 |
| 44 | TCACGCTGACTCTTATCAGG | 1292 |
| 45 | GCGCGCTCGAAGTACAACAT | 1293 |
| 46 | ACAGCCAGATGCGTTGTTCC | 1294 |
| 47 | GGAGCTCTGACCTGCAAGAA | 1295 |
| 48 | AACATTAGCCTCAAGTAAGA | 1296 |
| 49 | TGTGATTATGCCGAATGAGG | 1297 |
| 50 | GAGTAATAATCCAATCAGTA | 1298 |
| 51 | CTCCTTGGCGACAGCTGAAC | 1299 |
| 52 | TTACGCACACATACACAGAC | 1300 |
| 53 | ACGCCGTATGGCGACTTAGG | 1301 |
| 54 | AGAACGACAATTACGATGGC | 1302 |
| 55 | TGCTAACGTACCACTGCCAC | 1303 |
| 56 | CATCCAGAATGTCTATCATA | 1304 |
| 57 | GGAGAACGCCTATAGCACTC | 1305 |
| 58 | ACCTCTTGTGACGGCCAGTC | 1306 |
| 59 | TGCCATAACTTGGCATAAGA | 1307 |
| 60 | ACAATTGTCTGACCACGCTC | 1308 |
| 61 | TCGTCACCTTCACAGAACGA | 1309 |
| 62 | AGCAGCAGATGATGATCCAA | 1310 |
| 63 | TCGTGCCTTGGATTCCAGGA | 1311 |
| 64 | TGTTATAGCCACGATACTAT | 1312 |
| 65 | AATCTCACCTGTACCTTCCG | 1313 |
| 66 | GAGTAGCGGAAGCGTTAGCG | 1314 |
| 67 | AATACTCCGGCGAGGTATAC | 1315 |
| 68 | TTCGCATCCTTGCACGAACA | 1316 |
| 69 | AACCGGCTAATACTACTGGC | 1317 |
| 70 | CTAGCATCTTAGACACCAGA | 1318 |
| 71 | TAGTTGCGTGATACAAGATA | 1319 |
| 72 | TCGTCTCGACACAGTTGGTC | 1320 |
| 73 | TCCGTTCGCGTGCGAACTGA | 1321 |
| 74 | TCTGACTCTGGTGTACAGTC | 1322 |
| 75 | ACAGCGCAATTATATCCTGT | 1323 |
| 76 | AGATCCGTACGTGAGACTAG | 1324 |
| 77 | TACATTGAAGCATCCGAACA | 1325 |
| 78 | CTCCTGAGAGATCAACGCCA | 1326 |
| 79 | TCACCTCGAATGAGTTCGTT | 1327 |
| 80 | TAGCGACTTAAGGTCCAAGC | 1328 |
| 81 | AGTACGTATTGCCGTGCAAG | 1329 |
| 82 | AGCCACGAACCGACGTCATA | 1330 |
| 83 | TGATGTGTACGCTACTACTA | 1331 |
| 84 | CCACTGTGTGCAGCAGACGA | 1332 |
| 85 | CTATTGTACAGCGAACGCTG | 1333 |
| 86 | CTCCGATATCGCACGGATCG | 1334 |
| 87 | AACTTATCGTCGGACGCATG | 1335 |
| 88 | TATCCTAATTCGTGCCGGTC | 1336 |
| 89 | ACAGCCTTCCTGTGTGGACT | 1337 |
| 90 | CCTCCGTGAGGATCGTACCA | 1338 |
| 91 | GCTCTAAGTAACAGAACTAA | 1339 |
| 92 | GACTTACCGCGCGTTCTGGT | 1340 |
| 93 | TCTGAGGATACACATGTGGA | 1341 |
| 94 | TGTAATCACACTGGTGTCGG | 1342 |
| 95 | CACTAGGCGGCAGACATACA | 1343 |
| 96 | CTAGAGCACAGTACCACGTT | 1344 |

TABLE 15
Random primer list (22-nucleotide)

| No. | Primer sequence | SEQ ID NO: |
|---|---|---|
| 1 | TTCAGAGGTCTACGCTTCCGGT | 1345 |
| 2 | AACACAGACTGCGTTATGCCAA | 1346 |
| 3 | TGCTGAGTTCTATACAGCAGTG | 1347 |
| 4 | ACCTATTATATGATAGCGTCAT | 1348 |
| 5 | ATCGTGAGCTACAGTGAATGCA | 1349 |
| 6 | CGTGATGTATCCGGCCTTGCAG | 1350 |
| 7 | TCTTCTGGTCCTAGAGTTGTGC | 1351 |
| 8 | TGATGTCGGCGGCGGATCAGAT | 1352 |
| 9 | TCGGCCTTAGCGTTCAGCATCC | 1353 |
| 10 | TTAAGTAGGTCAGCCACTGCAC | 1354 |
| 11 | CCAGGTGAGTTGATCTGACACC | 1355 |
| 12 | TATACTATTACTGTGTTCGATC | 1356 |
| 13 | CCGCAGTATGTCTAGTGTTGTC | 1357 |
| 14 | GTCTACCGCGTACGAAGCTCTC | 1358 |
| 15 | ATGCGAGTCCGTGGTCGATCCT | 1359 |
| 16 | TGGTAGATTGGTGTGAGAACTA | 1360 |
| 17 | AGGTTCGTCGATCAACTGCTAA | 1361 |
| 18 | ACGACAAGCATCCTGCGATATC | 1362 |
| 19 | TTGAATCACAGAGAGCGTGATT | 1363 |
| 20 | GTACTTAGTGCTTACGTCAGCT | 1364 |
| 21 | GATTATTAAGGCCAAGCTCATA | 1365 |
| 22 | GCATGCAGAGACGTACTCATCG | 1366 |
| 23 | TAGCGGATGGTGTCCTGGCACT | 1367 |
| 24 | TACGGCTGCCAACTTAATAACT | 1368 |
| 25 | CTCATATGACAACTTCTATAGT | 1369 |
| 26 | CAAGCAATAGTTGTCGGCCACC | 1370 |
| 27 | TTCAGCAATCCGTACTGCTAGA | 1371 |
| 28 | TGAGACGTTGCTGACATTCTCC | 1372 |
| 29 | GTTCCGATGAGTTAGATGTATA | 1373 |
| 30 | TTGACGCTTGGAGGAGTACAAG | 1374 |
| 31 | TTCATGTTACCTCCACATTGTG | 1375 |
| 32 | GAGCACGTGCCAGATTGCAACC | 1376 |
| 33 | GGTCGACAAGCACAAGCCTTCT | 1377 |
| 34 | TAGGCAGGTAAGATGACCGACT | 1378 |
| 35 | CGAGGCATGCCAAGTCGCCAAT | 1379 |
| 36 | AGTGTTGATAGGCGGATGAGAG | 1380 |
| 37 | TTCGGTCTAGACCTCTCACAAT | 1381 |
| 38 | GTGACGCTCATATCTTGCCACC | 1382 |
| 39 | GATGTAATTCTACGCGCGGACT | 1383 |
| 40 | GATGGCGATGTTGCATTACATG | 1384 |
| 41 | TATGCTCTGAATTAACGTAGAA | 1385 |
| 42 | AGGCAATATGGTGATCCGTAGC | 1386 |
| 43 | TGACAGCGATGCATACAGTAGT | 1387 |
| 44 | TTCTGCTAACGGTATCCAATAC | 1388 |
| 45 | GAGTCGTCCATACGATCTAGGA | 1389 |
| 46 | AGACGGACTCAACGCCAATTCC | 1390 |
| 47 | GTAGTGTTGAGCGGACCGAGCT | 1391 |
| 48 | AATATAACTAGATCATAGCCAG | 1392 |
| 49 | TCAATCGGAGAATACAGAACGT | 1393 |
| 50 | ATCTCCGTCGTCCGAACCAACA | 1394 |
| 51 | TAGGCGTTCAGCGGTATGCTTA | 1395 |
| 52 | TGCGTGCTATACAACCTATACG | 1396 |
| 53 | ATGGCCGGCATACATCTGTATG | 1397 |
| 54 | TGATGCTGACATAACACTGAAT | 1398 |
| 55 | ATCCAAGGTACCTGAACATCCT | 1399 |
| 56 | TAGTGACGACCAGGTGAGCCTC | 1400 |
| 57 | AGGAGGATCCGTCAAGTCGACC | 1401 |
| 58 | AGAGTATGCCAGATCGTGAGGC | 1402 |
| 59 | CCACTCACTAGGATGGCTGCGT | 1403 |
| 60 | TATCCAACCTGTTATAGCGATT | 1404 |
| 61 | TCTTGCAGTGAGTTGAGTCTGC | 1405 |
| 62 | CCACTGTTGTACATACACCTGG | 1406 |
| 63 | ATGCGCGTAGGCCACTAAGTCC | 1407 |
| 64 | ACAGCGGTCTACAACCGACTGC | 1408 |
| 65 | TCGCGCTCCAGACAATTGCAGC | 1409 |
| 66 | CCGGTAGACCAGGAGTGGTCAT | 1410 |
| 67 | ATCTCCTAACCTAGAGCCATCT | 1411 |
| 68 | CCACATCGAATCTAACAACTAC | 1412 |
| 69 | TAGTCTTATTGAATACGTCCTA | 1413 |
| 70 | TCCTTAAGCCTTGGAACTGGCG | 1414 |
| 71 | CCGTGATGGATTGACGTAGAGG | 1415 |
| 72 | GCCTGGATAACAGATGTCTTAG | 1416 |
| 73 | CTCGACCTATAATCTTCTGCCA | 1417 |
| 74 | AGCTACTTCTCCTTCCTAATCA | 1418 |
| 75 | ACACGCTATTGCCTTCCAGTTA | 1419 |
| 76 | AAGCCTGTGCATGCAATGAGAA | 1420 |
| 77 | TCGTTGGTTATAGCACAACTTC | 1421 |
| 78 | GCGATGCCTTCCAACATACCAA | 1422 |
| 79 | CCACCGTTAGCACGTGCTACGT | 1423 |
| 80 | GTTACCACAATGCCGCCATCAA | 1424 |
| 81 | GGTGCATTAAGAACGAACTACC | 1425 |
| 82 | TCCTTCCGGATAATGCCGATTC | 1426 |
| 83 | AACCGCAACTTCTAGCGGAAGA | 1427 |
| 84 | TCCTTAAGCAGTTGAACCTAGG | 1428 |
| 85 | TACTAAGTCAGATAAGATCAGA | 1429 |
| 86 | TTCGCCATAACTAGATGAATGC | 1430 |
| 87 | AAGAAGTTAGACGCGGTGGCTG | 1431 |
| 88 | GTATCTGATCGAAGAGCGGTGG | 1432 |
| 89 | TCAAGAGCTACGAAGTAAGTCC | 1433 |
| 90 | CGAGTACACAGCAGCATACCTA | 1434 |
| 91 | CTCGATAAGTTACTCTGCTAGA | 1435 |
| 92 | ATGGTGCTGGTTCTCCGTCTGT | 1436 |
| 93 | TCAAGCGGTCCAAGGCTGAGAC | 1437 |
| 94 | TGTCCTGCTCTGTTGCTACCGT | 1438 |
| 95 | AGTCATATCGCGTCACACGTTG | 1439 |
| 96 | GGTGAATAAGGACATGAGAAGC | 1440 |

TABLE 16
Random primer list (24-nucleotide)

| No. | Primer sequence | SEQ ID NO: |
|---|---|---|
| 1 | CCTGATCTTATCTAGTAGAGACTC | 1441 |
| 2 | TTCTGTGTAGGTGTGCCAATCACC | 1442 |
| 3 | GACTTCCAGATGCTTAAGACGACA | 1443 |
| 4 | GTCCTTCGACGGAGAACATCCGAG | 1444 |
| 5 | CTTGGTTAGTGTACCGTCAACGTC | 1445 |
| 6 | AAGCGGCATGTGCCTAATCGACGT | 1446 |
| 7 | CGACCGTCGTTACACGGAATCCGA | 1447 |
| 8 | TCGCAAGTGTGCCGTTCTGTTCAT | 1448 |
| 9 | CGTACTGAAGTTCGGAGTCGCCGT | 1449 |
| 10 | CCACTACAGAATGGTAGCAGATCA | 1450 |
| 11 | AGTAGGAGAGAGGCCTACACAACA | 1451 |
| 12 | AGCCAAGATACTCGTTCGGTATGG | 1452 |
| 13 | GTTCCGAGTACATTGAATCCTGGC | 1453 |
| 14 | AGGCGTACGAGTTATTGCCAGAGG | 1454 |
| 15 | GTGGCATCACACATATCTCAGCAT | 1455 |
| 16 | GAGACCGATATGTTGATGCCAGAA | 1456 |
| 17 | CAACTGTAGCCAGTCGATTGCTAT | 1457 |
| 18 | TATCAATGCAATGAGAGGATGCAG | 1458 |
| 19 | GTATGCTCGGCTCCAAGTACTGTT | 1459 |
| 20 | AGAGACTCTTATAGGCTTGACGGA | 1460 |
| 21 | ACTTAACAGATATGGATCATCGCC | 1461 |
| 22 | AATCAGAGCGAGTCTCGCTTCAGG | 1462 |
| 23 | ACCACCGAGGAACAGGTGCGACAA | 1463 |
| 24 | TGGTACATGTCAACCGTAAGCCTG | 1464 |
| 25 | CGTGCCGCGGTGTTCTTGTATATG | 1465 |
| 26 | GACAAGCGCGCGTGAGACATATCA | 1466 |
| 27 | AGTGCACTCCGAACAAGAGTTAGT | 1467 |
| 28 | CCTCATTACCGCGTTAGGAGTCCG | 1468 |
| 29 | TGCTTATTGCTTAGTTGCTATCTC | 1469 |
| 30 | GCGTGATCCTGTTCTATTCGTTAG | 1470 |
| 31 | GGCCAGAACTATGACGAGTATAAG | 1471 |
| 32 | GATGGCGACTATCTAATTGCAATG | 1472 |
| 33 | TAGTAACCATAGCTCTGTACAACT | 1473 |
| 34 | CGTGATCGCCAATACACATGTCGC | 1474 |
| 35 | TAATAACGGATCGATATGCACGCG | 1475 |
| 36 | ATCATCGCGCTAATACTATCTGAA | 1476 |
| 37 | CACGTGCGTGCAGGTCACTAGTAT | 1477 |
| 38 | AGGTCCAATGCCGAGCGATCAGAA | 1478 |
| 39 | CAGCATAACAACGAGCCAGGTCAG | 1479 |
| 40 | ATGGCGTCCAATACTCCGACCTAT | 1480 |
| 41 | AGGAACATCGTGAATAATGAAGAC | 1481 |
| 42 | TCTCGACGTTCATGTAATTAAGGA | 1482 |
| 43 | TCGCGGTTAACCTTACTTAGACGA | 1483 |
| 44 | ATCATATCTACGGCTCTGGCGCCG | 1484 |
| 45 | GCAGATGGAGACCAGAGGTACAGG | 1485 |
| 46 | AGACAGAAGATTACCACGTGCTAT | 1486 |
| 47 | CCACGGACAACATGCCGCTTAACT | 1487 |
| 48 | CTTGAAGTCTCAAGCTATGAGAGA | 1488 |
| 49 | ACAGCAGTCGTGCTTAGGTCACTG | 1489 |
| 50 | AGGTGTTAATGAACGTAGGTGAGA | 1490 |
| 51 | AGCCACTATGTTCAAGGCTGAGCC | 1491 |
| 52 | GCAGGCGGTGTCGTGTGACAATGA | 1492 |
| 53 | AGCCATTGCTACAGAGGTTACTTA | 1493 |
| 54 | ACAATCGAACCTACACTGAGTCCG | 1494 |
| 55 | CCGATCTCAATAGGTACCACGAAC | 1495 |
| 56 | GATACGTGGCGCTATGCTAATTAA | 1496 |
| 57 | AGAGAGATGGCACACATTGACGTC | 1497 |
| 58 | CTCAACTCATCCTTGTAGCCGATG | 1498 |
| 59 | GTGGAATAACGCGATACGACTCTT | 1499 |
| 60 | ATCTACCATGCGAATGCTCTCTAG | 1500 |
| 61 | ATACGCACGCCTGACACAAGGACC | 1501 |
| 62 | GTCCACTCTCAGTGTGTAGAGTCC | 1502 |
| 63 | AATATATCCAGATTCTCTGTGCAG | 1503 |
| 64 | CCTTCCGCCACATGTTCGACAAGG | 1504 |
| 65 | ACTGTGCCATCATCCGAGGAGCCA | 1505 |
| 66 | TCTATGCCGCTATGGCGTCGTGTA | 1506 |
| 67 | CGTAACCTAAGGTAATATGTCTGC | 1507 |
| 68 | TACTGACCGTATCAAGATTACTAA | 1508 |
| 69 | TCATCGGAGCGCCATACGGTACGT | 1509 |
| 70 | GCAAGAGGAATGAACGAAGTGATT | 1510 |
| 71 | GGCTGATTGACATCCTGACTTAGT | 1511 |
| 72 | AAGGCGCTAGATTGGATTAACGTA | 1512 |
| 73 | GCTAGCTAGAAGAATAGGATTCGT | 1513 |
| 74 | CAGGTGACGGCCTCTATAACTCAT | 1514 |
| 75 | CAGGTTACACATACCACTATCTTC | 1515 |
| 76 | TTGCTACGTACCGTCTTAATCCGT | 1516 |
| 77 | CTCAACATGTCTTGCAAGCTTCGA | 1517 |
| 78 | GGTGCGGTACGTAGAACCAGATCA | 1518 |
| 79 | AATGCTCTCCAAGATCCTGACCTA | 1519 |
| 80 | GCTTCGCAGGTCTGGATGATGGAG | 1520 |
| 81 | ACATTGACCAGACAGCACCTTGCG | 1521 |
| 82 | AGGTATCAATGTGCTTAATAGGCG | 1522 |
| 83 | TCCGGACACACGATTAGTAACGGA | 1523 |
| 84 | TACGAAGTACTACAGATCGGTCAG | 1524 |
| 85 | AATTGTCAGACGAATACTGCTGGA | 1525 |
| 86 | TGAATCATGAGCCAGAGGTTATGC | 1526 |
| 87 | CACAAGACACGTCATTAACATCAA | 1527 |
| 88 | GAATGACTACATTACTCCGCCAGG | 1528 |
| 89 | AGCCAGAGATACTGGAACTTGACT | 1529 |
| 90 | TATCAGACACATCACAATGGATAC | 1530 |
| 91 | CTAGGACACCGCTAGTCGGTTGAA | 1531 |
| 92 | GTATAACTGCGTGTCCTGGTGTAT | 1532 |
| 93 | ATGCAATACTAAGGTGGACCTCCG | 1533 |
| 94 | ATGCAGACGCTTGCGATAAGTCAT | 1534 |
| 95 | TTGCTCGATACACGTAGACCAGTG | 1535 |
| 96 | TACTGGAGGACGATTGTCTATCAT | 1536 |

TABLE 17
Random primer list (26-nucleotide)

| No. | Primer sequence | SEQ ID NO: |
|---|---|---|
| 1 | ACTAAGGCACGCTGATTCGAGCATTA | 1537 |
| 2 | CGGATTCTGGCACGTACAAGTAGCAG | 1538 |
| 3 | TTATGGCTCCAGATCTAGTCACCAGC | 1539 |
| 4 | CATACACTCCAGGCATGTATGATAGG | 1540 |
| 5 | AGTTGTAAGCCAACGAGTGTAGCGTA | 1541 |
| 6 | GTATCAGCTCCTTCCTCTGATTCCGG | 1542 |
| 7 | AACATACAGAATGTCTATGGTCAGCT | 1543 |
| 8 | GACTCATATTCATGTTCAGTATAGAG | 1544 |
| 9 | AGAGTGAACGAACGTGACCGACGCTC | 1545 |
| 10 | AATTGGCGTCCTTGCCACAACATCTT | 1546 |
| 11 | TCGTAGACGCCTCGTACATCCGAGAT | 1547 |
| 12 | CCGGCTCGTGAGGCGATAATCATATA | 1548 |
| 13 | AGTCCTGATCACGACCACGACTCACG | 1549 |
| 14 | GGCACTCAATCCTCCATGGAGAAGCT | 1550 |
| 15 | TCATCATTCCTCACGTTCACCGGTGA | 1551 |
| 16 | TCAACTCTGTGCTAACCGGTCGTACA | 1552 |
| 17 | TGTTCTTATGCATTAATGCCAGGCTT | 1553 |
| 18 | GATTCACGACCTCAACAGCATCACTC | 1554 |
| 19 | GGCGAGTTCGACCAGAATGCTGGACA | 1555 |
| 20 | TTCCGTATACAATGCGATTAAGATCT | 1556 |
| 21 | GAGTAATCCGTAACCGGCCAACGTTG | 1557 |
| 22 | CGCTTCCATCATGGTACGGTACGTAT | 1558 |
| 23 | CCGTCGTGGTGTGTTGACTGGTCAAC | 1559 |
| 24 | TATTCGCATCTCCGTATTAGTTGTAG | 1560 |
| 25 | TATTATTGTATTCTAGGCGGTGCAAC | 1561 |
| 26 | AGGCTGCCTACTTCCTCGTCATCTCG | 1562 |
| 27 | GTAACATACGGCTCATCGAATGCATC | 1563 |
| 28 | TTATGGCACGGATATTACCGTACGCC | 1564 |
| 29 | ATAGCACTTCCTCTAATGCTCTGCTG | 1565 |
| 30 | TCACAGGCAATAGCCTAATATTATAT | 1566 |
| 31 | GGCGGATGTTCGTTAATATTATAAGG | 1567 |
| 32 | TGCAATAGCCGTTGTCTCTGCCAGCG | 1568 |
| 33 | TACAGCGCGTTGGCGAGTACTGATAG | 1569 |
| 34 | TGCAGTTAGTACCTTCTCACGCCAAC | 1570 |
| 35 | CCATTGGCTACCTAGCAGACTCTACC | 1571 |
| 36 | AACAGTAGCTCGCGTCTTGCTCTCGT | 1572 |
| 37 | GCAGTCCATCAGCTCTCGCTTATAGA | 1573 |
| 38 | TATCTCTCTGTCGCCAGCTTGACCAA | 1574 |
| 39 | CAGACTGTTCAAGCTTGCTGTAGGAG | 1575 |
| 40 | TAACCGGAACTCGTTCAGCAACATTC | 1576 |
| 41 | TCAATTATGCATGTCGTCCGATCTCT | 1577 |
| 42 | TTGTCTAAGTCAACCTGTGGATAATC | 1578 |
| 43 | TCTAAGAGTGGTATGACCAGGAGTCC | 1579 |
| 44 | TCGTAGTACTACTGGAACAGGTAATC | 1580 |
| 45 | ATGTCAACATTCTAATCATCTCTCGG | 1581 |
| 46 | AGCGCGCAACTGTTACGGTGATCCGA | 1582 |
| 47 | GCGATAGAATAATGGTGTCACACACG | 1583 |
| 48 | AAGGCTGCGATGAGAGGCGTACATCG | 1584 |
| 49 | GGTTCATGGTCTCAGTCGTGATCGCG | 1585 |
| 50 | TAGTGACTCTATGTCACCTCGGAGCC | 1586 |
| 51 | ATGTGATAGCAATGGCACCTCTAGTC | 1587 |
| 52 | TCGCGAAGTGTAATGCATCATCCGCT | 1588 |
| 53 | ATGTGGCGACGATCCAAGTTCAACGC | 1589 |
| 54 | ACCTTGTATGAGTCGGAGTGTCCGGC | 1590 |
| 55 | ACCTCAAGAGAGTAGACAGTTGAGTT | 1591 |
| 56 | GGTGTAATCCTGTGTGCGAAGCTGGT | 1592 |
| 57 | ATAGCGGAACTGTACGACGCTCCAGT | 1593 |
| 58 | AAGCACGAGTCGACCATTAGCCTGGA | 1594 |
| 59 | ATTCCGGTAACATCAGAAGGTACAAT | 1595 |
| 60 | GTGCAACGGCAGTCCAGTATCCTGGT | 1596 |
| 61 | CCATCTTATACACGGTGACCGAAGAT | 1597 |
| 62 | GCACTTAATCAAGCTTGAGTGATGCT | 1598 |
| 63 | AGTATTACGTGAGTACGAAGATAGCA | 1599 |
| 64 | TTCTTAGGTTAAGTTCCTTCTGGACC | 1600 |
| 65 | GTCCTTGCTAGACACTGACCGTTGCT | 1601 |
| 66 | GCCGCTATGTGTGCTGCATCCTAAGC | 1602 |
| 67 | CCATCAATAACAGACTTATGTTGTGA | 1603 |
| 68 | CGCGTGTGCTTACAAGTGCTAACAAG | 1604 |
| 69 | CGATATGTGTTCGCAATAAGAGAGCC | 1605 |
| 70 | CGCGGATGTGAGCGGCTCAATTAGCA | 1606 |
| 71 | GCTGCATGACTATCGGATGGAGGCAT | 1607 |
| 72 | CTATGCCGTGTATGGTACGAGTGGCG | 1608 |
| 73 | CCGGCTGGAGTTCATTACGTAGGCTG | 1609 |
| 74 | TGTAGGCCTACTGAGCTAGTATTAGA | 1610 |
| 75 | CCGTCAAGTGACTATTCTTCTAATCT | 1611 |
| 76 | GGTCTTACGCCAGAGACTGCGCTTCT | 1612 |
| 77 | CGAAGTGTGATTATTAACTGTAATCT | 1613 |
| 78 | GCACGCGTGGCCGTAAGCATCGATTA | 1614 |
| 79 | ATCCTGCGTCGGAACGTACTATAGCT | 1615 |
| 80 | AGTATCATCATATCCATTCGCAGTAC | 1616 |
| 81 | AGTCCTGACGTTCATATATAGACTCC | 1617 |
| 82 | CTTGCAGTAATCTGAATCTGAAGGTT | 1618 |
| 83 | ATAACTTGGTTCCAGTAACGCATAGT | 1619 |
| 84 | GATAAGGATATGGCTGTAGCGAAGTG | 1620 |
| 85 | GTGGAGCGTTACAGACATGCTGAACA | 1621 |
| 86 | CGCTTCCGGCAGGCGTCATATAAGTC | 1622 |
| 87 | ATAACATTCTAACCTCTATAAGCCGA | 1623 |
| 88 | ACGATCTATGATCCATATGGACTTCC | 1624 |
| 89 | TGAAGCTCAGATATCATGCCTCGAGC | 1625 |
| 90 | AGACTTCACCGCAATAACTCGTAGAT | 1626 |
| 91 | AGACTAAGACATACGCCATCACCGCT | 1627 |
| 92 | TGTAGCGTGATGTATCGTAATTCTGT | 1628 |
| 93 | TGTGCTATTGGCACCTCACGCTGACC | 1629 |
| 94 | TGTAGATAAGTATCCAGCGACTCTCT | 1630 |
| 95 | AATTCGCCAATTGTGTGTAGGCGCAA | 1631 |
| 96 | CGATTATGAGTACTTGTAGACCAGCT | 1632 |

TABLE 18
Random primer list (28-nucleotide)

| No. | Primer sequence | SEQ ID NO: |
|---|---|---|
| 1 | TTGCAAGAACAACGTATCTCATATGAAC | 1633 |
| 2 | CACCGTGCTGTTATTACTTGGTATTCGG | 1634 |
| 3 | CACGTGTATTGTTGCACCAGAACGACAA | 1635 |
| 4 | ATGCACGTAATTACTTCCGGAGAAGACG | 1636 |
| 5 | TATGTTGTCTGATATGGTTCATGTGGCA | 1637 |
| 6 | AGCGCGACTAGTTGATGCCAACATTGTA | 1638 |
| 7 | ATAGGCAGGTCCAGGCTCGGAACAAGTC | 1639 |
| 8 | GCGGTAGTCGGTCAAGAACTAGAACCGT | 1640 |
| 9 | ACTATACACTCTAGCTATTAGGAAGCAT | 1641 |
| 10 | GATCATCTTGCTTCTCCTGTGGAGATAA | 1642 |
| 11 | CTACTACGAGTCCATAACTGATAGCCTC | 1643 |
| 12 | GCACAGACACCTGTCCTATCTAGCAGGA | 1644 |
| 13 | AAGCGAGGCGCGAAGGAGATGGAAGGAT | 1645 |
| 14 | CTGAAGACGCCAGTCTGGATAGGTGCCT | 1646 |
| 15 | GTAAGCTCTGTCCTTCGAGATTGATAAG | 1647 |
| 16 | GGTTAGAGAGATTATTGTGCGCATCCAT | 1648 |
| 17 | CCAGGAGGACCTATGATCTTGCCGCCAT | 1649 |
| 18 | ACTATTCGAGCTACTGTATGTGTATCCG | 1650 |
| 19 | GACATCGCGATACGTAACTCCGGAGTGT | 1651 |
| 20 | CCGCAATTCGTCTATATATTCTAGCATA | 1652 |
| 21 | CTACACTTGAGGTTGATGCTCAAGATCA | 1653 |
| 22 | CGATCAGTTCTAGTTCACCGCGGACAAT | 1654 |
| 23 | AAGAATGATGATTGGCCGCGAACCAAGC | 1655 |
| 24 | CACGACCGGAACTAGACTCCTACCAATT | 1656 |
| 25 | AGTTGCCTGTGAGTGAGGCTACTATCTC | 1657 |
| 26 | GATTCTTCCGATGATCATGCCACTACAA | 1658 |
| 27 | CGCTGAAGTGAACTATGCAAGCACCGCA | 1659 |
| 28 | ATTATCGTGATGGTGAGACTGAGCTCGT | 1660 |
| 29 | CGAGGCCACTCTGAGCCAGGTAAGTATC | 1661 |
| 30 | TGCCGAGGACAGCCGATCACATCTTCGT | 1662 |
| 31 | GTTGACATGAAGGTTATCGTCGATATTC | 1663 |
| 32 | GTGGTCCAGGTCAAGCTCTGATCGAATG | 1664 |
| 33 | CCAGTCCGGTGTACTCAGACCTAATAAC | 1665 |
| 34 | CGAGACACTGCATGAGCGTAGTCTTATT | 1666 |
| 35 | GACGGCTTGTATACTTCTCTACGGTCTG | 1667 |
| 36 | TTAGCTGGATGGAAGCCATATTCCGTAG | 1668 |
| 37 | CAGCCTACACTTGATTACTCAACAACTC | 1669 |
| 38 | GTACGTAGTGTCACGCGCCTACGTTCGT | 1670 |
| 39 | CTACAACTTCTCAATCATGCCTCTGTTG | 1671 |
| 40 | CGAGGACAGAATTCGACATAAGGAGAGA | 1672 |
| 41 | GCCGAACGACACAGTGAGTTGATAGGTA | 1673 |
| 42 | GAACACTATATGCTGTCGCTGTCTGAGG | 1674 |
| 43 | GTTAAGTTCTTCGGCGGTCATGCTCATT | 1675 |
| 44 | TTGCTTACAGATCGCGTATCCATAGTAT | 1676 |
| 45 | GAGGACCACCTCTGCGAAGTTCACTGTG | 1677 |
| 46 | AATCCTAGCATATCGAGAACGACACTGA | 1678 |
| 47 | TGAATACTATAGCCATAGTCGACTTCCG | 1679 |
| 48 | GACATCCACGAAGCTGGTAATCGGAACC | 1680 |
| 49 | TTAGCCGTCTTAGAAGTGTCTGACCGGC | 1681 |
| 50 | CTATTCTGCCGTAATTGATTCCTTCGTT | 1682 |
| 51 | ACGCCTCTGGTCGAAGGTAGATTAGCTC | 1683 |
| 52 | CAGCCTATTGATCGTAAGTAGATGGTCC | 1684 |
| 53 | TTAAGTGAGGTGGACAACCATCAACTTC | 1685 |
| 54 | AAGGCCTTGCGGCTAAGTAGTATTCATC | 1686 |
| 55 | TTGTGATACTAATTCTTCTCAAGAGTCA | 1687 |
| 56 | GCATTAGGTGACGACCTTAGTCCATCAC | 1688 |
| 57 | GCGGATGGACGTATACAGTGAGTCGTGC | 1689 |
| 58 | GAACATGCCAGCCTCAACTAGGCTAAGA | 1690 |
| 59 | TCCGTCATTAGAGTATGAGTGACTACTA | 1691 |
| 60 | AACACTTAGTAACCAGTTCGGACTGGAC | 1692 |
| 61 | CGCTAACTATTGCGTATATTCGCGGCTT | 1693 |
| 62 | GCCATCTACGATCTTCGGCTTATCCTAG | 1694 |
| 63 | CCTGAGAATGTTGACTAAGATCTTGTGA | 1695 |
| 64 | TCGGTTAGTCTAATCATCACGCAACGGA | 1696 |
| 65 | ATTATCTATTGAAGCAGTGACAGCGATC | 1697 |
| 66 | GAGGAGAATCACGGAACACGGTCACATG | 1698 |
| 67 | GCTGCAAGCATTATGACCATGGCATCTG | 1699 |
| 68 | GAACAACCTATAACGACGTTGTGGACAA | 1700 |
| 69 | TTAATCATCGATAGACGACATGGAATCA | 1701 |
| 70 | TCGAGTGTAAGCACACTACGATCTGGAA | 1702 |
| 71 | GCTACGCACAGTCTCTGCACAGCTACAC | 1703 |
| 72 | CCTGTATGTACGTTCTGGCTAATACCTT | 1704 |
| 73 | TGAAGCACCGGTACATGGTGTATCCGGA | 1705 |
| 74 | TGCTGGAACCTAACTCGGTGATGACGAT | 1706 |
| 75 | CGCTATCTTACTGCCAAGTTCTCATATA | 1707 |
| 76 | AACGCGCGCGTATCGGCAATAATCTCAA | 1708 |
| 77 | CCATTAGGATGACCATCGACTATTAGAG | 1709 |
| 78 | TACTGCTAGACTGCGTGCATTCATGGCG | 1710 |
| 79 | CATTGCGCGCTCCACGAACTCTATTGTC | 1711 |
| 80 | GACGCGCCTAGAACTGTATAGCTCTACG | 1712 |
| 81 | CATTGCAACTTGTCGGTGATGGCAATCC | 1713 |
| 82 | TTAATGCACATGCAGTACGGCACCACAG | 1714 |
| 83 | AGCGGTACGTGGACGAGTGGTAATTAAT | 1715 |
| 84 | GACGTATTGCTATGCATTGGAAGATGCT | 1716 |
| 85 | AACACTTCGACCATTGCGCCTCAATGGT | 1717 |
| 86 | CGGTACGCTCTAGCGGTCATAAGATGCA | 1718 |
| 87 | CCTGAATAACAGCCGCGCCTAATTAGAT | 1719 |
| 88 | AAGCGTCTAATGTGCCTTAAGTCACATG | 1720 |
| 89 | GCTCTCCAAGAACCAGAAGTAAGCATCG | 1721 |
| 90 | GAGGAGAGTTGTCCGAGTGGTGTGATGT | 1722 |
| 91 | TAACGAGTGGTGCGTCTAAGCAATTGAG | 1723 |
| 92 | CCAACAGTATGCTGACATAACTATGATA | 1724 |
| 93 | GATCCTTGCCACGCCTATGAGATATCGC | 1725 |
| 94 | AACGCGCTACCGTCCTTGTGCATAGAGG | 1726 |
| 95 | CTACATGTGCCTTATAGTACAGAGGAAC | 1727 |
| 96 | CAGCCTCGTAGTTAGCGTGATTCATGCG | 1728 |

TABLE 19
Random primer list (29-nucleotide)

| | | |
|---|---|---|
| No. | Primer sequence | SEQ ID NO: |

| | | |
|---|---|---|
| 1 | CTCCTCGCCGATTGAAGTGCGTAGAACTA | 1729 |
| 2 | CAGCAGGCCTCAATAGGATAAGCCAACTA | 1730 |
| 3 | GACCATCAATCTCGAAGACTACGCTCTGT | 1731 |
| 4 | GGTTGCTCCGTCTGTTCAGCACACTGTTA | 1732 |
| 5 | AATGTCGACTGGCCATTATCGCCAAGTGT | 1733 |
| 6 | GATAGCTTGCCATGCGAATGGATCTCCAG | 1734 |
| 7 | CCAGACCGGAGCCAATTGGCTGCCAATAT | 1735 |
| 8 | AACGTCGCTCCATACGTTACCTAATGCAG | 1736 |
| 9 | GAATATGACGCGAACAGTCTATTCGGATC | 1737 |
| 10 | GACGAGAATGTATTAAGGATAAGCAAGGT | 1738 |
| 11 | AAGTCGTATGAATCGCTATCACATGAGTC | 1739 |
| 12 | GTCGTGGAGACTACAATTCTCCTCACGTT | 1740 |
| 13 | GTTGCCACCGTTACACGACTATCGACAGT | 1741 |
| 14 | AGGATAGGCTACGCCTTACTCTCCTAAGC | 1742 |
| 15 | TAATCATCCTGTTCGCCTCGAGGTTGTTA | 1743 |
| 16 | GACAAGCAGTAATAATTACTGAGTGGACG | 1744 |
| 17 | TACAGCGTTACGCAGGTATATCAAGGTAG | 1745 |
| 18 | CTAACATCACTTACTATTAGCGGTCTCGT | 1746 |
| 19 | CCGCGCTTCTTGACACGTTCTCCACTAGG | 1747 |
| 20 | CAAGTAACATGAGATGCTATCGGTACATT | 1748 |
| 21 | CGACCACTAGGCTGTGACCACGATACGCT | 1749 |
| 22 | CAGGTCATGTGACGCAGTCGGCAGTCAAC | 1750 |
| 23 | ACTCCATCGTTAGTTCTTCCGCCGTGCTG | 1751 |
| 24 | CTCACCACGTATGCGTCACTCGGTTACGT | 1752 |
| 25 | TGCCTATGCTATGGACCTTGCGCGACTCT | 1753 |
| 26 | AATGAAGGTCAACGCTCTGTAGTTACGCG | 1754 |
| 27 | CACCATTGATTCATGGCTTCCATCACTGC | 1755 |
| 28 | GACACGCAAGGTAATTCGAGATTGCAGCA | 1756 |
| 29 | CACCGAGAGGAAGGTTCGATCGCTTCTCG | 1757 |
| 30 | CAGTTATCGGATTGTGATATTCACTCCTG | 1758 |
| 31 | ATACTGTAACGCCTCAACCTATGCTGACT | 1759 |
| 32 | ATCTGTCTTATTCTGGCACACTCAGACTT | 1760 |
| 33 | TCCAACCGGTGACGTGCTCTTGATCCAAC | 1761 |
| 34 | CACACTCAGTTCGGCTATCTCTGCGATAG | 1762 |
| 35 | AGCTGTAAGTCAGGTCTACGACTCGTACT | 1763 |
| 36 | GTCGGCGGCACGCACAGCTAACATTCGTA | 1764 |
| 37 | ATATGGTAGCCAGCCACGTATACTGAACA | 1765 |
| 38 | TGGACAATCCGACTCTAACACAGAGGTAG | 1766 |
| 39 | TCCGCCGCTGACAGTTCAATCTATCAATT | 1767 |
| 40 | GGTTCCTTAGAATATGCACCTATCAGCGA | 1768 |
| 41 | CGGCTGTACGACATGGATCATAAGAGTGT | 1769 |
| 42 | TGCAGATGTACGCTGTGGCCAGTGGAGAG | 1770 |
| 43 | CCTACTCACTTAACAATAATCGGTTCGGT | 1771 |
| 44 | CGCTTCCTACTGCCTGTGCCGCGACATAA | 1772 |
| 45 | CTAGACCGACCGGTTATGCGCTATTGTTC | 1773 |
| 46 | TTGTGAGCACGTCTGCGGCAAGCCTATGG | 1774 |
| 47 | TCATCGGCCGGCGCTGTTGTTGTTACCAT | 1775 |
| 48 | GCGGTTAGGTGCAGTTAGGAAGACTATCA | 1776 |
| 49 | TATGCGGTCGTGAGGCGTAGCATTCTAGA | 1777 |
| 50 | CCATCTATTCGTCGAACTCTCAGCTCGTA | 1778 |
| 51 | ATCAGATCTACTGATCGCGGTAGAGTATC | 1779 |
| 52 | TACACATAGGCGGCGCAGCCTTCTAATTA | 1780 |
| 53 | TTAACCGTAGTTCTTAGCTTACGCCGCTC | 1781 |
| 54 | ACTATAGAGGACATGGCACTCCTCTTCTA | 1782 |
| 55 | CAGTTCGTATTAAGATTGAATGTAGCGGT | 1783 |
| 56 | AGTTATCGGTATCCGCTTATCCGTACGTA | 1784 |
| 57 | AGCTTATTCATACACTGCACCACAGCAAG | 1785 |
| 58 | CCGTCGGCTAGTCTATCCTCTAATTAGAA | 1786 |
| 59 | GTCCGCTTCCATGCCTGCTGTACGAACAC | 1787 |
| 60 | TCTCTTCCTCCTTCATTGTTCGCTAGCTC | 1788 |
| 61 | TCTCTTGAGCGGTCCTCATACAGGTCTGC | 1789 |
| 62 | GACCAAGTGTAGGTGATATCACCGGTACT | 1790 |
| 63 | AAGATTGTGATAGGTTGGTAGTTACCACA | 1791 |
| 64 | TCGCCTCCGAAGAGTATAGCATCGGCAGA | 1792 |
| 65 | GAGGTAGTTATGAGCATCGAGGTCCTGTT | 1793 |
| 66 | GGACGCAAGATCGCAGGTACTTGTAAGCT | 1794 |
| 67 | ACTCGTACACGTCATCGTGCAGGTCTCAG | 1795 |
| 68 | TAATCCGTCAGGAGTGAGATGGCTCGACA | 1796 |
| 69 | AAGATGGTTCCGCGCATTGACTAGCAAGT | 1797 |
| 70 | TCCGCGATCTGCGGATCTTGAATGCTCAC | 1798 |
| 71 | TTCACGAGAGTCAACTGCTAGTATCCTAG | 1799 |
| 72 | TTCCAACTGGATTCTTCCAACTCCTCGAA | 1800 |
| 73 | CACTACTACTCAAGTTATACGGTGTTGAC | 1801 |
| 74 | CAACTGGATTCTCAGGATGCGTCTCTAGC | 1802 |
| 75 | TGGACTAGAGTGGAGCGATTACGTAATAT | 1803 |
| 76 | GAGGTCATTCAACTGGACTCGCCACGGAC | 1804 |
| 77 | CAGGTGTGTAACGCTGCAATCACATGAAT | 1805 |
| 78 | TATGCTGAGGTATTAGTTCTAACTATGCG | 1806 |
| 79 | CGTCTGAGTCGGATAAGGAAGGTTACCGC | 1807 |
| 80 | GTACTATCGTCGCAGGCACTATCTCTGCC | 1808 |
| 81 | GCTTCCTCCTTGCAACTTCATTGCTTCGA | 1809 |
| 82 | TGTCTACGAAGTAGAAGACACGAATAATG | 1810 |
| 83 | CCGTCATCTAAGGCAGAGTACATCCGCGA | 1811 |
| 84 | CCGGAGGCGTACTAACTGACCACAACACC | 1812 |
| 85 | AACTCGTCGCTGCCTGAATAGGTCAGAGT | 1813 |
| 86 | TTATAAGATTAATGTCGGTCAGTGTCGGA | 1814 |
| 87 | CGTCTCGATGGATCCACACGAACCTGTTG | 1815 |
| 88 | ATGCCATCATGGTCGTCCTATCTTAAGGC | 1816 |
| 89 | GCGCTTCAGCGATTCGTCATGCAAGGCAC | 1817 |
| 90 | CCAAGCGATACCGAGGTACGGTTAACGAG | 1818 |
| 91 | ATATGACAGACAGGTGGACCTAAGCAAGC | 1819 |
| 92 | CACTACATCGTCAGGCCTGGAAGCCTCAG | 1820 |
| 93 | GCCGTGTAGACGAGGACATTATGTCGTAT | 1821 |
| 94 | CAACGTATATACACACCTTGTGAAGAGAA | 1822 |
| 95 | TCCAACGTAATTCCGCCGTCTGTCGAGAC | 1823 |
| 96 | AATTCGTGCTTCGATCACCGTAGACTCAG | 1824 |

TABLE 20
Random primer list (30-nucleotide)

| No. | Primer sequence | SEQ ID NO: |
|---|---|---|
| 1 | ACTATATTGTATTCACGTCCGACGACTCGC | 1825 |
| 2 | GACGAGCTTGTGGTACACTATACCTATGAG | 1826 |
| 3 | TGATTCAAGCACCAGGCATGCTTAAGCTAG | 1827 |
| 4 | CGGTCTCCTATAGGAAGGCTCATTCTGACG | 1828 |
| 5 | AGTCAGTGTCGAATCAATCAAGGCGTCCTT | 1829 |
| 6 | CGAACGTAATGGCCATCACGCGCTGGCCTA | 1830 |
| 7 | CGAACCTGGACCACCTGGCATTACCATTAC | 1831 |
| 8 | ACATTAGGTTCCTGTAATGTCTTATCAACG | 1832 |
| 9 | CGTCTAATGCACCGTATCGTCTTCGCGCAT | 1833 |
| 10 | TCTATGACTTACAACGGAATCTTACTTCGT | 1834 |
| 11 | GTAACCGATCGGTACCGTCTGCTATTGTTC | 1835 |
| 12 | GGTGATTGATAAGCAACACATATTAGGAGG | 1836 |
| 13 | AATTATCGACGCTAATAGGCGAGCTGTTCA | 1837 |
| 14 | GGAGGTACATGACGAGTGGACAGACAGACC | 1838 |
| 15 | CTCTAATCCGTTATGCGGTGATGTAATCCG | 1839 |
| 16 | GCAAGCACGCGGCTTGGCGAACTTCTATGC | 1840 |
| 17 | TAGATGTAGGCCTGGTAGGCAGAGGAGTAA | 1841 |
| 18 | CCGAGTGGCGACCACACAGGTACGCATTAA | 1842 |
| 19 | GTCCTGGCTCAGATTAGTGCACTTAGTTAT | 1843 |
| 20 | GCGGTACCTACATGTTATGACTCAGACGAC | 1844 |
| 21 | TCTCTGCCAATGCTGGTCTCATCGAATCCA | 1845 |
| 22 | TCTCTACACAGCTACATACTATACTGTAAC | 1846 |
| 23 | TACGACGGACGCTGGTGGTGTAAGAGAAGG | 1847 |
| 24 | GCCTCGATATATCTACGTATAGTTCAAGTT | 1848 |
| 25 | GGCTCCTGCATTCATTGAAGGTCGGCCTTG | 1849 |
| 26 | CAGTTCGGTGATTCAAGAGAACAATGGTGG | 1850 |
| 27 | TATAACGAAGCCGGCTGGAACGGTAACTCA | 1851 |
| 28 | CTGTATCAATTCAAGTGACAGTGGCACGTC | 1852 |
| 29 | AGCAATTGCGGTTCATAGGCGTAATTATAT | 1853 |
| 30 | CATATGGACCTGGAGATCACCGTTCAGTCC | 1854 |
| 31 | GAAGGCCGTTGGTCTATCTCTTACTGGAGC | 1855 |
| 32 | GTGCGTTCATCTAGCCTAAGACGCTGACCT | 1856 |
| 33 | GAGTAACTTATATCCTCTCTACGACATCGA | 1857 |
| 34 | ATTCTACGCTGATGTCTCCGCTGAACAGGA | 1858 |
| 35 | TCATCAACGTTACTCACTAGTACCACGGCT | 1859 |
| 36 | AACCATTCTTGAACGTTGAGAACCTGGTGG | 1860 |
| 37 | ACGACACCTCCGCGGAACATACCTGATTAG | 1861 |
| 38 | GCGCACTTATTGAAGTAATCTCATGGCCAA | 1862 |
| 39 | GCGCCAATTCAGCCAGTTAGCGTCTCCGTG | 1863 |
| 40 | AGCAACAAGTCGCTGTATATCGACTGGCCG | 1864 |
| 41 | CCTTACAATAGACCTCGCGGCGTTCATGCC | 1865 |
| 42 | GGATCCAACTTCAGCGAAGCACCAACGTCG | 1866 |
| 43 | GCGCCAGTTCTCGTACTCTCGAGAAGCGAC | 1867 |
| 44 | GAGTGCGGCCAATCTGGAACTCATGACGTT | 1868 |
| 45 | CCTGAGAGTGATTCGTGTCTGCGAAGATGC | 1869 |
| 46 | GTGACTGGTTAAGGCAATATTGGTCGACCG | 1870 |
| 47 | CTATCAAGCCTTACAAGGTCACGTCCACTA | 1871 |
| 48 | ACTGCGTCCTTGCGTCGGAACTCCTTGTGT | 1872 |
| 49 | TGCAACTCAGTGGCGGCGACACCAAGAGCT | 1873 |
| 50 | TTCGGTTCTACTAGGATCTCTATCTGAGCT | 1874 |
| 51 | AGCTAATCTATTAAGACAGATTAGACAGGA | 1875 |
| 52 | GGACCGCTCTTAGGTTATGCACCTGCGTAT | 1876 |
| 53 | CTCTAATACTAGTCCACAGGTTAGTACGAA | 1877 |
| 54 | ATCCATATATGCTCGTCGTCAGCCAGTGTT | 1878 |
| 55 | GCTATTACTGTGTTGATGTCCACAGGAGAA | 1879 |
| 56 | GCTACGGCGCAGATCTAGACAACTGGAAGT | 1880 |
| 57 | GCCTCTTGTGTTAGCCGAATACCAATGACC | 1881 |
| 58 | TGAGGACGATAACATTACCTCTCGAGTCGC | 1882 |
| 59 | CGATTACCAATCCGACGACTTCGCAGCAGC | 1883 |
| 60 | ATGACACGAGTCCAGTACATATGCGAAGAC | 1884 |
| 61 | GCGCTCGCATGCACTAGTGTAGACTGACGA | 1885 |
| 62 | GCACATCTCAGAATTGATGGTCTATGTCGC | 1886 |
| 63 | TTCTTCGACGCCGCGTACTAATAGGTCAAT | 1887 |
| 64 | GGAAGCGCCTCTAACAACCGATGCTTGTGG | 1888 |
| 65 | CTCTAGACGCGTCGTGACTCCAATCTGTTG | 1889 |
| 66 | GTAGTTCGTCGGAGTGACCTCGTACTCACT | 1890 |
| 67 | ATGCTGTCGAGTGTCCGGCATAGAGCACAC | 1891 |
| 68 | GCGCATCTTGCAGCGTCCTGTAGTTCTGAA | 1892 |
| 69 | GCGATTGTTGAGGAACCACAGCGGCACCTA | 1893 |
| 70 | CACGCGTACTCTGCTTGCTGTGTGGTCGGT | 1894 |
| 71 | CATCCAACGCAGGACCTAGTAGTCATGCTT | 1895 |
| 72 | TTCTAGTTGTGATGAGAATCGCTAGCGTGC | 1896 |
| 73 | CATTCTGAATCTGGTCTCTCTCGATCATCC | 1897 |
| 74 | ATTAATGTAGAGGATAGTTCCGTTCTCTCC | 1898 |
| 75 | GTATCGCGCTTACGAATGAGGTGTGGCTTC | 1899 |
| 76 | GCTGGTGAGAGAGCCAGATTATCGGTGGAG | 1900 |
| 77 | GGCACGAGCAGGTAGAACTAGAACCTAGAT | 1901 |
| 78 | TGTATTATCTCGAAGCGGTGCGTTAGAGTC | 1902 |
| 79 | CACGTGTTCTAGCTACTAATGGCGTCAATT | 1903 |
| 80 | CGCGCTACATTACTTCCTACACCATGCGTA | 1904 |
| 81 | TGAGGCAACTAGTGTTCGCAAGATGACGGA | 1905 |
| 82 | TTATTATTGTCTGTGGAACGCACGCCAGTC | 1906 |
| 83 | GCTATAGTATTATCCATGAATTCCGTCGGC | 1907 |
| 84 | GTATCAATAGCTCAATTCGTCAGAGTTGTG | 1908 |
| 85 | TAGTCCATGCGTGGATATATTGAGAGCTGA | 1909 |
| 86 | GCACAGTACGACTTATAACAGGTCTAGATC | 1910 |
| 87 | ACTCAATGGTGGCACGCTCGGCGCAGCATA | 1911 |
| 88 | GTAGTACCACTCCGCCTTAGGCAGCTTAAG | 1912 |
| 89 | CGCTCAACTGATGCGTGCAACCAATGTTAT | 1913 |
| 90 | GCAGCTTGACTGCCTAGACAGCAGTTACAG | 1914 |
| 91 | GCAACTTCTTAGTACGAATTCATCGTCCAA | 1915 |
| 92 | ATCCGTATGCTGCGGCAGTGGAGGTGGCTT | 1916 |
| 93 | TGCGGATCAATCCAGTTCTGTGTACTGTGA | 1917 |
| 94 | TTATGATTATCACCGGCGTAACATTCCGAA | 1918 |
| 95 | GCTACCTAGATTCTTCAACTCATCGCTACC | 1919 |
| 96 | CAGTGTTAGAATGGCGGTGTGTAGCCGCTA | 1920 |

TABLE 21
Random primer list (35-nucleotide)

| No. | Primer sequence | SEQ ID NO: |
|---|---|---|
| 1 | GCTTATAGACTACAGCTGCGAGGTATAAGGTCACT | 1921 |
| 2 | CGCTCAGCAGGATGCTATCCTAAGTTAATGTGGTG | 1922 |
| 3 | GAACTGAGCGGACATCAGCTAGGCCTACAATACAT | 1923 |
| 4 | TCGTGAACTTCTGCGTTGGTCTCTACCAAGGCGGT | 1924 |
| 5 | TAAGTCAGGTATCTTATCAGTGGTACACGGTACGA | 1925 |
| 6 | TAATAATGTTGCGCGTGACCGAGGAGGAATCCACT | 1926 |
| 7 | CTAGGAGTTCTCGTAAGCTGGAGTACCGTAACGTG | 1927 |
| 8 | GGACTCTCCTCAGAGGATCCTTCTTGCGCAGGCAT | 1928 |
| 9 | GCTAGAGGCCTGAGTACACCTTCTCGCATCAGGAT | 1929 |
| 10 | ATATCGCGAGCACTAACGTCGTTGTCGTTCTAGGA | 1930 |
| 11 | AGCGGTTACTATACCTGGCGGCTGACGTTGTTAGT | 1931 |
| 12 | GAGCTAGGTAGATCTCCAAGTGTAGCTAAGAAGAG | 1932 |
| 13 | GGAGTCGCTGGTGACGTATGCCGAGGATGAGCTTC | 1933 |
| 14 | CGCCGACCTCCTGTTCACGAAGCCGCCTGATGTAA | 1934 |
| 15 | AGTAGGCACTTAGTTATCGATTACGTTAGTTAGTC | 1935 |
| 16 | GGATGACGTCTCAGTCTACCTCGCAGTGTCGTCTA | 1936 |
| 17 | CTGGTTCGCGTTAGCAATACTAAGGCAGTCAGGAG | 1937 |
| 18 | ATATGGTCATATTGGCCTCTTCGAACACAGACTGT | 1938 |
| 19 | TATCAGAGGATAGCAGGTCTGAGTTGCAAGGCTAA | 1939 |
| 20 | GGTGGTCTGACCATAGCTGTTCTTCTCACAGAGAC | 1940 |
| 21 | GCAATACCAACGAGATGAGTATTCGTTGAAGCTCT | 1941 |
| 22 | CCAAGTCGACGCTGCATGAATGAGCGCTATTCACT | 1942 |
| 23 | CCATTAGATCGCTTCGAGACAATTAGGAGACATGA | 1943 |
| 24 | GATGACTGTACCTCCTATCATTGAGTGTGGACCAA | 1944 |
| 25 | ATATCTGGATGAATAGTGGTTAGGTAAGCAAGTAA | 1945 |
| 26 | ACCGACTATGTTAATTCGTGTCTGGATGGCAGAAT | 1946 |
| 27 | GTGGCAGTCTTGCTAGTATCTTAGACCATCACCAA | 1947 |
| 28 | CGCTATCTTAGTCGAGCACAATGTCTTCGTATAGG | 1948 |
| 29 | ATTAGTACGGCACGAACCGGCCATTCATGGCAGCT | 1949 |
| 30 | AGTACGACTATCAAGACTCCAGCGCTCTCCTTGGA | 1950 |
| 31 | ATGAGCCTCGGAGCGAACGTTATCGATCAGGCTGT | 1951 |
| 32 | TTGCGTGCAGTAGCACCGATACACAGCGCTTGTAT | 1952 |
| 33 | AACGGCTGCATCACCTACACTATACTCAACATCTA | 1953 |
| 34 | GTCGCTATGCGAGAAGTGGCGTGGAATGCTATGGT | 1954 |
| 35 | CATGGATACCTACTGACTTGACTTCTAGAGGACCG | 1955 |
| 36 | GAGTGACGCAGACACCGTAACGTCGAATCTTCTAG | 1956 |
| 37 | AGTACCGTCTGTGTGAATATTGTTCCTACGTTACA | 1957 |
| 38 | GGCTAATCGATAGTGACGAGTTCTGCACGCCTGAA | 1958 |
| 39 | GGCGAGCGCTCGTGGTTCTGAGTCGCTGTTAGATG | 1959 |
| 40 | TATCTCCAGCGTTATAAGCTACTGGAGCCGCTCGG | 1960 |
| 41 | CCTTCTGCGCAAGTCAAGGATTCGCTTAGATGGAC | 1961 |
| 42 | GTTGCTGACAGCCGTTGCGTACTTGCCTTAAGAAC | 1962 |
| 43 | GTGGCCTAATCACTCGCGCTTCATAGGCCGATAGG | 1963 |
| 44 | TGCATCTAGCCTACATCGGACCTTGTTATGGTAAT | 1964 |
| 45 | GGACAGCTACTGGACACCACCGAACTGGTAGTGTC | 1965 |
| 46 | AACTGGCGATGGACGGCCGCTCTTCCGCTACATAG | 1966 |
| 47 | GGAGCAGTTAGCTATGGAGCAGGCCGATAACCTGA | 1967 |
| 48 | ACTCTACGGTGCACCTCAGCCTTCATGCAATAGGC | 1968 |
| 49 | CTTGTAGCACAATACATTACTCTCCACGTGATAGC | 1969 |
| 50 | GGACGCTATCGATACCGTTATTCCTACTCTGTCGG | 1970 |
| 51 | GGATGATCGTCAACGATCAACTGACAGTTAGTCGA | 1971 |
| 52 | TGACAGTAGCAATGTCTCACGTCTGCACAACGGAA | 1972 |
| 53 | GTCGCAGGACCTCACGGATAGTAGTGCGAGGTCTA | 1973 |
| 54 | ATATCGGCGGACGCAATGACAGTTGTTGGCTGATG | 1974 |
| 55 | AAGCACCAAGGAGGTATGTTCCATCGAGGCGCTCG | 1975 |
| 56 | GACCGCACCTTATAGCTATATCCTGGTCTAGTACT | 1976 |
| 57 | TCTCAGAGGAAGGTTGAGCGTCTGACCAGGTTGGC | 1977 |
| 58 | TGGACCTAGAGACCTAGCTCGTCTCTTCGCGATCG | 1978 |
| 59 | CGGAGTGGTTCCACGCGACCTCGCAACTAATCCTT | 1979 |
| 60 | GGAGCCGCGCGCAGACTGACCTTGCTTGATCTACT | 1980 |
| 61 | ACTCTAAGTATATGCGCAGTTAGTATACTGAACCA | 1981 |
| 62 | GAGCATTGCTTCGCTTCGATGTCTATTCTGATCAG | 1982 |
| 63 | GCTTGTATTGCCACTCGAGTAGGTCGTGGCAGTAG | 1983 |
| 64 | ATCTGGACATTGCATTCGGTGTGTATACAGAAGGC | 1984 |
| 65 | GGTTGCGATCAGCTTGATAGCAGGTCATATCCTCA | 1985 |
| 66 | GCAGGTACTAACCTGAGATGCGTAGCTAACACAGG | 1986 |
| 67 | ATCTGCAAGGACGTAACGTCCTCGGAAGGTGAGGT | 1987 |
| 68 | ATAATCTTACGAGCCTCCAGTGAATAATGCAAGCA | 1988 |
| 69 | CAATCTCCGCACAGTCTTGTTCAGGTACAGACTTA | 1989 |
| 70 | ATGTGCGCAATTCAGCGTAAGTGCCTATTCATAAT | 1990 |
| 71 | TCGGACGCACACATCCTGTTGTCGAGAAGAGGAAG | 1991 |
| 72 | TCGGAAGCATCACATGAGCATCAGGAGTTCATTGC | 1992 |
| 73 | ATCTGGTTGTGGACTTCTATACAGTACCAGAGTGG | 1993 |
| 74 | CGTCTGAATATAGTTAGCTAGTAGTGTAATCCAGG | 1994 |
| 75 | TAATATCTGATCCGACCTATTATCTAGGACTACTC | 1995 |
| 76 | TATGCGGCCGTCCGTACCTCGTCTGCTTCAGTTGG | 1996 |
| 77 | TGGCTCAAGTTCCATATTGCCAAGACGACCTGGAG | 1997 |
| 78 | GCAGTTCTGCTAGGCGGTCCGAGGCAATTGAAGAG | 1998 |
| 79 | CATGGCACAGACGAAGTATGCACCACGCTCATTAA | 1999 |
| 80 | GGAGCGTACTACGACCATTCAACCGAATATGTTAC | 2000 |
| 81 | GCGTAGATCTCGCGACAGAGACAAGGTGCGAATGG | 2001 |
| 82 | TGGACTGAGGTTCTCCGGTCTATACTCCTGTAGGA | 2002 |
| 83 | TGGCTATAGCAACGGCTTCTTGTGATCGCATTGCA | 2003 |
| 84 | GGCGAAGAATCATGCGAGACGGAGTAGACGGACGT | 2004 |
| 85 | GAGCATTGCGAGTTGCACACGTGATATCAGACTGT | 2005 |
| 86 | CTGTTGACCTATGCCAGAATCAATACCTCAGATTA | 2006 |
| 87 | GTTAACAAGTAGATGCCAAGATACAACGAGAGACC | 2007 |
| 88 | GAGCAAGATTATAGTTAGGAAGATAGTTAACTCGC | 2008 |
| 89 | TCCGGAGTCGAGCATATGTGACCAACTCTCAACGC | 2009 |
| 90 | GGAGCTGCGATGCCGTTACCGACGTCATCTTCAAG | 2010 |
| 91 | GCTCTATCTTACACATTGGCGTACTGGACTCGCGA | 2011 |
| 92 | TTCTACATATTCATCGCCTACCGAGTTGCGCGAAG | 2012 |
| 93 | TGGACGTCTGACCTGTGTCTACATCGGTGGTGCTA | 2013 |
| 94 | GGCAGGACAGCTCCGTGTTCTACTCGAACCGCACT | 2014 |
| 95 | TGACAACCTCATGTCTCCGACCGCAGGCATACAAT | 2015 |
| 96 | GCAGGCCTAACAAGTGGTCACGAGGAGTCCTTATT | 2016 |

### 3.1.2 Standard PCR

To the genomic DNA described in 2, above (30 ng, NiF8-derived genomic DNA), random primers (final concentration: 0.6 μM; 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. In this example, numerous nucleic acid fragments obtained via PCR using random primers, including the standard PCR described above, are referred to as a DNA library.

### 3.1.3 Purification of DNA Library and Electrophoresis

The DNA library obtained in 3.1.2 above was purified with the use of the MinElute PCR Purification Kit (QIAGEN) and subjected to electrophoresis with the use of the Agilent 2100 bioanalyzer (Agilent Technologies) to obtain a fluorescence unit (FU).

### 3.1.4 Examination of Annealing Temperature

To the genomic DNA described in 2, above (30 ng, NiF8-derived genomic DNA), random primers (final concentration: 0.6 μM, 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, different annealing temperatures for 15 seconds, and 72° C. for 20 seconds, followed by storage at 4° C. In this Example, 37° C., 40° C., and 45° C. were examined as annealing temperatures. The DNA library obtained in this experiment was subjected to purification and electrophoresis in the same manner as in 3.1.3.

### 3.1.5 Examination of Enzyme Amount

To the genomic DNA described in 2, above (30 ng, NiF8-derived genomic DNA), random primers (final concentration: 0.6 μM, 10-nucleotide primer A), a 0.2 mM dNTP mixture, 1.0 mM MgCl₂, and 2.5 units or 12.5 units of DNA polymerase (PrimeSTAR, TAKARA) were added, and a reaction solution was prepared while adjusting the final reaction level to 50 pd. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3.

### 3.1.6 Examination of MgCl₂ Concentration

To the genomic DNA described in 2, above (30 ng, NiF8-derived genomic DNA), random primers (final concentration: 0.6 μM, 10-nucleotide primer A), a 0.2 mM dNTP mixture, MgCl₂ at a given concentration, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. In this Example, two-, three- and four-fold concentrations of a usual concentration were examined as MgCl₂ concentrations. The DNA library obtained in this experiment was subjected to purification and electrophoresis in the same manner as in 3.1.3.

### 3.1.7 Examination of Nucleotide Length of Random Primer

To the genomic DNA described in 2, above (30 ng. NiF8-derived genomic DNA), random primers (final concentration: 0.6 μM), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. In this Example, primers having 8 nucleotides (Table 7), 9 nucleotides (Table 8), 11 nucleotides (Table 9), 12 nucleotides (Table 10), 14 nucleotides (Table 11), 16 nucleotides (Table 12), 18 nucleotides (Table 13), and 20 nucleotides (Table 14) were examined as random primers. The DNA library obtained in this experiment was subjected to purification and electrophoresis in the same manner as in 3.1.3.

### 3.1.8 Examination of Random Primer Concentration

To the genomic DNA described in 2, above (30 ng, NiF8-derived genomic DNA), random primers at a given concentration (10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. In this Example, 2, 4, 6, 8, 10, 20, 40, 60, 100, 200, 300, 400, 500, 600, 700, 800, 900, and 1000 μM were examined as random concentrations. The DNA library obtained in this experiment was subjected to purification and electrophoresis in the same manner as in 3.1.3. Also, in this experiment, the reproducibility of the repeated data was evaluated on the basis of the Spearman's rank correlation (p>0.9).

### 3.2 Verification of Reproducibility Via MiSeq

### 3.2.1 Preparation of DNA Library

To the genomic DNA described in 2, above (30 ng, NiF8-derived genomic DNA), random primers (final concentration: 60 μM, 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3.

### 3.2.2 Preparation of Sequence Library

From the DNA library obtained in 3.2.1, a sequence library for MiSeq analysis was prepared using the KAPA Library Preparation Kit (Roche).

### 3.2.3 MiSeq Analysis

With the use of the MiSeq Reagent Kit V2 500 Cycle (Illumina), the sequence library for MiSeq analysis obtained in 3.2.2 was analyzed via 100 base paired-end sequencing.

### 3.2.4 Read Data Analysis

Random primer sequence information was deleted from the read data obtained in 3.2.3, and the read patterns were identified. The number of reads was counted for each read pattern, the number of reads of the repeated analyses, and the reproducibility was evaluated using the correlational coefficient.

### 3.3 Analysis of Rice Variety Nipponbare

### 3.3.1 Preparation of DNA Library

To the genomic DNA described in 2, above (30 ng, Nipponbare-derived genomic DNA), random primers (final concentration: 60 μM, 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3.

### 3.3.2 Preparation of Sequence Library, MiSeq Analysis, and Read Data Analysis

Preparation of a sequence library using the DNA library prepared from Nipponbare-derived genomic DNA, MiSeq analysis, and analysis of the read data were performed in accordance with the methods described in 3.2.2, 3.2.3, and 3.2.4. respectively.

### 3.3.3 Evaluation of Genomic Homogeneity

The read patterns obtained in 3.3.2 were mapped to the genomic information of Nipponbare (NC_008394 to NC_008405) using bowtie2, and the genomic positions of the read patterns were identified.

### 3.3.4 Non-Specific Amplification

On the basis of the positional information of the read patterns identified in 3.3.3, the sequences of random primers were compared with the genome sequences to which such random primers would anneal, and the number of mismatches was determined.

### 3.4 Detection of Polymorphism and Identification of Genotype

### 3.4.1 Preparation of DNA Library

To the genomic DNA described in 2, above (30 ng, NiF8-derived genomic DNA, Ni9-derived genomic DNA, hybrid progeny-derived genomic DNA, or Nipponbare-derived genomic DNA), random primers (final concentration: 60 μM, 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3.

### 3.4.2 HiSeq Analysis

Analysis of the DNA libraries prepared in 3.4.1 was consigned to TakaraBio under conditions in which the number of samples was 16 per lane via 100 base paired-end sequencing, and the read data were obtained.

### 3.4.3 Read Data Analysis

Random primer sequence information was deleted from the read data obtained in 3.4.2, and the read patterns were identified. The number of reads was counted for each read pattern.

### 3.4.4 Detection of Polymorphism and Identification of Genotype

On the basis of the read patterns and the number of reads obtained as a results of analysis conducted in 3.4.3. polymorphisms peculiar to NiF8 and Ni9 were detected, and the read patterns thereof were designated as markers. On the basis of the number of reads, the genotypes of the 22 hybrid progeny lines were identified. The accuracy for genotype identification was evaluated on the basis of the reproducibility attained by the repeated data concerning the 22 hybrid progeny lines.

3.5 Experiment for Confirmation with PCR Marker

### 3.5.1 Primer Designing

Primers were designed for a total of 6 markers (i.e., 3 NiF8 markers and 3 Ni9 markers) among the markers identified in 3.4.4 based on the marker sequence information obtained via paired-end sequencing (Table 22).

TABLE 22
Marker sequence information and PCR marker primer information

| Genotype | Marker name | Marker sequence (1)* | Marker sequence (2)* |
|---|---|---|---|
| NiF8 type | N80521152 | CCCATACACACACCATGAAGCTTGAACTA | ATGGGTGAGGGCGCAGAGGCAAAGACAT |
| | | ATTAACATTCTCAAACTAATTAACAAGCAT | GGAGGTCCGGAAGGGTAGAAGCTCACAT |
| | | GCAAGCATGTTTTTACACAATGACAATATAT | CAAGTCGAGTATGTTGAATCCAATCCCATA |
| | | (SEQ ID NO: 2017) | TATA |
| | | | (SEQ ID NO: 2018) |
| | N80987192 | AATCACAGAACGAGGTCTGGACGAGAAC | GATGCTGAGGGCGAAGTTGTGAGCCAAG |
| | | AGAGCTGGACATCTACACGCACCGCATG | TCCTCAATGTCATAGGCGAGATCGCAGTA |
| | | GTAGTAGAGCATGTACTGCAAAAGCTTGA | GTTCTGTAACCATTCCCTGCTAAACTGGT |
| | | AGCGC | CCAT |
| | | (SEQ ID NO: 2021) | (SEQ ID NO: 2022) |
| | N80533142 | AGACCAACAAGCAGCAAGTAGTCAGAGA | GGAGGAGCACAACTAGGCGTTTATCAAGA |
| | | AGTACAAGAGAAGGAGAGGAAGAAGGAT | TGGGTCATCGAGCTCTTGGTGTCTTGAAC |
| | | AGTAAGTTGCAAGCTTACCGTTACAAAGA | CTTCTTGACATCAACTTCTCCAATCTTCGT |
| | | TGATA | CT |
| | | (SEQ ID NO: 2025) | (SEQ ID NO: 2026) |
| Ni9 type | N91552391 | TGGGGTAGTCCTGAAGCTCTAGGTATGCC | GGATAGTGATGTAGCTTTCACCCGGGAGT |
| | | TCTTCATCTCCCTGCACCTCTGGTGCTAG | ATTCGAAGGTATCGATTTTCCACGGGGAA |
| | | CACCTCCTGCTCTTCGGGCACCTCTACC | CGCGAAGTGCACTAGTTGAGGTTTAGATT |
| | | GGGG | GCC |
| | | (SEQ ID NO: 2029) | (SEQ ID NO: 2030) |
| | N91653962 | TCGGGAAAACGAACGGGCGAACTACAGA | AGCAGGAGGGAGAAAGGAAACGTGGCAT |
| | | TGTCAGTACGAAGTAGTCTATGGCAGGAA | TCATCGGCTGTCTGCCATTGCCATGTGAG |
| | | ATACGTAGTCCATACGTGGTGCCAGCCCA | ACAAGGAAATCTACTTCACCCCCATCTATC |
| | | AGCC | GAG |
| | | (SEQ ID NO 2033) | (SEQ ID NO: 2034) |
| | N91124801 | AGACATAAGATTAACTATGAACAAATTGAC | TTAAGTTGCAGAATTTGATACGAAGAACTT |
| | | GGGTCCGATTCCTTTGGGATTTGCAGCTT | GAAGCATGGTGAGGTTGCCGAGCTCATT |
| | | GCAAGAACCTTCAAATACTCATTATATCTT | GGGGATGGTTCCAGAAAGGCTATTGTAG |
| | | (SEQ ID NO: 2037) | CTTA |
| | | | (SEQ ID NO: 2038) |

| Genotype | Marker name | Primer (i) | Primer (2) |
|---|---|---|---|
| NiF8 type | N80521152 | CCCATACACACAC | GGTAGAAGCTCAC |
| | | CATGAAGCTTG | ATGAAGTCGAG |
| | | (SEQ ID NO: 2019) | (SEQ ID NO: 2020) |
| | N80987192 | ACGAGAACAGAGC | TCAATGTCATAGGC |
| | | TGGACATCTAC | GAGATCGCAG |
| | | (SEQ ID NO: 2023) | (SEQ ID NO: 2024) |
| | N80533142 | GGAGAGCAAGAAG | CGAGCTCTTGGTG |
| | | GATAGTAAGTTGC | TCTTCAACCTTC |
| | | (SEQ ID NO: 2027) | (SEQ ID NO: 2028) |
| Ni9 type | N91552391 | GAAGCTCTAGGTA | GTGCACTAGTTGA |
| | | TGGCTCTTCATC | GGTTTAGATTGC |
| | | (SEQ ID NO: 2031) | (SEQ ID NO: 2032) |
| | N91653962 | GGGCGAACTACAG | CTGTGTGCCATTG |
| | | ATGTCAGTACG | CCATGTGAGAC |
| | | (SEQ ID NO: 2035) | (SEQ ID NO: 2036) |
| | N91124801 | GAACAAATTCACG | CGAAGAACTTGAA |
| | | GGTCCGATTCC | GCATGGTGAGG |
| | | (SEQ ID NO: 2039) | (SEQ ID NO: 2040) |

*Marker sequence: Paired-end sequence

### 3.5.2 PCR and Electrophoresis

With the use of the TaKaRa Multiplex PCR Assay Kit Ver. 2 (TAKARA) and the genomic DNA described in 2, above (15 ng. NiF8-derived genomic DNA, Ni9-derived genomic DNA, or hybrid progeny-derived genomic DNA) as a template, 1.25 μl of Multiplex PCR enzyme mix, 12.5 μl of 2× Multiplex PCR buffer, and the 0.4 μM primer designed in 3.5.1 were added, and a reaction solution was prepared while adjusting the final reaction level to 25 μl. PCR was carried out under thermal cycle conditions comprising 94° C. for 1 minute, 30 cycles of 94° C. for 30 seconds, 60° C. for 30 seconds, and 72° C. for 30 seconds, and retention at 72° C. for 10 minutes, followed by storage at 4° C. The amplified DNA fragment was subjected to electrophoresis with the use of TapeStation (Agilent Technologies).

### 3.5.3 Comparison of Genotype Data

On the basis of the results of electrophoresis obtained in 3.5.2, the genotype of the marker was identified on the basis of the presence or absence of a band, and the results were compared with the number of reads of the marker.

### 3.6 Correlation Between Random Primer Density and Length

### 3.6.1 Influence of Random Primer Length at High Concentration

To the genomic DNA described in 2, above (30 ng. NiF8-derived genomic DNA), random primers having given lengths (final concentration: 10 μM), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. In this experiment, 9 nucleotides (Table 8), 10 nucleotides (Table 1, 10-nucleotide primer A), 11 nucleotides (Table 9), 12 nucleotides (Table 10), 14 nucleotides (Table 11), 16 nucleotides (Table 12), 18 nucleotides (Table 13), and 20 nucleotides (Table 14) were examined as random primer lengths. PCR was carried out under thermal cycling conditions comprising 98° C. for 2 minutes and 30 cycles of 98° C. for 10 seconds, 50° C. for 15 seconds, and 72° C. for 20 seconds, followed by storage at 4° C. In the reaction system using random primers each comprising 10 or more nucleotides, PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3.

### 3.6.2 Correlation Between Random Primer Density and Length

To the genomic DNA described in 2, above (30 ng, NiF8-derived genomic DNA), random primers of a given length were added to a given concentration therein, a 0.2 mM dNTP mixture, 1.0 mM MgCl₂, and 1.25 units of DNA polymerase (PrimeSTAR, TAKARA) were added thereto, and a reaction solution was prepared while adjusting the final reaction level to 50 μl. In this experiment, random primers comprising 8 to 35 nucleotides shown in Tables 1 to 21 were examined, and the random primer concentration from 0.6 to 300 μM was examined.

In the reaction system using random primers comprising 8 nucleotides and 9 nucleotides, PCR was carried out under thermal cycle conditions comprising 98° C. for 2 minutes and 30 cycles of 98° C. for 10 seconds, 37° C. for 15 seconds, and 72° C. for 20 seconds, followed by storage at 4° C. In the reaction system using a random primer of 10 or more nucleotides, PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3. Also, the reproducibility of the repeated data was evaluated on the basis of the Spearman's rank correlation (p>0.9).

### 3.7 Number of Random Primers

To the genomic DNA described in 2, above (30 ng, NiF8-derived genomic DNA), 1, 2, 3, 12, 24, or 48 types of random primers selected from the 96 types of random primers comprising 10 nucleotides (10-nucleotide primer A) shown in Table 1 were added to the final concentration of 60 μM therein, a 0.2 mM dNTP mixture, 1.0 mM MgCl₂, and 1.25 units of DNA polymerase (PrimeSTAR, TAKARA) were added thereto, and a reaction solution was prepared while adjusting the final reaction level to 50 μl. In this experiment, as the 1, 2, 3, 12, 24, or 48 types of random primers, random primers were selected successively from No. 1 shown in Table 1, and the selected primers were then examined. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3. Also, the reproducibility of the repeated data was evaluated on the basis of the Spearman's rank correlation (p>0.9).

### 3.8 Random Primer Sequence

To the genomic DNA described in 2, above (30 ng, NiF8-derived genomic DNA), a set of primers selected from the 5 sets of random primers shown in Tables 2 to 6 was added to the final concentration of 60 μM therein, a 0.2 mM dNTP mixture, 1.0 mM MgCl₂, and 1.25 units of DNA polymerase (PrimeSTAR, TAKARA) were added thereto, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3. Also, the reproducibility of the repeated data was evaluated on the basis of the Spearman's rank correlation (p>0.9).

### 3.9 DNA Library Using Human-Derived Genomic DNA

To the genomic DNA described in 2, above (30 ng, human-derived genomic DNA), random primers (final concentration: 60 μM, 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was subjected to purification and electrophoresis in the same manner as in 3.1.3. Also, the reproducibility of the repeated data was evaluated on the basis of the Spearman's rank correlation (p>0.9).

### 4. Results and Examination

### 4.1 Correlation Between PCR Conditions and DNA Library Size

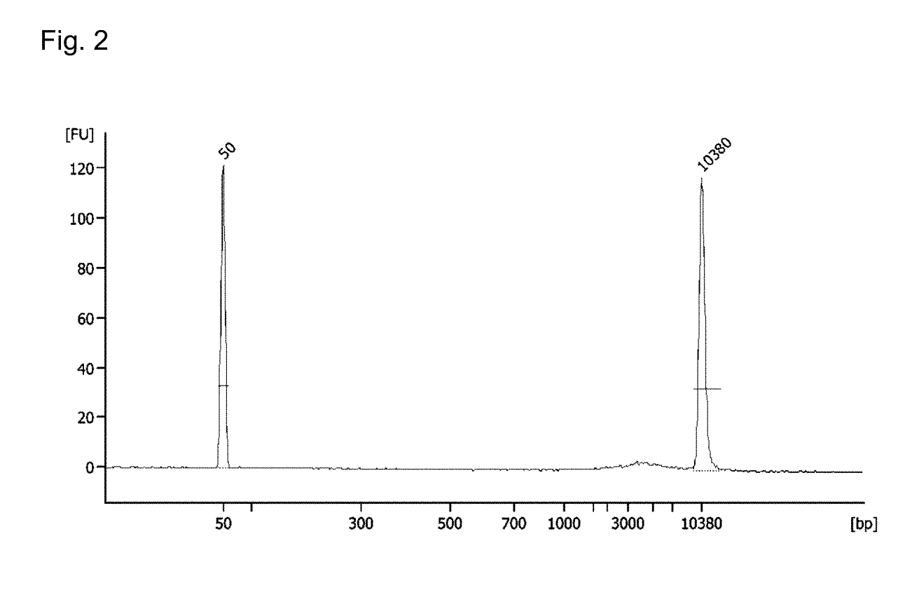

When PCR was conducted with the use of random primers in accordance with conventional PCR conditions (3.1.2 described above), the amplified DNA library size was as large as 2 kbp or more, but amplification of the DNA library of a target size (i.e., 100-bp to 500-bp) was not observed (FIG. 2). A DNA library of 100 bp to 500 bp could not be obtained because it was highly unlikely that a random primer would function as a primer in a region of 500 bp or smaller. In order to prepare a DNA library of the target size (i.e., 100 bp to 500 bp), it was considered necessary to induce non-specific amplification with high reproducibility.

The correlation between the annealing temperature (3.1.4 above), the enzyme amount (3.1.5 above), the MgCl₂ concentration (3.1.6 above), the primer length (3.1.7 above), and the primer concentration (3.18 above), which are considered to affect PCR specificity, and the DNA library size were examined.

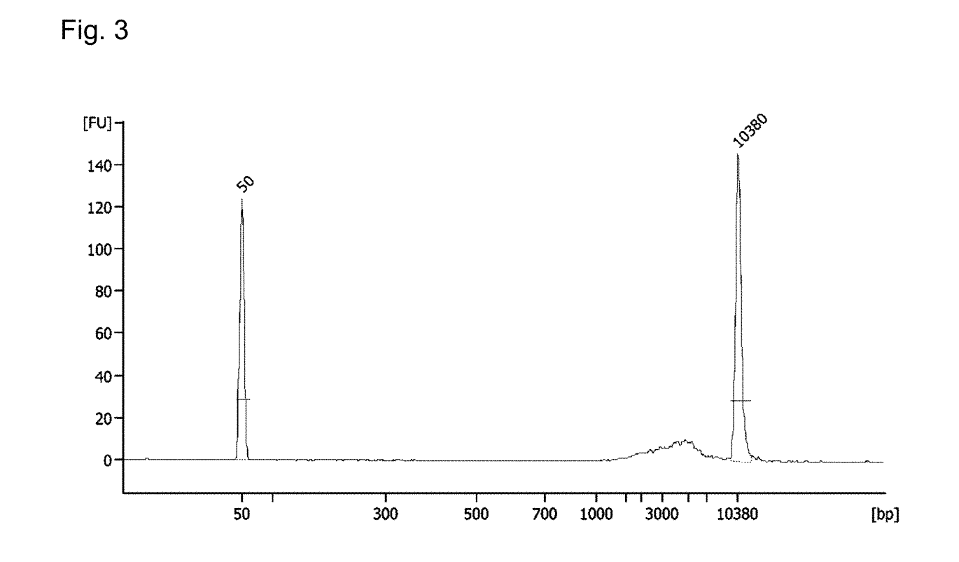

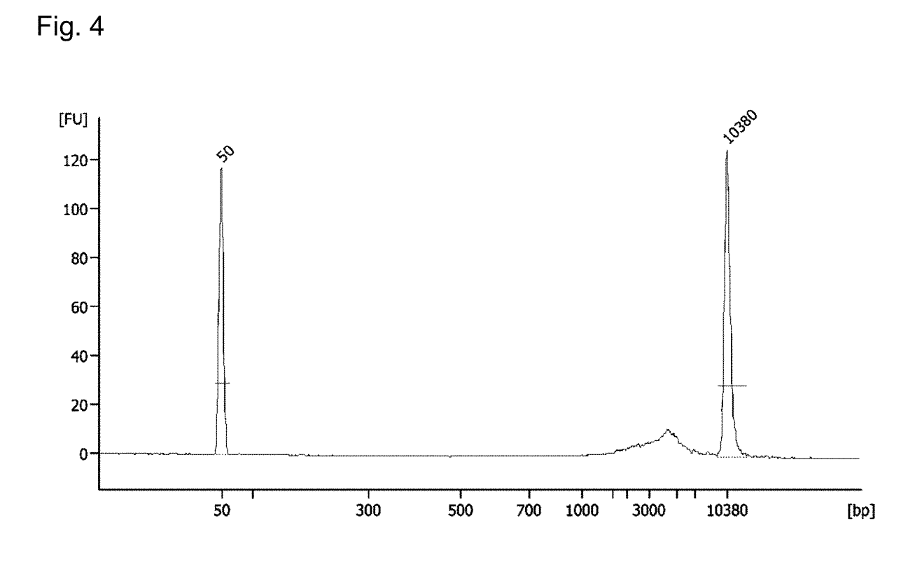

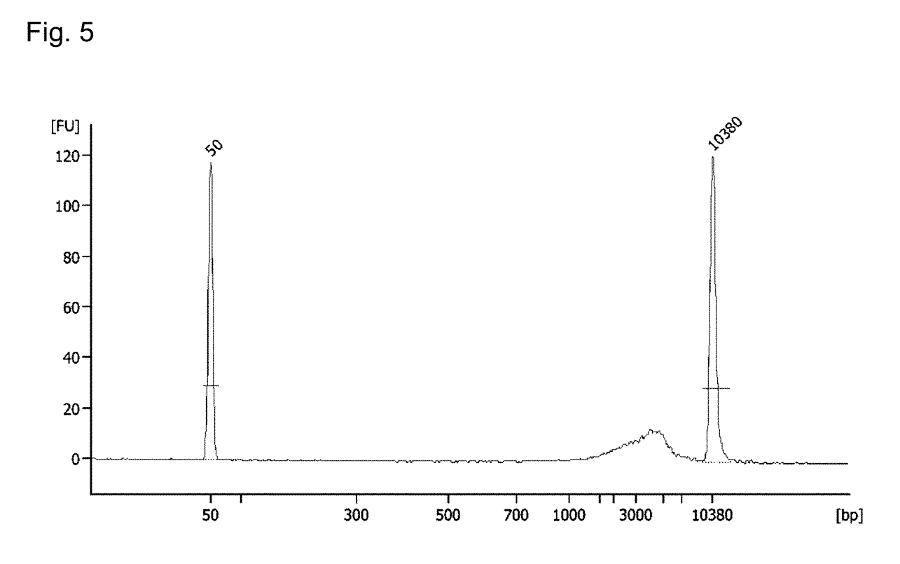

FIG. 3 shows the results of the experiment described in 3.1.4 attained at an annealing temperature of 45° C., FIG. 4 shows the results attained at an annealing temperature of 40° C., and FIG. 5 shows the results attained at an annealing temperature of 37° C. By reducing the annealing temperature from 45° C., 40° C., to 37° C., as shown in FIGS. 3 to 5, the amounts of high-molecular-weight DNA library amplified increased, although amplification of low-molecular-weight DNA library was not observed.

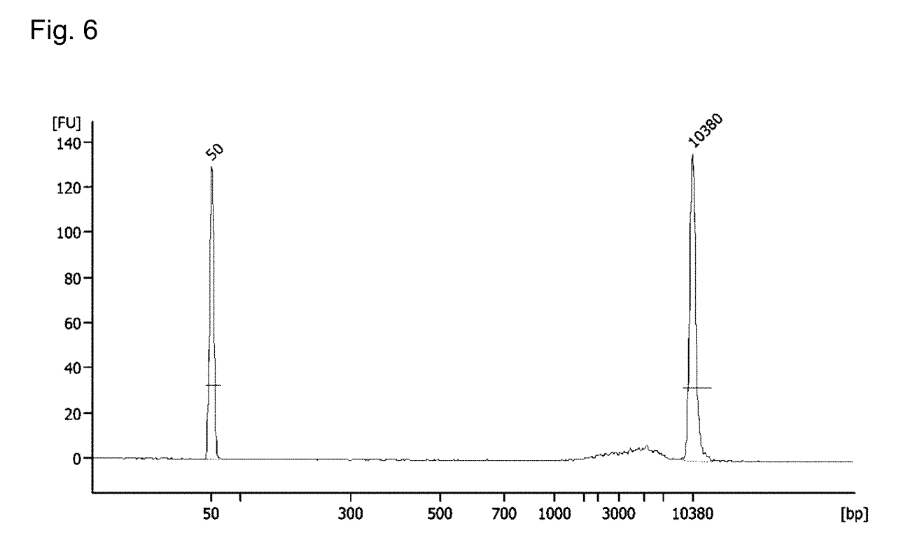

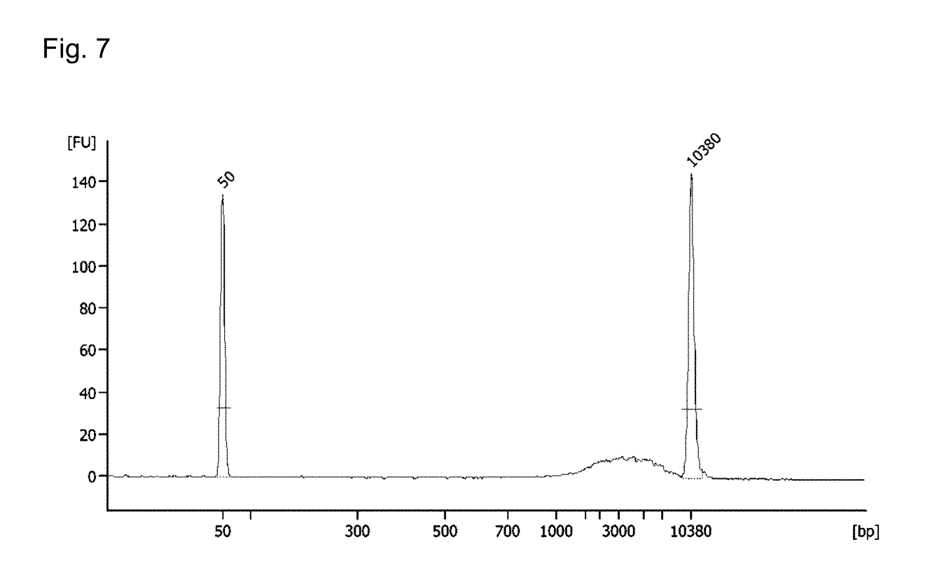

FIG. 6 shows the results of the experiment described in 3.1.5 attained when the enzyme amount is increased by 2 times, and FIG. 7 shows the results attained when the enzyme amount is increased by 10 times the original amount. By increasing the enzyme amount by 2 times or 10 times a common amount, as shown in FIGS. 6 and 7, the amounts of high-molecular-weight DNA library amplified increased, although amplification of low-molecular-weight DNA library was not observed.

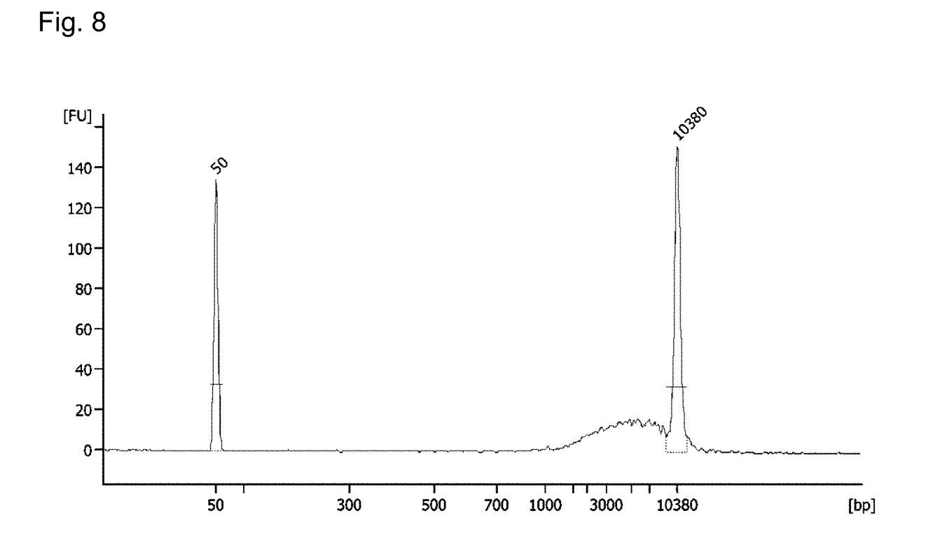

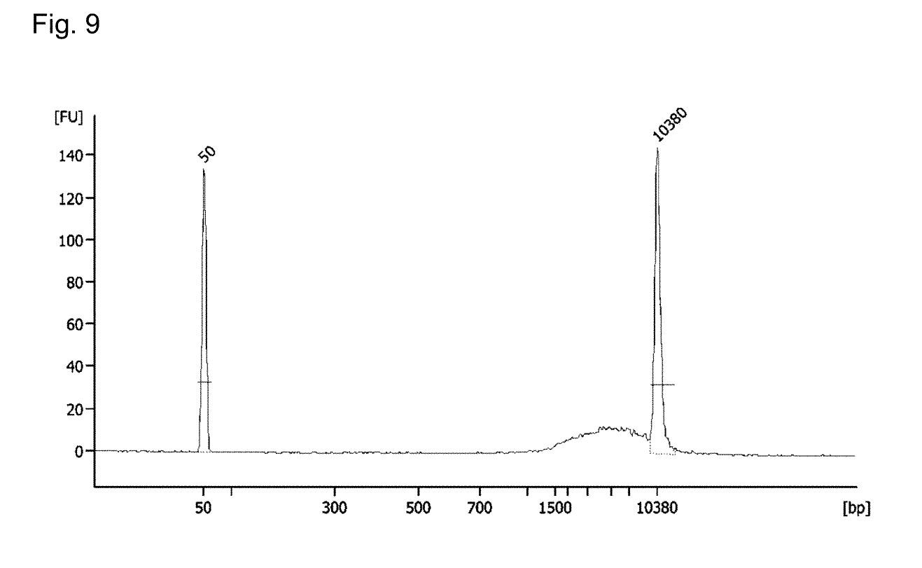

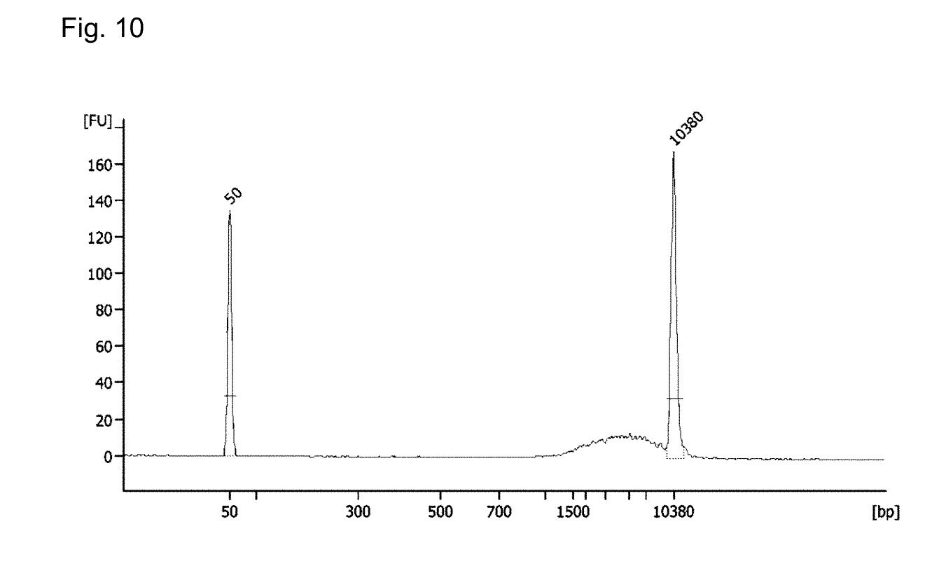

FIG. 8 shows the results of the experiment described in 3.1.6 attained when the MgCl₂ concentration is increased by 2 times a common amount, FIG. 9 shows the results attained when the MgCl₂ concentration is increased by 3 times, and FIG. 10 shows the results attained when the MgCl₂ concentration is increased by 4 times. By increasing the MgCl₂ concentration by 2 times, 3 times, and 4 times the common amount, as shown in FIGS. 8 to 10, the amounts of high-molecular-weight DNA library amplified varied, although amplification of a low-molecular-weight DNA library was not observed.

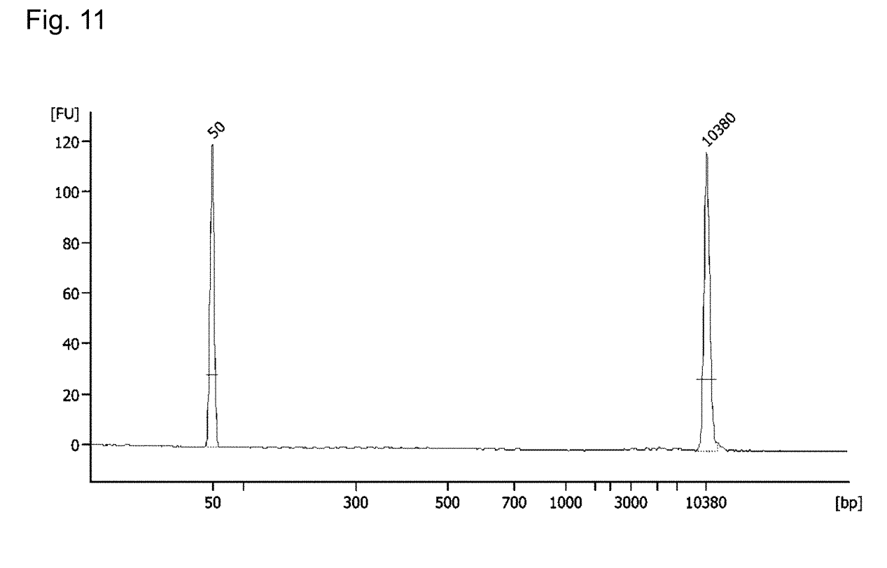

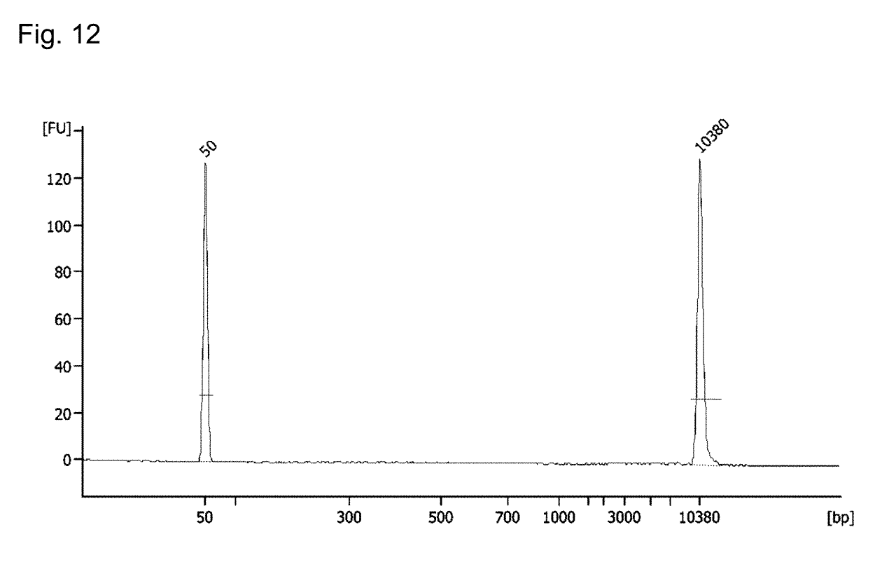

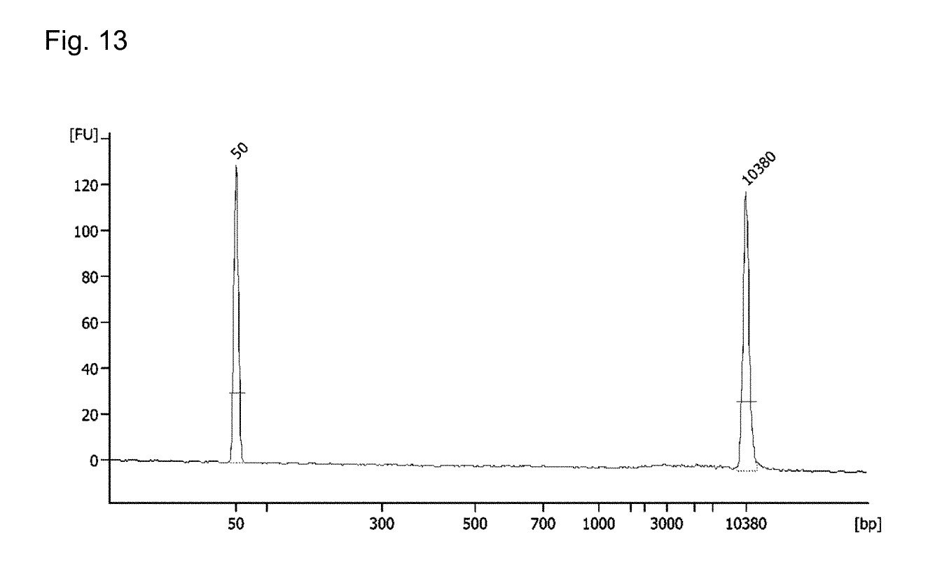

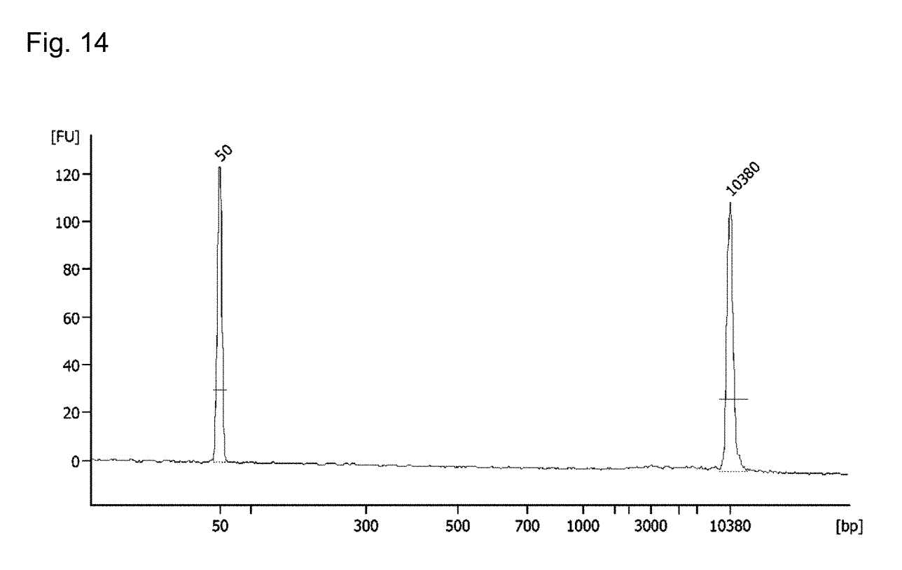

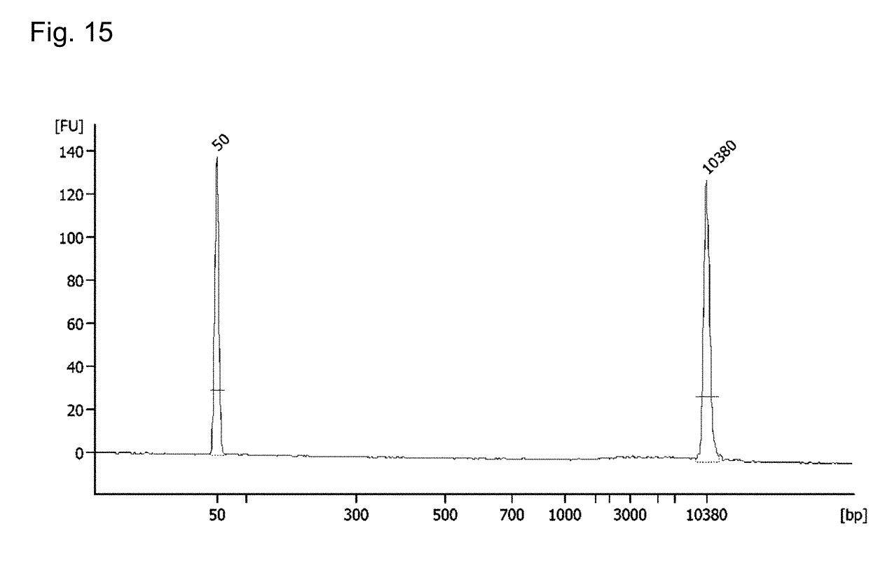

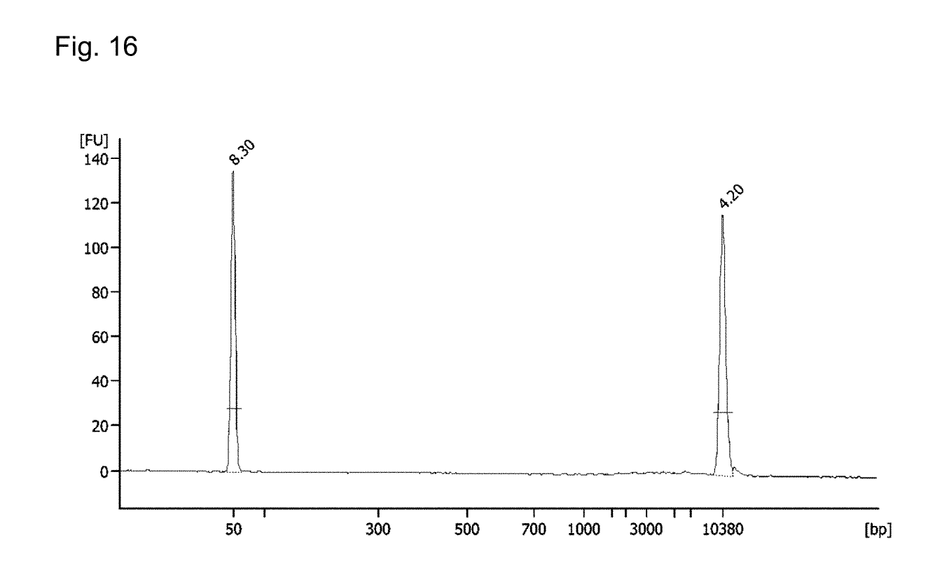

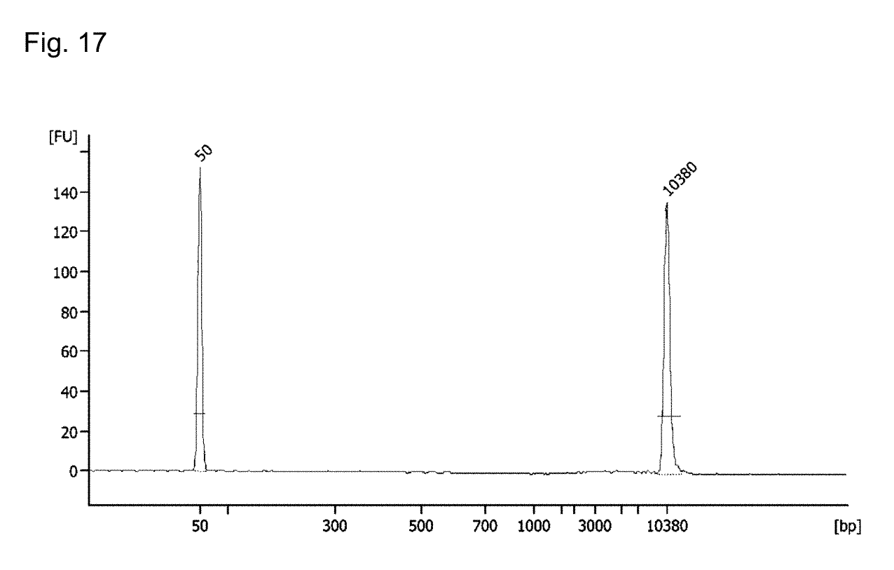

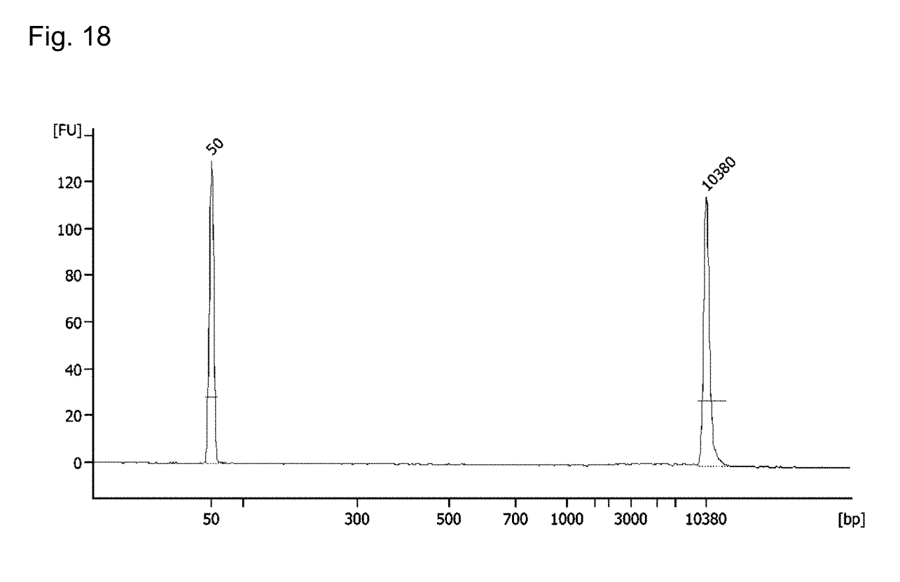

FIGS. 11 to 18 show the results of the experiment described in 3.1.7 attained at the random primer lengths of 8 nucleotides, 9 nucleotides, 11 nucleotides, 12 nucleotides, 14 nucleotides, 16 nucleotides, 18 nucleotides, and 20 nucleotides, respectively. Regardless of the length of a random primer, as shown in FIGS. 11 to 18, no significant change was observed in comparison with the results shown in FIG. 2 (a random primer comprising 10 nucleotides).

The results of experiment described in 3.1.8 are summarized in Table 23.

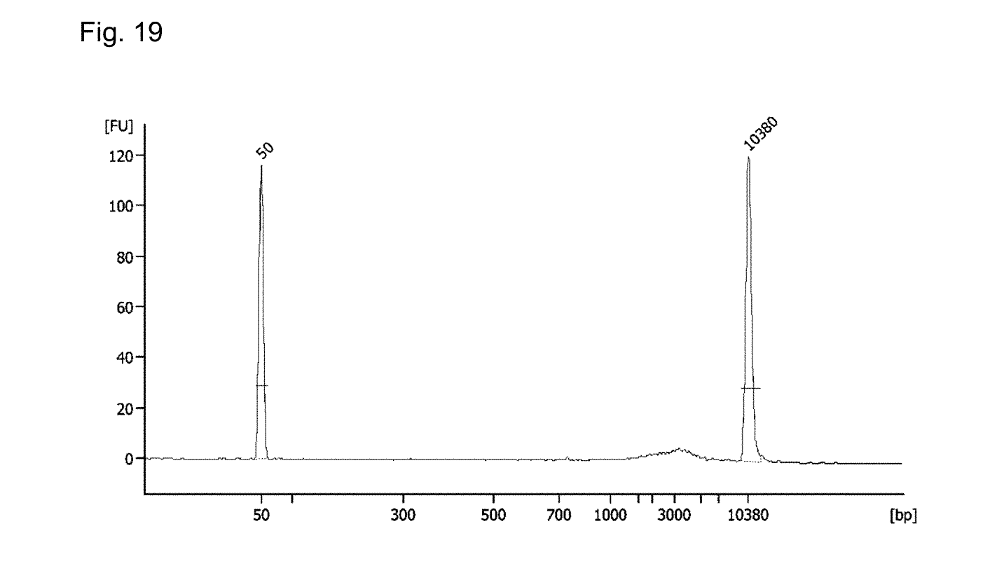

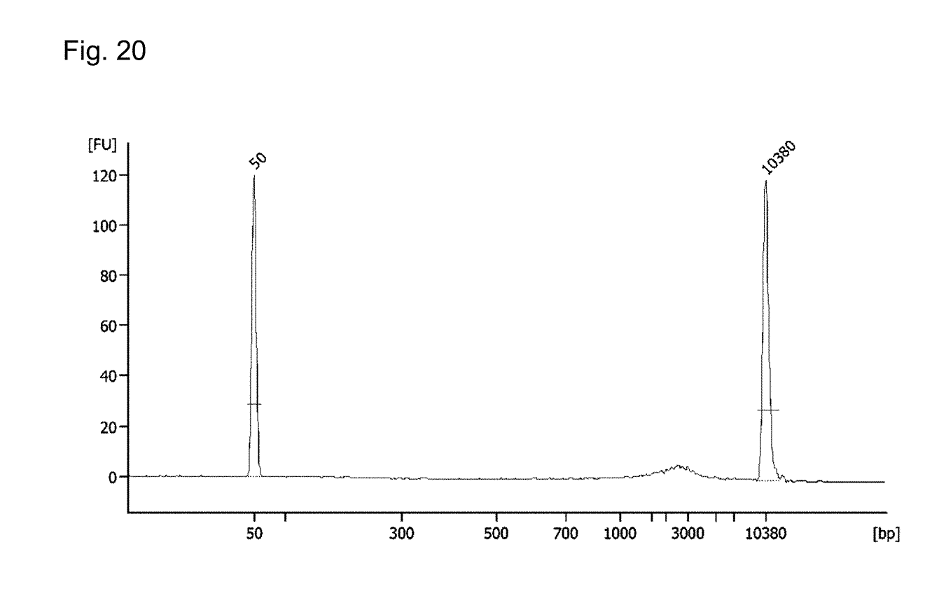

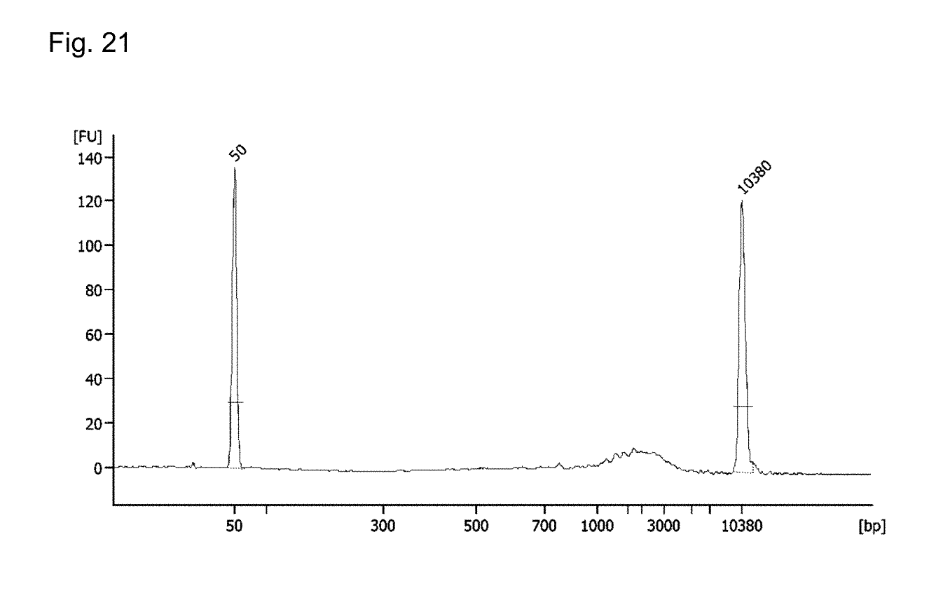

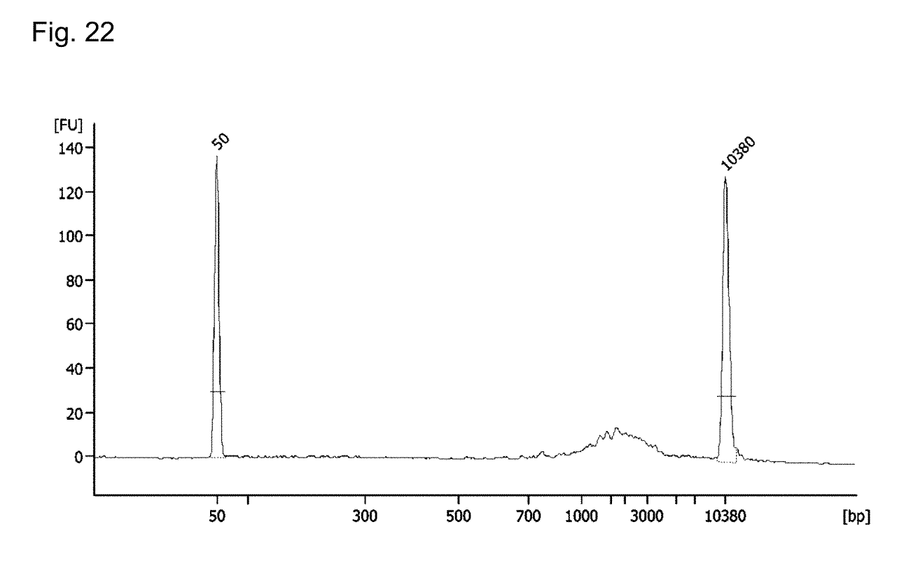

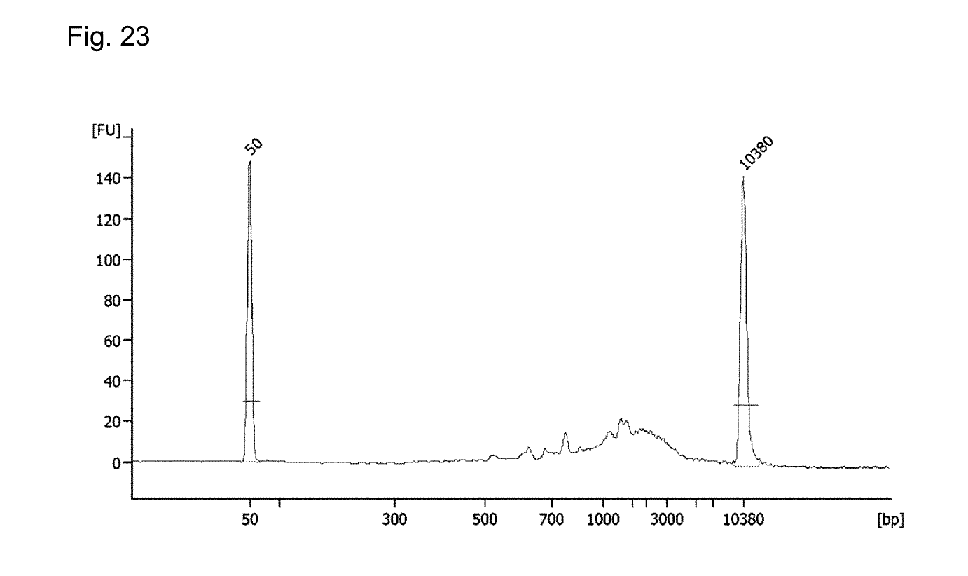

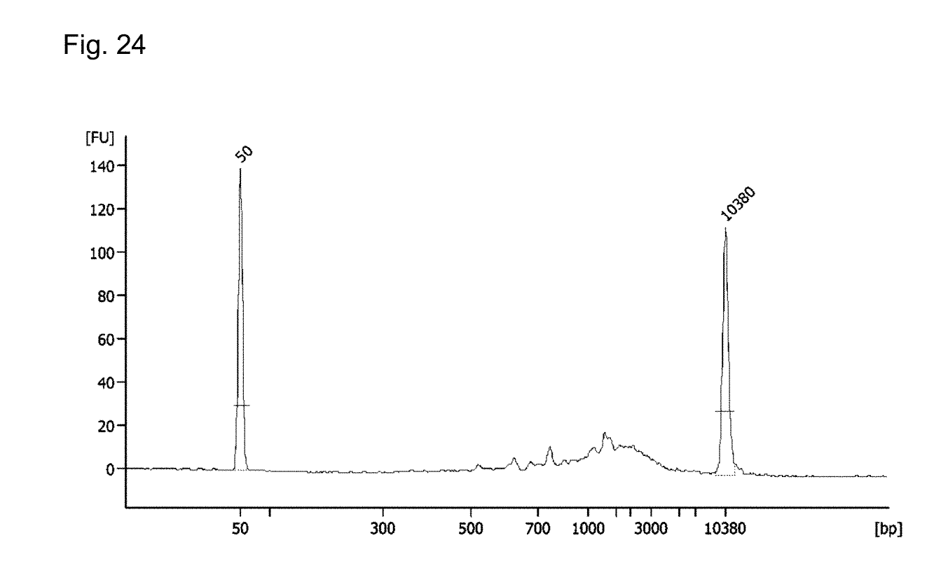

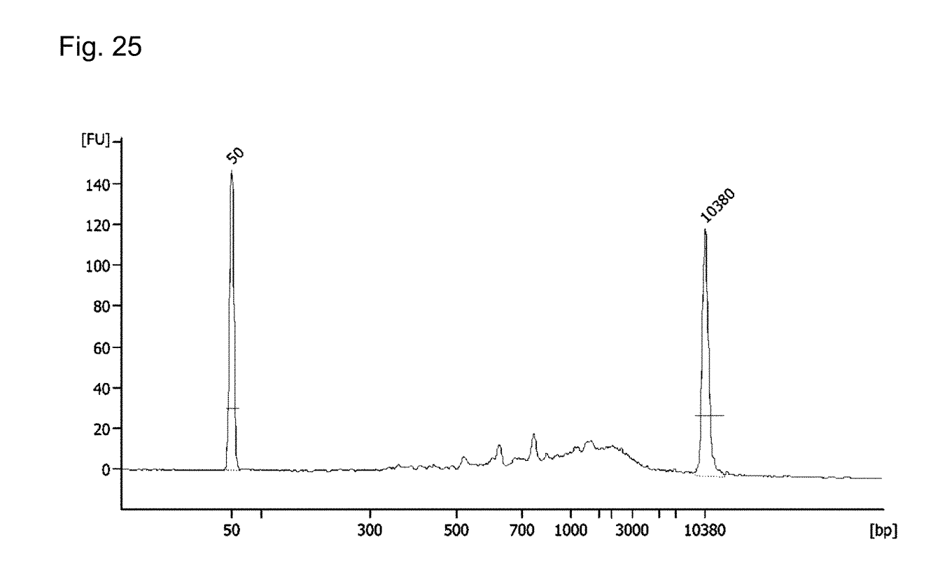

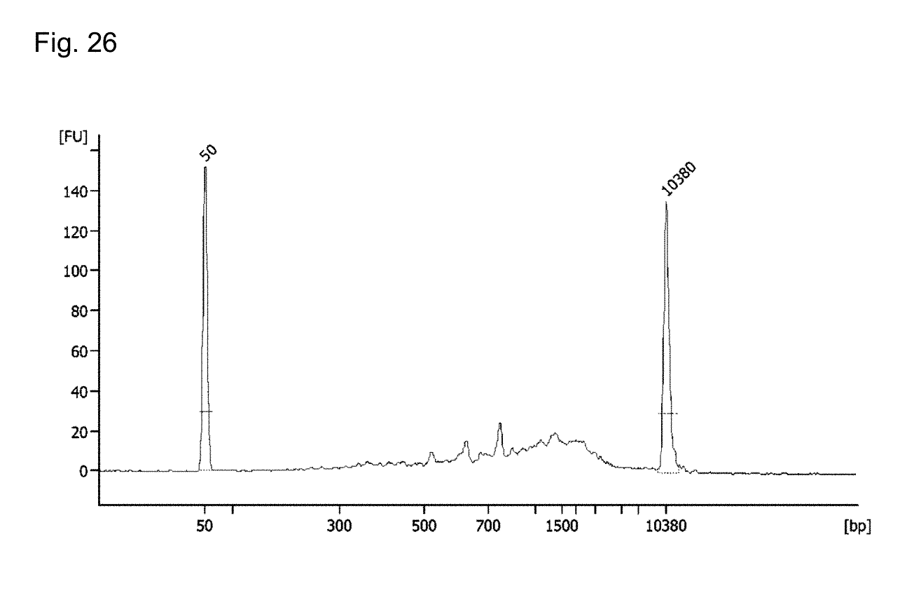

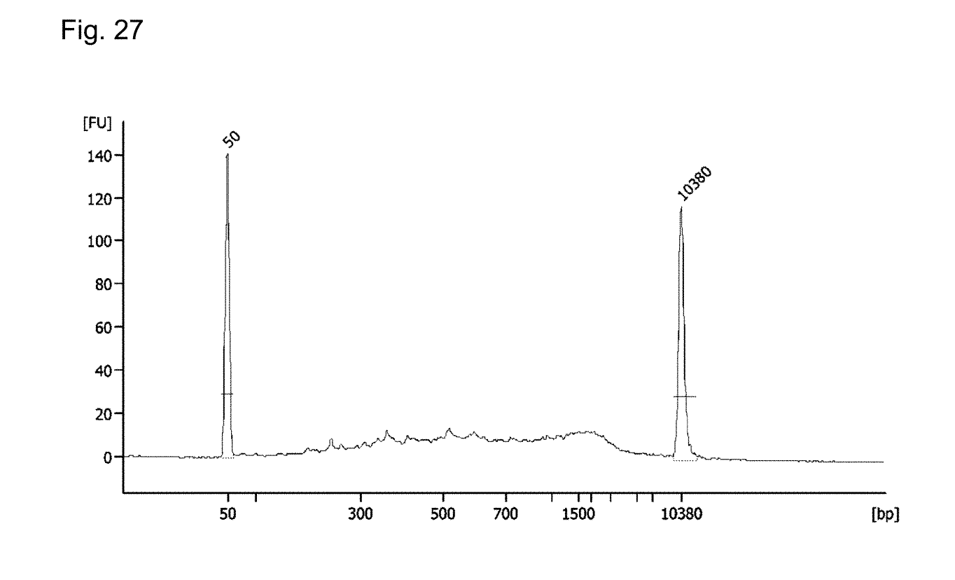

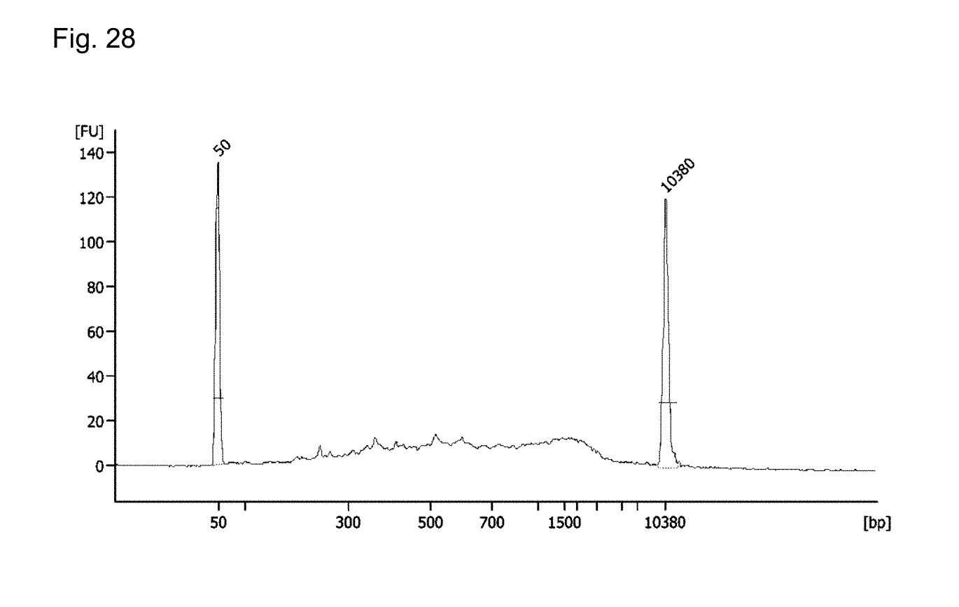

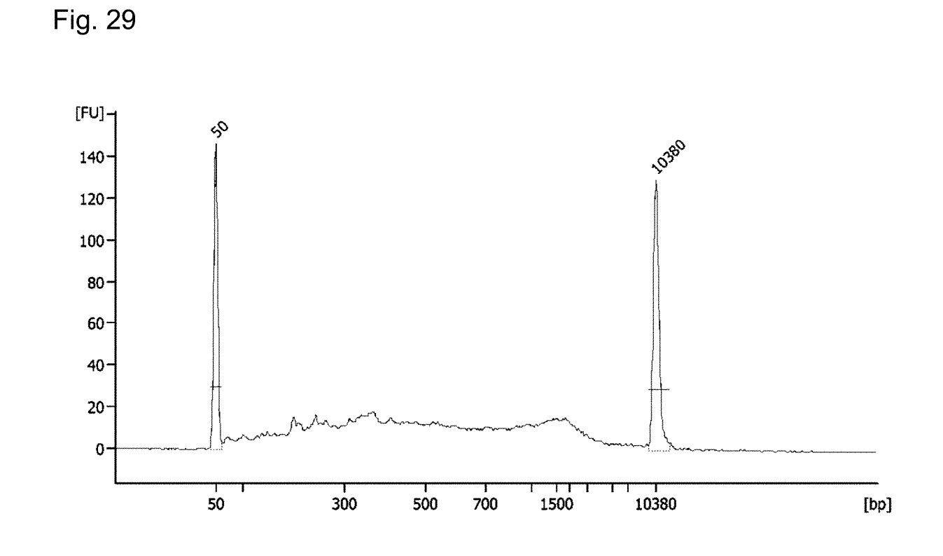

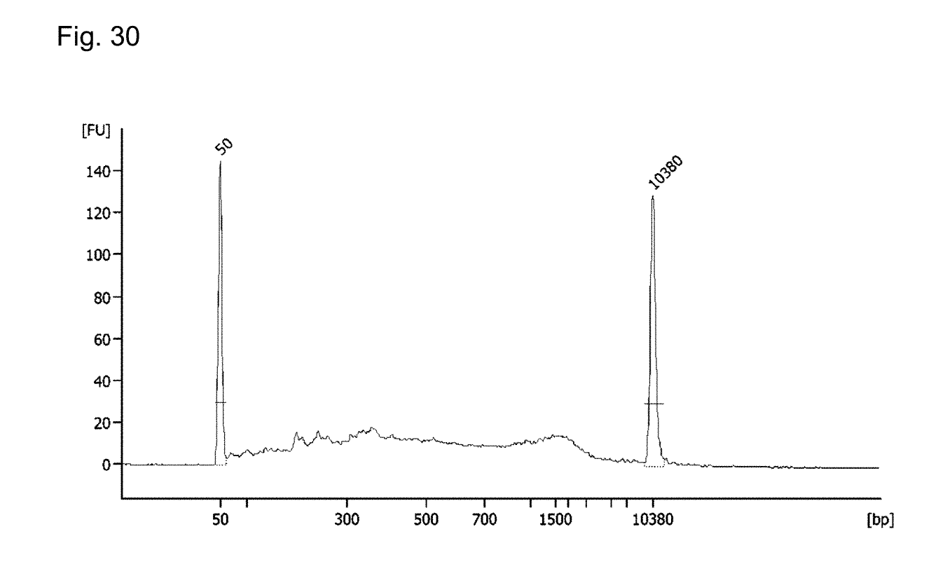

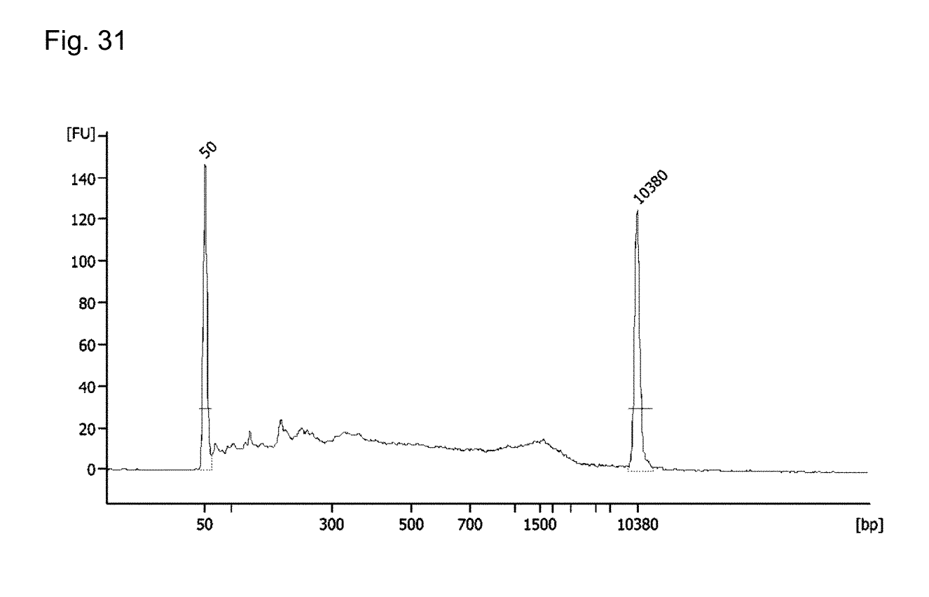

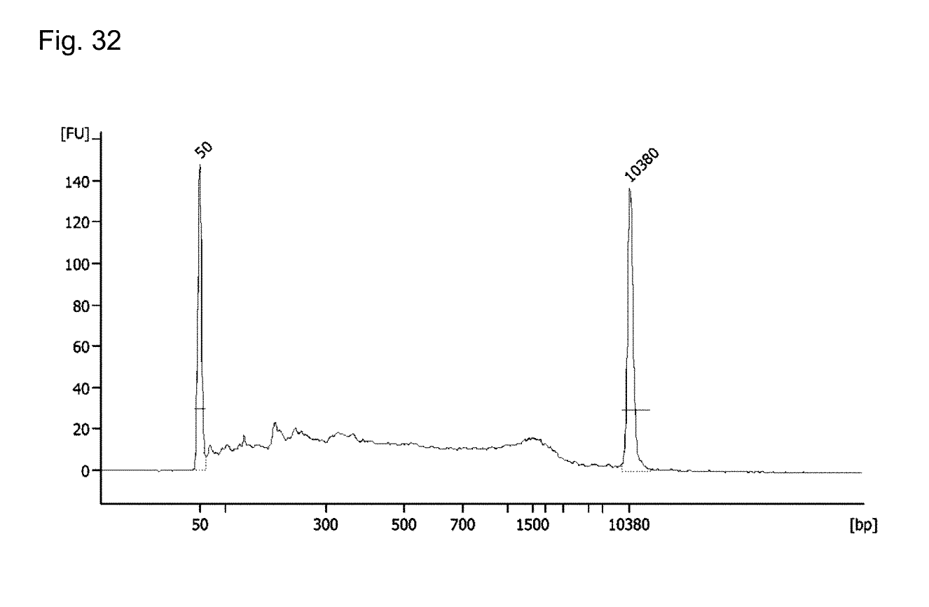

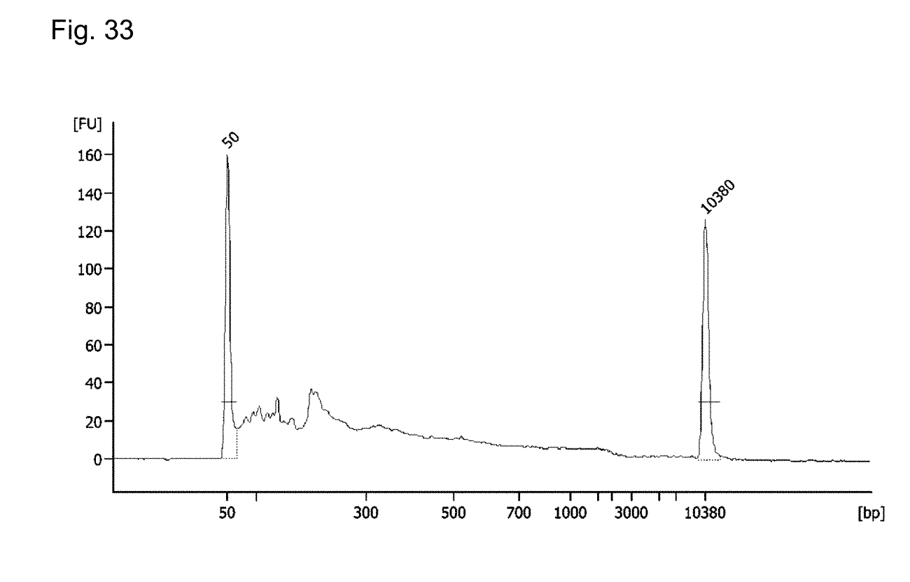

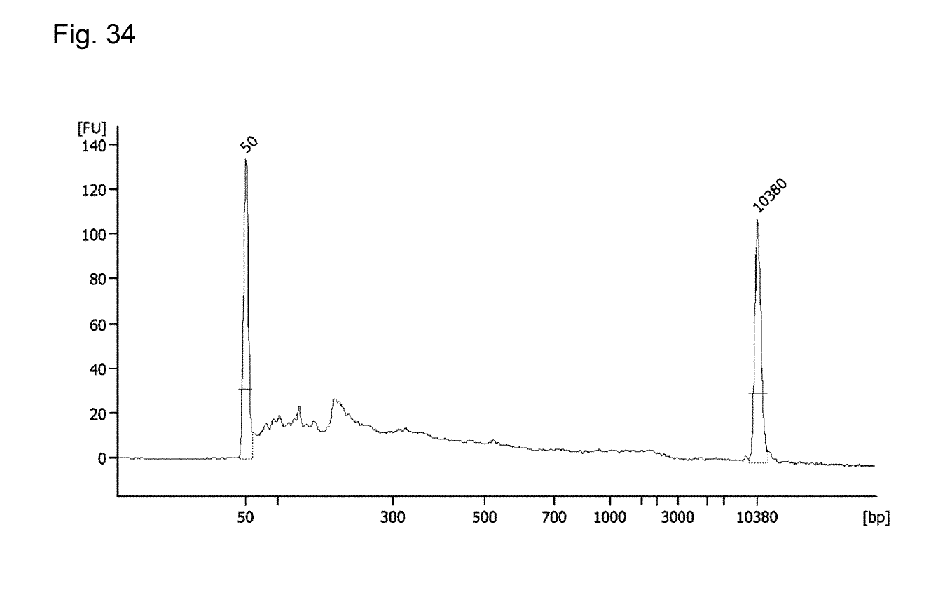

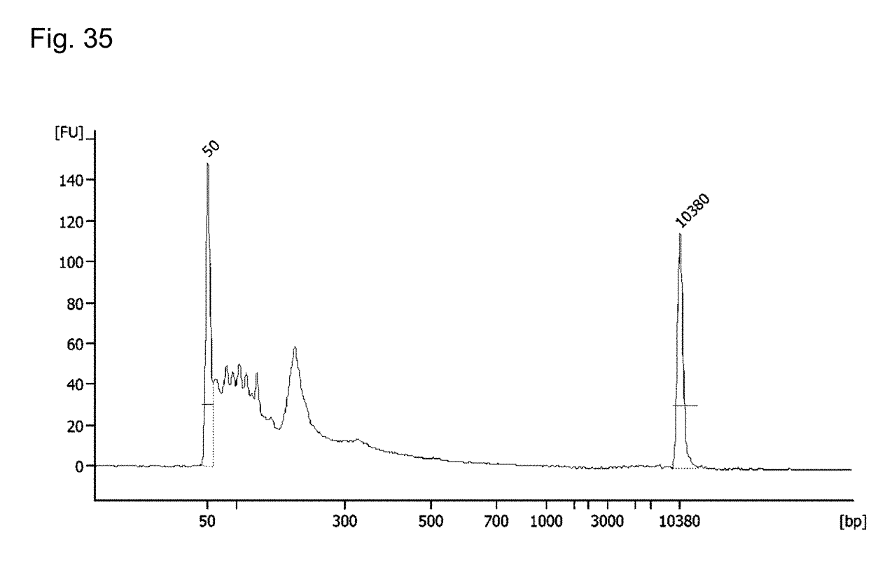

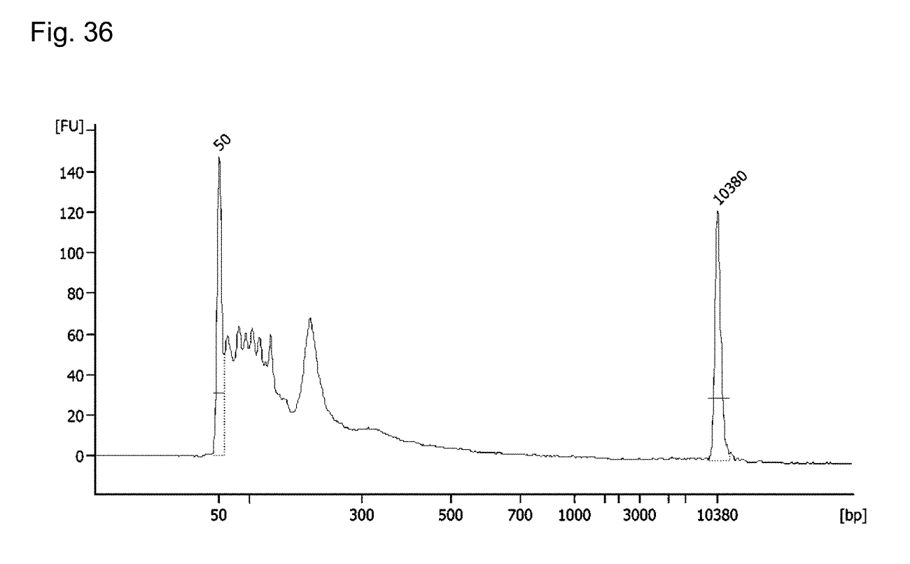

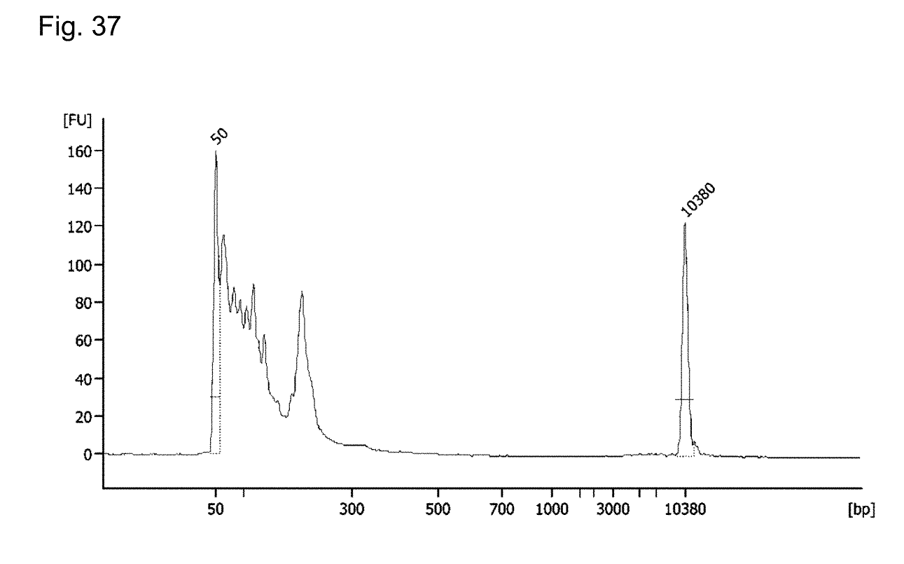

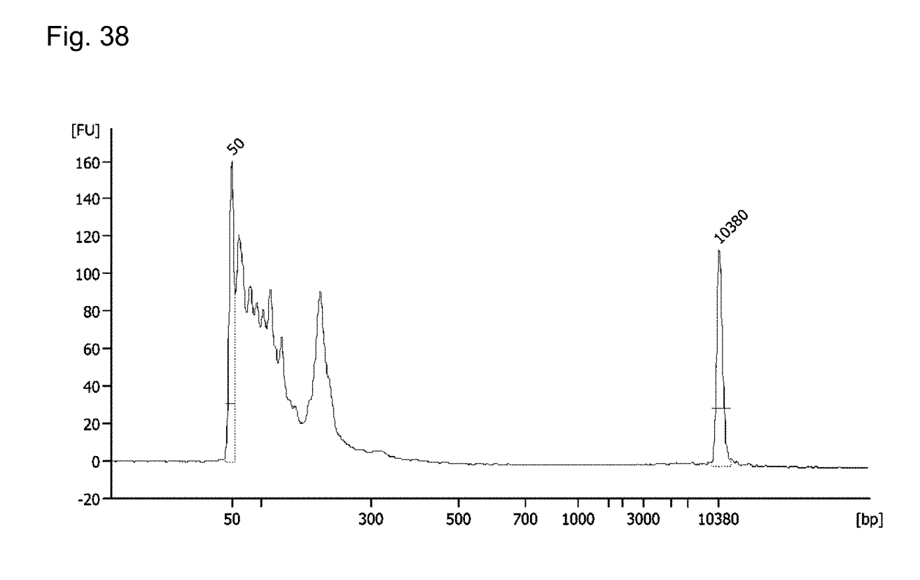

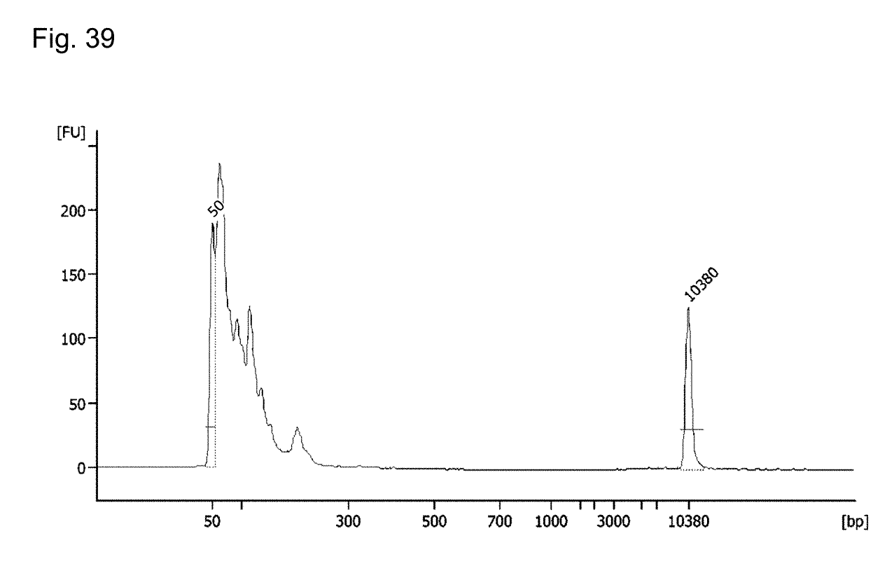

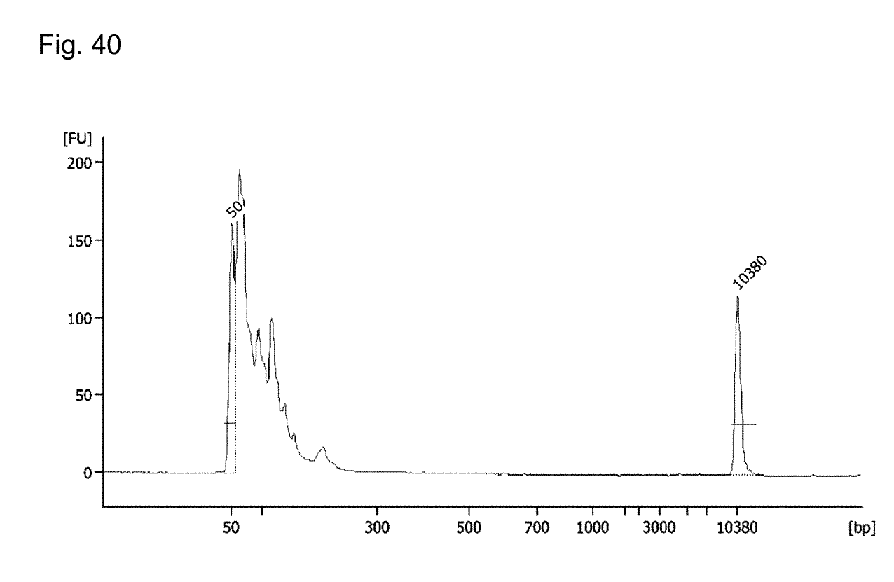

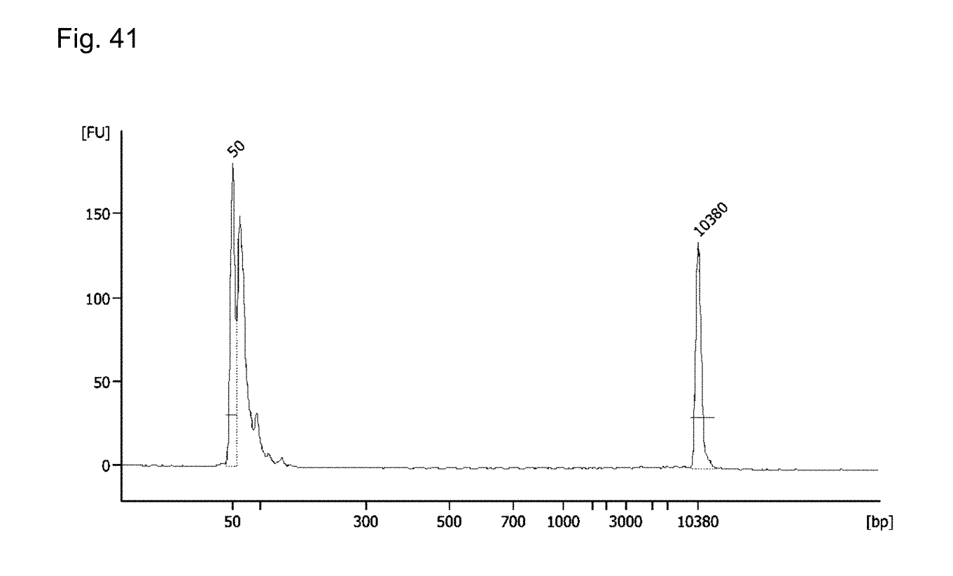

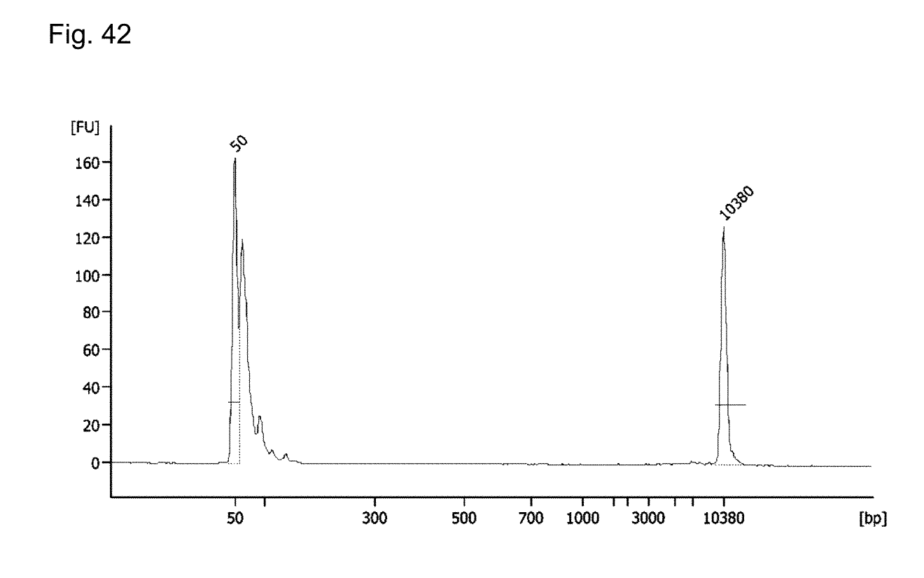

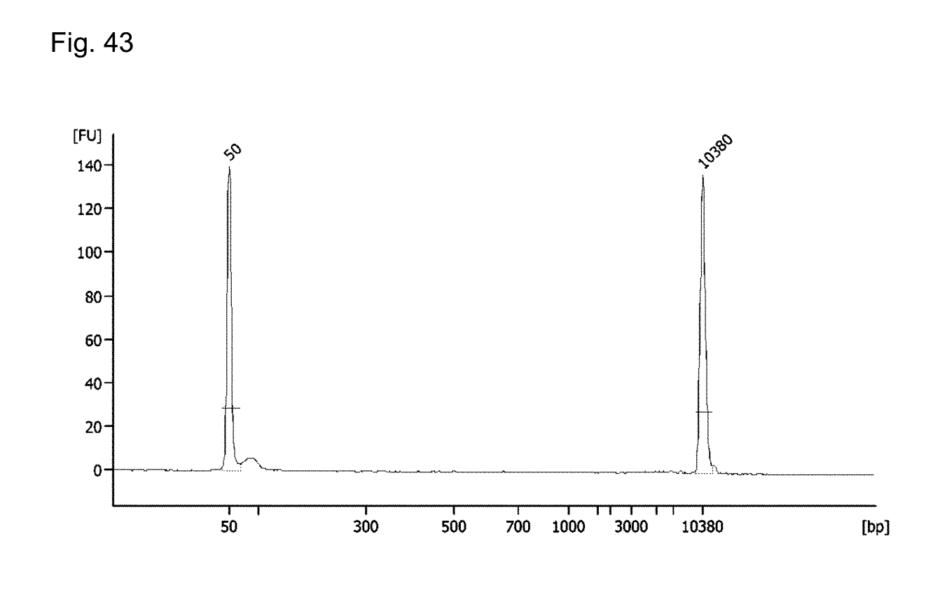

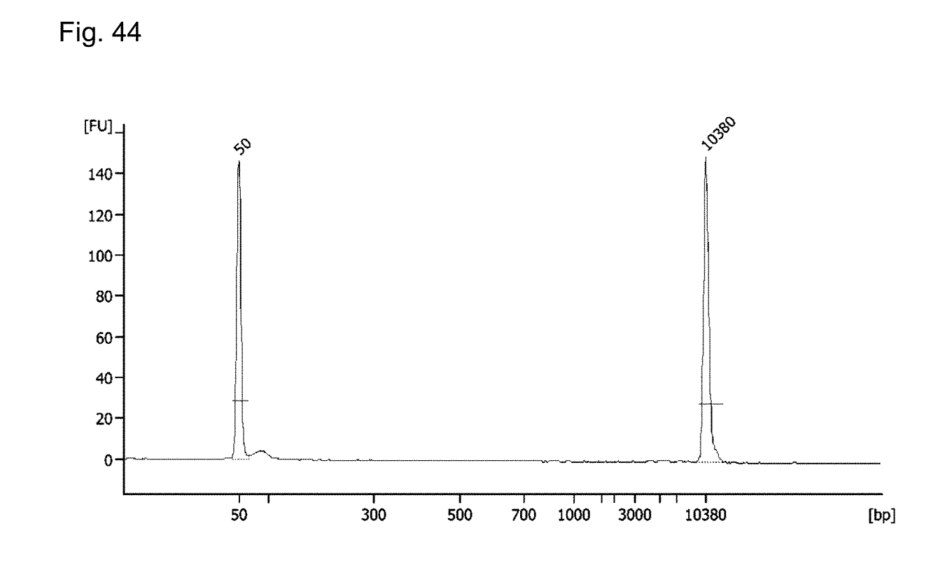

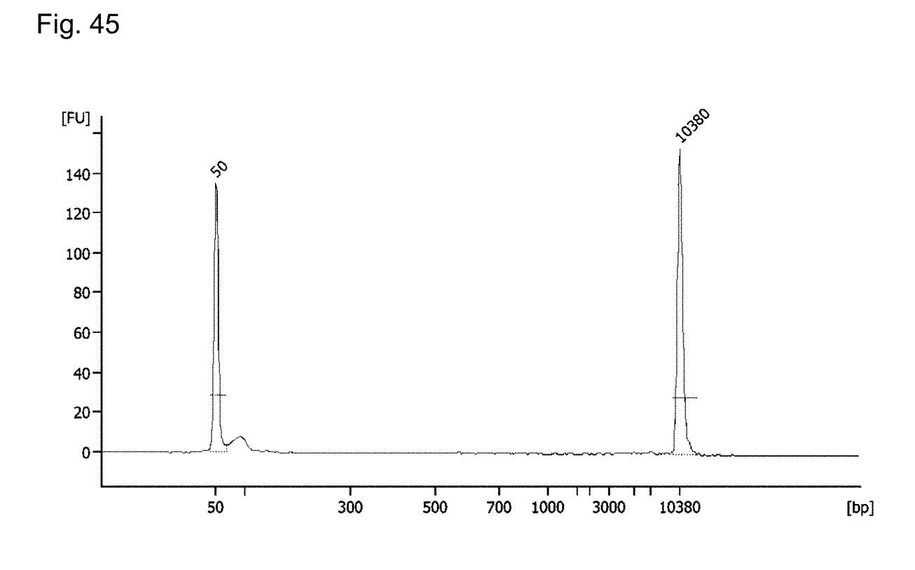

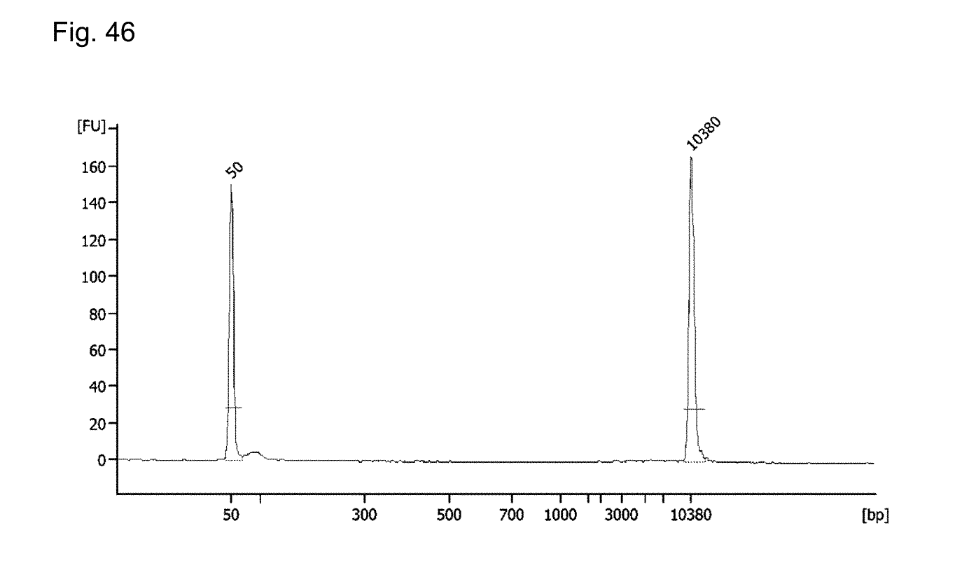

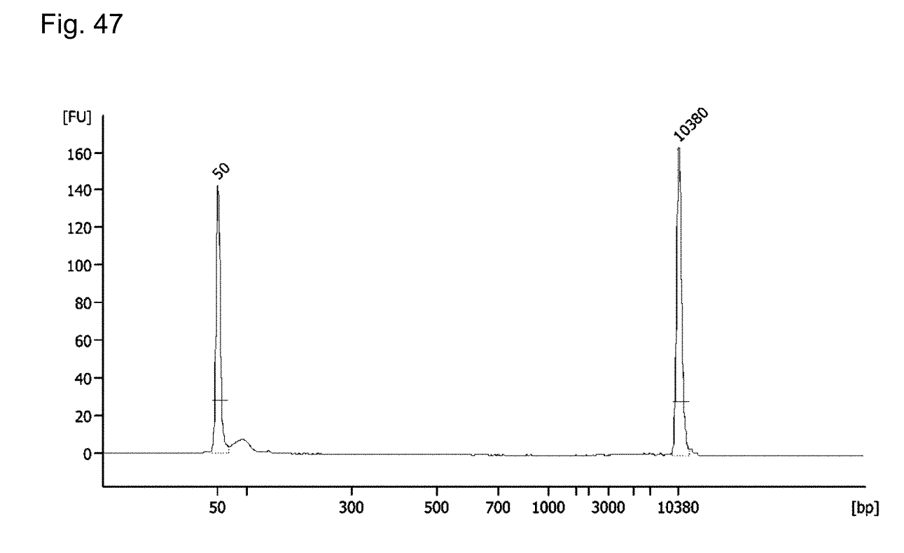

TABLE 23

| Concentration | | | Correlation |
|---|---|---|---|
| (μM) | Repeat | FIG. | coefficient (ρ) |
| 2 | — | FIG. 19 | — |
| 4 | — | FIG. 20 | — |
| 6 | 1st | FIG. 21 | 0.889 |
| | 2nd | FIG. 22 | |
| 8 | 1st | FIG. 23 | 0.961 |
| | 2nd | FIG. 24 | |
| 10 | 1st | FIG. 25 | 0.979 |
| | 2nd | FIG. 26 | |
| 20 | 1st | FIG. 27 | 0.950 |
| | 2nd | FIG. 28 | |
| 40 | 1st | FIG. 29 | 0.975 |
| | 2nd | FIG. 30 | |
| 60 | 1st | FIG. 31 | 0.959 |
| | 2nd | FIG. 32 | |
| 100 | 1st | FIG. 33 | 0.983 |
| | 2nd | FIG. 34 | |
| 200 | 1st | FIG. 35 | 0.991 |
| | 2nd | FIG. 36 | |
| 300 | 1st | FIG. 37 | 0.995 |
| | 2nd | FIG. 38 | |
| 400 | 1st | FIG. 39 | 0.988 |
| | 2nd | FIG. 40 | |
| 500 | 1st | FIG. 41 | 0.971 |
| | 2nd | FIG. 42 | |
| 600 | — | FIG. 43 | — |
| 700 | — | FIG. 44 | — |
| 800 | — | FIG. 45 | — |
| 900 | — | FIG. 46 | — |
| 1000 | — | FIG. 47 | — |

With the use of random primers comprising 10 nucleotides, as shown in FIGS. 19 to 47, amplification was observed in a 1-kbp DNA fragment at the random primer concentration of 6 μM. As the concentration increased, the molecular weight of a DNA fragment decreased. Reproducibility at the random primer concentration of 6 to 500 μM was examined. As a result, a relatively low p value of 0.889 was attained at the concentration of 6 μM, which is 10 times higher than the usual level. At the concentration of 8 μM, which is equivalent to 13.3 times higher than the usual level, and at 500 μM, which is 833.3 times higher than the usual level, a high p value of 0.9 or more was attained. The results demonstrate that a DNA fragment of 1 kbp or smaller can be amplified while achieving high reproducibility by elevating the random primer concentration to a level significantly higher than the concentration employed under general PCR conditions. When the random primer concentration is excessively higher than 500 μM, amplification of a DNA fragment of a desired size cannot be observed. In order to amplify a low-molecular-weight DNA fragment with excellent reproducibility, accordingly, it was found that the random primer concentration should fall within an optimal range, which is higher than the concentration employed in a general PCR procedure and equivalent to or lower than a given level.

### 4.2 Confirmation of Reproducibility Via MiSeq

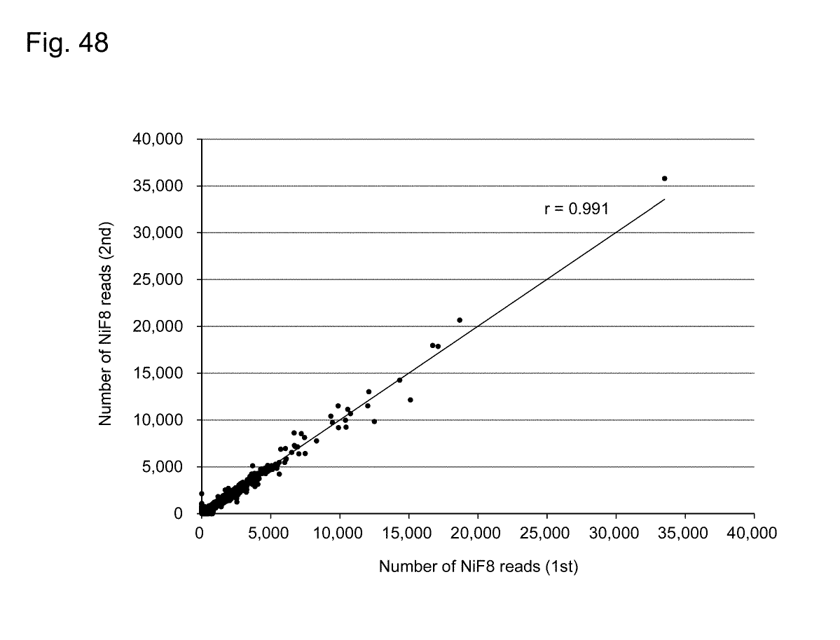

In order to confirm the reproducibility for DNA library production, as described in 3.2 above, the DNA library amplified with the use of the genomic DNA extracted from NiF8 as a template and random primers was analyzed with the use of a next-generation sequencer (MiSeq), and the results are shown in FIG. 48. As a result of 3.2.4 above, 47,484 read patterns were obtained. As a result of comparison of the number of reads obtained through repeated measurements, a high correlation (i.e., a correlational coefficient “r” of 0.991) was obtained, as with the results of electrophoresis. Accordingly, it was considered that a DNA library could be produced with satisfactory reproducibility with the use of random primers.

### 4.3 Analysis of Rice Variety Nipponbare

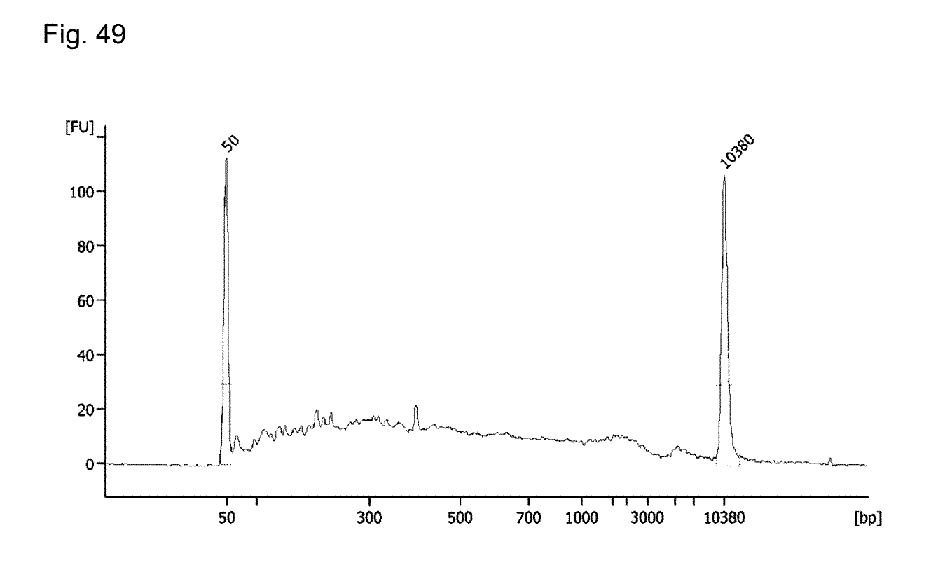

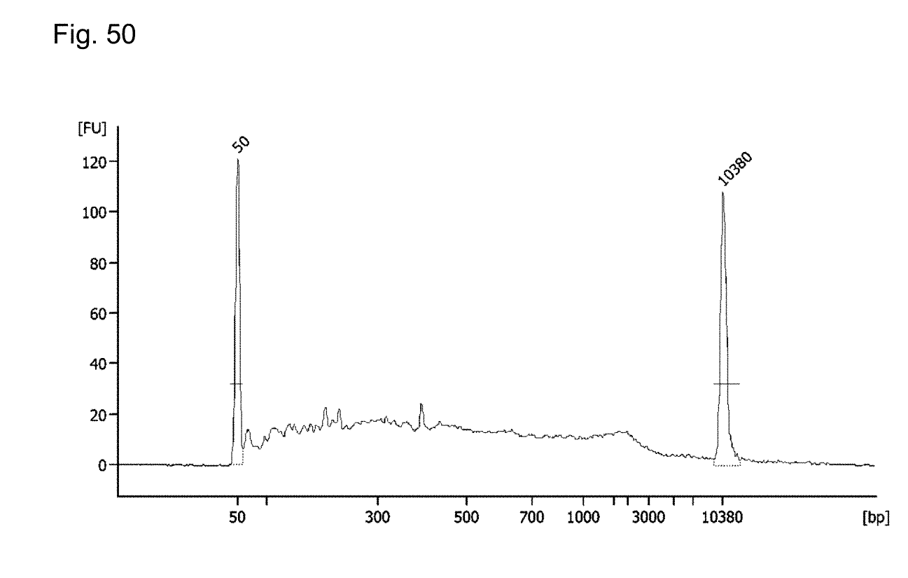

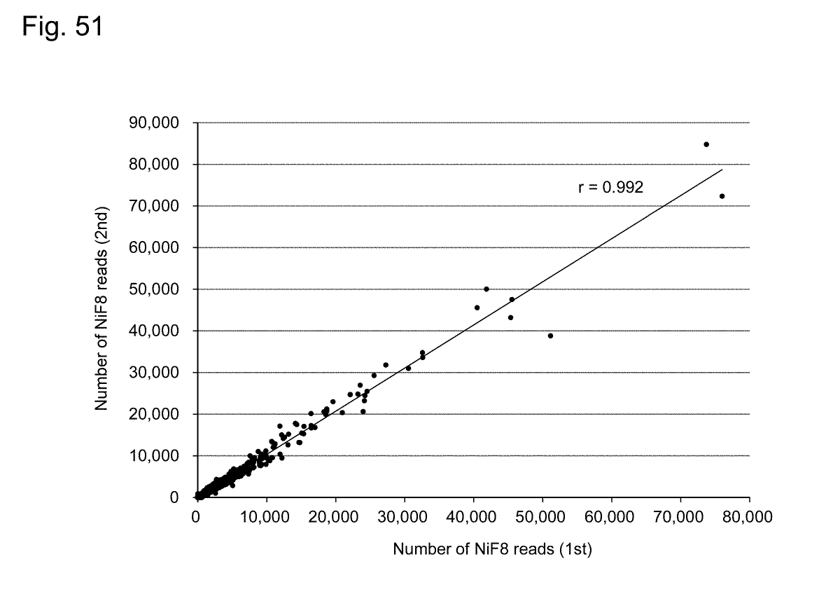

As described in 3.3 above, a DNA library was prepared with the use of genomic DNA extracted from the rice variety Nipponbare, the genomic information of which has been disclosed, as a template, and random primers and subjected to electrophoresis, and the results are shown in FIGS. 49 and 50. On the basis of the results shown in FIGS. 49 and 50, the p value was found to be as high as 0.979. Also, FIG. 51 shows the results of analysis of the read data with the use of MiSeq. On the basis of the results shown in FIG. 51, the correlational coefficient “r” was found to be as high as 0.992. These results demonstrate that a DNA library of rice could be produced with very high reproducibility with the use of random primers.

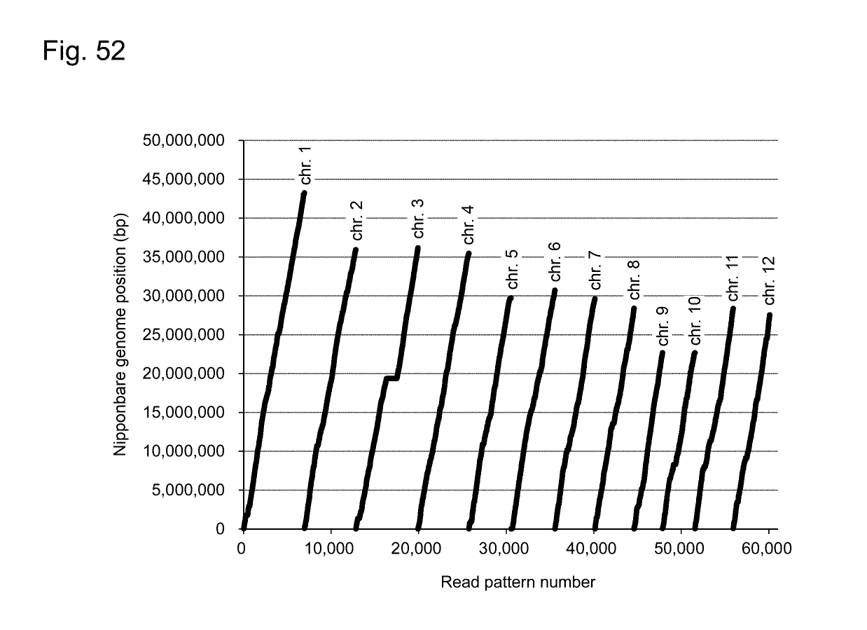

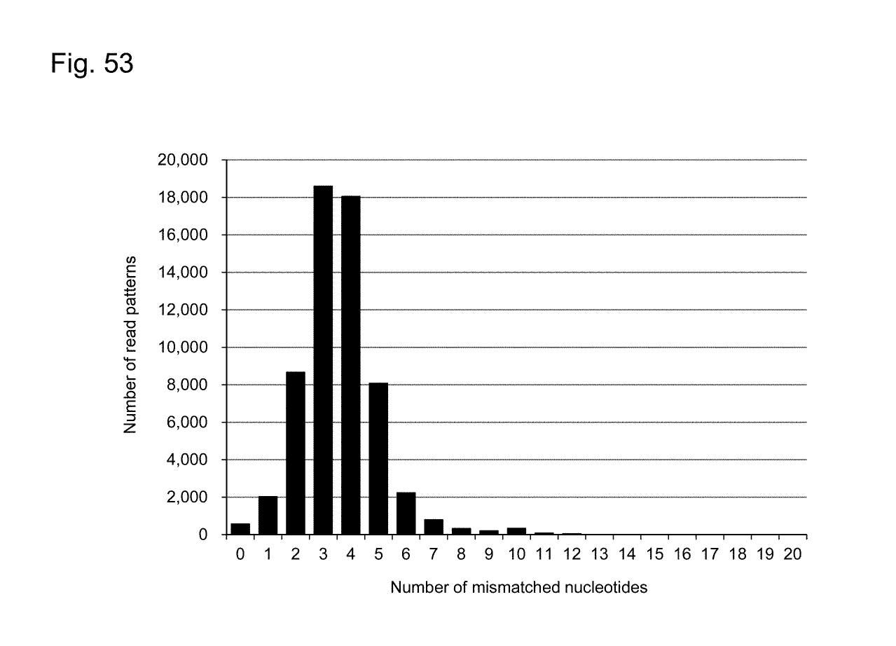

As described in 3.3.3, the obtained read pattern was mapped to the genomic information of Nipponbare. As a result, DNA fragments were found to be evenly amplified throughout the genome at intervals of 6.2 kbp (FIG. 52). As a result of comparison of the sequence and genome information of random primers, 3.6 mismatches were found on average, and one or more mismatches were observed in 99.0% of primer pairs (FIG. 53). The results demonstrate that a DNA library involving the use of random primers is produced with satisfactory reproducibility via non-specific amplification evenly throughout the genome.

### 4.4 Detection of Polymorphism and Genotype Identification of Sugarcane

As described in 3.4. DNA libraries of the sugarcane varieties NiF8 and Ni9 and 22 hybrid progeny lines were produced with the use of random primers, the resulting DNA libraries were analyzed with the next-generation sequencer (HiSeq), the polymorphisms of the parent varieties were detected, and the genotypes of the hybrid progenies were identified on the basis of the read data. Table 24 shows the results.

TABLE 24
Number of markers and genotyping accuracy of sugarcane varieties NiF8 and Ni9

| | Number | | | | | | |
|---|---|---|---|---|---|---|---|
| | of | F1_01 | | F1_02 | | Total | |
| | markers | Consistency | Reproducibility | Consistency | Reproducibility | Consistency | Reproducibility |
| NiF8 | 8,683 | 8,680 | 99.97% | 8,682 | 99.99% | 17,362 | 99.98% |
| type | | | | | | | |
| Ni9 | 11,655 | 11,650 | 99.96% | 11,651 | 99.97% | 23,301 | 99.96% |
| type | | | | | | | |
| Total | 20,338 | 20,330 | 99.96% | 20,333 | 99.98% | 40,663 | 99.97% |

As shown in Table 24, 8,683 markers for NiF8 and 11,655 markers for Ni9; that is, a total of 20,338 markers, were produced. In addition, reproducibility for genotype identification of hybrid progeny lines was as high as 99.97%. This indicates that the accuracy for genotype identification is very high. In particular, sugarcane is polyploid (8x+n), the number of chromosomes is as large as 100 to 130, and the genome size is as large as 10 Gbp, which is at least 3 times greater than that of humans. Accordingly, it is very difficult to identify the genotype throughout the genomic DNA. As described above, numerous markers can be produced with the use of random primers, and the sugarcane genotype can thus be identified with high accuracy.

4.5 Experiment for Confirmation with PCR Marker

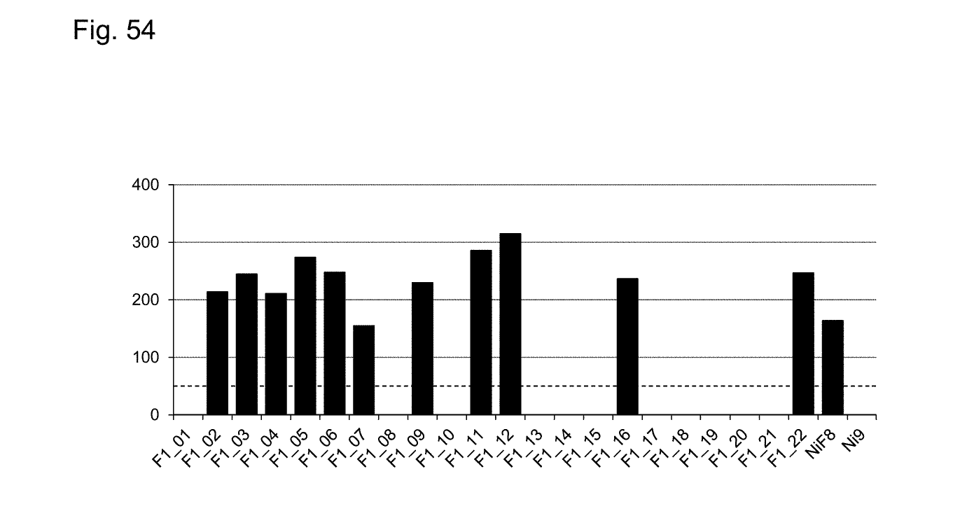

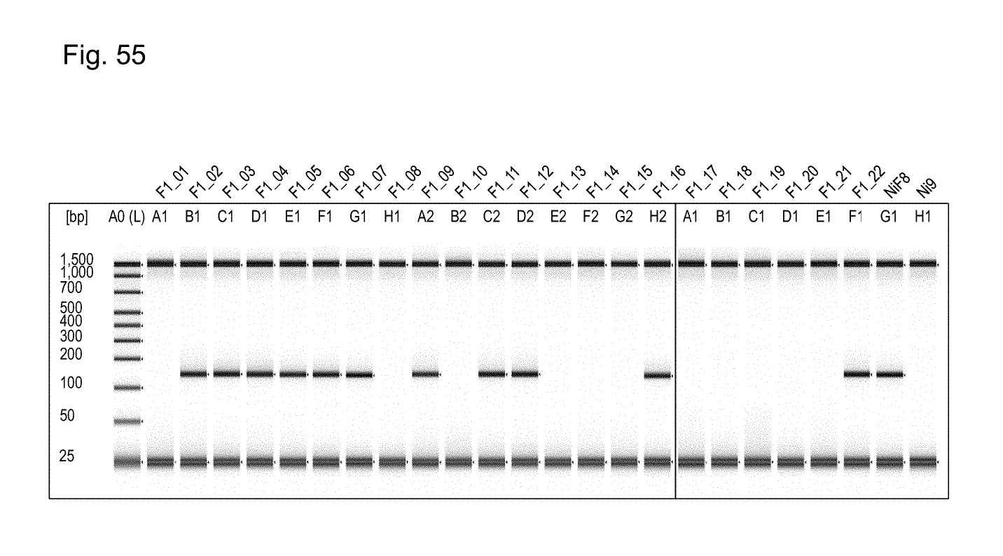

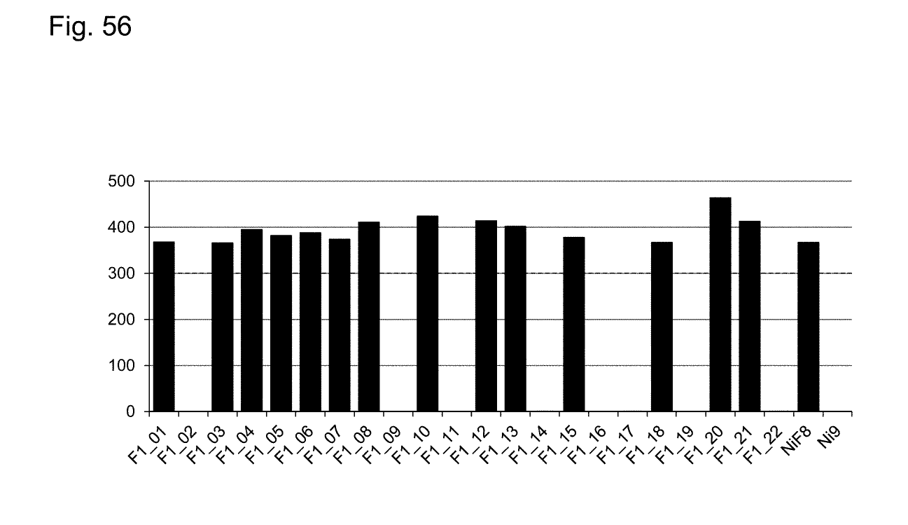

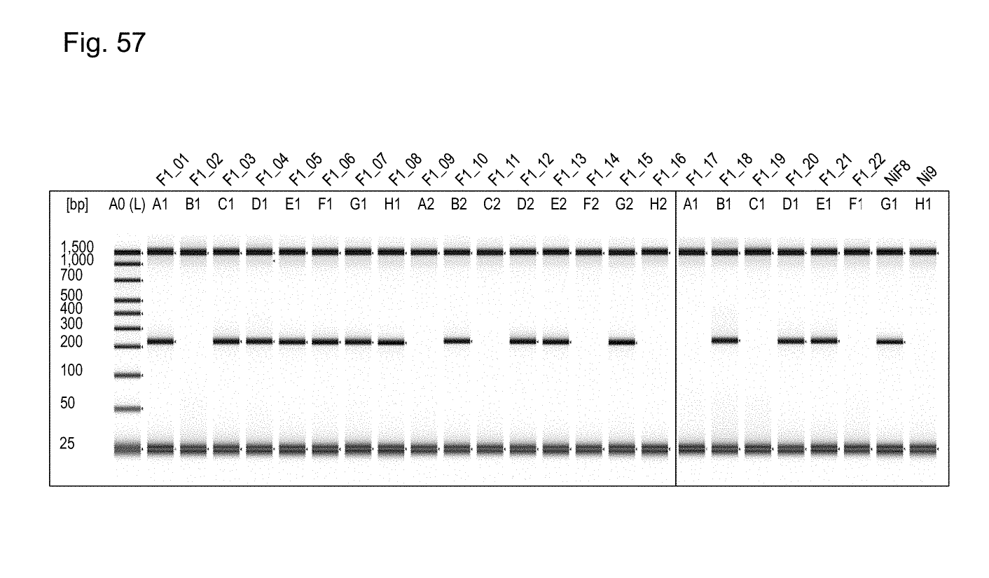

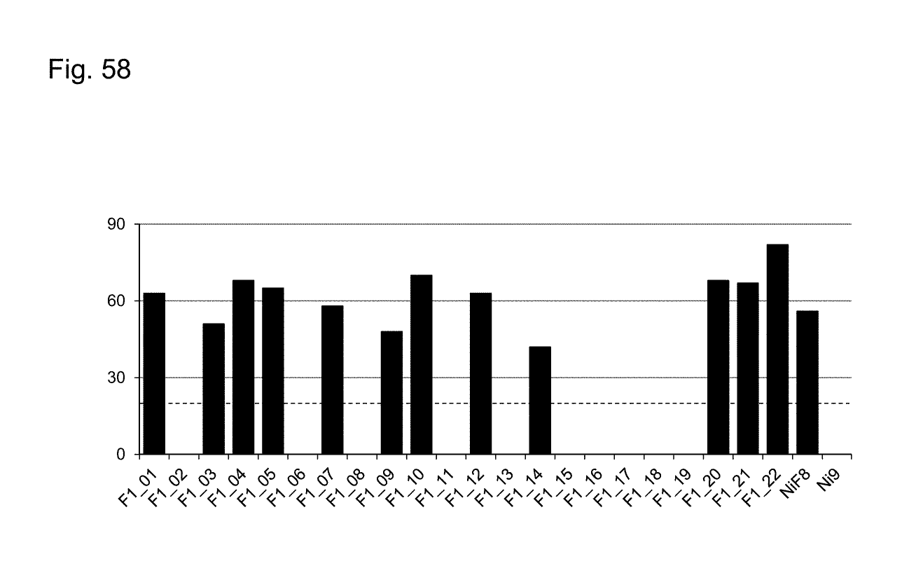

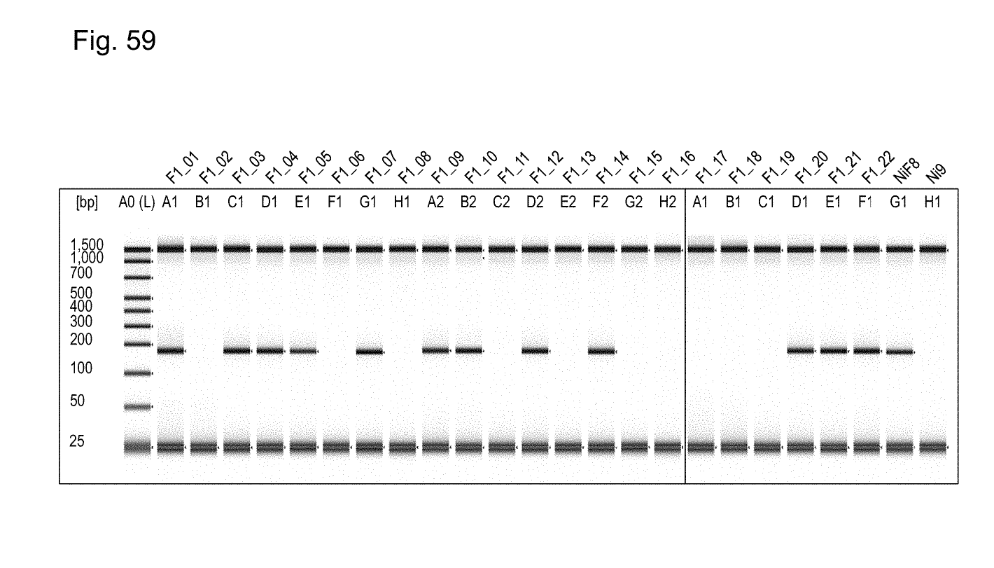

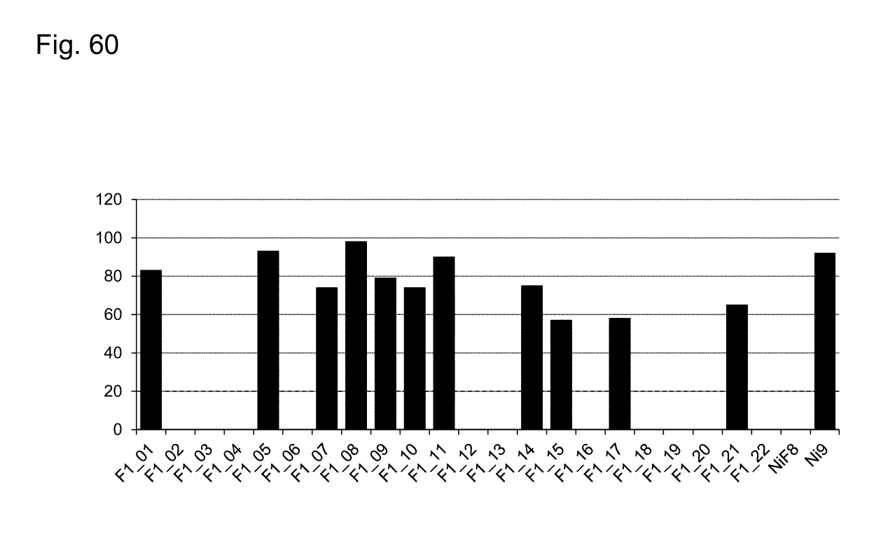

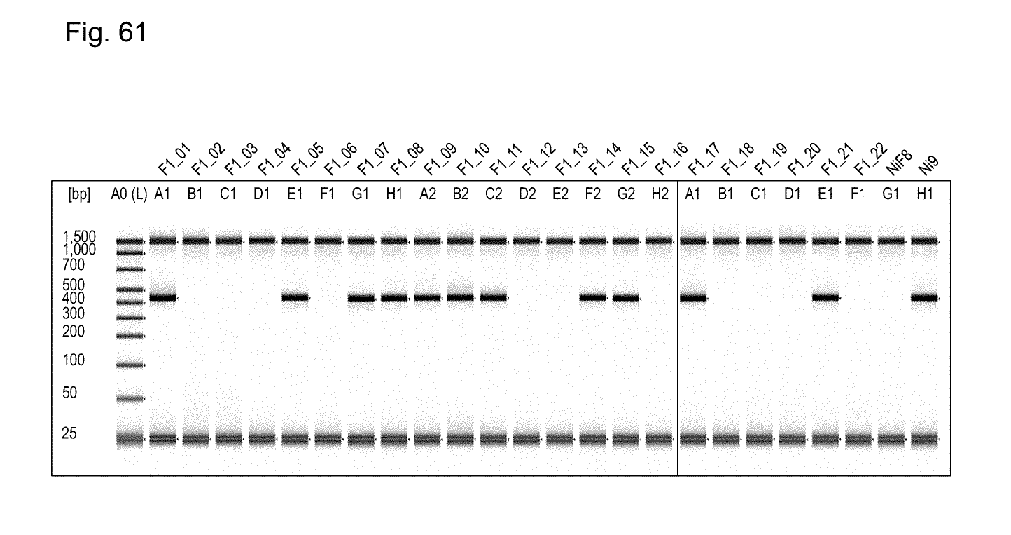

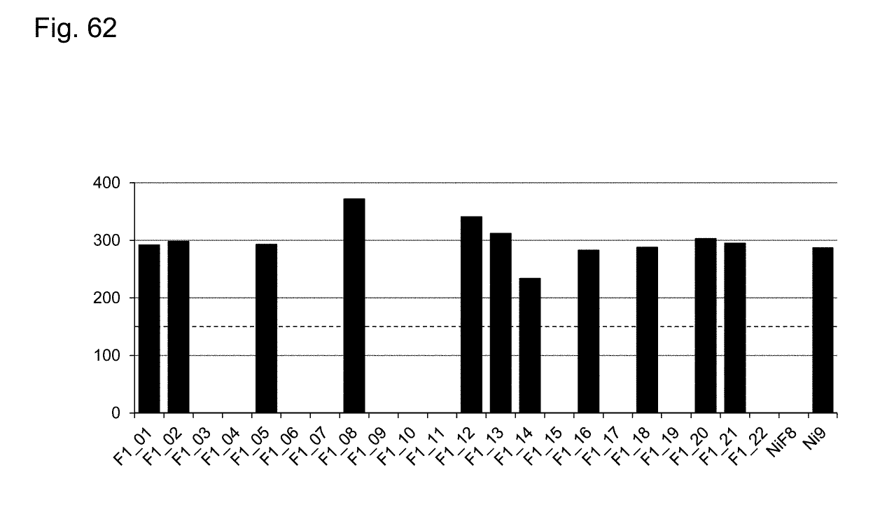

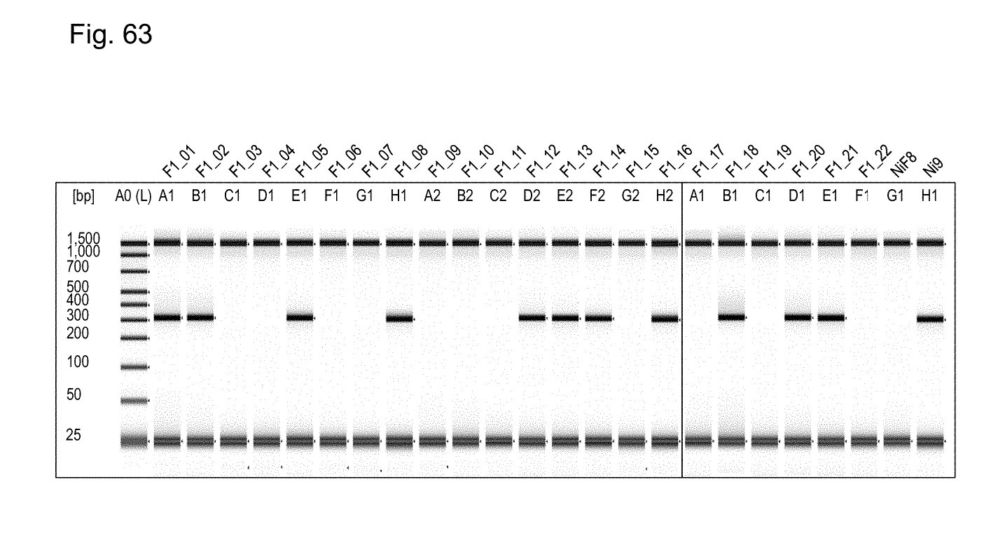

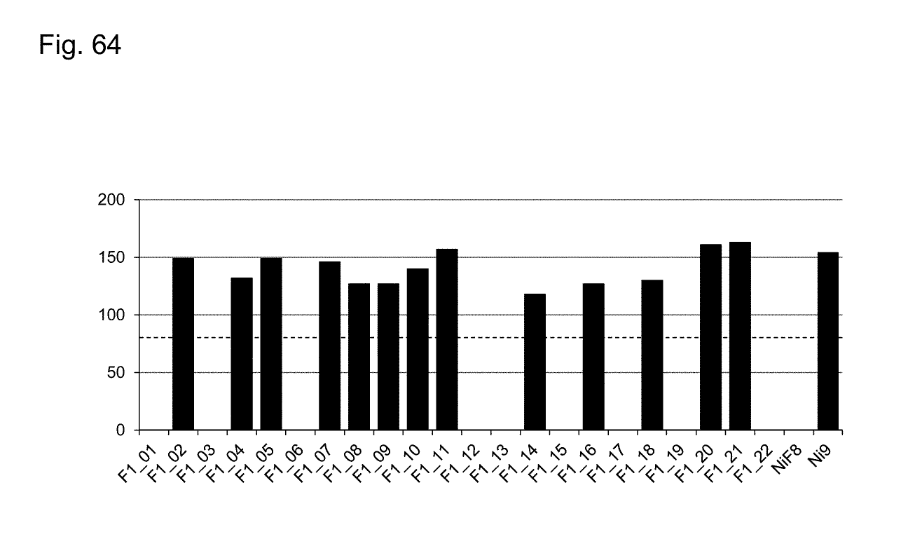

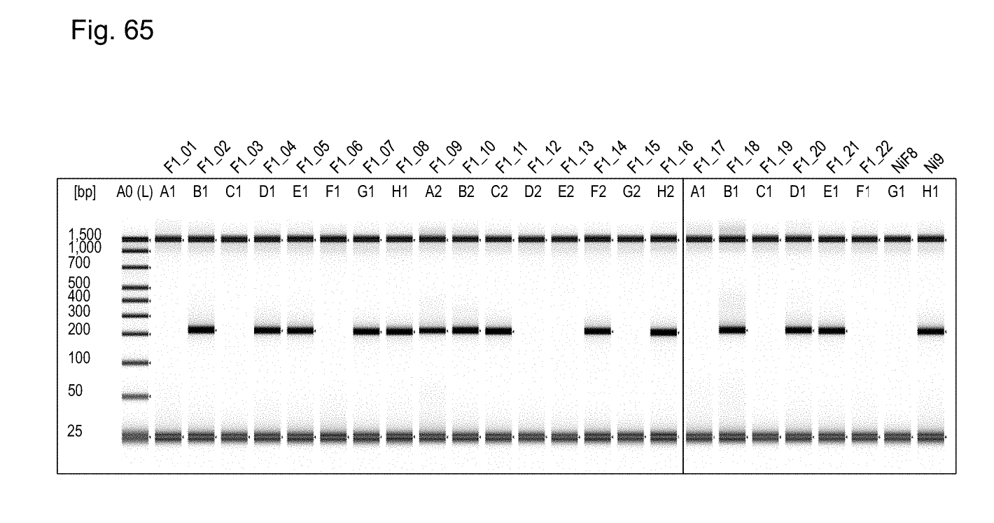

As described in 3.5 above, the sugarcane varieties NiF8 and Ni9 and 22 hybrid progeny lines were subjected to PCR with the use of the primers shown in Table 22, genotypes were identified via electrophoresis, and the results were compared with the number of reads. FIGS. 54 and 55 show the number of reads and the electrophoretic pattern of the NiF8 marker N80521152, respectively. FIGS. 56 and 57 show the number of reads and the electrophoretic pattern of the NiF8 marker N80997192, respectively. FIGS. 58 and 59 show the number of reads and the electrophoretic pattern of the NiF8 marker N80533142, respectively. FIGS. 60 and 61 show the number of reads and the electrophoretic pattern of the Ni9 marker N91552391, respectively. FIGS. 62 and 63 show the number of reads and the electrophoretic pattern of the Ni9 marker N91653962, respectively. FIGS. 64 and 65 show the number of reads and the electrophoretic pattern of the Ni9 marker N91 124801, respectively.

As shown in FIGS. 54 to 65, the results for all the PCR markers designed in 3.5 above were consistent with the results of analysis with the use of a next-generation sequencer. It was thus considered that genotype identification with the use of a next-generation sequencer would be applicable as a marker technique.

### 4.6 Correlation Between Random Primer Density and Length

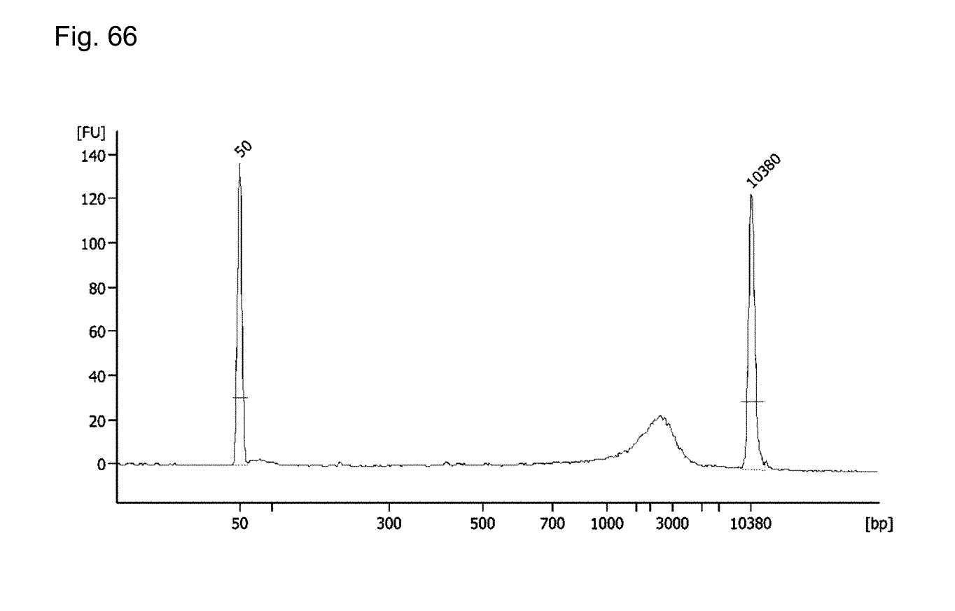

As described in 3.6.1, the results of DNA library production with the use of random primers comprising 9 nucleotides (Table 8), 10 nucleotides (Table 1, 10-nucleotide primer A), 11 nucleotides (Table 9), 12 nucleotides (Table 10), 14 nucleotides (Table 11), 16 nucleotides (Table 12), 18 nucleotides (Table 13), and 20 nucleotides (Table 14) are shown in FIGS. 66 to 81. The results are summarized in Table 25.

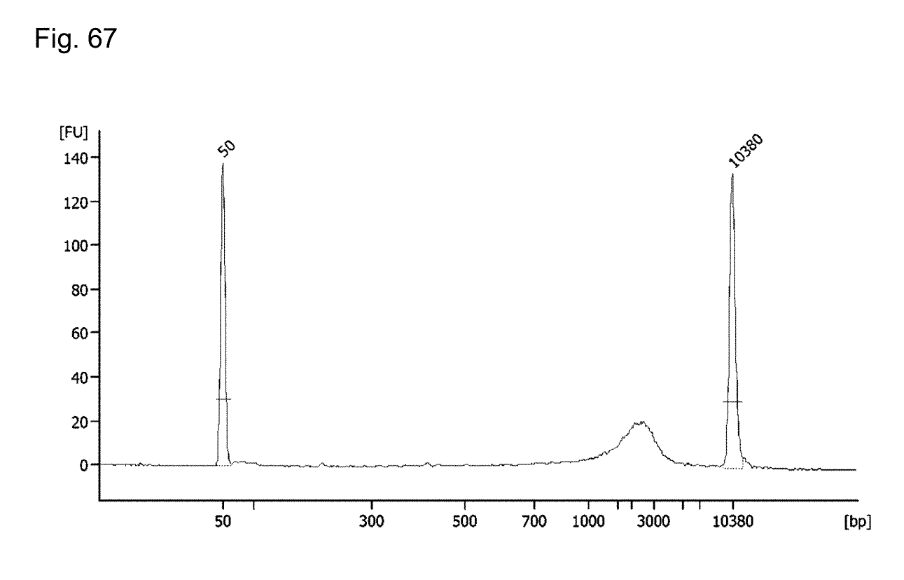

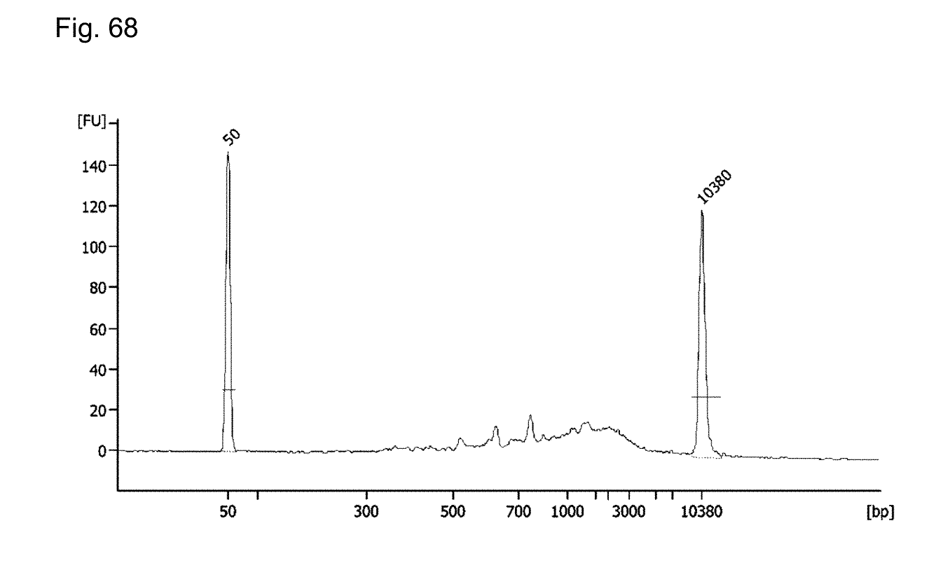

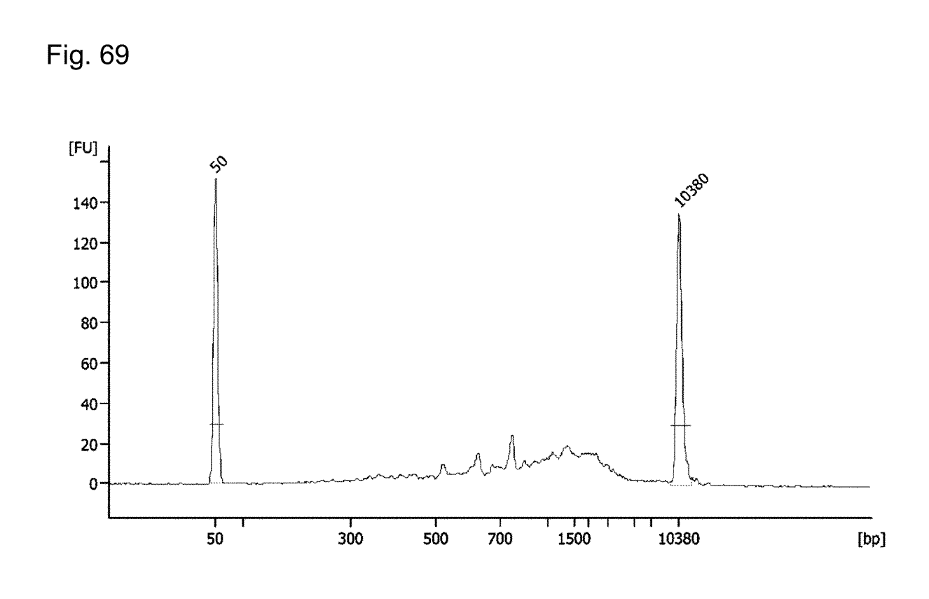

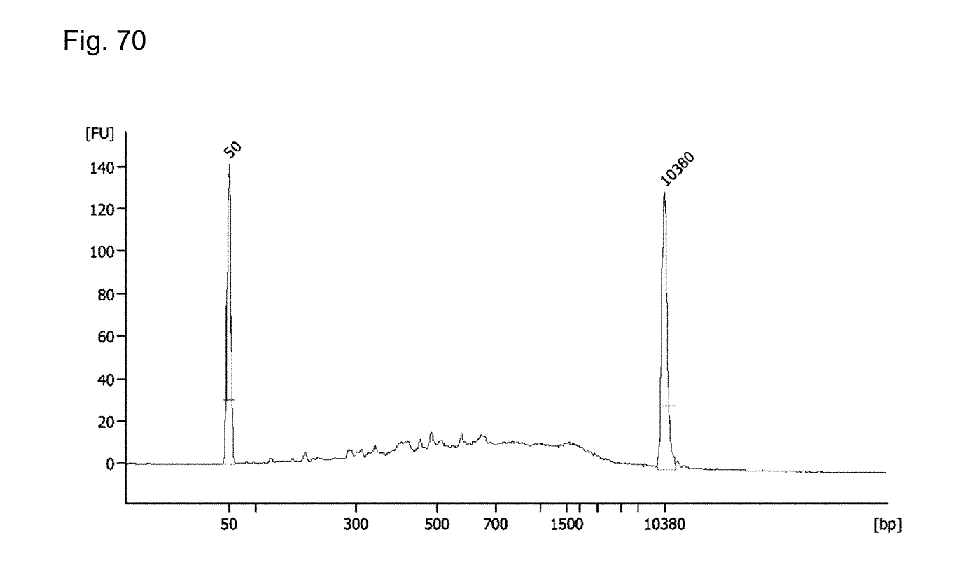

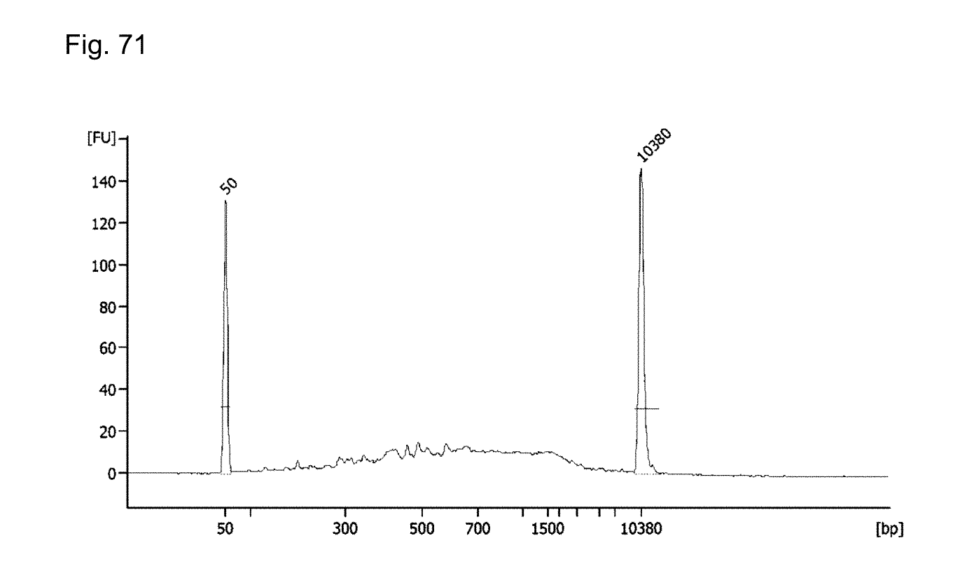

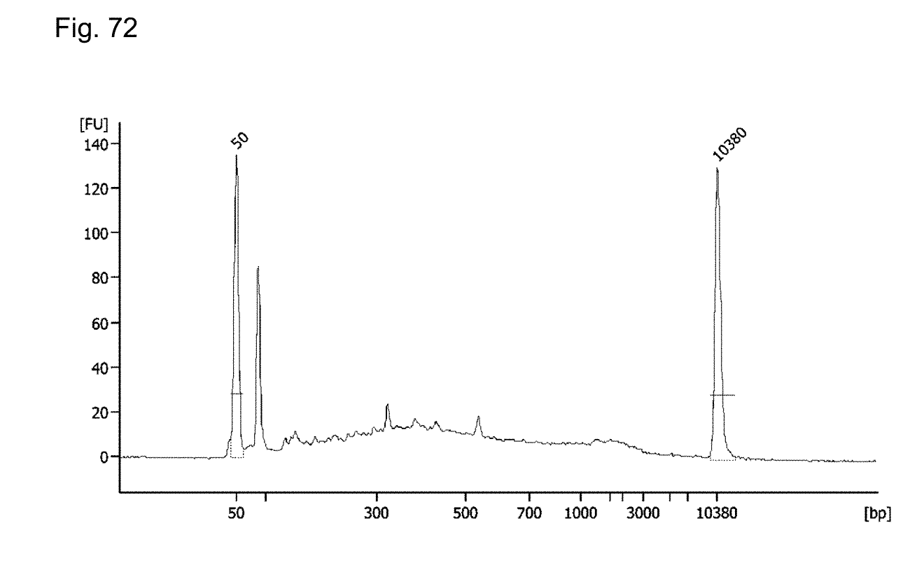

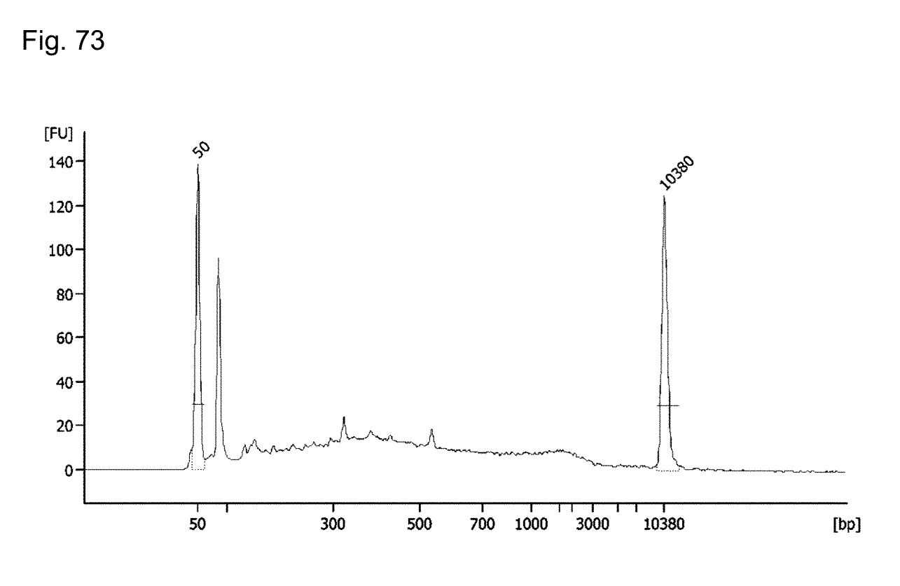

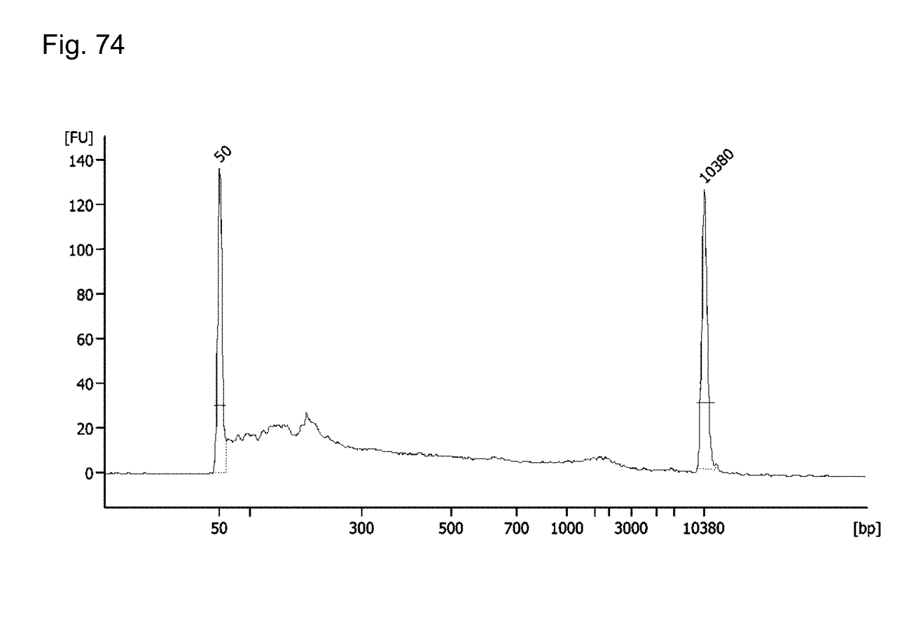

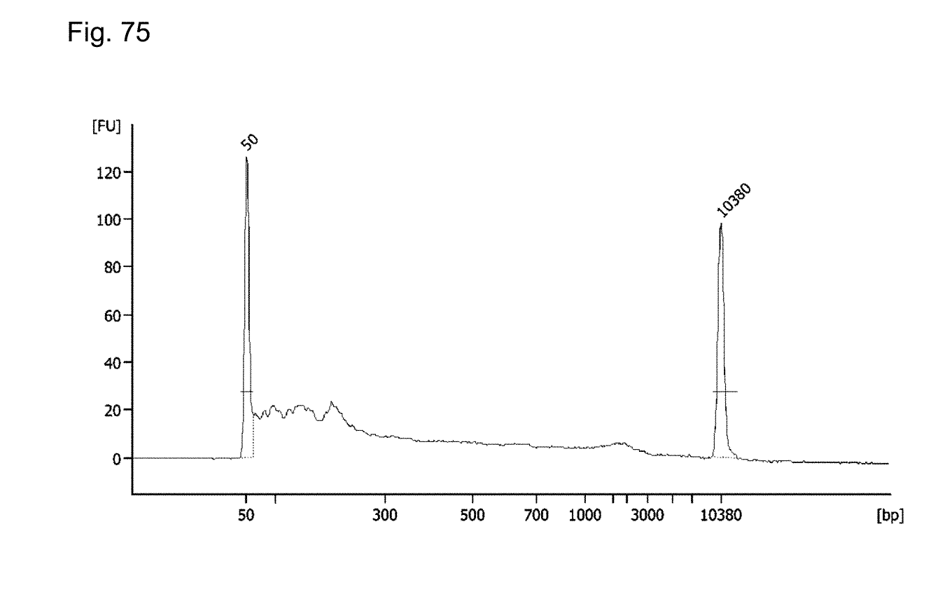

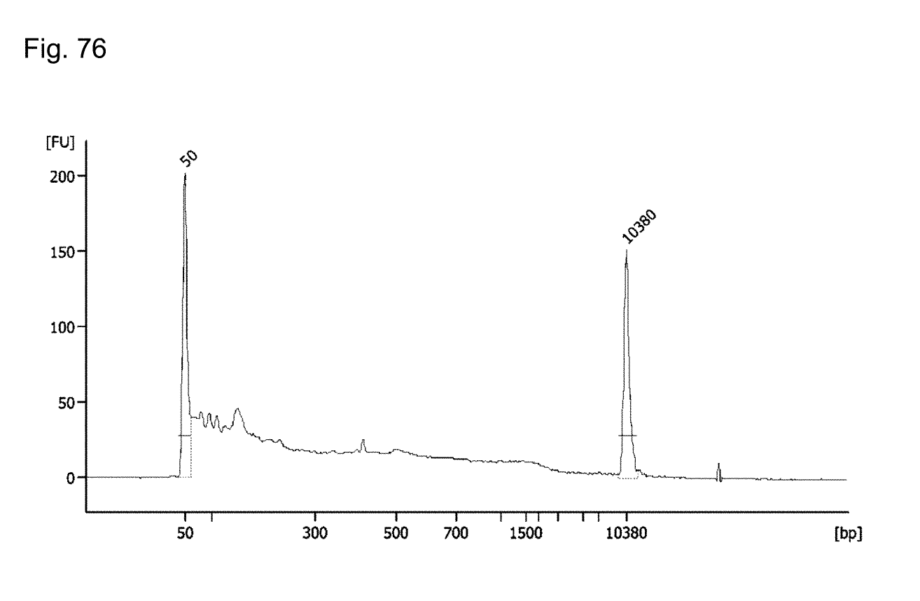

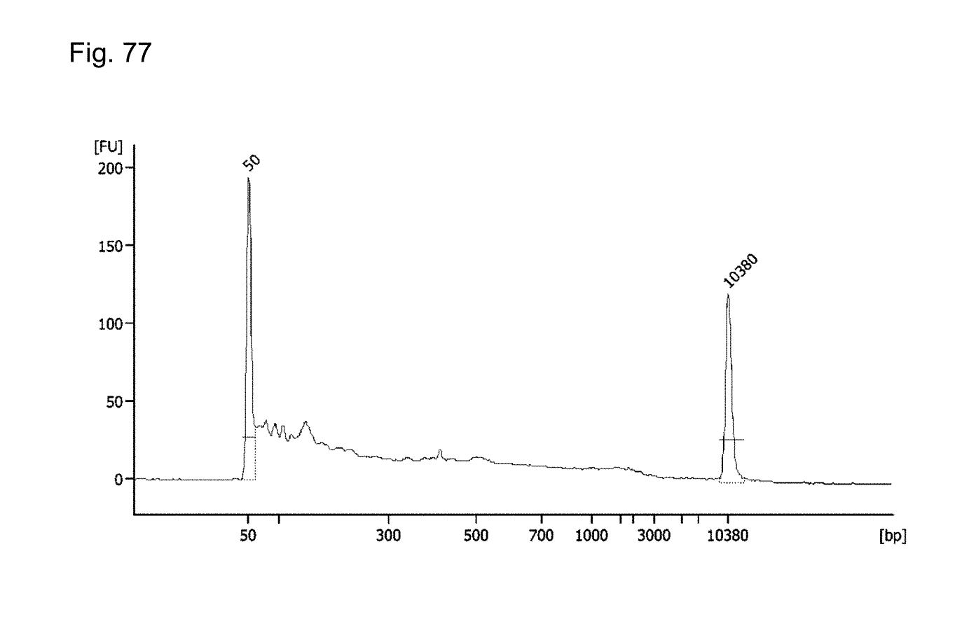

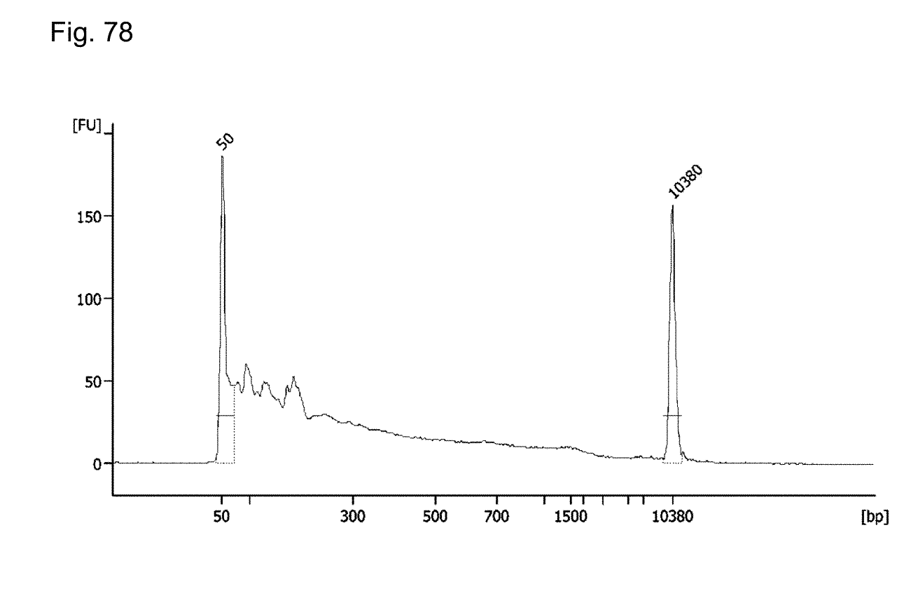

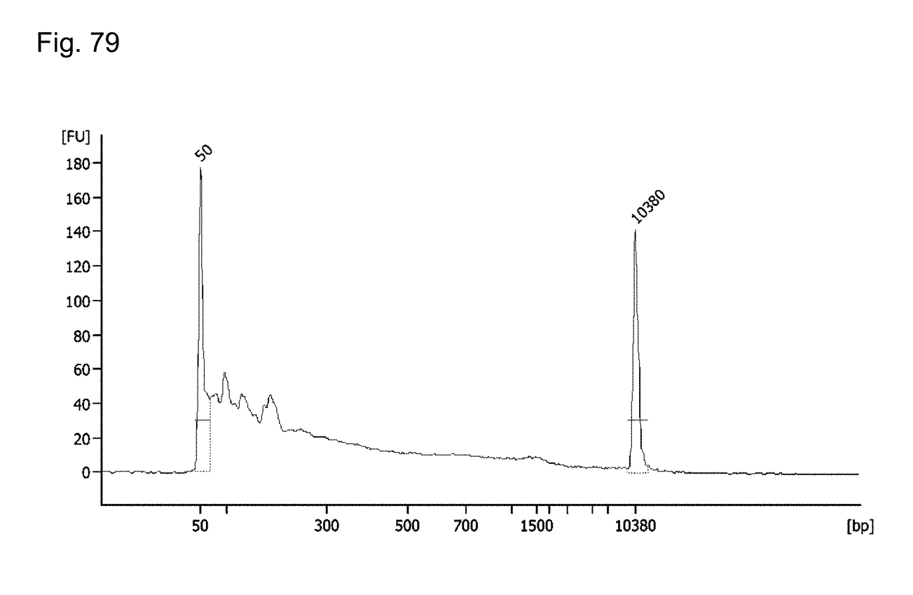

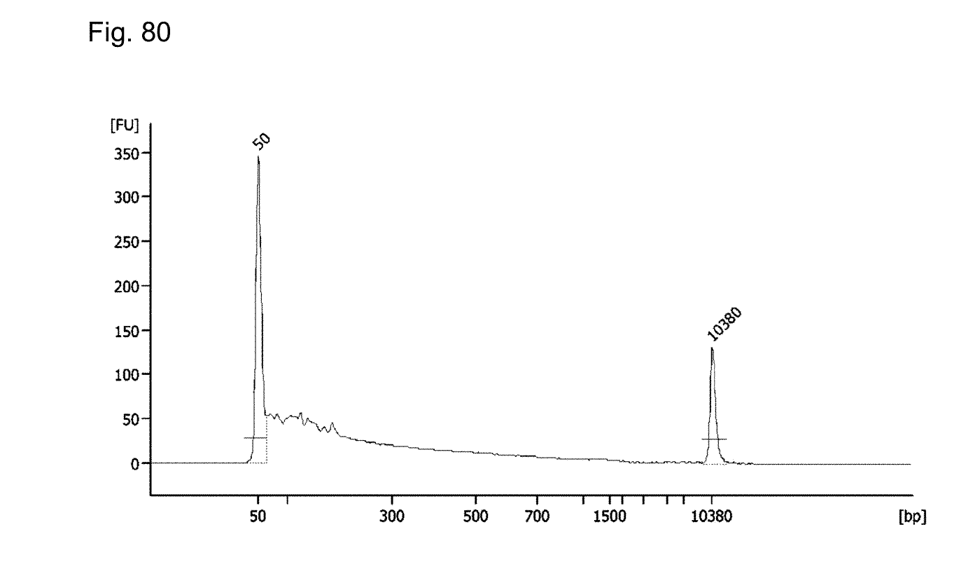

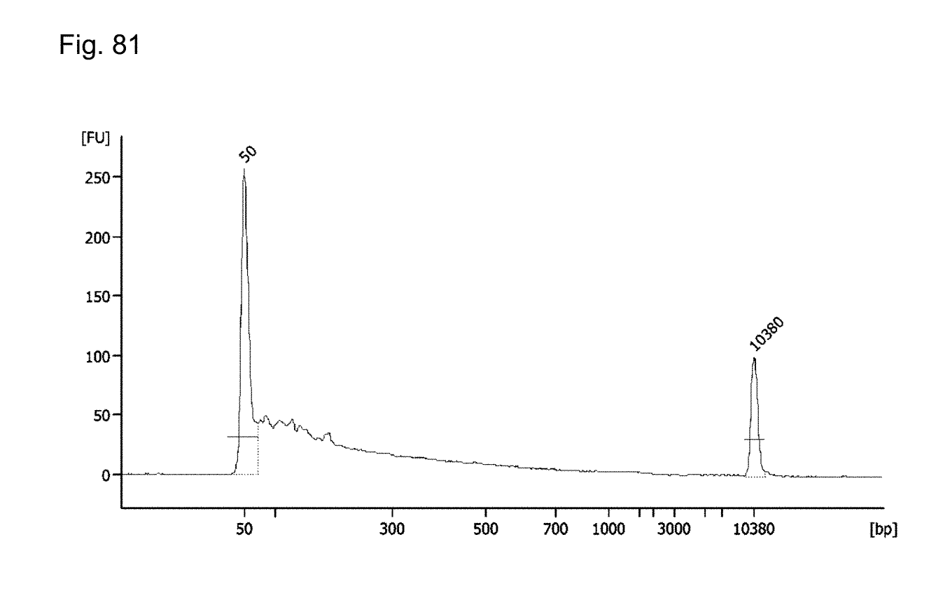

TABLE 25

| Random primer | | | Correlation |
|---|---|---|---|
| length | Repeat | FIG. | coefficient (ρ) |
| 9 | 1st | FIG. 66 | 0.981 |
| | 2nd | FIG. 67 | |
| 10 | 1st | FIG. 68 | 0.979 |
| | 2nd | FIG. 69 | |
| 11 | 1st | FIG. 70 | 0.914 |
| | 2nd | FIG. 71 | |
| 12 | 1st | FIG. 72 | 0.957 |
| | 2nd | FIG. 73 | |
| 14 | 1st | FIG. 74 | 0.984 |
| | 2nd | FIG. 75 | |
| 16 | 1st | FIG. 76 | 0.989 |
| | 2nd | FIG. 77 | |
| 18 | 1st | FIG. 78 | 0.995 |
| | 2nd | FIG. 79 | |
| 20 | 1st | FIG. 80 | 0.999 |
| | 2nd | FIG. 81 | |

When random primers were used at a high concentration of 10.0 μM, which is 13.3 times greater than the usual level, as shown in FIGS. 66 to 81, it was found that a low-molecular-weight DNA fragment could be amplified with the use of random primers comprising 9 to 20 nucleotides while achieving very high reproducibility. As the nucleotide length of a random primer increased (12 nucleotides or more, in particular), the molecular weight of the amplified fragment was likely to be decreased. When random primers comprising 9 nucleotides were used, the amount of the DNA fragment amplified was increased by setting the annealing temperature at 37° C.

In order to elucidate the correlation between the density and the length of random primers, as described in 3.6.2 above, PCR was carried out with the use of random primers comprising 8 to 35 nucleotides at the concentration of 0.6 to 300 μM, so as to produce a DNA library. The results are shown in Table 26.

TABLE 26
The correlation between the concentration and the length of
random primer tor DNA library

| | Concentration | | | | | | | | | | | | | | | | |
|---|---|---|---|---|---|---|---|---|---|---|---|---|---|---|---|---|---|
| Primer | Factor relative | Primer length | | | | | | | | | | | | | | | |
| μM | to reference | 8 | 9 | 10 | 11 | 12 | 14 | 16 | 18 | 20 | 22 | 24 | 26 | 28 | 29 | 30 | 35 |
| 0.6 | Reference | x | x | x | x | x | x | x | x | x | x | x | x | x | x | x | x |
| 2 | 3.3-fold | x | x | x | x | x | x | x | x | x | x | x | x | x | x | x | x |
| 4 | 6.7-fold | x | x | x | x | x | ∘ | ∘ | ∘ | ∘ | ∘ | ∘ | ∘ | ∘ | x | x | x |
| 6 | 10.0-fold | x | x | x | x | x | ∘ | ∘ | ∘ | ∘ | ∘ | ∘ | ∘ | ∘ | ∘ | ∘ | x |
| 8 | 13.3-fold | x | x | x | x | ∘ | ∘ | ∘ | ∘ | ∘ | ∘ | ∘ | ∘ | ∘ | ∘ | x | x |
| 10 | 16.7-fold | x | x | x | x | ∘ | ∘ | ∘ | ∘ | ∘ | ∘ | ∘ | ∘ | ∘ | ∘ | x | x |
| 20 | 33.3-fold | x | x | x | ∘ | ∘ | ∘ | ∘ | ∘ | ∘ | ∘ | ∘ | x | x | x | x | x |
| 40 | 66.7-fold | x | ∘ | ∘ | ∘ | ∘ | ∘ | ∘ | ∘ | ∘ | x | x | x | x | x | x | x |
| 60 | 100.0-fold | x | ∘ | ∘ | ∘ | ∘ | ∘ | ∘ | ∘ | ∘ | x | x | x | x | x | x | x |
| 100 | 166.7-fold | — | x | ∘ | ∘ | ∘ | ∘ | ∘ | ∘ | x | — | — | — | — | — | — | — |
| 200 | 333.3-fold | — | x | ∘ | ∘ | x | x | x | x | x | — | — | — | — | — | — | — |
| 300 | 500.0-fold | — | x | x | x | x | x | x | x | x | — | — | — | — | — | — | — |

∘: DNA library covering 100 to 500 nucleotides could be amplified assuredly with high reproducibility (ρ > 0.9)
x: DNA library did not cover 100 to 500 nucleotides, or the reproducibility was low (ρ <= 0.9)
—: Not carried out

As shown in Table 26, it was found that a low-molecular-weight (100 to 500 nucleotides) DNA fragment could be amplified with high reproducibility with the use of random primers comprising 9 to 30 nucleotides at 4.0 to 200 μM. In particular, it was confirmed that low-molecular-weight (100 to 500 nucleotides) DNA fragments could be amplified assuredly with high reproducibility with the use of random primers comprising 9 to 30 nucleotides at 4.0 to 100 μM.

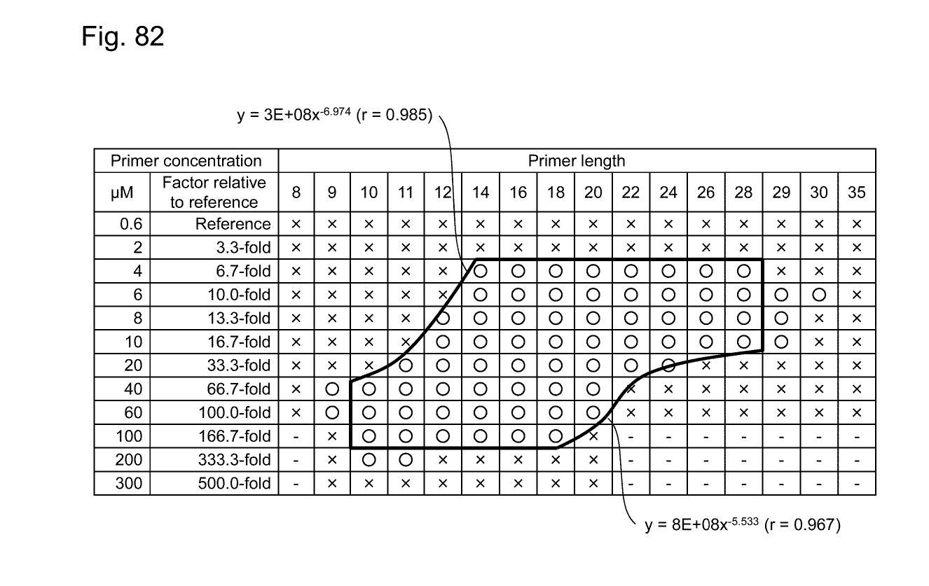

The results shown in Table 26 are examined in greater detail. As a result, the correlation between the length and the concentration of random primers is found to be preferably within a range surrounded by a frame as shown in FIG. 82. More specifically, the random primer concentration is preferably 40 to 60 μM when the random primers comprise 9 to 10 nucleotides. It is preferable that a random primer concentration satisfy the condition represented by an inequation: y>3E+08x^{-6.974}, provided that the nucleotide length of the random primer is represented by y and the random primer concentration is represented by x, and 100 μM or lower, when the random primer comprises 10 to 14 nucleotides. The random primer concentration is preferably 4 to 100 mM when the random primer comprises 14 to 18 nucleotides. When a random primer comprises 18 to 28 nucleotides, the random primer concentration is preferably 4 μM or higher, and it satisfies the condition represented by an inequation: y<8E+08x^{-5.533}. When a random primer comprises 28 to 29 nucleotides, the random primer concentration is preferably 4 to 10 μM. The inequations y>3E+08x^{-6.974} and y<8E+08x^{-5.533} are determined on the basis of the Microsoft Excel power approximation.

By prescribing the number of nucleotides and the concentration of random primers within given ranges as described above, it was found that low-molecular-weight (100 to 500 nucleotides) DNA fragments could be amplified with high reproducibility. For example, the accuracy of the data obtained via analysis of high-molecular-weight DNA fragments with the use of a next-generation sequencer is known to deteriorate to a significant extent. As described in this Example, the number of nucleotides and the concentration of random primers may be prescribed within given ranges, so that a DNA library with a molecular size suitable for analysis with a next-generation sequencer can be produced with satisfactory reproducibility, and such DNA library can be suitable for marker analysis with the use of a next-generation sequencer.

### 4.7 Number of Random Primers

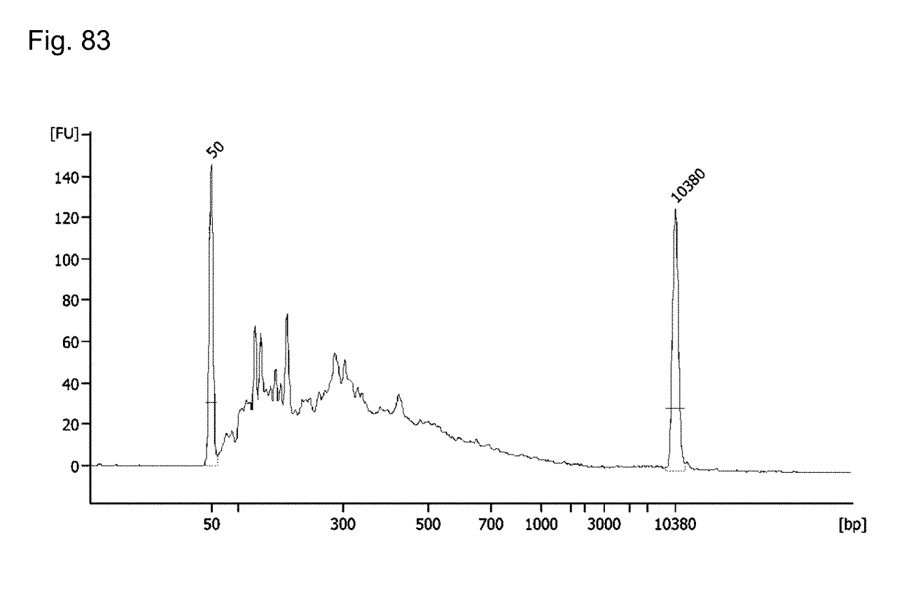

As described in 3.7 above, 1, 2, 3, 12, 24, or 48 types of random primers (concentration: 60 μM) were used to produce a DNA library, and the results are shown in FIGS. 83 to 94. The results are summarized in Table 27.

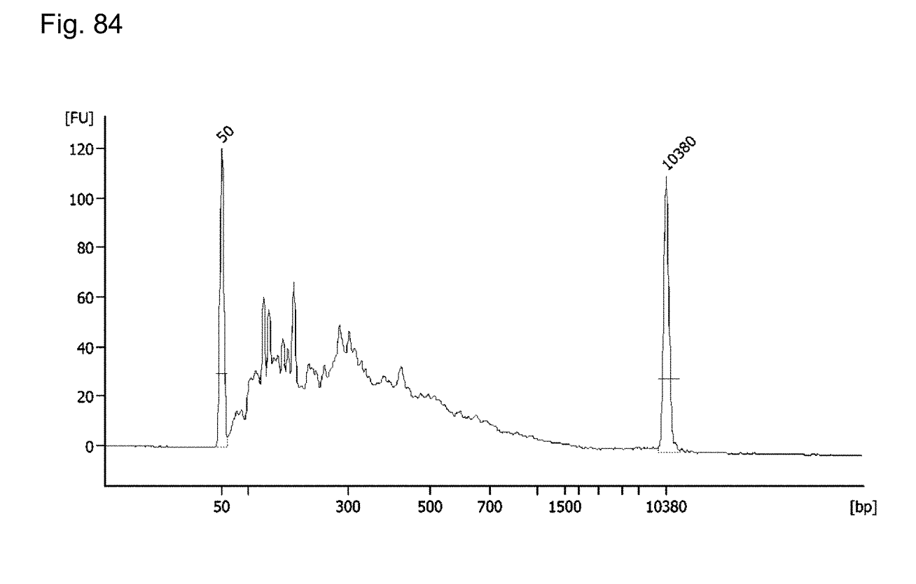

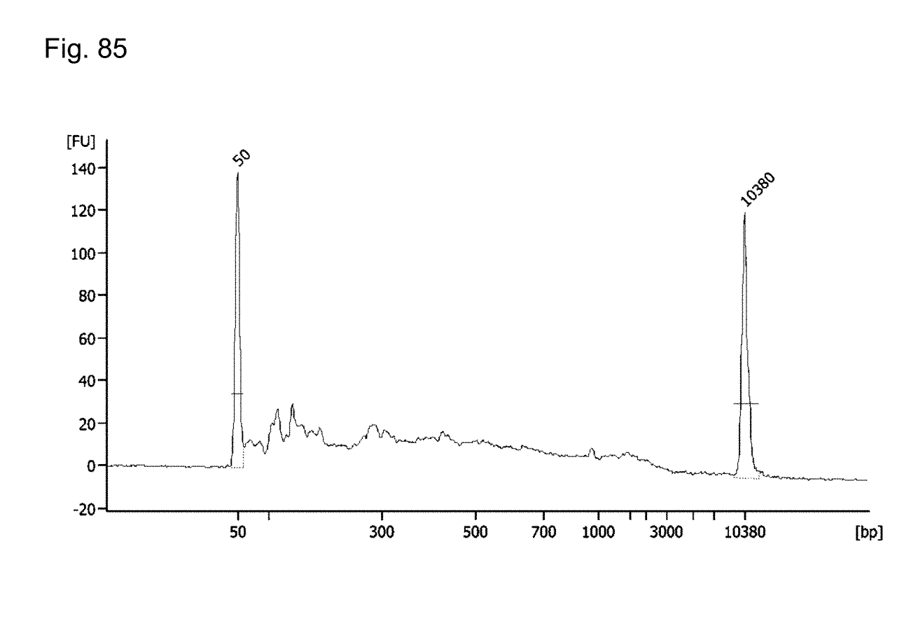

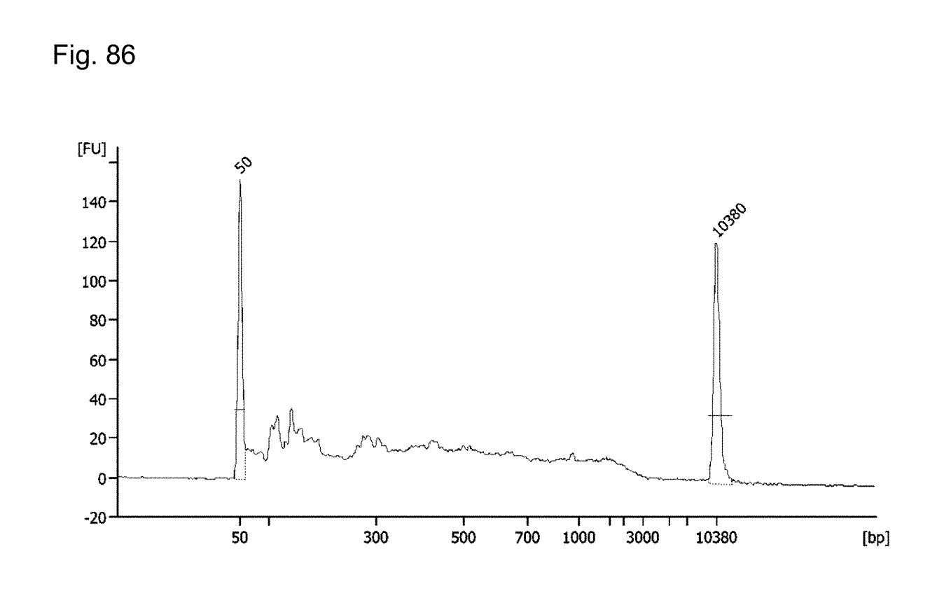

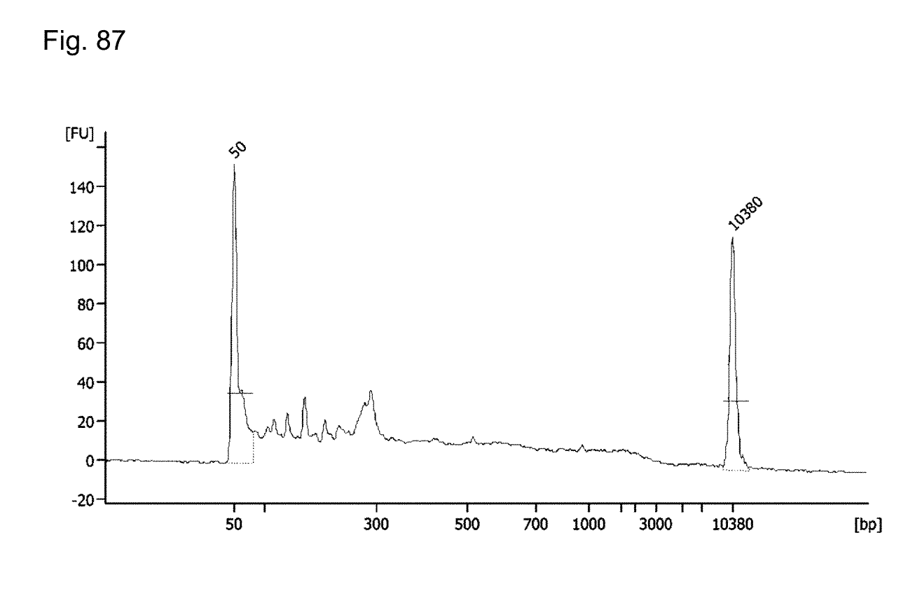

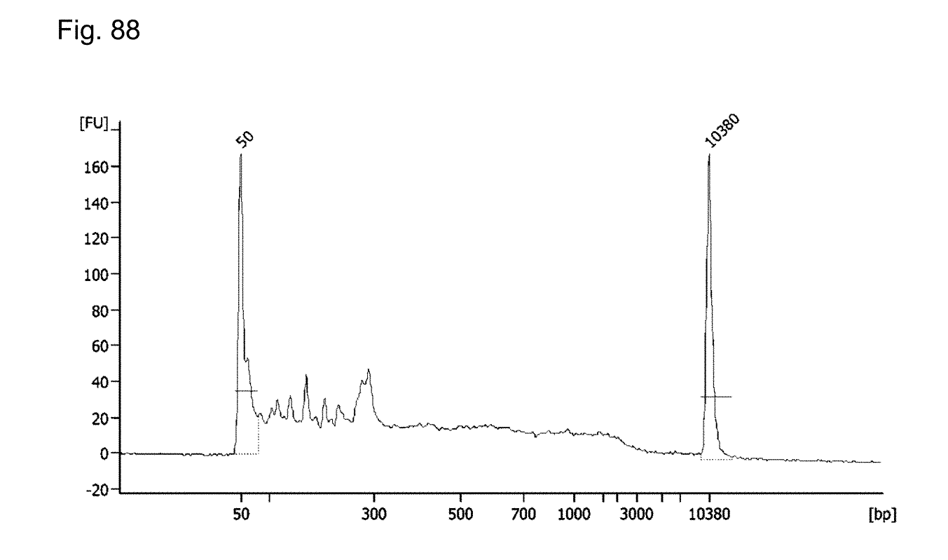

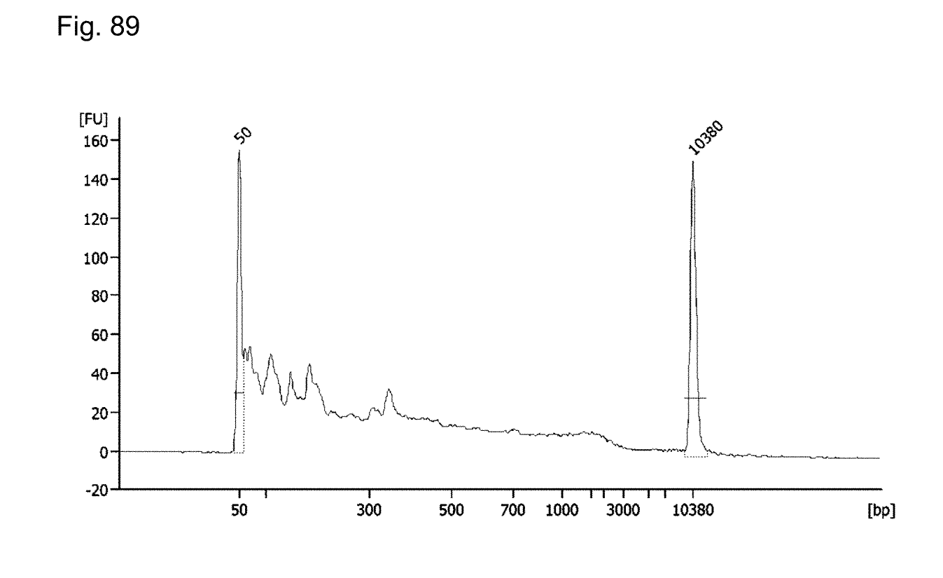

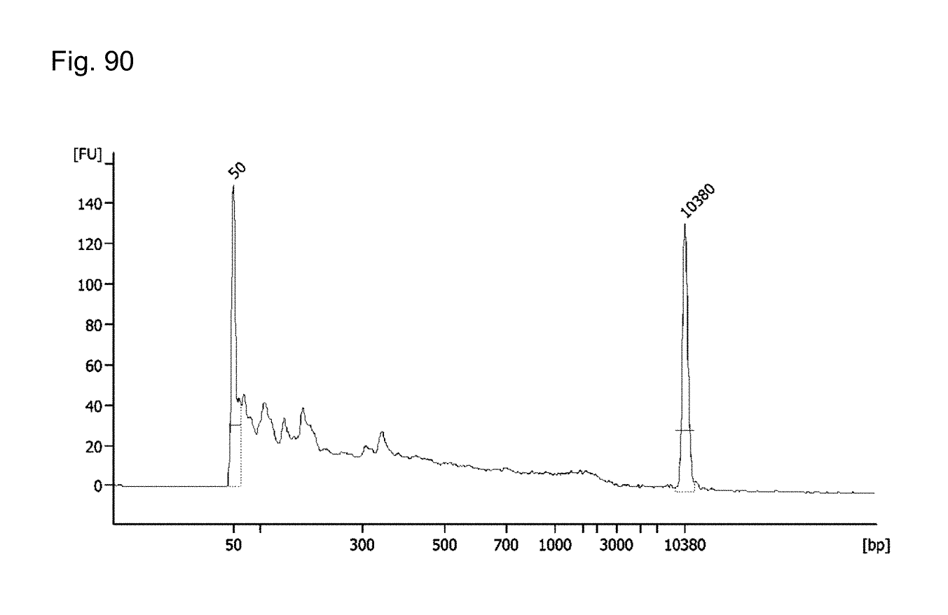

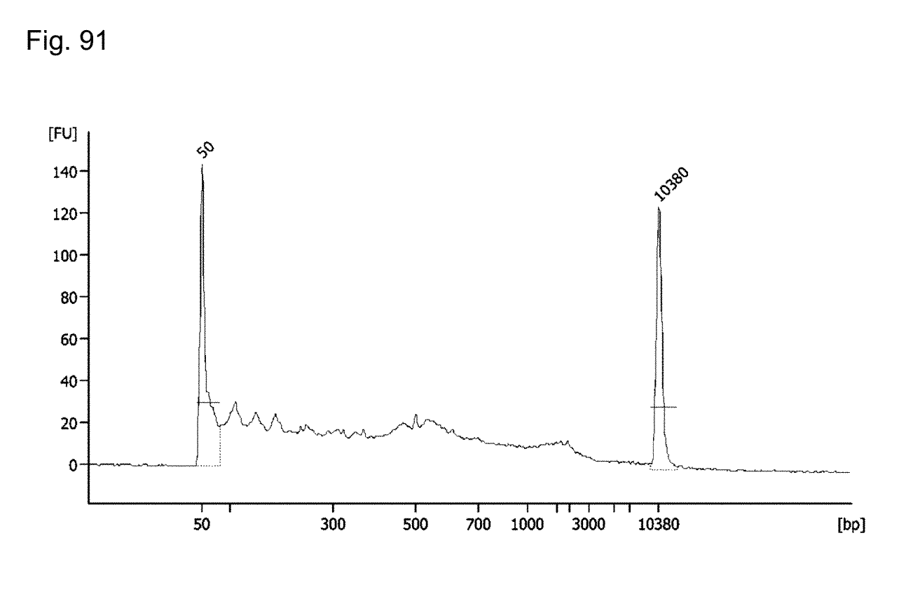

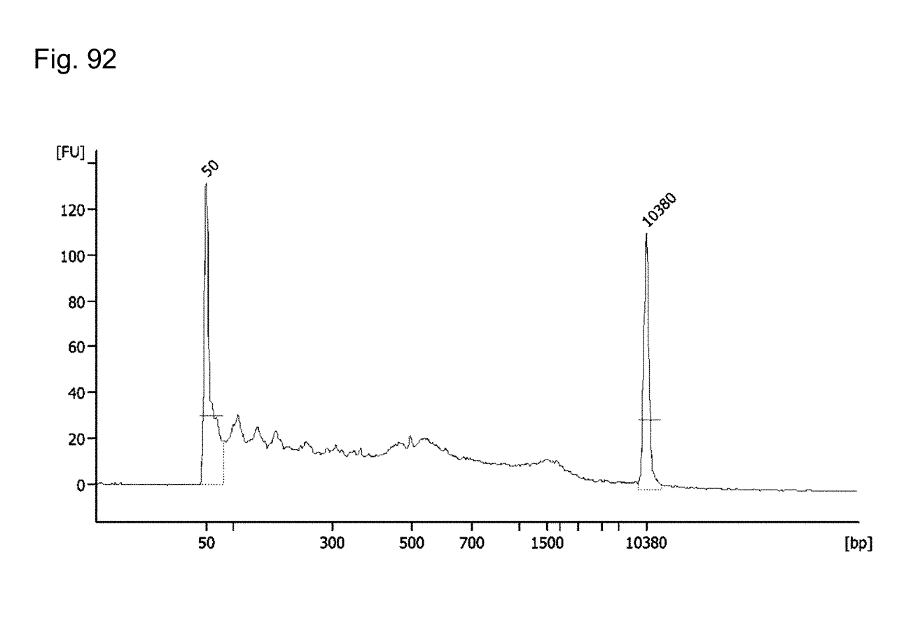

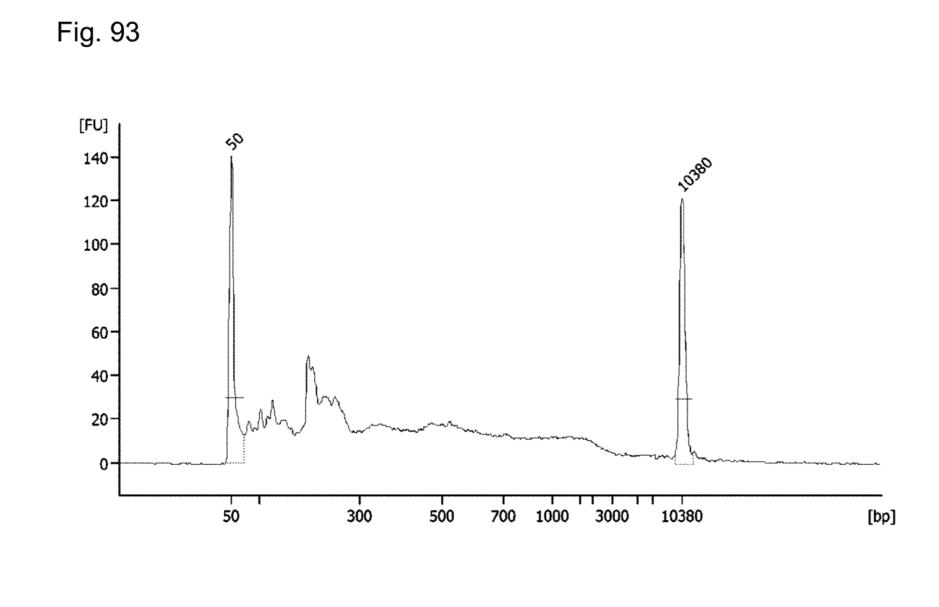

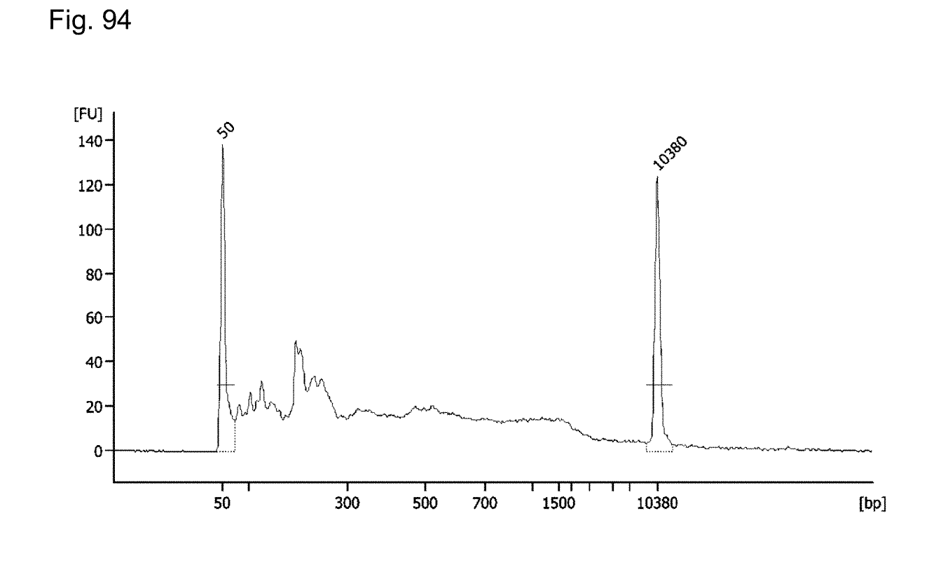

TABLE 27

| Number of | | | Correlation |
|---|---|---|---|
| random primers | Repeat | FIG. | coefficient (ρ) |
| 1 | 1st | FIG. 83 | 0.984 |
| | 2nd | FIG. 84 | |
| 2 | 1st | FIG. 85 | 0.968 |
| | 2nd | FIG. 86 | |
| 3 | 1st | FIG. 87 | 0.974 |
| | 2nd | FIG. 88 | |
| 12 | 1st | FIG. 89 | 0.993 |
| | 2nd | FIG. 90 | |
| 24 | 1st | FIG. 91 | 0.986 |
| | 2nd | FIG. 92 | |
| 48 | 1st | FIG. 93 | 0.978 |
| | 2nd | FIG. 94 | |

As shown in FIGS. 83 to 94, it was found that low-molecular-weight DNA fragments could be amplified with the use of any of 1, 2, 3, 12, 24, or 48 types of random primers while achieving very high reproducibility. In particular, it is understood that as the number of types of random primers increases, a peak in the electrophoretic pattern decreases, and a deviation is likely to disappear.

### 4.8 Random Primer Sequence

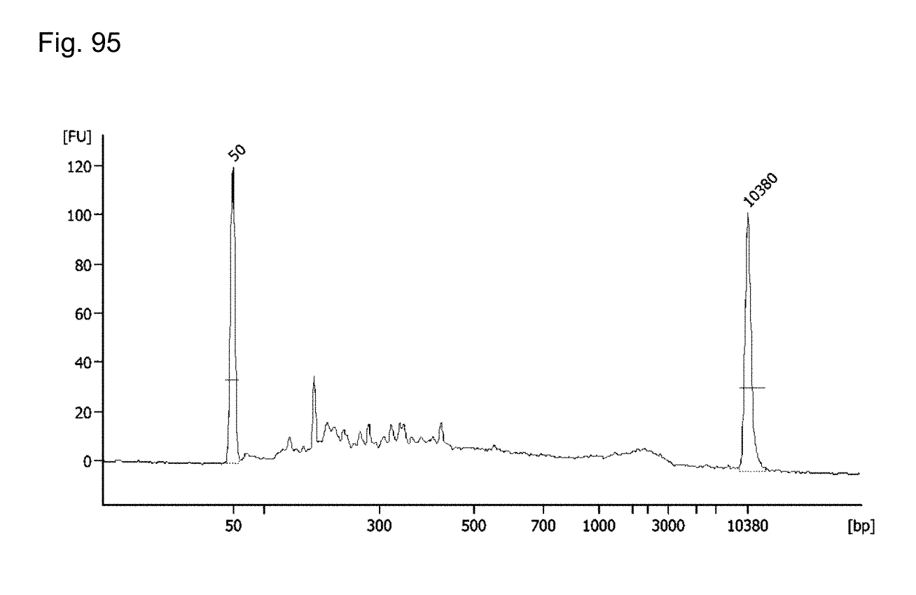

As described in 3.8 above, DNA libraries were produced with the use of sets of random primers shown in Tables 2 to 6 (i.e., 10-nucleotide primer B, 10-nucleotide primer C, 10-nucleotide primer D, 10-nucleotide primer E, and 10-nucleotide primer F), and the results are shown in FIGS. 95 to 104. The results are summarized in Table 28.

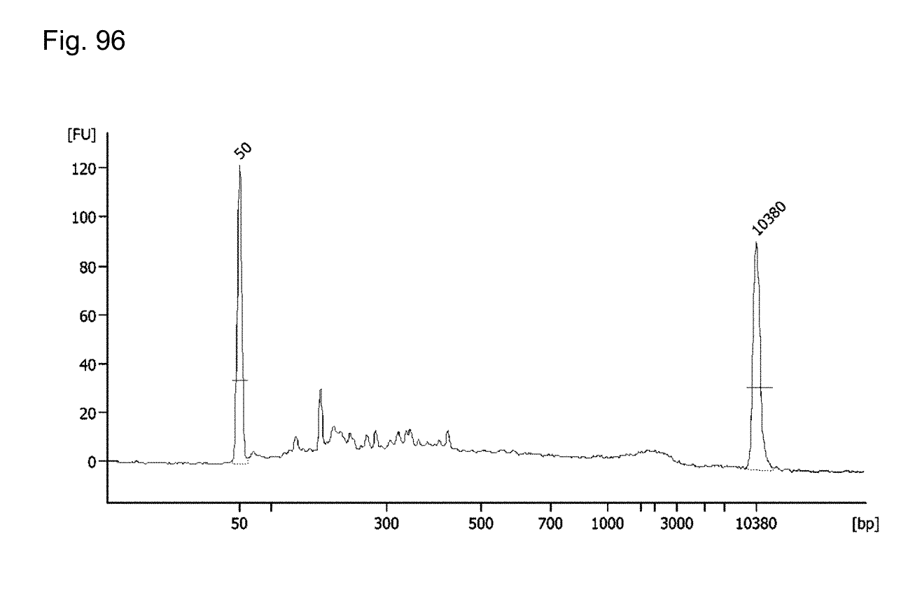

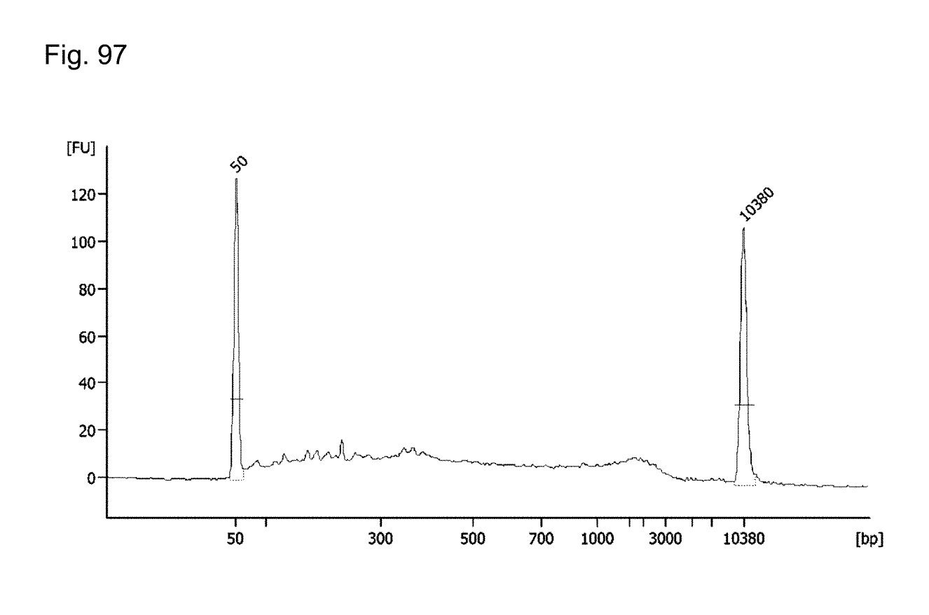

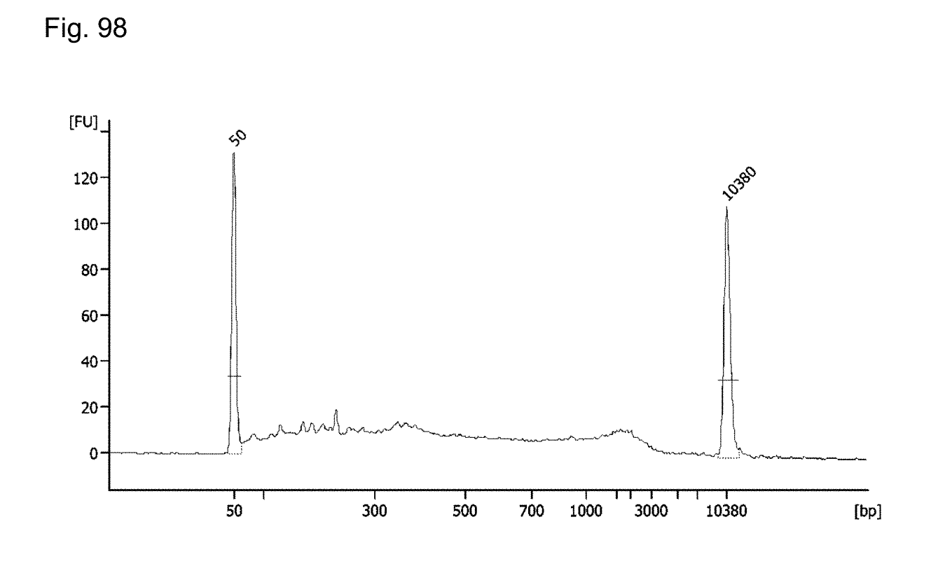

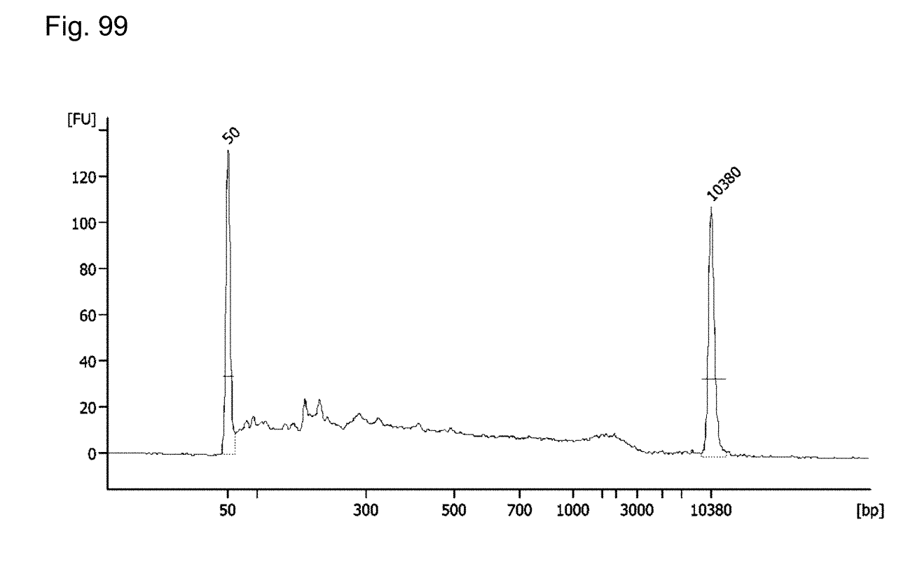

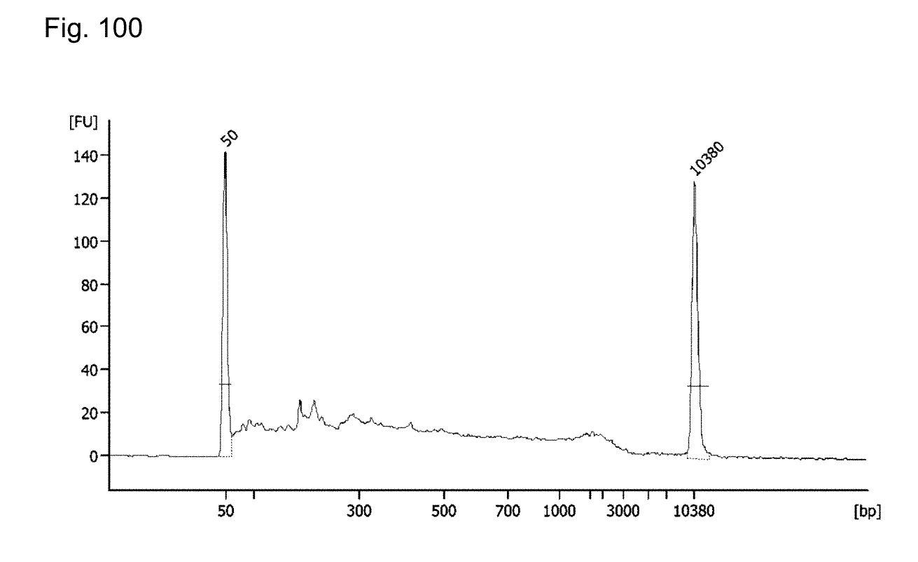

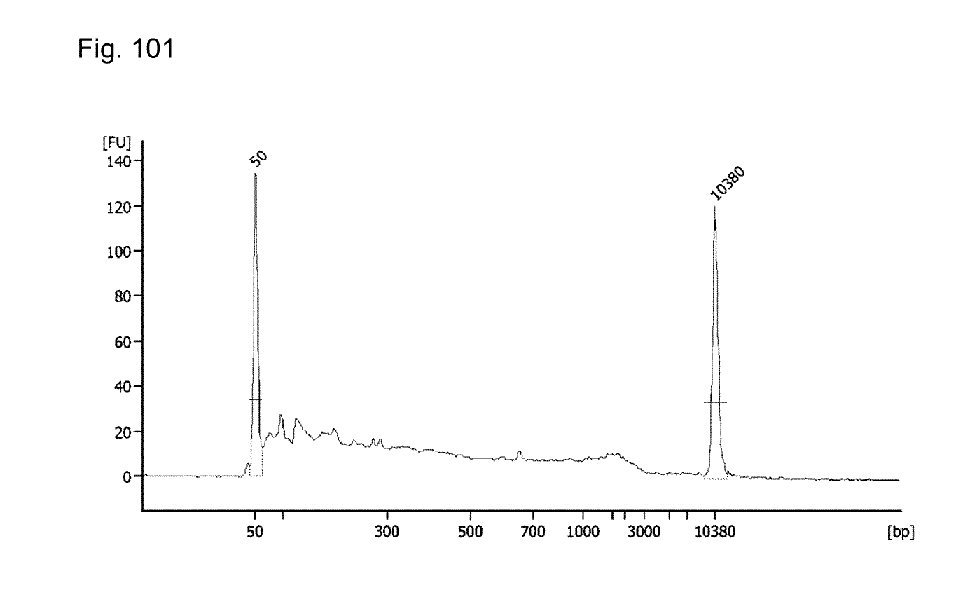

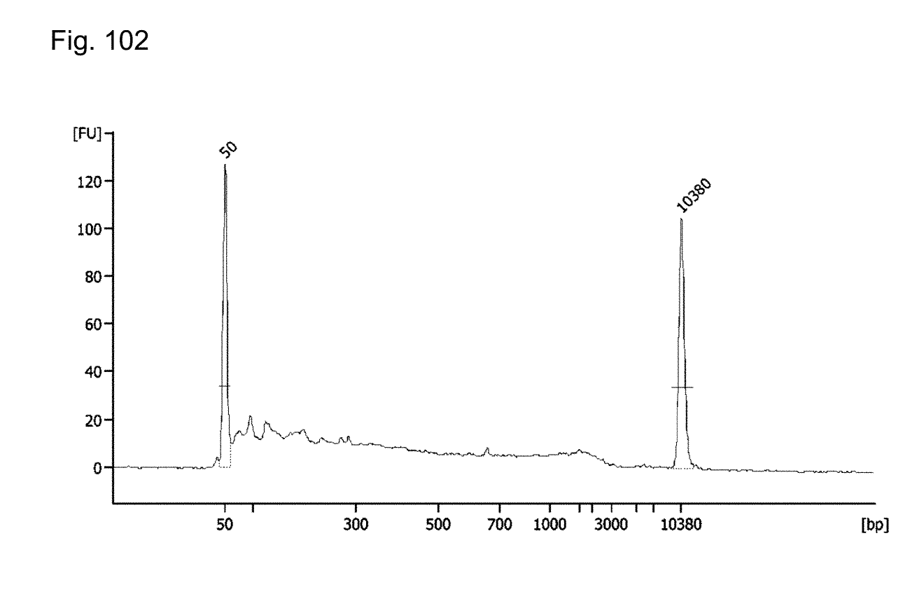

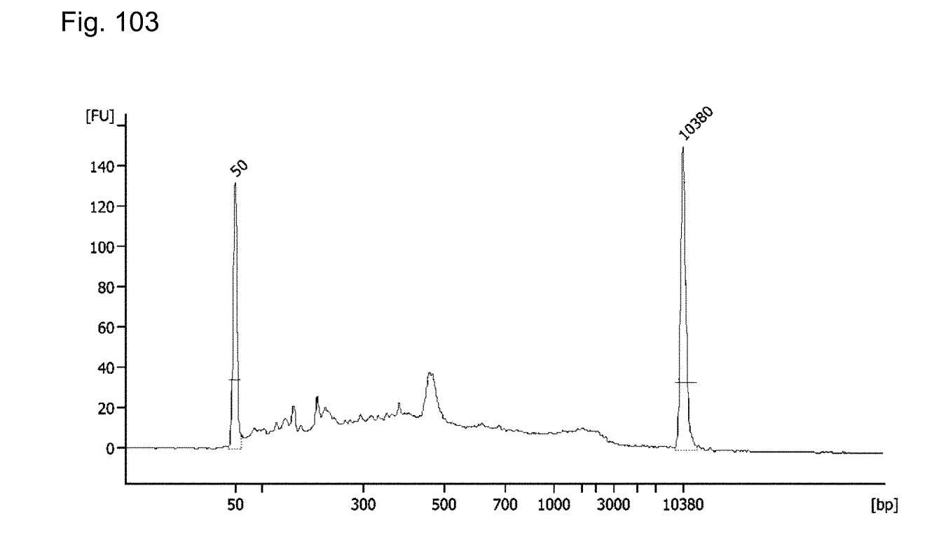

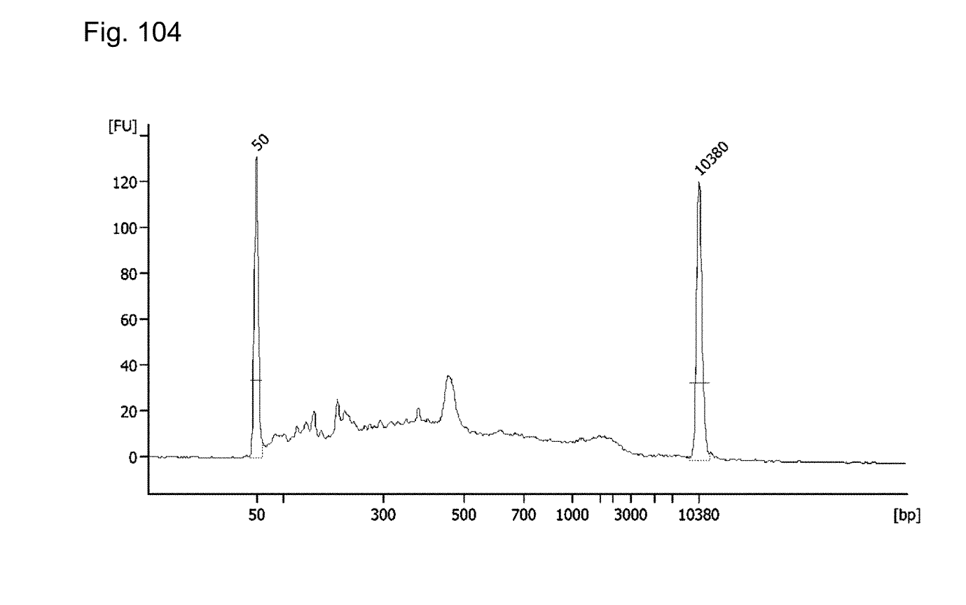

TABLE 28

| | | | Correlation |
|---|---|---|---|
| Random primer set | Repeat | FIG. | coefficient (ρ) |
| 10-nucleotide B | 1st | FIG. 95 | 0.916 |
| | 2nd | FIG. 96 | |
| 10-nucieotide C | 1st | FIG. 97 | 0.965 |
| | 2nd | FIG. 98 | |
| 10-nucleotide D | 1st | FIG. 99 | 0.986 |
| | 2nd | FIG. 100 | |
| 10-nucieotide E | 1st | FIG. 101 | 0.983 |
| | 2nd | FIG. 102 | |
| 10-nucleotide F | 1st | FIG. 103 | 0.988 |
| | 2nd | FIG. 104 | |

As shown in FIGS. 95 to 104, it was found that low-molecular-weight DNA fragments could be amplified with the use of any sets of 10-nucleotide primer B, 10-nucleotide primer C, 10-nucleotide primer D, 10-nucleotide primer E, or 10-nucleotide primer F while achieving very high reproducibility.

### 4.9 Production of Human DNA Library

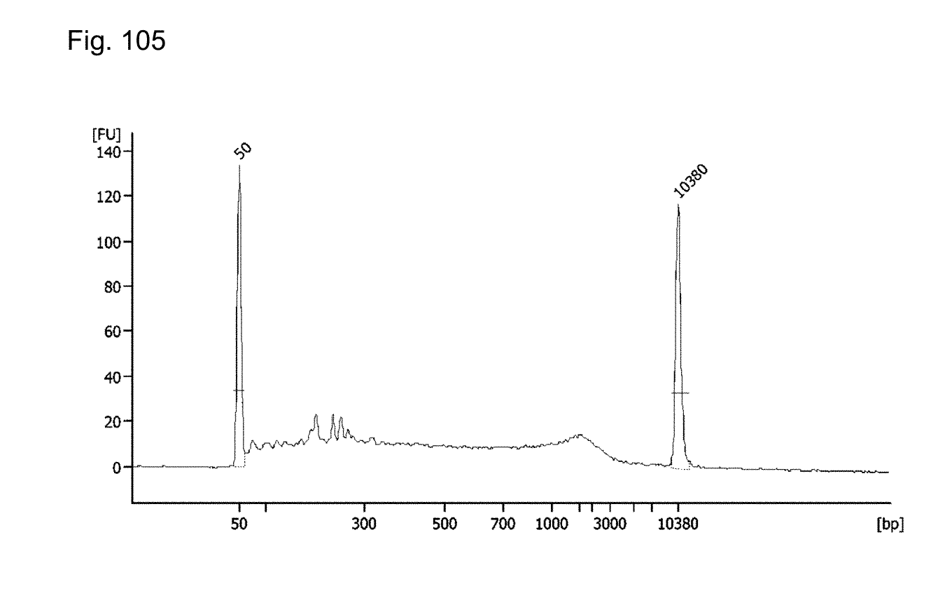

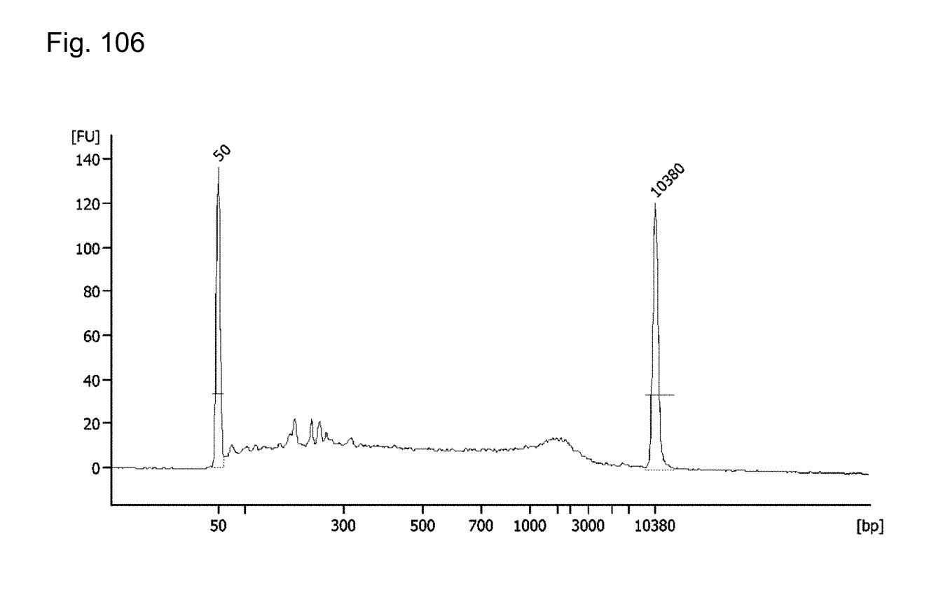

As described in 3.9 above, a DNA library was produced with the use of human-derived genomic DNA and random primers at a final concentration of 60 μM (10-nucleotide primer A), and the results are shown in FIGS. 105 and 106. FIG. 105 shows the results of the first repeated experiment, and FIG. 106 shows the results of the second repeated experiment. As shown in FIGS. 105 and 106, it was found that low-molecular-weight DNA fragments could be amplified while achieving very high reproducibility even if human-derived genomic DNA was used.

### Example 2

### 1. Flowchart

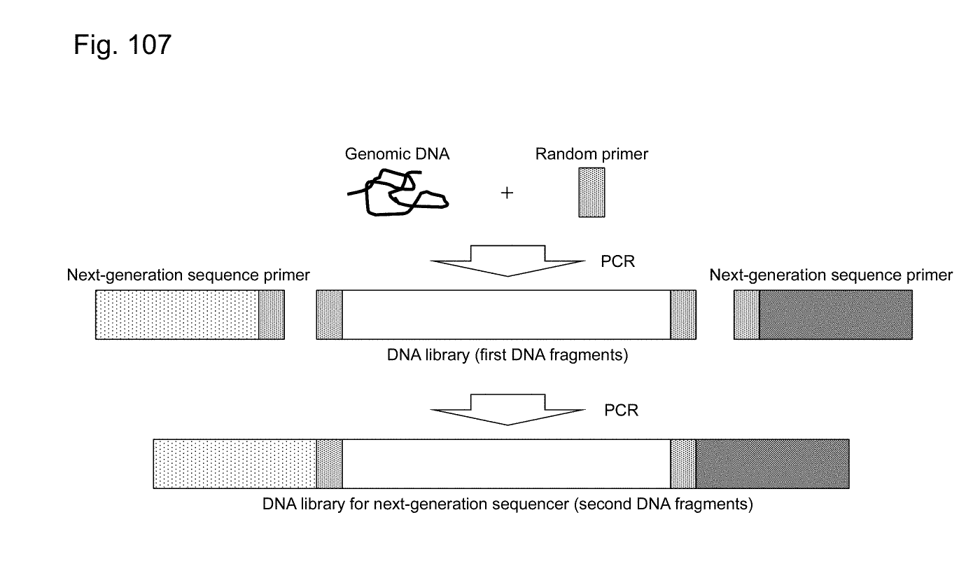

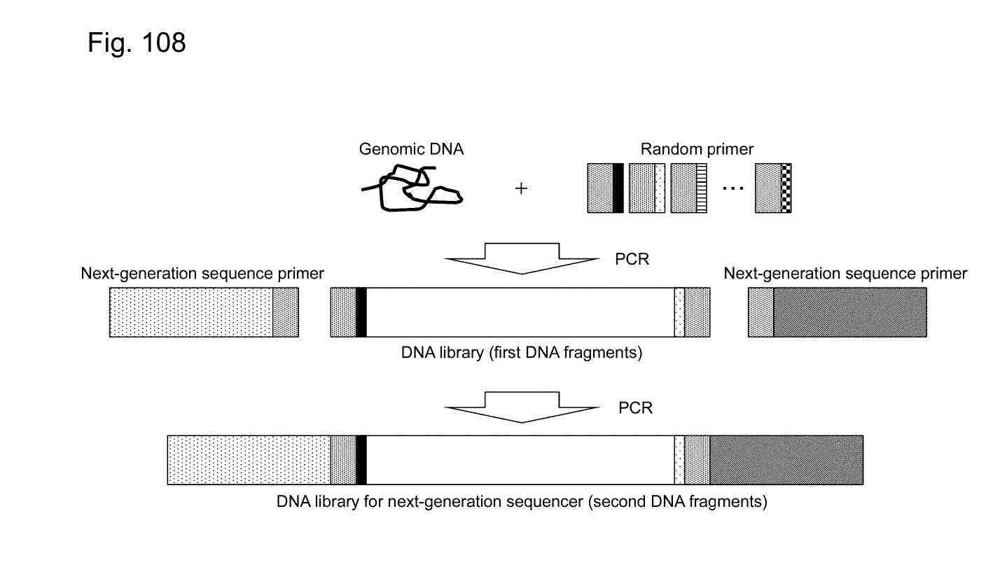

In this Example, first DNA fragments were prepared by PCR using genomic DNA as a template and random primers according to the schematic diagrams shown in FIGS. 107 and 108. Subsequently, second DNA fragments were prepared by PCR using the first DNA fragments as templates and next-generation sequencer primers. The prepared second DNA fragments were used as a sequencer library for conducting sequence analysis using a so-called next generation sequencer. Genotype was analyzed based on the obtained read data.

### 2. Materials

In this Example, genomic DNAs were extracted from the sugarcane variety NiF8 and the rice variety Nipponbare using the DNeasy Plant Mini Kit (QIAGEN), and the extracted genomic DNAs were purified. The purified genomic DNAs were used as NiF8-derived genomic DNA and Nipponbare-derived genomic DNA, respectively.

### 3. Method

### 3.1 Examination of Sugarcane Variety NiF8

### 3.1.1 Designing of Random Primers and Next-Generation Sequencer Primers

In this Example, random primers were designed based on 3′-end 10 nucleotides of the next-generation sequencer adapter (Nextera adapter, Illumina, Inc.). Specifically, in this Example, GTTACACACG (SEQ ID NO: 2041, 10-nucleotide G) was used as a random primer. In addition, next-generation sequencer primers were designed based on the sequence information on the Nextera adapter of Illumina, Inc. in the above manner (Table 29).

TABLE 29

| No. | Primer sequence | SEQ ID NO: |
|---|---|---|
| 1 | AATGATACGGCGACCACCGAGATCTACA | 2042 |
| | CCTCTCTATTCGTCGGCAGCGTCAGATG | |
| | TGTATAAGAGACAG | |
| 2 | CAAGCAGAAGACGGCATACGAGATTAAG | 2043 |
| | GCGAGTCTCGTGGGCTCGGAGATGTGT | |
| | ATAAGAGACAG | |

### 3.1.2 Preparation of DNA Library

A dNTP mixture at a final concentration of 0.2 mM, MgCl₂ at a final concentration of 1.0 mM, and DNA Polymerase (TAKARA, PrimeSTAR) at a final concentration of 1.25 units, and a random primer (10-nucleotide G) at a final concentration of 60 μM were added to NiF8-derived genomic DNA (30 ng) described in 2, above. A DNA library (first DNA fragments) was prepared by PCR (treatment at 98° C. for 2 minutes, reaction for 30 cycles of 98° C. for 10 seconds, 50° C. for 15 seconds, and 72° C. for 20 seconds, and storage at 4° C.) in a final reaction volume of 50 μl.

### 3.1.3 Purification and Electrophoresis

The DNA library obtained in 3.1.2 above was purified with the use of the MinElute PCR Purification Kit (QIAGEN) and subjected to electrophoresis with the use of the Agilent 2100 bioanalyzer (Technologies) to obtain a fluorescence unit (FU). Also, the reproducibility of the repeated data was evaluated on the basis of the Spearman's rank correlation (p>0.9).

### 3.1.4 Preparation of Next-Generation Sequencer DNA Library

A dNTP mixture at a final concentration of 0.2 mM, MgCl₂ at a final concentration of 1.0 mM, DNA Polymerase (TAKARA, PrimeSTAR) at a final concentration of 1.25 units, and a next-generation sequencer primer at a final concentration of 0.5 μM were added to the first DNA fragment (100 ng) purified in 3.1.3 above. A next-generation sequencer DNA library (second DNA fragments) was prepared by PCR (treatment at 95° C. for 2 minutes, reaction for 25 cycles of 98° C. for 15 seconds, 55° C. for 15 seconds, 72° C. for 20 seconds, treatment at 72° C. for 1 minutes, and storage at 4° C.) in a final reaction volume of 50 μl. The DNA library for a next-generation sequencer was subjected to purification and electrophoresis in the same manner as in 3.1.3.

### 3.1.5 MiSeq Analysis

The next-generation sequencer DNA library (a second DNA fragment) in 3.1.4 above was analyzed by MiSeq via 100 base paired-end sequencing using MiSeq Reagent Kit V2 500 Cycle (Illumina).

### 3.1.6 Read Data Analysis

The read patterns were identified from the read data obtained in 3.1.5. The number of reads was counted for each read pattern, the number of reads of the repeated analyses, and the reproducibility was evaluated using the correlational coefficient.

### 3.2 Examination of Rice Variety Nipponbare

### 3.2.1 Designing of Random Primers and Next-Generation Sequencer Primers

In this Example, random primers were designed based on 10 nucleotides of the 3′ end of the next-generation sequencer adapter Nextera adapter of Illumina, Inc. That is, in this Example, a sequence of 10 nucleotides positioned at the 3′ end of the Nextera adapter and 16 types of nucleotide sequences prepared by adding an arbitrary nucleotide sequence of 2 nucleotides to the 3′ end of the sequence of 10 nucleotides to results in a full length of 12 nucleotides were designed as random primers (Table 30, 12-nucleotide B).

TABLE 30

| No. | Primer sequence | SEQ ID NO: |
|---|---|---|
| 1 | TAAGAGACAGAA | 2044 |
| 2 | TAAGAGACAGAT | 2045 |
| 3 | TAAGAGACAGAC | 2046 |
| 4 | TAAGAGACAGAG | 2047 |
| 5 | TAAGAGACAGTA | 2048 |
| 6 | TAAGAGACAGTT | 2049 |
| 7 | TAAGAGACAGTC | 2050 |
| 8 | TAAGAGACAGTG | 2051 |
| 9 | TAAGAGACAGCA | 2052 |
| 10 | TAAGAGACAGCT | 2053 |
| 11 | TAAGAGACAGCC | 2054 |
| 12 | TAAGAGACAGCG | 2055 |
| 13 | TAAGAGACAGGA | 2056 |
| 14 | TAAGAGACAGGT | 2057 |
| 15 | TAAGAGACAGGC | 2058 |
| 16 | TAAGAGACAGGG | 2059 |

In addition, in this Example, a next-generation sequencer primer designed based on the sequence information on the Nextera adapter of Illumina. Inc. in the same manner as in 3.1.1.

### 3.2.2 Preparation of DNA Library

A dNTP mixture at a final concentration of 0.2 mM, MgCl₂ at a final concentration of 1.0 mM, and DNA Polymerase (TAKARA, PrimeSTAR) at a final concentration of 1.25 units, and a random primer (12-nucleotide B) at a concentration of 40 μM were added to Nipponbare-derived genomic DNA (30 ng) described in 2, above. A DNA library (first DNA fragments) was prepared by PCR (treatment at 98° C. for 2 minutes, reaction for 30 cycles of 98° C. for 10 seconds, 50° C. for 15 seconds, 72° C. for 20 seconds, and storage at 4° C.) in a final reaction volume of 50 μl.

### 3.2.3 Purification and Electrophoresis

The DNA library obtained in 3.2.2 above was purified with the use of the MinElute PCR Purification Kit (QIAGEN) and subjected to electrophoresis with the use of the Agilent 2100 bioanalyzer (Technologies) to obtain a fluorescence unit (FU). Also, the reproducibility of the repeated data was evaluated on the basis of the Spearman's rank correlation (p>0.9).

### 3.2.4 Preparation of Next-Generation Sequencer DNA Library

A dNTP mixture at a final concentration of 0.2 mM. MgCl₂ at a final concentration of 1.0 mM, DNA Polymerase (TAKARA, PrimeSTAR) at a final concentration of 1.25 units, and a next-generation sequencer primer at a concentration of 0.5 j±M were added to the first DNA fragment (100 ng) purified in 3.2.3 above. A next-generation sequencer DNA library (second DNA fragments) was prepared by PCR (treatment at 95° C. for 2 minutes, reaction for 25 cycles of 98° C. for 15 seconds, 55° C. for 15 seconds, 72° C. for 20 seconds, treatment at 72° C. for 1 minutes, and storage at 4° C.) in a final reaction volume of 50 μl. Purification of the DNA library for next-generation sequencers and electrophoresis were conducted in the same manner as in 3.1.3.

### 3.2.5 MiSeq Analysis

The next-generation sequencer DNA library (second DNA fragment) in 3.2.4 above was analyzed by MiSeq via 100 base paired-end sequencing using MiSeq Reagent Kit V2 500 Cycle (Illumina).

### 3.2.6 Read Data Analysis

The read patterns in 3.2.5 were mapped to the genomic information of Nipponbare (NC_008394 to NC_008405) using bowtie2, the degree of consistency between the random primer sequence and genomic DNA was confirmed. The read patterns were identified from the read data obtained in 3.2.5. The number of reads was counted for each read pattern, the number of reads of the repeated analyses, and the reproducibility was evaluated using the correlational coefficient.

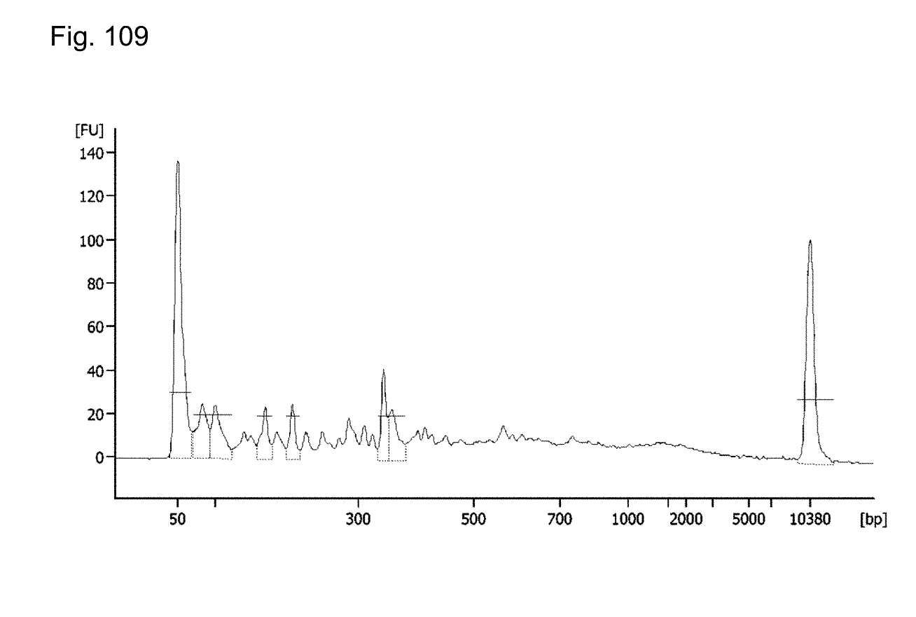

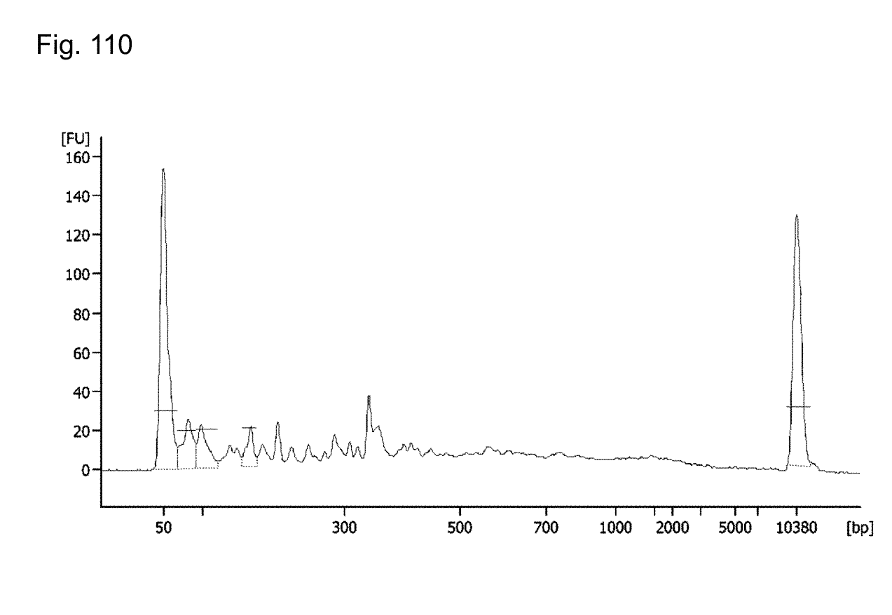

4. Results and Examination 4.1 Results of examination of the sugarcane variety NiF8 FIGS. 109 and 110 show the results of electrophoresis after conducting PCR using a random primer consisting of 10 nucleotides (10-nucleotide G) of the 3′ end of the next-generation sequencer adapter (Nextera adapter, Illumina, Inc.) at a high concentration of 60 μl. As shown in FIGS. 109 and 110, amplification was observed in a wide region ranging from 100 bp to 500 bp (the first DNA fragment). It was considered that amplification could be observed in a wide region because amplification was observed also in a region other than the genomic DNA region corresponding to the random primer. In addition, since the rank correlation coefficient among the repeated data was 0.957 (>0.9), reproducibility was confirmed in the amplification pattern.

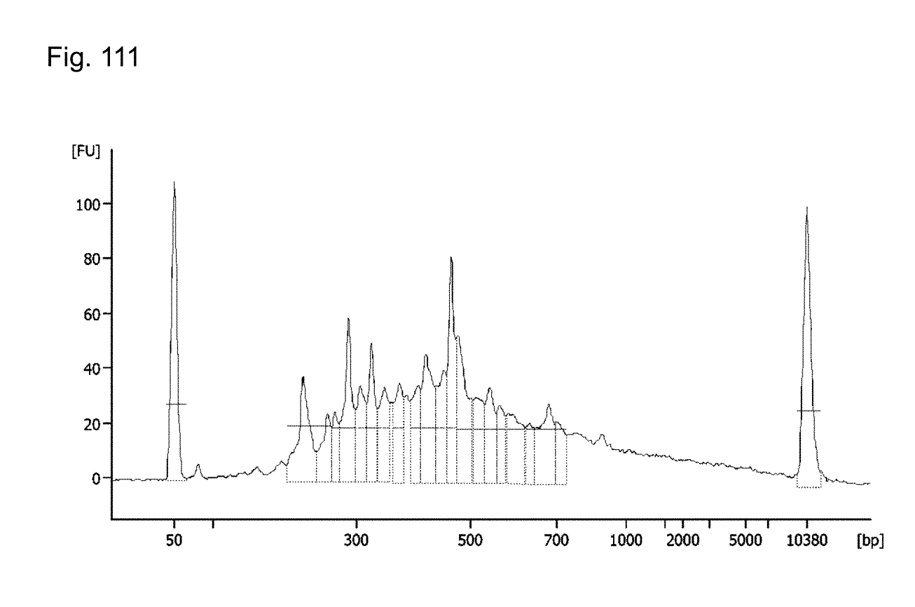

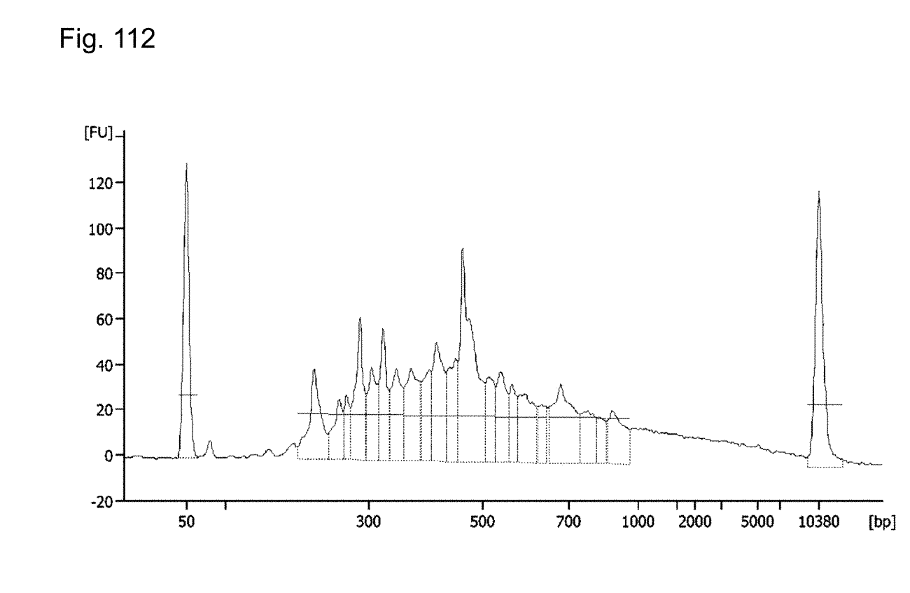

Next, FIGS. 111 and 112 shows the results of electrophoresis after conducting PCR using the next-generation sequencer primer in the manner described in 3.1.4. That is, in order to prepare a DNA library (second DNA fragments) bound to a next-generation sequencer adapter (Nextera adapter). PCR was conducted using a next-generation sequencer primer comprising the sequence of the Nextera adapter of Illumina, Inc. and the first DNA fragment as a template. Accuracy of analysis with the use of the next-generation sequencer of Illumina, Inc. is significantly reduced in a case in which the DNA library includes may short fragments having lengths of 100) bp or less or long fragments having lengths of 1 kbp or more. Since the next-generation sequencer DNA library (second DNA fragments) prepared in this Example was distributed mainly in a range of 150 bp to 1 kbp with a peak around 500 bp as illustrated in FIGS. 111 and 112, the DNA library was considered to be an appropriate next-generation sequencer DNA library. In addition, since the rank correlation coefficient among the repeated data was 0.989 (>0.9), reproducibility was confirmed in the amplification pattern.

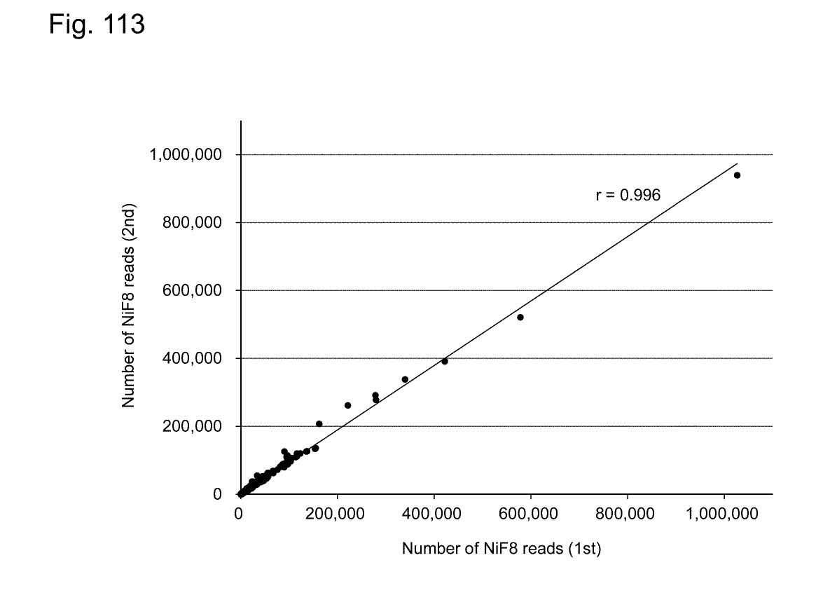

In addition, as a result of analysis of the DNA library (second DA fragment) by next-generation sequencer MiSeq, 3.5-Gbp read data and 3.6-Gbp read data were obtained. The values indicating accuracy of MiSeq data (>=Q30) were 93.3% and 93.1%. Since the values recommended by the manufacturer were 3.0 Gbp or more for read data and 85.0% or more for >=Q30, the next-generation sequencer DNA library (second DNA fragments) prepared in this Example was considered to be applicable to next-generation sequencer analysis. In order to confirm reproducibility, the number of reads of the repeated analyses were compared for 34,613 read patterns obtained by MiSeq. FIG. 113 shows the results. As shown in FIG. 113, there was a high correlation of r=0.996 in terms of the number of reads of the repeated analyses as with the results of electrophoresis.

As described above, a DNA library (first DNA fragments) was obtained by conducting PCR using random primer comprising 10 nucleotides at the 3′ end of a next-generation sequencer adapter (Nextera Adaptor, Illumina, Inc.) at a high concentration, and then. PCR was conducted using a next-generation sequencer primer comprising the sequence of Nextera Adaptor. Accordingly, it was possible to conveniently produce a next-generation sequencer DNA library (second DNA fragments) comprising many fragments with favorable reproducibility.

### 4.2 Results of Examination of Rice Variety Nipponbare

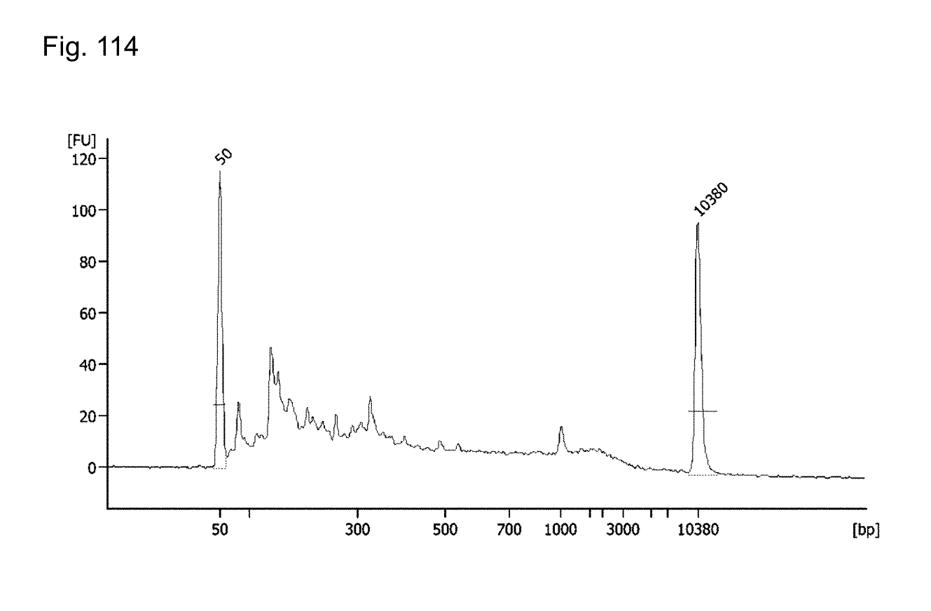

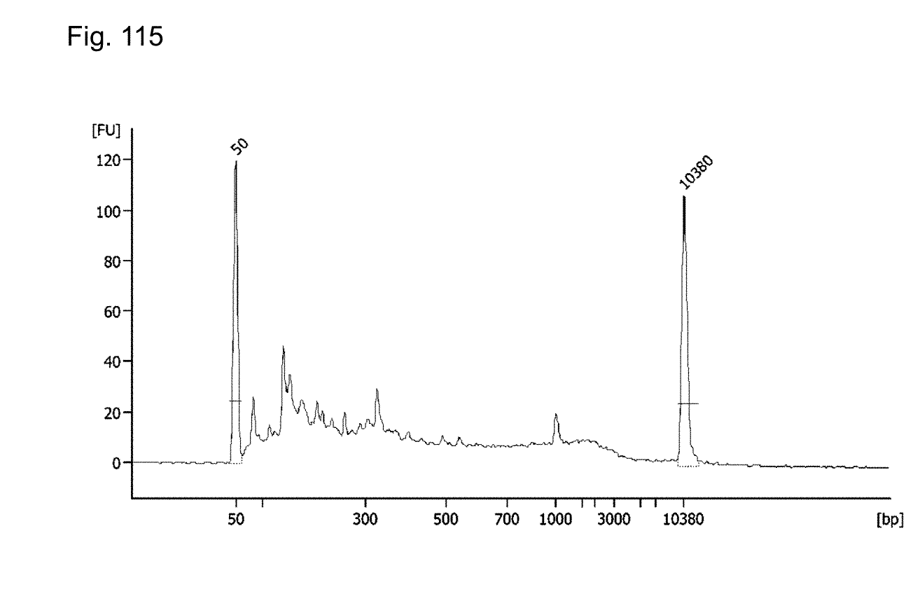

FIGS. 114 and 115 show the results of electrophoresis after conducting PCR using 10 nucleotides positioned at the 3′ end of the next-generation sequencer adopter (Nextera adaptor, Illumina. Inc.) and 16 types of random primers (12-nucleotide B) having a full length of 12 nucleotides obtained by adding an arbitrary sequence of 2 nucleotides to the sequence of 10 nucleotides at the 3′ end at a high concentration of 40 μl. As shown in FIGS. 114 and 115, amplification was observed in a wide region ranging from 100 bp to 500 bp (the first DNA fragment). It was considered that amplification could be observed in a wide region because amplification was observed also in a region other than the genomic DNA region corresponding to the random primer as in the case of 4.1. In addition, since the rank correlation coefficient was 0.950 (>0.9), reproducibility was confirmed in the amplification pattern.

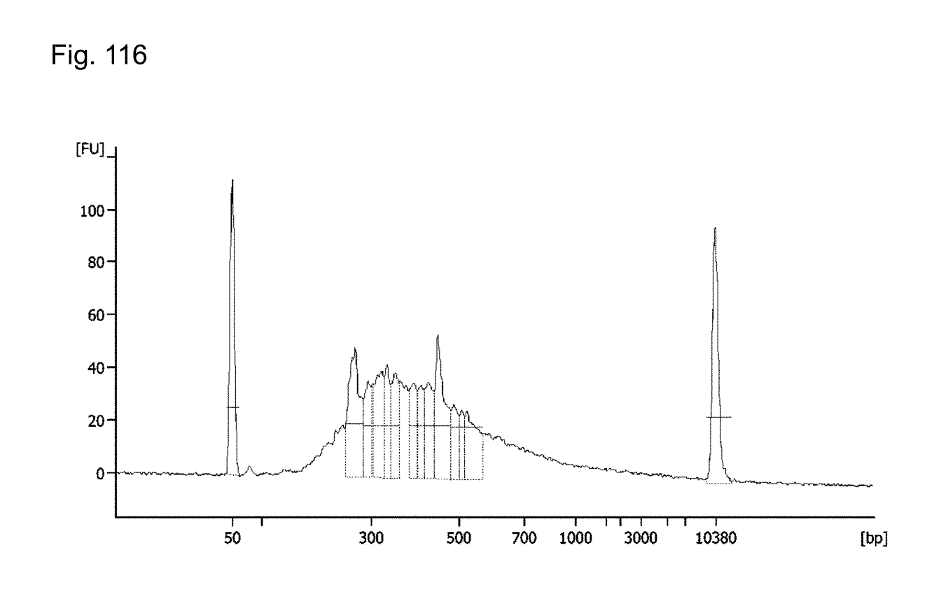

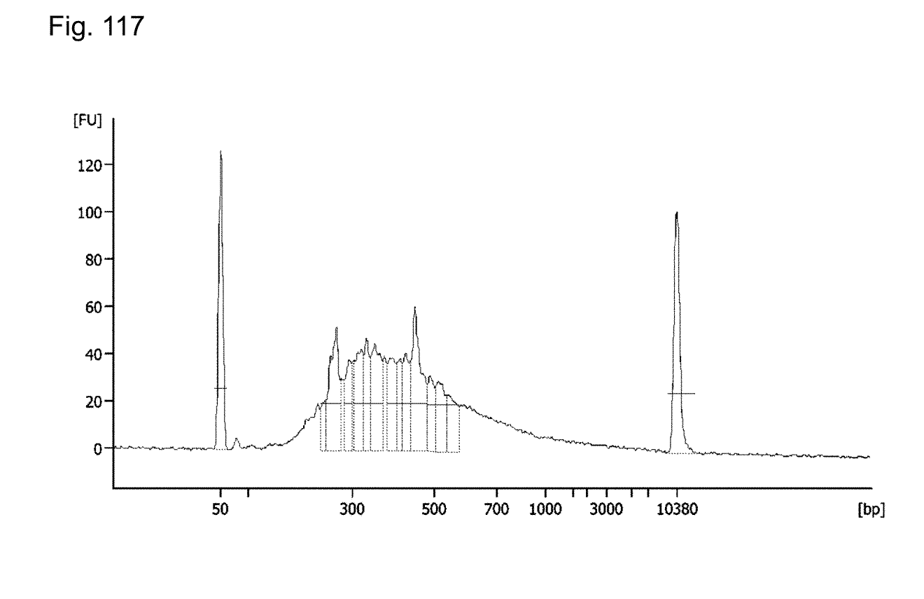

Next, FIGS. 116 and 117 shows the results of electrophoresis after conducting PCR using the next-generation sequencer primer in the manner described in 3.2.4. That is, in order to prepare a DNA library (second DNA fragments) bound to a next-generation sequencer adapter (Nextera adapter), PCR was conducted using a next-generation sequencer primer comprising the sequence of the Nextera adapter of Illumina, Inc. and the first DNA fragment as a template. As a result, since the next-generation sequencer DNA library (the second DNA fragment) prepared in this Example was distributed mainly in a range of 150 bp to 1 kbp with a peak around 300 bp as illustrated in FIGS. 116 and 117, the DNA library was considered to be an appropriate next-generation sequencer DNA library. In addition, since the rank correlation coefficient among the repeated data was 0.992 (>0.9), reproducibility was confirmed in the amplification pattern.

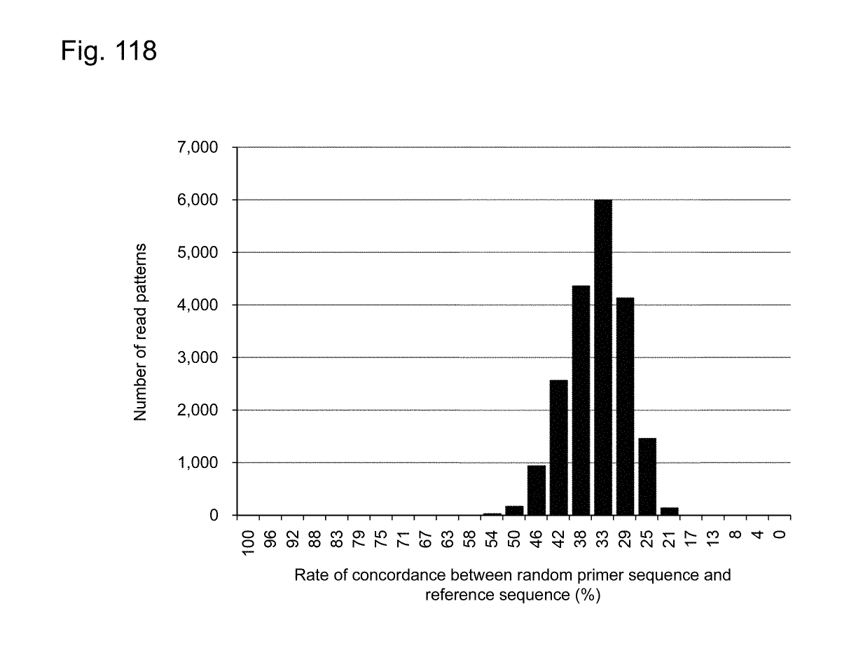

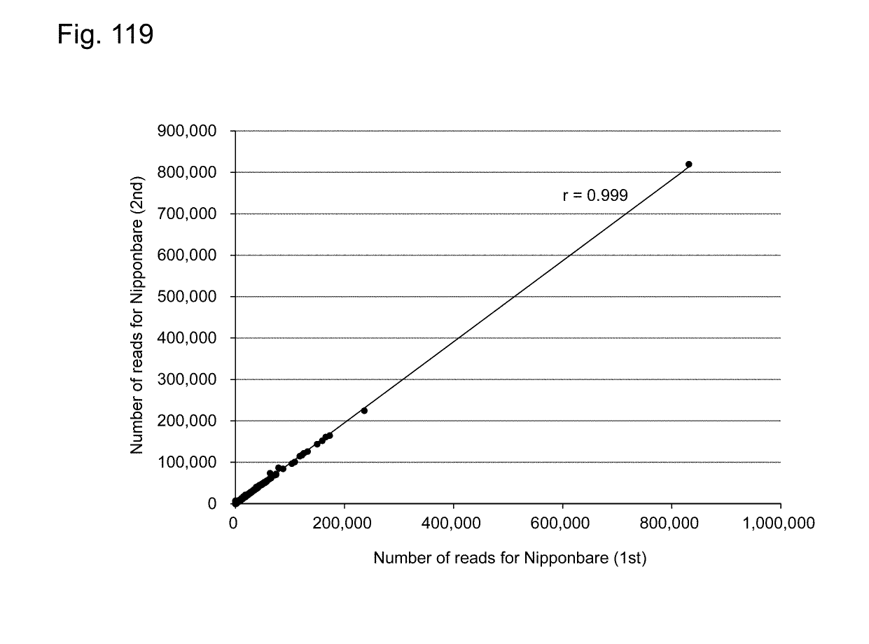

In addition, as a result of analysis of the obtained DNA library (second DNA fragments) by next-generation sequencer MiSeq, 4.0-Gbp read data and 3.8-Gbp read data were obtained. The values indicating accuracy of MiSeq data (>=Q30) were 94.0% and 95.3%. As in the case of 4.1.1, in view of the above results, the next-generation sequencer DNA library (second DNA fragments) prepared in this Example was considered to be applicable to next-generation sequencer analysis. FIG. 118 shows the results obtained by comparing random primer sequences and the reference sequence of rice variety Nipponbare in order to evaluate the degree of consistency between the random primer sequences of 19,849 read patterns obtained by MiSeq and the genome. As shown in FIG. 118, the average degree of consistency between the random primer sequences and the reference sequence of rice variety Nipponbare was 34.5%. In particular, since there was no identical read pattern between the random primer sequences and the reference sequence of rice variety Nipponbare, it was considered that any read pattern indicated that a random primer was bound to a sequence not corresponding to the random primer, and the resulting sequence was amplified. The above results were considered to correspond to the results obtained by the bioanalyzer. In order to confirm read pattern reproducibility, the number of reads of the repeated analyses were compared. FIG. 119 shows the results. As shown in FIG. 119, there was a high correlation of r=0.999 in terms of the number of reads of the repeated analyses as with the results of electrophoresis.

As described above, a DNA library (first DNA fragments) was obtained by conducting PCR using 16 types of random primers having a full length of 12 nucleotides obtained by adding an arbitrary sequence of 2 nucleotides to the 3′ end of 10 nucleotides at high concentrations, where the 10 nucleotides position at the 3′ end of a next-generation sequencer adapter (Nextera Adaptor, Illumina, Inc.) and then, PCR was conducted using a primer comprising the sequence of Nextera Adaptor. Accordingly, it was possible to conveniently produce a next-generation sequencer DNA library (second DNA fragments) comprising many fragments with favorable reproducibility.

### Example 3

### 1. Materials and Method

### 1.1 Materials

In this Example, genomic DNA was extracted from the rice variety Nipponbare using the DNeasy Plant Mini kit (QIAGEN), and the extracted genomic DNAs were purified. The purified genomic DNA was used as Nipponbare-derived genomic DNA.

### 1.2 Preparation of DNA Library

To the genomic DNA described in 1.1 above (30 ng, Nipponbare-derived genomic DNA), random primers (final concentration: 60 μM, 10-nucleotide primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 μl. PCR was carried out under thermal cycle conditions comprising 98° C. for 2 minutes and 30 cycles of 98° C. for 10 seconds, 50° C. for 15 seconds, and 72° C. for 20 seconds, followed by storage at 4° C. The DNA library obtained in this experiment was purified by the MinElute PCR Purification Kit (QIAGEN).

### 1.3 Preparation of Sequence Library

From the DNA library obtained in 1.2, a sequence library for MiSeq analysis was prepared using the KAPA Library Preparation Kit (Roche).

### 1.4 MiSeq Analysis

With the use of the MiSeq Reagent Kit V2 500 Cycle (Illumina), the sequence library for MiSeq analysis obtained in 1.3 was analyzed via 100 base paired-end sequencing.

### 1.5 Analysis of Nucleotide Sequence Information

Random primer sequence information was deleted from the read data obtained in 1.4, and nucleotide sequence information of each read was identified. Mapping of nucleotide sequence information of each read on genomic information of rice Kasalath (kasalath_genome) was conducted by bowtie2, and single nucleotide polymorphism (SNP) and insertion or deletion mutation (InDel) were identified as markers for each chromosome.

### 2. Results and Examination

Table 31 shows the results of mapping of nucleotide sequence information of the DNA library prepared using random primers based on the genomic DNA from the rice variety Nipponbare on the genomic information of rice Kasalath.

TABLE 31

| Chromosome | SNP | InDel | Total |
|---|---|---|---|
| 1 | 5,579 | 523 | 6,102 |
| 2 | 4,611 | 466 | 5,077 |
| 3 | 4,916 | 569 | 5,485 |
| 4 | 3,859 | 364 | 4,223 |
| 5 | 4,055 | 373 | 4,428 |
| 6 | 4,058 | 375 | 4,433 |
| 7 | 3,848 | 286 | 4,134 |
| 8 | 3,303 | 294 | 3,597 |
| 9 | 2,694 | 227 | 2,921 |
| 10 | 2,825 | 229 | 3,054 |
| 11 | 3,250 | 246 | 3,496 |
| 12 | 2,753 | 239 | 2,992 |
| Total | 45,751 | 4,191 | 49,942 |

As shown in Table 31, it was possible to identify 2,694 to 5,579 SNPs (3,812.6 SNPs on average, 45,751 SNPs in total) for each chromosome. As shown in Table 31, it was also possible to identify insertion/deletion (InDel) of 227 to 569 SNPs (349.3 SNPs on average, 4,191 SNPs in total) for each chromosome. The above results revealed that it is possible to identify a DNA marker as a characteristic nucleotide sequence present in the genome of a test organism by comparing nucleotide sequence information on a DNA library prepared using random primers and known nucleotide sequence information in the manner shown in this Example.

All publications, patents and patent applications cited in the present description are incorporated herein by reference in their entirety.

1. A method for producing a DNA library, comprising conducting a nucleic acid amplification reaction in a reaction solution comprising genomic DNA and a random primer at a high concentration using genomic DNA as a template to obtain DNA fragments by the nucleic acid amplification reaction.

2. The method for producing a DNA library according to claim 1, wherein the reaction solution comprises the random primer at a concentration of 4 to 200 μM.

3. The method for producing a DNA library according to claim 1, wherein the reaction solution comprises the random primer at a concentration of 4 to 100 μM.

4. The method for producing a DNA library according to claim 1, wherein the random primer comprises 9 to 30 nucleotides.

5. The method for producing a DNA library according to claim 1, wherein the DNA fragments each comprise 100 to 500 nucleotides.

6. A method for analyzing genomic DNA, comprising using a DNA library produced by the method for producing a DNA library according to claim 1 as a DNA marker.

7. The method for analyzing genomic DNA according to claim 6, which comprises determining the nucleotide sequence of the DNA library produced by the method for producing a DNA library and confirming the presence or absence of the DNA marker based on the nucleotide sequence.

8. The method for analyzing genomic DNA according to claim 7, wherein the presence or absence of the DNA marker is confirmed based on the number of reads of the nucleotide sequence of the DNA library in the step of confirming the presence or absence of the DNA marker.

9. The method for analyzing genomic DNA according to claim 7, wherein the nucleotide sequence of the DNA library is compared with known sequence information or with the nucleotide sequence of a DNA library produced using genomic DNA from a different organism or tissue, and the presence or absence of the DNA marker is confirmed based on differences in the nucleotide sequences.

10. The method for analyzing genomic DNA according to claim 6, which comprises:
a step of preparing a pair of primers for specifically amplifying the DNA marker based on the nucleotide sequence of the DNA marker;
a step of conducting a nucleic acid amplification reaction using genomic DNA extracted from a target organism as a template and the pair of primers; and a step of confirming the presence or absence of the DNA marker in the genomic DNA based on the results of the nucleic acid amplification reaction.

11. A method for producing a DNA library, comprising:
a step of conducting a nucleic acid amplification reaction in a first reaction solution comprising genomic DNA and a random primer at a high concentration to obtain first DNA fragments by the nucleic acid amplification reaction using the genomic DNA as a template; and
a step of conducting a nucleic acid amplification reaction in a second reaction solution comprising the obtained first DNA fragments and a nucleotide, as a primer, which has a 3′-end nucleotide sequence having 70% identity to at least a 5′-end nucleotide sequence of the random primer to ligate the nucleotides to the first DNA fragments, thereby obtaining second DNA fragments.

12. The method for producing a DNA library according to claim 11, wherein the first reaction solution comprises the random primer at a concentration of 4 to 200 μM.

13. The method for producing a DNA library according to claim 11, wherein the first reaction solution comprises the random primer at a concentration of 4 to 100 μM.

14. The method for producing a DNA library according to claim 11, wherein the random primer comprises 9 to 30 nucleotides.

15. The method for producing a DNA library according to claim 11, wherein the first DNA fragments each comprise 100 to 500 nucleotides.

16. The method for producing a DNA library according to claim 11, wherein the primer for amplifying the second DNA fragments comprises a region used for a nucleotide sequencing reaction, or the primer used for a nucleic acid amplification reaction using the second DNA fragments as templates or a nucleic acid amplification reaction to be conducted repeatedly comprises a region used for a nucleotide sequencing reaction.

17. A method for analyzing a DNA library, comprising a step of determining a nucleotide sequence for a second DNA fragment obtained by the method for producing a DNA library according to claim 11.

18. A method for analyzing genomic DNA, comprising using the DNA library produced by the method for producing a DNA library according to claim 11 as a DNA marker.

19. The method for analyzing genomic DNA according to claim 18, which comprises determining the nucleotide sequence of the DNA library produced by the method for producing a DNA library and confirming the presence or absence of the DNA marker based on the nucleotide sequence.

20. The method for analyzing genomic DNA according to claim 19, wherein the presence or absence of the DNA marker is confirmed based on the number of reads of the nucleotide sequence of the DNA library in the step of confirming the presence or absence of the DNA marker.

21. The method for analyzing genomic DNA according to claim 19, wherein the nucleotide sequence of the DNA library is compared with known sequence information or with the nucleotide sequence of a DNA library produced using genomic DNA from a different organism or tissue, and the presence or absence of the DNA marker is confirmed based on differences in the nucleotide sequences.

22. The method for analyzing genomic DNA according to claim 18, which comprises: a step of preparing a pair of primers for specifically amplifying the DNA marker based on the nucleotide sequence of the DNA marker; a step of conducting a nucleic acid amplification reaction using genomic DNA extracted from a target organism as a template and the pair of primers; and a step of confirming the presence or absence of the DNA marker in the genomic DNA based on the results of the nucleic acid amplification reaction.

23. A DNA library, which is produced by the method for producing a DNA library according to claim 1.
